(19) United States
(12) Patent Application Publication
(10) Pub. No.: US 2005/0196851 A1
(43) Pub. Date: Sep. 8, 2005

(54) Crystal structure of the BTK kinase domain

(75) Inventor: Fatih M. Uckun, White Bear Lake, MN (US)

Correspondence Address: MERCHANT & GOULD PC, P.O. BOX 2903, MINNEAPOLIS, MN, 55402-0903, US

(73) Assignee: Parker Hughes Institute, Roseville, MN (US)

(21) Appl. No.: 10/779,399

(22) Filed: Feb. 13, 2004

Related U.S. Application Data
(63) Continuation of application No. PCT/US02/26200, filed on Aug. 15, 2002.
(60) Provisional application No. 60/312,597, filed on Aug. 15, 2001.
(60) Provisional application No. 60/339,206, filed on Dec. 7, 2001.

(51) Int. Cl. G06F 19/00; G01N 33/48; G01N 33/50; C12N 9/12

(52) U.S. Cl. 435/194; 702/19

(57) ABSTRACT

The invention provides crystal structure of the kinase domain of BTK, as well as use of the crystal structure in the design, identification, and verification of ligands that modulate BTK activity.

This application claims priority to U.S. Provisional Application Ser. No. 60/339,206, filed Dec. 7, 2001 and U.S. Provisional Application Ser. No.60/312,597, filed Aug. 15, 2001.

### FIELD OF THE INVENTION

This invention relates to the crystal structure of the Bruton's Tyrosine Kinase (BTK) kinase domain (KD).

### BACKGROUND OF THE INVENTION

Bruton's tyrosine kinase (BTK), a member of the BTK/TEC family of cytoplasmic protein tyrosine kinases (PTKs), is intimately involved in signal transduction pathways regulating survival, activation, proliferation, and differentiation of B-lineage lymphoid cells (30, 34, 50, 61, 64). Signal transduction pathways are initiated by the binding of a variety of extracellular ligands, such as antigens, to cell surface receptors (30). Following ligation of the B-cell antigen receptor, BTK activation by the concerted actions of the PTKs LYN and SYK is required for induction of phospholipase C-γ2 mediated calcium mobilization (30). Mutations in the human BTK gene are the cause of X-linked agammaglobulinemia (XLA), a male immune deficiency disorder characterized by a lack of mature, immunoglobulin-producing, peripheral B-cells (58, 65). In mice, mutations in the btk gene have been identified as the cause of murine X-linked immune deficiency (51).

BTK is a dual-function regulator of apoptosis, in that it promotes radiation-induced apoptosis, but inhibits Fas-activated apoptosis in B-cells (59, 61). BTK promotes apoptosis when B-cells are exposed to reactive oxygen intermediates, partly by down-regulating the anti-apoptotic activity of STAT3 transcription factor. In contrast, BTK inhibits apoptosis when it associates with the death receptor Fas and impairs its interaction with FADD. The interaction between Fas and FADD is essential for the recruitment and activation of FLICE by Fas during the apoptotic signal, thereby preventing the assembly of a proapoptotic death-inducing signaling complex (DISC) after Fas-ligation.

The amino acid sequence of BTK has been determined for human and mouse and functional domains have been assigned (5, 48, 58). The N-terminal region contains a pleckstrin homology (PH) domain followed by a proline-rich TEC homology (TH) domain. The PH domain is the site of both activation (by phosphatidylinositol phosphates and G-protein βγ subunits) and inhibition (by protein kinase C) (58). The remaining portion of BTK contains SRC homology (SH) domains SH3, SH2, and a C-terminal kinase domain, also known as the SH1 domain. The SH2 domain mediates binding to trosine-phosphorylated peptide motifs on other molecules, while the SH3 domain mediates binding to proline-rich motifs on other molecules. Mutations in the SH 1 domain, SH2 domain, and the PH domain of human BTK have been found to cause maturational blocks at early stages of B-cell ontogeny leading to XLA (67). BTK-deficient mice generated by introducing PH domain or SH1 domain mutations into the BTK gene of embryonic stem cells exhibit defective B-cell development and function (25). The crystal structure of the PH domain has been determined and has contributed to a structural understanding of how point mutations of the PH domain can inactivate BTK and cause XLA (21). However, the crystal structure of the kinase domain has not been resolved.

The BTK polypeptide includes two regulatory tyrosine residues, Y223 and Y551, which participate in kinase activation (52). An SRC family PTK, such as LYN, initially activates BTK through transphosphorylation of Y551 on the presumed “activation loop” (A-loop) of the kinase domain. This activation, in turn, stimulates autophosphorylation of the Y223 residue within the SH3 domain ligand-binding site (43, 44, 46, 49, 50, 52, 68). Phosphorylation of Y223 may function to disrupt an intramolecular TH-SH3 domain interaction, allowing the BTK TH domain to bind SH3 domains of SRC family PTKs, and the BTK PH domain to bind a proline-rich region of CBL (2, 30, 33). In other members of the PTK and PSK families, the A-loop serves as a negative regulator of kinase activity by blocking access of substrates to the ATP and substrate peptide binding sites that lie in the catalytic cleft of the kinase domain (20, 23). In some members, including apo-IRK, the catalytic site is sterically blocked by amino residues in the A loop acting as a substrate peptide mimic (18, 19).

The mechanism by which phosphorylation of Y551 brings about conformational changes in the kinase domain as well as kinase activation has not been elucidated. This structural question begs the illustration of a solved crystal structure. Furthermore the dimensions, conformation and binding properties of the substrate binding sites for ATP and substrates within the kinase domain are not known. Molecular interaction with BTK inducing phosphorylation of Y551 within the kinase domain and initial activation of BTK is not understood. Similarly, there is no structural understanding of how XLA is caused by various mutations within the BTK kinase domain.

In addition to providing answers to the above questions, determining the crystal structure of the kinase domain of BTK is needed, for a host of applied purposes, such as: assays for BTK-ligand interaction and function, modeling the structure-function relationship of BTK and other molecules, diagnostic assays for mutation-induced pathologies, and rational design of agents useful in modulating BTK activity.

Modulators of BTK are useful, for example, to promote or induce apoptosis in a BTK-expressing cell by inhibiting or preventing the action of BTK, to treat a disease (pathologic condition) where BTK is implicated and inhibition of its action is desired (e.g. cancer, such as leukemia or lymphoma), and to lower the resistance of a BTK expressing cell to drug therapy by inhibiting or preventing the action of BTK.

### SUMMARY OF THE INVENTION

The X-ray crystal structure of the kinase domain of BTK (BTK-KD) has now been determined by multiple isomorphous replacement. Coordinates of the crystal structure are listed in Table 1.

The invention provides the crystal structure of the BTK-KD, as well as use of the crystal structure to model BTK activity. This use of the structure includes modeling the interaction of ligands with the BTK-KD; activation and inhibition of BTK; and the rational design of modulators of BTK activity. For example, these modulators include ligands that interact with BTK-KD and modulate BTK activities, such as the survival, activation, proliferation, and differentiation of B-lineage lymphoid cells.

### BRIEF DESCRIPTION OF FIGURES

FIG. 1 is a ribbon representation of the dimeric crystal structure of the BTK-KD.

FIG. 2 is a 2Fo-Fc electron density map (contoured at 1.0 σ) surrounding the R544, E445, and Y551 residues of the BTK-KD crystal, shown in stereo view.

FIG. 3 is a computer image showing the backbone positions of the kinase loop A-loop and helix αC of BTK-KD, phospho-LCK, and c-SRC, superimposed to illustrate their conformational differences and similarities.

FIG. 4 is a computer image showing the non-inhibitory conformation of the A-loop of BTK-KD.

FIG. 5 is a computer image showing the inhibitory conformation of the A-loop of IRK.

FIG. 6 is a model image of a proposed activation mechanism based on superimposed crystal structures of the kinase domains of BTK-KD and phospho-LCK.

FIG. 7 is a diagrammatic representation of a proposed pathway for BTK catalysis activation, whereby R544 releases E445 to interact with Y551 upon trans-phosphorylation, while E445 subsequently becomes bound to ATP.

FIG. 8 is a computer image of a backbone model of phosphorylated BTK associated with ATP, Mg⁺⁺ and the substrate Igα peptide.

FIG. 9 is a computer image of a space-filling model of phosphorylated BTK associated with ATP, Mg⁺⁺ and the substrate Igα peptide.

FIG. 10 is a backbone model of the BTK kinase domain, shown in stereo view, showing X-linked agammaglobulinaemia (XLA) related mutations of the BTK kinase domain.

### BRIEF DESCRIPTION OF SEQUENCES

SEQ ID NO:1 is an amino acid sequence of human BTK.

SEQ ID NO:2 is an amino acid sequence of murine BTK.

SEQ ID NO:3 is an amino acid sequence of the kinase domain of human BTK (I397-S659).

SEQ ID NO:4 is an amino acid sequence of the kinase domain of murine BTK (i397-S659).

SEQ ID NO:5 is a nucleotide sequence of human BTK.

SEQ ID NO:6 is a nucleotide sequence of murine BTK.

SEQ ID NO:7 is an amino acid sequence of human BTK.

Abbreviations:
(Å) Ångström
(AA or aa) Amino acids
(AMP-PNP) 5″-Adenylylimidodiphosphate, Adenosine 5′[β,γ imido]triphosphate (cAPK) cAMP-dependent kinase
(BCNU) 1,3-Bis(2-chloroethyl)-1-nitrosourea, or carmustine
(BLK) Tyrosine kinase, B-cell specific
(BMX) Tyrosine kinase, bone marrow, BTK/TEC family
(BTK) Bruton's tyrosine kinase, non-receptor tyrosine kinase
(CD28) T-cell surface antigen
(CD20) B 1, B-lymphocyte surface antigen
(DISC) Death-inducing signaling complex; i.e. FAS, FADD, and FLICE (caspase-8)
(DMSO) Dimethyl sulfoxide
(DTT) Dithiothreitol
(EBV) Epstein Barr virus
(EDTA) Ethylene diamine tetraacetic acid
(EMP) Ethylene mercury phosphate
(EMT) ITK, Tyrosine kinase, expressed in T-cells and natural killer (NK) cells
(FADD) MORT1, FAS associating protein with death domain
(FAS) Transmembrane protein, intracellular death domain, mediates apoptosis
(FFT) Fast Fourier transform
(FGFRK) FGF receptor, tyrosine kinase domain
(FLICE) FADD-like ICE, Caspase-8, aspartate-specific cysteine protease
(HCK) Tyrosine kinase, hematopoetic cell-specific
(IDEC 8) Anti-CD20 monoclonal antibody
(IRK) Insulin receptor, tyrosine kinase domain
(KD) Kinase domain
(LCK) Tyrosine kinase, lymphocyte-specific
(LYN) Tyrosine kinase, T-cell expression
(NCS) Non-crystallographic symmetry
(NIDDM) Non-insulin-dependent diabetes mellitus
(PAGE) Polyacrylamide gel electrophoresis
(PEG) Polyethylene glycol
(PHK) Phosphorylase kinase
(PMSF) Phenylmethylsulfonyl fluoride
(PH) Pleckstrin homology
(PTK) Protein tyrosine kinase
(SDS) Sodium dodecyl sulfate
(SH1) SRC homology 1 domain, or kinase domain
(SH2) SRC homology 2 domain
(SH3) SRC homology 3 domain
(c-SRC) Tyrosine kinase homologous to v-SRC gene of Rous sarcoma virus
(SYK) Tyrosine kinase, spleen and thymus, widely expressed in hematopoetic cells
(TEC) Non-receptor tyrosine kinase, widely expressed in hematopoetic cells
(TXK) Tyrosine kinase, BTK/TEC family
(TRIS) 2-Amino-2-(hydroxymethyl)-1,3-propanediol
(XLA) X-linked agammaglobulinemia

### DETAILED DESCRIPTION OF THE INVENTION

Definitions:

The following definitions are used herein, unless otherwise described:

“Crystal” means the periodic arrangement of the unit cell (filled with the motif and its symmetry generated equivalents) into a lattice.

“Complementary or complement” as used herein, means the fit or relationship between two molecules that permits interaction, including for example, space, charge, three-dimensional configuration, and the like.

“Heavy atom derivative”, as used herein, means a derivative produced by chemically modifying a crystal with a heavy atom such as Hg or Au.

“Kinase domain”, as used herein, means the catalytic domain of BTK which has a consensus sequence in common with other protein tyrosine kinases, including TEC, BMX, BLK, EMT, and TXK. The catalytic activity of BTK refers to the tyrosine phosphorylation of ligands such as the signal transduction protein, Igα. It is also termed the SH1 domain, in reference to the SRC homology domain 1.

“Ligand”, as used herein, refers to an agent that associates with the BTK kinase domain, and may be an inhibitor or stimulator of BTK activity.

“Molecular complex”, as used herein, refers to a combination of bound substrate or ligand with polypeptide, such as BTK with bound ATP, or BTK with bound Igα and ATP.

“Machine-readable data storage medium”, as used herein, means a data storage material encoded with machine-readable data, wherein a machine programmed with instructions for using such data displays a graphical three-dimensional representation of molecules or molecular complexes.

“RD” kinase, as used herein, refers to protein kinases that have an arginine residue followed by an aspartic acid residue at the positions equivalent to the R520 and D521 residues in BTK. Examples include LCK (lymphocyte specific tyrosine kinase) and c-SRC.

“Scalable”, as used herein, means the increasing or decreasing of distances between coordinates (configuration of points) by a scalar factor while keeping the angles essentially the same.

“Space group symmetry”, as used herein, means the whole symmetry of the crystal that combines the translational symmetry of a crystalline lattice with the point group symmetry. A space group is designated by a capital letter identifying the lattice type (P, A, F, etc.) followed by the point group symbol in which the rotation and reflection elements are extended to include screw axes and glide planes. Note that the point group symmetry for a given space group can be determined by removing the cell centering symbol of the space group and replacing all screw axes by similar rotation axes and replacing all glide planes with mirror planes. The point group symmetry for a space group describes the true symmetry of its reciprocal lattice.

“Unit cell”, as used herein, means the atoms in a crystal that are arranged in a regular repeating pattern, in which the smallest repeating unit is called the unit cell. The entire structure can be reconstructed from knowledge of the unit cell, which is characterized by three lengths (a, b and c) and three angles (α, β and γ). The quantities a and b are the lengths of the sides of the base of the cell and γ is the angle between these two sides. The quantity c is the height of the unit cell. The angles α and β describe the angles between the base and the vertical sides of the unit cell.

“X-ray diffraction pattern” means the pattern obtained from X-ray scattering of the periodic assembly of molecules or atoms in a crystal. X-ray crystallography is an experimental technique that exploits the fact that X-rays are diffracted by crystals. It is not an imaging technique. X-rays have the proper wavelength (in the Ångström (Å) range, approximately 10⁻⁸ cm) to be scattered by the electron cloud of an atom of comparable size. Based on the diffraction pattern obtained from X-ray scattering of the periodic assembly of molecules or atoms in the crystal, the electron density can be reconstructed. Additional phase information must be extracted either from the diffraction data or from supplementing diffraction experiments to complete the reconstruction (the phase problem in crystallography). A model is then progressively built into the experimental electron density, refined against the data to produce an accurate molecular structure.

BTK/TEC Family of Proteins

The TEC family of non-receptor tyrosine kinases is composed of six proteins designated TEC, EMT (also designated ITK or TSK), BTK (previously designated ATK, BPK or EMB), BMX, TXK (also designated RLK) and Dsrc28C. All members of the family contain SH3 and SH2 domains and, with the exception of TXK and Dsrc28C, also contain pleckstrin homology (PH) and TEC homology (TH) domains in their amino termini. TEC shares the highest degree of amino acid homology with BTK (54%). Four alternatively spliced forms of TEC are expressed broadly in cells of hematopoietic lineage and hepatocytes. The 72 kDa EMT gene product associates with CD28 and becomes activated subsequent to CD28 ligation. The 80 kDa BMX protein seems to be expressed at highest levels in the heart. TXK expression is T-cell specific, while expression of the Drosophila TEC homolog, Dsrc28C, is developmentally regulated.

BTK

Bruton's tyrosine kinase (BTK) is a member of the SRC family of protein tyrosine kinases (PTKs), and in particular, the BTK/TEC family. It is a cytoplasmic PTK of 659 amino acids (aa) [SEQ ID NO:1, human]. The numbering of amino acids for BTK, as used herein, represents the numbering of the human BTK sequence [SEQ ID NO:1].

The pleckstrin repeat homology (PH) domain (consensus, approximately 100 aa) is found in an N-terminal region at amino acid residues A2-R133, followed by a BTK motif (consensus, approximately 36 aa) at amino acid residues N135-N170. The BTK motif is a zinc-binding motif containing conserved cysteines and a histidine, found C-terminal to the PH domain. The PH/Btk motif module has been called the TEC homology (TH) region. The SH3 domain (consensus, approximately 57 aa) spans the sequence of amino acid residues A221-I269, while the SH2 homology domain (consensus, approximately 77 aa) spans the sequence of amino acid residues W281-V377. SH3 (SRC homology 3) domains are often indicative of a protein involved in signal transduction related to cytoskeletal organization, which was first described in the SRC cytoplasmic tyrosine kinase. The structure is a partly opened beta barrel. The protein kinase homology domain (KD, approximately 256 aa) spans amino acid residues K400-E658, while the ATP binding motif covers spans amino acid residues L408-V416. The human BTK protein shares amino acid sequence identity with the SRC family of protein tyrosine kinases: TEC (54% amino acid conservation), BMX (48%), ITK (50%), and TXK (53%).

The BTK protein is approximately 98% conserved across its length (659 aa) between the human amino acid sequence [SEQ ID NO:1] and murine (M. musculus) amino acid sequence [SEQ ID NO:2], while it is approximately 99% conserved over the kinase domain. The amino acid changes across the kinase domain are conservative (K432R, K625R, T643S). The amino acid sequence of the BTK kinase domain is shown in Table 3, comparing the published human [SEQ ID NO:3] and murine [SEQ ID NO:4] kinase domains.

Published BTK reference sequences include:
Human mRNA: H. sapiens GenBank (GB) access. no. NM_{—}000061 [SEQ ID NO:5]
Murine mRNA: M. musculus, GB access. no. NM_{—}013482 [SEQ ID NO:6]
Human protein: H. sapiens, GB access. no. XP_{—}037089 [SEQ ID NO:7]
Human protein: H. sapiens, GB access. no. NP_{—}000052 [SEQ ID NO:1]

Murine protein: M. musculus, GB access. no. NP_{—}038510 [SEQ ID NO:2].

TABLE 3
BTK-Kinase Domain

| | | |
|---|---|---|
| IDPKDLTFLK ELGTGQFGVV KYGKWRGQYD VAIKMIKEGS | 436 | SEQ ID NO:3 |
| ---------- ---------- ---------- ------R--- | | SEQ ID NO:4 |
| MSEDEFIEEA KVMMNLSHEK LVQLYGVCTK QRPIFIITEY | 476 | |
| ---------- ---------- ---------- ---------- | | |
| MANGCLLNYL REMRHRFQTQ QLLEMCKDVC EAMEYLESKQ | 516 | |
| ---------- ---------- ---------- ---------- | | |
| FLHRDLAARN CLVNDQGVVK VSDFGLSRYV LDDEYTSSVG | 556 | |
| ---------- ---------- ---------- ---------- | | |
| SKFPVRWSPP EVLMYSKFSS KSDIWAFGVL MWEIYSLGKM | 596 | |
| ---------- ---------- ---------- ---------- | | |
| PYERFTNSET AEHIAQGLRL YRPHLASEKV YTIMYSCWHE | 636 | |
| ---------- ---------- --------R- ---------- | | |
| KADERPTFKI LLSNILDVMD EES [hBTK-KD] | 659 | |
| ------S--- ---------- --- [mBTK-KD] | | |

Crystal Structure

The three-dimensional structure of BTK-KD was solved using X-ray crystallography to 2.1 Å resolution. Accordingly, the invention includes a BTK-KD crystal, as well as BTK-KD co-crystallized with a ligand, such as an inhibitor. The crystal has an orthorhombic space group symmetry, P2₁2₁2₁, and includes orthorhombic-shaped unit cells. Each unit cell has the approximate dimensions of: a=45±5 Å, b=104±10 Å, c=116±10 Å, α=β=γ=90°.

BTK-KD crystal structures according to the invention can be resolved using the methods described in the Examples below. BTK-KD can be crystallized in a non-complexed form or as a molecular complex with a ligand, for example an inhibitor that binds the kinase domain.

X-Ray Crystallographic Analysis

Each of the constituent amino acids of BTK-KD is defined by a set of structure coordinates as set forth in Table 1. The term “structure coordinates” refers to Cartesian coordinates derived from mathematical equations related to the patterns obtained on diffraction of a monochromatic beam of X-rays by the atoms (scattering centers) of a BTK-KD complex in crystal form. The diffraction data are used to calculate an electron density map of the repeating unit of the crystal. The electron density maps are then used to establish the positions of the individual atoms of the BTK-KD protein or protein/ligand complex.

Slight variations in structure coordinates can be generated by mathematically manipulating the BTK-KD or BTK-KD/ligand structure coordinates. For example, the structure coordinates as set forth in Table 1 could be manipulated by crystallographic permutations of the structure coordinates, fractionalization of the structure coordinates, integer additions or subtractions to sets of the structure coordinates, inversion of the structure coordinates, or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the BTK-KD would not be expected to significantly alter the nature of chemical entities Such as ligands that could associate with a kinase domain binding pocket. In this context, the phrase “associating with” refers to a condition of proximity between a ligand, or portions thereof, and a BTK molecule or portions thereof. The association may be non-covalent, wherein the juxtaposition is energetically favored by hydrogen bonding, van der Waals forces, or electrostatic interactions, or it may be covalent.

It will be readily apparent that the numbering of amino acids in other isoforms of BTK-KD may be different than that of the BTK-KD numbering disclosed herein.

Structural Features

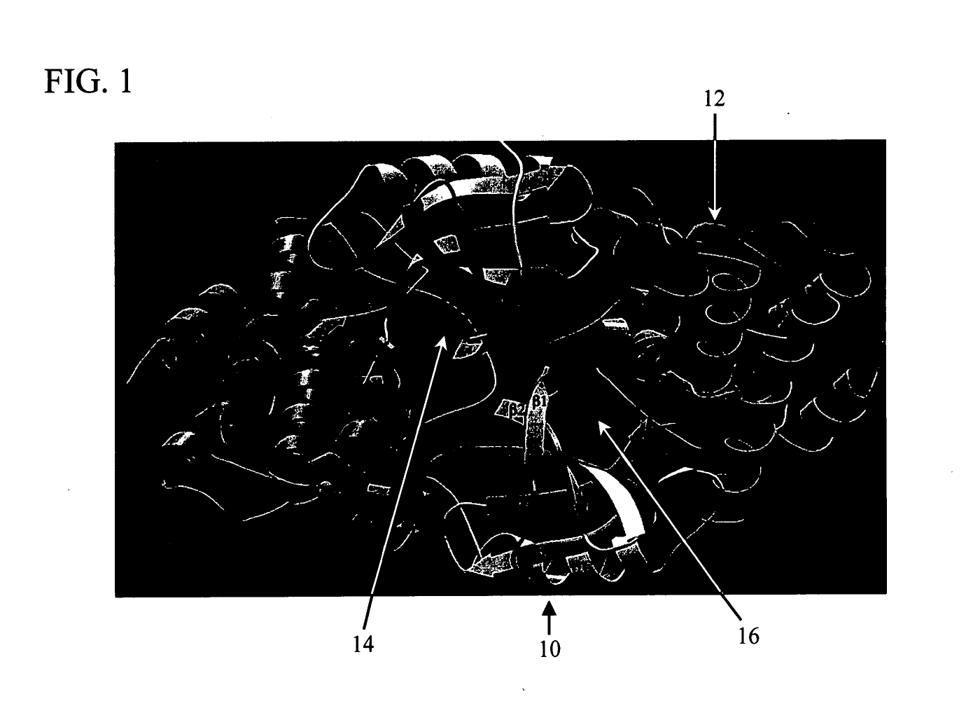

Resolution of the BTK-KD crystal structure revealed several structural features of the kinase domain as shown in FIG. 1. The N-terminal lobe 10 (amino acid residues I397-E475) contains five strands of anti-parallel β sheets (β1-β5) and one α-helix (αC). The C-terminal lobe 12 (amino acid residues N479-S659) contains a 4-helix bundle flanked by a short antiparallel β sheet and four additional helices. The N— and C-lobes are connected by a linker region 14 (amino acid residues E475-N479) and form a cleft 16 at the ATP binding site.

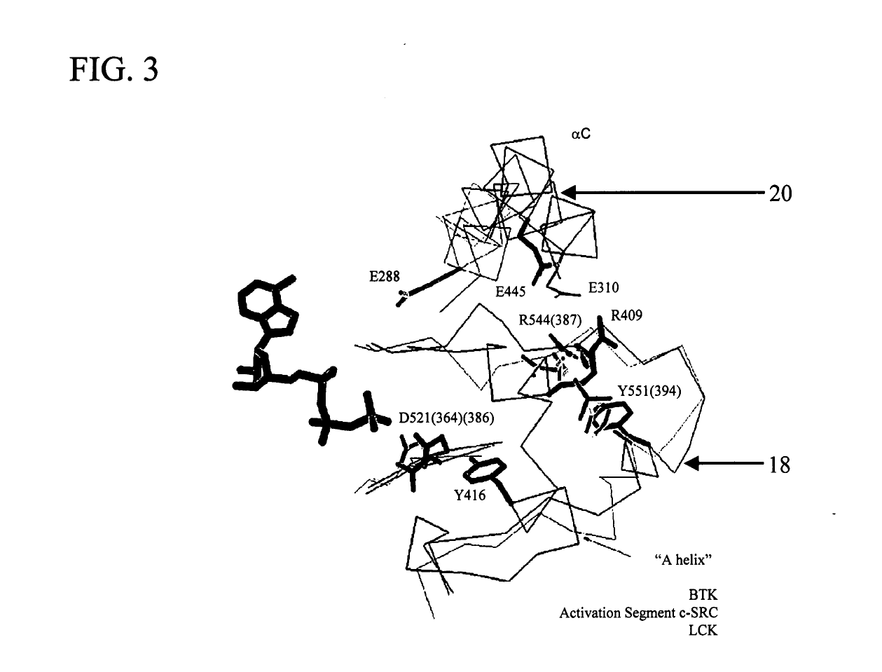

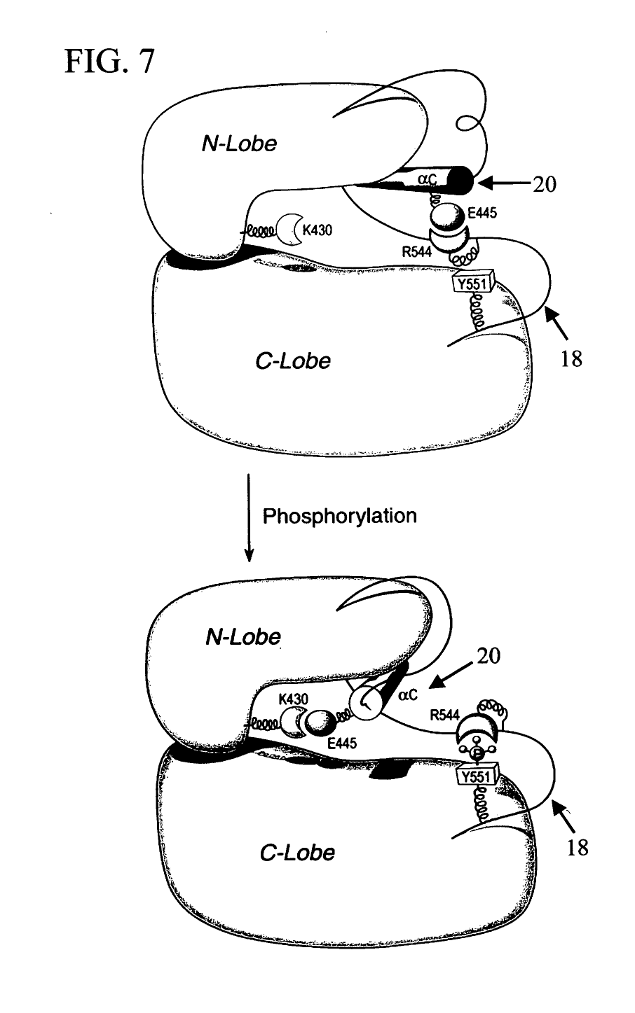

In contrast to previous predictions, the catalytic cleft of the BTK-KD is not occluded by the A-loop 18 or by any other portion of the KD. The A-loop 18 in the unphosphorylated BTK-KD structure adopts a unique non-inhibitory conformation very similar to the active state conformation of the A-loop in phosphorylated LCK-KD and hence does not limit substrate access to the active site (see FIG. 3). Due to the inactive conformation of helix αC 20, however, the enzyme is not in the active state. Based on the BTK-KD structure here disclosed, transphosphorylation of Y551 appears to trigger an exchange of hydrogen-bonded pairs from E445/R544 to E445/K430 causing subsequent relocation of helix αC 20 of the N-lobe 10, thereby inducing BTK activation (see FIG. 7).

Modeling of the BTK Kinase Domain

Peptide Substrate Binding Site

As shown in the examples, modeling of the crystal structure with a peptide substrate revealed a novel peptide substrate-binding site for BTK-KD.

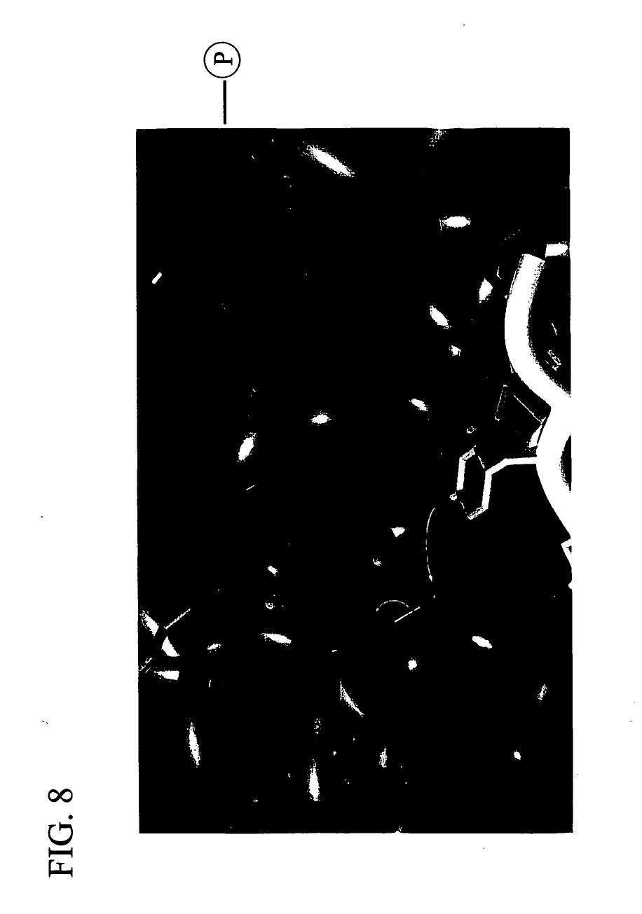

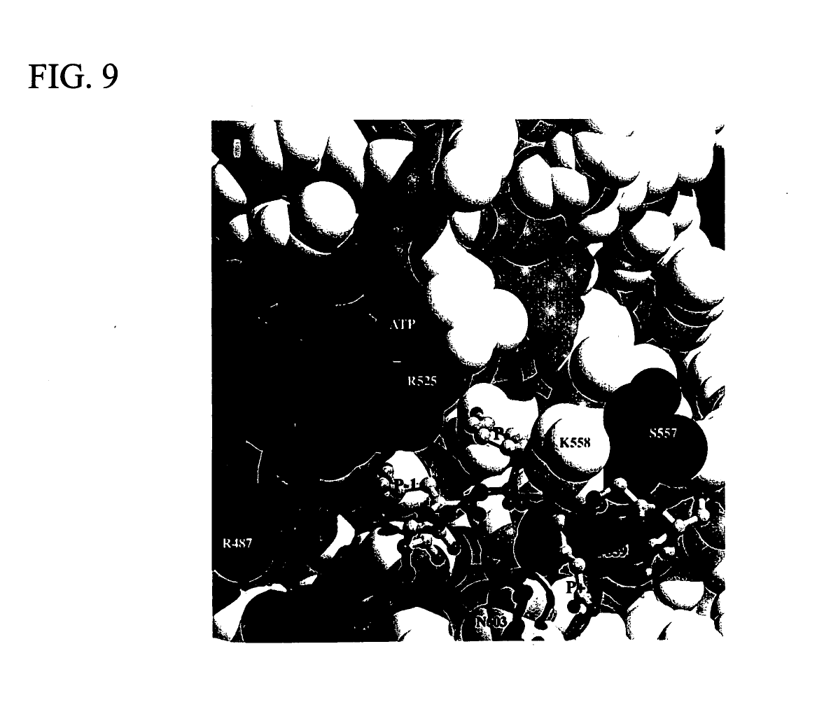

The peptide substrate-binding site of BTK-KD is a shallow groove 16 Å long on the protein surface and can accommodate the binding of a portion of the target peptide substrate between the P−2 to P+3 positions. The first half of the binding site is a circular region of 5.8 Å in radius, centered around the P−1 carbonyl group. This region can bind the residues from P−2 to P, with the side chain groups of the P tyrosine and the P−1 residue being surrounded by the BTK-KD residues (FIGS. 8 and 9). The remaining atoms of the residues from P−2 to P are mostly exposed to the solvent environment.

An “enclosed” binding site is generally highly selective for peptide substrates whereas an “open” binding site is not. The cAPK crystal structure revealed an enclosed and negatively charged binding site for the Arg (P−1) residue (39). The PHK crystal structure demonstrated an enclosed polar binding site for Gln (P−2) formed by the P+1 loop which contains a Ser, Thr and Pro (32). In contrast, the BTK complex model suggests a half-buried and spacious P−1 binding site like the substrate binding site in IRK (18) (FIG. 9). However, L483 of BTK contributes to a preference for a hydrophobic P−1 residue whereas K1085 in IRK can be associated with a preference for a negatively charged P−1 residue.

SYK has an asparagine residue corresponding to L483 and preferentially selects an aspartic acid residue for the P−1 residue over other types of residues (e.g. SYK selects a DYE motif (53)). Overall, the aliphatic portion of R525 and the side chain groups of L483, C481, R487 and M596 likely define the P−1 binding subsite. The sequence alignment of these residues with those of SRC family PTKs indicates a similar binding environment and therefore a similar recognition pattern for the P−1 position. c-SRC, BLK and LYN all preferentially select a leucine or isoleucine as the P−1 residue (54). Similarly, BTK is also likely to preferentially select a leucine or isoleucine as the P−1 residue, which is consistent with the notion that LY(223)D is a more favored BTK autophosphorylation site than EY(551)TSS (52).

The side chain group of the tyrosine targeted for phosphorylation on the substrate peptide is in contact with P560, R525, D521, γ-phosphate and possibly with the side chain groups of Q412 and K558. The enclosed binding environment is consistent with a highly discriminating binding pocket for the P tyrosine.

The Glu (P+1) residue in BTK is close to F559 and interacts with N603. Residues larger than Glu can potentially interact with residue S604. Previous crystal structures for PTK kinase domains have not shown a specific and enclosed binding site for the P+1 residue, as well as in this current BTK-KD crystal structure. Therefore the selection for the P+1 residue is unlikely to be strict. However, in light of the differences for these three residues at the P+1 position, BTK is more similar to SRC family PTKs than it is to IRK or SYK. Residues that correspond to F559, N603 and S604 of BTK are specified in parentheses: IRK (L,N,E), SYK (K,G,S), SRC (F,N,R), LYN (F,N,A), BLK (F,N,P).

Our model is consistent with the phosphoryl transfer mechanism of an in-line nucleophilic attack. Specifically, D445, K430, a Mg ion, and α and β-phosphate groups form a network of electrostatic interactions and hydrogen bonds presumably to align the ATP molecule in a conformation suitable for reaction (FIG. 8). D539 and the second Mg ion orient the γ-phosphate group for the in-line attack. The model indicates that the hydroxyl group of the P tyrosine forms a hydrogen bond with D521, consistent with the activation mechanism in which the attacking group is deprotonated by an aspartic acid residue.

### Table 2

### Amino Acids in the BTK-KD Binding Pocket

Peptide binding loop (S557-P560)

AA interacting with the P−1 residue (R525, L483, C481, R487, M596)

AA interacting with the P residue (P560, R525, D521, Q412, K558)

AA interacting with the P+1 residue (F559 and N603)

AA interacting with Mg⁺⁺ and ATP (D445, K430, and D539)

Three-Dimensional Configurations

X-ray structure coordinates define a unique configuration of points in space. Those of skill in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a scalable configuration of points can be defined by increasing or decreasing the distances between coordinates by a scalar factor, while keeping the angles essentially the same.

The present invention thus includes the scalable three-dimensional configuration of points derived from the structure coordinates of at least a portion of a BTK-kinase domain molecule or molecular complex, as well as structurally equivalent configurations, as described below. The scalable three-dimensional configuration includes points derived from structure coordinates representing the locations of a plurality of the amino acids defining the BTK-kinase domain ligand binding pocket, a BTK-KD substrate binding pocket, and the BTK-KD ATP binding site.

In one embodiment, the scalable three-dimensional configuration includes points derived from structure coordinates representing the locations of the backbone atoms of a plurality of amino acids defining the BTK-KD ligand binding pocket, a BTK-KD substrate binding pocket, and the BTK-KD ATP binding site. Alternatively, the scalable three-dimensional configuration includes points derived from structure coordinates representing the locations of the side chain and the backbone atoms (other than hydrogens) of a plurality of the amino acids defining the BTK-KD ligand binding pocket, a BTK-KD substrate binding pocket, and the BTK-KD ATP binding site, preferably the amino acids listed in Table 2.

Specific amino acids defining a BTK-KD ligand binding pocket include those amino acids of the peptide binding loop (S557-P560), those amino acids interacting with the P−1 residue (R525, L483, C481, R487, and M596), those amino acids interacting with the P residue (P560, R525, D521, Q412, and K558), those amino acids interacting with the P+1 residue (F559 and N603), and those amino acids interacting with Mg⁺⁺ and ATP (D445, K430, and D539).

Likewise, the invention also includes the scalable three-dimensional configuration of points derived from structure coordinates of molecules or molecular complexes that are structurally homologous to BTK-KD, as well as structurally equivalent configurations. Structurally homologous molecules or molecular complexes are defined below. Advantageously, structurally homologous molecules can be identified using the structure coordinates of BTK-KD according to a method of the invention.

The configurations of points in space derived from structure coordinates according to the invention can be visualized as, for example, a holographic image, a stereodiagram, a model or a computer-displayed image, and the invention thus includes such images, diagrams or models.

Structurally Equivalent Crystal Structures

Various computational analyses can be used to determine whether a molecule or a ligand binding pocket portion thereof is “structurally equivalent,” defined in terms of its three-dimensional structure, to all or part of BTK-KD or its ligand binding pockets. Such analyses may be carried out in current software applications, such as the Molecular Similarity application of QUANTA (Molecular Simulations Inc., San Diego, Calif.), Version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: (1) load the structures to be compared; (2) define the atom equivalences in these structures; (3) perform a fitting operation; and (4) analyze the results.

One structure is identified as the target (i.e., the fixed structure); all remaining structures are working structures (i.e., moving structures). Since atom equivalency within QUANTA is defined by user input, for the purpose of this invention equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or ligand binding pocket thereof, or any portion thereof, that has a root mean square deviation of conserved residue backbone atoms (N, Cα, C, O) of less than about 0.70 Å, when superimposed on the relevant backbone atoms is considered “structurally equivalent” to the reference molecule. That is to say, the crystal structures of those portions of the two molecules are substantially identical, within acceptable error. For example, structurally equivalent molecules or molecular complexes are those that are defined by the entire set of structure coordinates listed in Table 1 ± a root mean square deviation from the conserved backbone atoms of those amino acids of not more than 0.70 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of BTK-KD or a ligand binding pocket portion thereof, as defined by the structure coordinates of BTK-KD described herein.

Machine-Readable Storage Media

Transformation of the structure coordinates for all or a portion of BTK-KD or the BTK-KD/ligand complex or one of its ligand binding pockets, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium including a data storage material encoded with machine-readable data wherein a machine programmed with instructions for using said data displays a graphical three-dimensional representation of any of the molecule or molecular complexes of this invention that have been described above. In a preferred embodiment, the machine-readable data storage medium includes a data storage material encoded with machine-readable data wherein a machine programmed with instructions for using said data displays a graphical three-dimensional representation of a molecule or molecular complex including all or any parts of a BTK-KD ligand binding pocket or a BTK-KD-like ligand binding pocket, as defined above. In another preferred embodiment, the machine-readable data storage medium includes a data storage material encoded with machine readable data wherein a machine programmed with instructions for using said data displays a graphical three-dimensional representation of a molecule or molecular complex±a root mean square deviation from the atoms of said amino acids of not more than 0.05 Å.

In an alternative embodiment, the machine-readable data storage medium includes a data storage material encoded with a first set of machine readable data which includes the Fourier transform of structure coordinates, and wherein a machine programmed with instructions for using said data is combined with a second set of machine readable data including the X-ray diffraction pattern of a molecule or molecular complex to determine at least a portion of the structure coordinates corresponding to the second set of machine readable data.

For example, a system for reading a data storage medium may include a computer including a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, track balls, touch pads, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding pocket of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

Structurally Homologous Molecules, Molecular Complexes, and Crystal Structures

Structure coordinates can be used to aid in obtaining structural information about another crystallized molecule or molecular complex. The method of the invention allows determination of at least a portion of the three-dimensional structure of molecules or molecular complexes that contain one or more structural features that are similar to structural features of BTK-KD. These molecules are referred to herein as “structurally homologous” to BTK-KD. Similar structural features can include, for example, regions of amino acid identity, conserved active site or binding site motifs, and similarly arranged secondary structural elements (e.g., α helices and β sheets). Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Two amino acid sequences are compared using the BLASTP program, version 2.0.9, of the BLAST 2 search algorithm, as described by Tatusova et al. (56), and available at URL http://www.ncbi.nlm.nih.gov/BLAST/. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.” Preferably, a structurally homologous molecule is a protein that has an amino acid sequence sharing at least 65% identity with a native or recombinant amino acid sequence of BTK-KD (for example, SEQ ID NO:3). More preferably, a protein that is structurally homologous to BTK-KD includes at least one contiguous stretch of at least 50 amino acids that shares at least 80% amino acid sequence identity with the analogous portion of the native or recombinant BTK-KD (for example, SEQ ID NO:3). Methods for generating structural information about the structurally homologous molecule or molecular complex are well known and include, for example, molecular replacement techniques.

Therefore, in another embodiment this invention provides a method of utilizing molecular replacement to obtain structural information about a molecule or molecular complex whose structure is unknown including the steps of:
(a) crystallizing the molecule or molecular complex of unknown structure;
(b) generating an X-ray diffraction pattern from said crystallized molecule or molecular complex; and
(c) applying at least a portion of the structure to the X-ray diffraction pattern to generate a three-dimensional electron density map of the molecule or molecular complex whose structure is unknown.

By using molecular replacement, all or part of the structure coordinates of BTK-KD or the BTK-KD/ligand complex as provided by this invention can be used to determine the unsolved structure of a crystallized molecule or molecular complex more quickly and efficiently than attempting to determine such information ab initio.

Molecular replacement can provide an accurate estimation of the phases for an unknown structure. Phases are one factor in equations that are used to solve crystal structures, and this factor cannot be determined directly. Obtaining accurate values for the phases, by methods other than molecular replacement, can be a time-consuming process that involves iterative cycles of approximations and refinements and greatly hinders the solution of crystal structures. However, when the crystal structure of a protein containing at least a structurally homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of BTK-KD or the BTK-KD/ligand complex within the unit cell of the crystal of the unknown molecule or molecular complex. This orientation or positioning is conducted so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure. This map, in turn, can be subjected to established and well-known model building and structure refinement techniques to provide a final, accurate structure of the unknown crystallized molecule or molecular complex (31).

Structural information about a portion of any crystallized molecule or molecular complex that is sufficiently structurally homologous to a portion of BTK-KD can be resolved by this method in addition to a molecule that shares one or more structural features with BTK-KD as described above, a molecule that has similar bioactivity, such as the same catalytic activity, substrate specificity or ligand binding activity as BTK-KD, may also be sufficiently structurally homologous to BTK-KD to permit use of the structure coordinates of BTK-KD to solve its crystal structure.

In one embodiment of the invention, the method of molecular replacement is utilized to obtain structural information about a molecule or molecular complex, wherein the molecule or molecular complex includes at least one BTK-KD subunit or homolog. A “subunit” of BTK-KD is a BTK-KD molecule that has been truncated at the N-terminus or the C-terminus, or both. In the context of the present invention, a “homolog” of BTK-KD is a protein that contains one or more amino acid substitutions, deletions, additions, or rearrangements with respect to the amino acid sequence of BTK-KD (SEQ ID NO:3), but that, when folded into its native conformation, exhibits or is reasonably expected to exhibit at least a portion of the tertiary (three-dimensional) structure of BTK-KD. For example, structurally homologous molecules can contain deletions or additions of one or more contiguous or noncontiguous amino acids, such as a loop or a domain. Structurally homologous molecules also include “modified” BTK-KD molecules that have been chemically or enzymatically derivatized at one or more constituent amino acid, including side chain modifications, backbone modifications, and N— and C-terminal modifications including acetylation, hydroxylation, methylation, amidation, and the attachment of carbohydrate or lipid moieties, cofactors, and the like.

A heavy atom derivative of BTK-KD is also included as a BTK-KD homolog. The term “heavy atom derivative” refers to derivatives of BTK-KD produced by chemically modifying a crystal of BTK-KD. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the protein (3).

The structure coordinates of BTK-KD as provided by this invention are particularly useful in solving the structure of BTK-KD mutants. Mutants may be prepared, for example, by expression of BTK-KD cDNA previously altered in its coding sequence by oligonucleotide-directed mutagenesis. Mutants may also be generated by site-specific incorporation of unnatural amino acids into BTK-KD proteins using the general biosynthetic method of Noren et al. (45). In this method, the codon encoding the amino acid of interest in wild-type BTK-KD is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant BTK-KD with the site-specific incorporated unnatural amino acid.

The structure coordinates of BTK-KD are also particularly useful to solve or model the structure of crystals of BTK-KD, BTK-KD mutants, or BTK-KD homologs co-complexed with a variety of ligands. This approach enables the determination of the optimal sites for interaction between ligand entities, including candidate BTK-KD ligands and BTK-KD. Potential sites for modification within the various binding sites of the molecule can also be identified. This information provides an additional tool for determining more efficient binding interactions, for example, increased hydrophobic interactions, between BTK-KD and a ligand. For example, high-resolution X-ray diffraction data collected from crystals exposed to different types of solvent allows the determination of where each type of solvent molecule resides. Small molecules that bind tightly to those sites can then be designed and synthesized and tested for their BTK inhibition activity.

All of the complexes referred to above may be studied using well-known X-ray diffraction techniques and may be refined versus 1.5-3.5 Å resolution X-ray data to an R-factor of about 0.30 or less using computer software, such as X-PLOR (Yale University, distributed by Molecular Simulations, Inc.; see, e.g., (3) and (37)). This information may thus be used to optimize known BTK modulators, and more importantly, to design new BTK modulators.

The invention also includes the unique three-dimensional configuration defined by a set of points defined by the structure coordinates for a molecule or molecular complex structurally homologous to BTK-KD as determined using the method of the present invention, structurally equivalent configurations, and magnetic storage media including such set of structure coordinates.

Further, the invention includes structurally homologous molecules as identified using the method of the invention.

Homology Modeling

Using homology modeling, a computer model of a BTK-KD homolog can be built or refined without crystallizing the homolog. First, a preliminary model of the BTK-KD homolog is created by sequence alignment with BTK-KD, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. If the BTK-KD homolog has been crystallized, the final homology model can be used to solve the crystal structure of the homolog by molecular replacement, as described above. Next, the preliminary model is subjected to energy minimization to yield an energy-minimized model. The energy-minimized model may contain regions where stereochemistry restraints are violated, in which case such regions are remodeled to obtain a final homology model. The homology model is positioned according to the results of molecular replacement, and subjected to further refinement including molecular dynamics calculations.

Specific Modulators of BTK That Interact with BTK-KD

Specific modulators of BTK include the inhibitor LFM-A13, a leflunomide metabolite, which docks within the ATP-binding pocket of the kinase domain (62). Using the IRK ternary structure as a template, a peptide hexamer derived from an ITAM motif of Igα (NLY*EGL), a known physiologic substrate of BTK, has been modeled into the peptide binding site of BTK-KD (33). See also U.S. Pat. No. 6,294,575 (issued Sep. 25, 2001) and U.S. Pat. No. 6,303,652 (issued Oct. 16, 2001). Other inhibitors of BTK include calanolide derivatives (U.S. Pat. No. 6,306,897, issued Oct. 23, 2001) and coumarin derivatives (U.S. Pat. No. 6,294,575, issued Sep. 25, 2001).

Identification of Modulators of BTK

Potent and selective ligands that modulate BTK activity (inhibitors and stimulators) are identified using the three-dimensional homology model of the BTK kinase domain produced using the coordinates of Table 1. Using this model, ligands that interact with the kinase domain are identified, and the result of the interactions is modeled. Agents identified as candidate molecules for modulating the activity of BTK are then screened against known bioassays. For example, the ability of an agent to inhibit the anti-apoptotic effects of BTK can be measured using assays known in the art, or for example, the assays disclosed in the Examples below. Using the modeling information and the assays described, one can identify agents that possess BTK-modulating properties.

Active Site and Other Structural Features

Applicants' invention provides information about the shape and structure of the substrate binding pocket of BTK-KD in the presence of a modulator.

Binding pockets are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or any part of the binding pocket. An understanding of such associations helps lead to the design of drugs having more favorable associations with their target, and thus improved biological effects. Therefore, this information is valuable in designing potential modulators of BTK-KD ligand binding pockets, as discussed in more detail below.

The term “binding pocket,” as used herein, refers to a region of a molecule or molecular complex that, as a result of its shape, favorably associates with a ligand. Thus, a binding pocket may include or consist of features such as cavities, surfaces, or interfaces between domains. Ligands that may associate with a binding pocket include, but are not limited to, cofactors, substrates, inhibitors, agonists, and antagonists.

The amino acid constituents of a BTK-KD ligand binding pocket as defined herein are positioned in three dimensions. In one aspect, the structure coordinates defining a ligand binding pocket of BTK-KD include structure coordinates of all atoms in the constituent amino acids; in another aspect, the structure coordinates of a ligand binding pocket include structure coordinates of just the backbone atoms of the constituent atoms.

The ligand binding pocket of BTK-KD for example, includes the amino acids listed in Table 2. Alternatively, the ligand binding pocket of BTK may be defined by those amino acids whose backbone atoms are situated within about 5 Å of one or more constituent atoms of a bound substrate or ligand. In yet another alternative, the ligand binding pocket can be defined by those amino acids whose backbone atoms are situated within a sphere centered on the coordinates representing the alpha carbon atom of amino acid residue D521, the sphere having a radius of about 5-6 Å, for example 5.8 Å.

The term “BTK-KD ligand binding pocket” includes all or a portion of a molecule or molecular complex whose shape is sufficiently similar to at least a portion of a ligand binding pocket of BTK-KD as to be expected to bind related structural analogues. A structurally equivalent ligand binding pocket is defined by a root mean square deviation from the structure coordinates of the backbone atoms of the amino acids that make up ligand binding pockets in BTK-KD of at most about 0.70 Å. This calculation is described below.

Accordingly, the invention provides molecules or molecular complexes including a BTK-KD ligand binding pocket or BTK-KD ligand binding pocket, as defined by the sets of structure coordinates described above.

Rational Drug Design

Computational techniques can be used to screen, identify, select and/or design ligands capable of associating with BTK-KD or structurally homologous molecules. Knowledge of the structure coordinates for BTK-KD permits the design and/or identification of synthetic compounds and/or other molecules that have a shape complementary to the conformation of the BTK-KD binding site. In particular, computational techniques can be used to identify or design ligands, such as inhibitors, agonists and antagonists, that associate with a BTK-KD ligand binding pocket or a BTK-KD ligand binding pocket. Inhibitors may bind to or interfere with all or a portion of an active site of BTK-KD, and can be competitive, non-competitive, or uncompetitive inhibitors. Once identified and screened for biological activity, these inhibitors, agonists, and/or antagonists may be used therapeutically or prophylactically, for example, to block BTK-KD activity and thus prevent the onset and/or further progression of diseases associated with BTK activity, such as XLA, and B-cell disorders, such as leukemia. Structure-activity data for analogues of ligands that bind to or interfere with BTK-KD ligand binding pockets can also be obtained computationally.

Data stored in a machine-readable storage medium that is capable of displaying a graphical three-dimensional representation of the structure of BTK-KD or a structurally homologous molecule, as identified herein, or portions thereof may thus be advantageously used for drug discovery. The structure coordinates of the ligand are used to generate a three-dimensional image that can be computationally fit to the three-dimensional image of BTK-KD or a structurally homologous molecule. The three-dimensional molecular structure encoded by the data in the data storage medium can then be computationally evaluated for its ability to associate with ligands. When the molecular structures encoded by the data is displayed in a graphical three-dimensional representation on a computer screen, the protein structure can also be visually inspected for potential association with ligands.

One embodiment of the method of drug design involves evaluating the potential association of a known ligand with BTK-KD or a structurally homologous molecule, particularly with a BTK-KD ligand binding pocket. The method of drug design thus includes computationally evaluating the potential of a selected ligand to associate with any of the molecules or molecular complexes set forth above. This method includes the steps of: (a) employing computational means to perform a fitting operation between the selected ligand and a ligand binding pocket or a pocket nearby the ligand binding pocket of the molecule or molecular complex; and (b) analyzing the results of said fitting operation to quantify the association between the ligand and the ligand binding pocket.

In another embodiment, the method of drug design involves computer-assisted design of ligand that associate with BTK-KD, its homologs, or portions thereof. Ligands can be designed in a step-wise fashion, one fragment at a time, or may be designed as a whole or de novo.

To be a viable drug candidate, the ligand identified or designed according to the method must be capable of structurally associating with at least part of a BTK-KD ligand binding pocket, and must be able, sterically and energetically, to assume a conformation that allows it to associate with the BTK-KD ligand binding pocket. Non-covalent molecular interactions important in this association include hydrogen bonding, van der Waals interactions, hydrophobic interactions, and electrostatic interactions. Conformational considerations include the overall three-dimensional structure and orientation of the ligand in relation to the ligand binding pocket, and the spacing between various functional groups of a ligand that directly interact with the BTK-KD ligand binding pocket or homologs thereof.

Optionally, the potential binding of a ligand to a BTK-KD ligand binding pocket is analyzed using computer modeling techniques prior to the actual synthesis and testing of the ligand. If these computational experiments suggest insufficient interaction and association between it and the BTK-KD ligand binding pocket, testing of the ligand is obviated. However, if computer modeling indicates a strong interaction, the molecule may then be synthesized and tested for its ability to bind to or interfere with a BTK-KD ligand binding pocket. Binding assays to determine if a compound actually modulates with BTK activity can also be performed and are well known in the art.

Several methods can be used to screen ligands or fragments for the ability to associate with a BTK-KD ligand binding pocket. This process may begin by visual inspection of, for example, a BTK-KD ligand binding pocket on the computer screen based on the BTK-KD structure coordinates or other coordinates which define a similar shape generated from the machine-readable storage medium. Selected ligands may then be positioned in a variety of orientations, or docked, within the ligand binding pocket. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting ligands. Examples include GRID (17); MCSS (38) available from Molecular Simulations, San Diego, Calif.); AUTODOCK (13) available from Scripps Research Institute, La Jolla, Calif.); and DOCK (29) available from University of California, San Francisco, Calif.).

BTK-KD binding ligands can be designed to fit a BTK-KD binding site, optionally as defined by the binding of a known modulator. There are many ligand design methods including, without limitation, LUDI (4); available from Molecular Simulations Inc., San Diego, Calif.); LEGEND (42); available from Molecular Simulations Inc., San Diego, Calif.); LeapFrog (available from Tripos Associates, St. Louis, Mo.); and SPROUT (10); available from the University of Leeds, UK).

Once a compound has been designed or selected by the above methods, the efficiency with which that ligand may bind to or interfere with a BTK-KD ligand binding pocket may be tested and optimized by computational evaluation. For example, an effective BTK-KD ligand binding pocket ligand should preferably demonstrate a relatively small difference in energy between its bound and free states (i.e., a small deformation energy of binding). Thus, an efficient BTK-KD ligand binding pocket ligands should preferably be designed with a deformation energy of binding of not greater than about 10 kcal/mole; more preferably, not greater than 7 kcal/mole. BTK-KD ligand binding pocket ligands may interact with the ligand binding pocket in more than one conformation that is similar in overall binding energy. In those cases, the deformation energy of binding is taken to be the difference between the free energy of the ligand and the average energy of the conformations observed when the ligand binds to the protein.

A ligand designed or selected as binding to or interfering with a BTK-KD ligand binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco,); QUANTA/CHARMM (Molecular Simulations, Inc., San Diego, Calif.); Insight II/Discover (Molecular Simulations, Inc., San Diego, Calif.); DelPhi (Molecular Simulations, Inc., San Diego, Calif.); and AMSOL (Quantum Chemistry Program Exchange, Indiana University). These programs can be implemented, for instance, using a Silicon Graphics workstation, such as an Indigo2 with IMPACT graphics. Other hardware systems and software packages will be known to those skilled in the art.

Another approach encompassed by this invention is the computational screening of small molecule databases for ligands or compounds that can bind in whole, or in part, to a BTK-KD ligand binding pocket. In this screening, the quality of fit of such ligands to the binding site may be judged either by shape complementarity or by estimated interaction energy (35).

A compound that is identified or designed as a result of any of these methods can be obtained (or synthesized) and tested for its biological activity, e.g., inhibition of BTK activity.

Therapeutic Use

B-cells and B-cell precursors expressing BTK have been implicated in the pathology of a number of diseases and conditions including B-cell malignancies (e.g., acute lymphoblastic leukemia, chronic lymphocytic leukemia, non-Hodgkin's lymphoma, EBV lymphomia, and myeloma), other cancers, B-cell lymphoproliferative disorders/autoimmune diseases (e.g., lupus, Crohn's disease, and chronic or graft-versus-host disease), mast cell disorders (e.g., allergies, and anaphylactic shock), conditions that relate to improper platelet aggregation, and rejection of xenotransplants (e.g., pig to human heart transplants).

BTK inhibitors designed or identified using the crystal structure of the BTK-KD can be used to treat disorders where inhibition or prevention of a TEC family kinase is indicated. For cancer cells expressing BTK, e.g. leukemia and lymphoma cells, BTK inhibitors may kill or chemosensitize. In autoimmune disease such as Lupus or autoimmune diabetes, BTK inhibitors may halt antibody production. In graft-versus-host disease after bone marrow transplantation, these inhibitors may abrogate the B-lymphocyte mediated component of the graft rejection. Similarly designed inhibitors may be useful in organ transplantation, especially in patients with chronic rejection of organs such as liver, pancreas, and kidney. And in xenotransplantation, in which rejection is mediated by B-cells, these inhibitors may also be useful. BTK inhibitors may prevent the formation of blood clots in thromboplilia (a tendency to develop blot clots). In allergy, anaphlaxis, and asthma, the goal would be to inhibit the mast cell response.

BTK inhibitors are also useful as chemosensitizing agents, useful in combination with other chemotherapeutic drugs, in particular, drugs that induce apoptosis. Examples of other chemotherapeutic drugs that can be used in combination with chemosensitizing BTK inhibitors include topoisomerase I inhibitors (e.g., camptothesin or topotecan), topoisomerase II inhibitors (e.g., daunomycin and etoposide), alkylating agents (e.g., cyclophosphamide, melphalan and BCNU), tubulin-directed agents (e.g., taxol and vinblastine), and biological agents (e.g., antibodies such as anti CD20 antibody, IDEC 8, immunotoxins, and cytokines).

BTK stimulators designed or identified using the crystal structure of the BTK-KD can be used to treat disorders, where induction of BTK activity is indicated, for example, in the treatment of B-cell disorders or immunodeficiencies, such as XLA, and in particular to stimulate differentiation of B-cells. In patients with infectious diseases, the goal would be to enhance natural B-cell mediated immunity during the course of infection. In cancer patients, BTK stimulators may boost the immune system by increasing the ability of B-cells to produce antibodies and thus the state of alertness of the B-cell (humoral) compartment of the immune system. By boosting the B-cell immune response through stimulation of BTK, the success of vaccination may be enhanced.

The invention is illustrated by the following non-limiting Examples.

### EXAMPLES

### Example 1

### Subcloning and Expression of the Murine BTK Kinase Domain

Subcloning

The murine BTK kinase domain (BTK-KD, residues I397 to S659) was amplified from the wild-type BTK gene (GB access. no. NM_{—}013482) by PCR. The PCR product was cloned into the pCR2.1 vector (BTK-KD/pCR2.1) using the Invitrogen TA cloning procedure (Invitrogen LTI, Carlsbad, Calif.). Subsequently, the BTK-KD/pCR2.1 was completely digested with NcoI and HindIII. The fragment was purified and ligated to the pFastBac HTb donor plasmid (Invitrogen LTI), generating pFastBac HTb/BTK-KD. The vector construct was then used to produce a baculoviral stock using the Bac-to-Bac baculovirus expression system (Invitrogen LTI). The expression was checked by the anti-BTK antibody, C-20 (Santa Cruz Biotechnology, Santa Cruz, Calif.) and by anti-6-Histidine antibody.

Expression

Spodoptera frugiperda (Sf9) cells were maintained at 27° C. in Sf-900 II SFM (GibcoBRL) containing 50 units/ml penicillin and 50 μg/ml streptomycin. Cells at a density of 1-1.5×10⁶ cells/mil were infected with the recombinant BTK-KD-containing virus at a 5-fold multiplicity of infection. After 54 hours post-infection, the cells were harvested by centrifugation at 800 g for 10 minutes, washed with phosphate-buffered saline, then flash-frozen in a dry ice/ethanol bath and finally stored at −80° C.

Purification

The frozen cells were thawed in a 37° C. water bath, resuspended in 5 volumes of lysis buffer (50 mM Tris/HCl pH=8.5, 100 mM KCl, 2mM DTT and 1 mM PMSF), and then sonicated for 1 minute and centrifuged at 30,000 g for 45 minutes. The supernatant was loaded onto a Ni²⁺ column, equilibrated with a solution containing 10 mM Tris/HCl buffer, pH=8.0, 500 mM KCl and 0.5 mM DTT at a flow rate of 0.75 ml/min. The column was washed with 20-bed volumes of buffer A (20 mM Tris/HCl, pH=8.5, 500 mM KCl, 15-20 mM imidazole and 2 mM DTT), and then washed with 10-bed volumes of buffer B (20 mM Tris/HCl, pH=8.5, 1 M KCl and 2 mM DTT). Afterwards, it was washed again with 2-bed volumes of buffer A. The protein was eluted with buffer C (20 mM Tris/HCl, 100 mM KCl, 150 mM imidazole and 2 mM DTT).

Fractions that contained BTK-KD were pooled and dialyzed against a solution containing 20 mM Tris/HCl, 100 mM NaCl, 2 mM DTT and 1 mM EDTA. The protein was concentrated to 5 mg/ml, digested with rTEV protease (6 μg/mg) at 4° C. overnight to remove the poly-His tag, and concentrated to 3 ml. Concentrated protein was then loaded onto a Superdex 200 column (26×60 cm, Amersham pharmacia Biotech AB, Uppsala, Sweden), equilibrated with a solution of 20 mM Tris/HCl, pH=8.5, 50 mM NaCl and 2 mM DTT. The purified BTK-KD fraction was concentrated to 12 mg/ml for crystallization.

The purity of the BTK-KD polypeptide was confirmed by PAGE analysis with Coomassie blue staining, and by Western blot analysis probed with an anti-BMK antibody. Western blot analysis probed with an anti-phosphotyrosine antibody indicated that the purified BTK-KD was not phosphorylated. In vitro kinase assays indicated that the nonphosphorylated BTK-KD was able to be phosphorylated and to transfer the phosphate group to a GST fusion protein of Igα in a 5 minute kinase reaction.

Materials and Methods: Immunoprecipitation and Kinase Assays of Recombinant Proteins from Insect Cells

Immunoprecipitations, immune-complex protein kinase assays, and immunoblotting using the ECL chemiluminescence detection system (Amersham Life Sciences, Oakbrook, Ill.) were conducted as described previously (33, 64, 12, 60). Following electrophoresis, kinase gels were dried onto Whatman 3M filter paper and subjected to phosphoimaging on a Molecular Imager (Bio-Rad, Hercules, Calif.) as well as autoradiography on film. Similarly, all chemiluminescent BTK Western blots were subjected to three dimensional densitometric scanning using the Molecular Imager and Imaging Densitometer with Molecular Analyst/Macintosh version 2.1 software following the specifications of the manufacturer (Bio-Rad). For each compound concentration, a BTK kinase activity index was determined by comparing the ratios of the kinase activity in phosphorimager units (PIU) and density of the protein bands in densitometric scanning units (DSU) to those of the baseline sample and using the formula: Activity Index=[PIU of kinase band/DSU of BTK protein band]_{test sample}:[PIU of kinase band/DSU of BTK protein band]_{baseline control sample}. GST-Igα was sometimes used as an exogenous substrate for BTK immune-complex protein kinase assays, as described (33). Horseradish peroxidase-conjugated sheep anti-mouse, donkey anti-rabbit secondary antibodies and ECL reagents were purchased from Amersham.

Sf21 cells were infected with a baculovirus expression vector for full-length BTK, as described briefly below. Cells were harvested, lysed (10 mM Tris pH7.6, 100 mM NaCl, 1% Nonidet P-40, 10% glycerol, 50 mM NaF, 100 mM Na₃VO₄, 50 μg/ml phenylmethylsulfonyl fluoride (PMSF), 10 μg/ml aprotonin, μg/ml leupeptin), and the kinases were immunoprecipitated from the lysates, as reported (64). The antibody used for immunoprecipitation of BTK from insect cells was polyclonal rabbit anti-BTK serum (33). Kinase assays were performed following a 1 hour exposure of the immunoprecipitated tyrosine kinases to the test compounds, as described below (33, 60). The immunoprecipitates were subjected to Western blot analysis as previously described (64).

### Example 2

### Crystallization of the BTK Kinase Domain

The pure protein at a concentration of 2 mg/ml was used for dynamic light scattering studies. The data indicated the BTK-KD sample was mono-disperse immediately after purification, but became poly-disperse within 30 minutes at room temperature. Therefore, all crystallization was performed at 4° C.

Crystallization

The purified BTK-KD was crystallized at 4° C. using the hanging drop method, with 2 μl of protein and 1 μl of reservoir solution in the drop and equilibrated with 600 μl of reservoir solution containing 20% PEG-1000, 100 mM Tris/HCl, pH=8.0 and 1 mM DTT using VDP trays (Hampton Research, Laguna Niguel, Calif.). The crystals began to appear within one week, which reached a full size of 200×200×200 μm in 2 to 3 weeks.

### Example 3

### Structural Analysis of the BTK Kinase Domain Crystal

Data Collection

Crystals were analyzed using a CCD detector and high-intensity synchrotron radiation source at the Cornell High Energy Synchrotron Source (CHESS, F2 Station, Cornell University, Ithaca, N.Y.). Data collected include two native data sets (Nat 1 and Nat 2) and two heavy-atom derivative data sets (EMP and AU). Data statistics are summarized in Table 4. One crystal was soaked for 24 hours in 5 mM K₂Au(CN)₂ or (AU), and another crystal was soaked in 0.1 mM ethylene mercury phosphate (EMP). All crystals were flash-cooled and kept in liquid nitrogen with 30% PEG-1000 solution as a natural cryoprotectant. All data sets were processed using DENZO and SCALEPACK programs (40), and analyzed and scaled using the CCP4 program suite (7). The data showed that the BTK-KD crystal belongs to the P2₁2₁2₁ space group with cell dimensions a=45 Å, b=104 Å and c=116 Å.

Molecular Replacement

The native data set was analyzed for molecular replacement solution using AMoRe (7, 40). The self-rotation function calculation failed to indicate any non-crystallographic symmetry (NCS), suggesting that there is either no NCS or that the NCS axis is too close to the crystallographic axis (in fact, the 2-fold axis was later found to be close to the X axis).

The search models of polyglycine and polyalanine from crystal structures of kinase domains, including LCK, IRK, FGFRK, HCK and SRC as well as the homology BTK kinase domain model (U.S. Pat. No. 6,294,575, issued Sep. 25, 2001), revealed a clear solution in rotation function searches with a top peak 10-25% higher than the second solution, depending on the search model used in the calculation. The search model of LCK with the activation loop yielded better results, but the apo-IRK model with the activation loop produced noisy results. This correlates with the refined BTK structure defined herein, where the activation loop of the BTK-KD is similar to that of phospho-LCK, but not IRK. Translational searches using both the polyglycine/polyalanine models and the models which maintain conserved residues failed to produce a convincing solution, likely due to the existence of two molecules in the asymmetric unit and the significant difference between the search models and the target structure.

Multiple Isomorphous Replacement

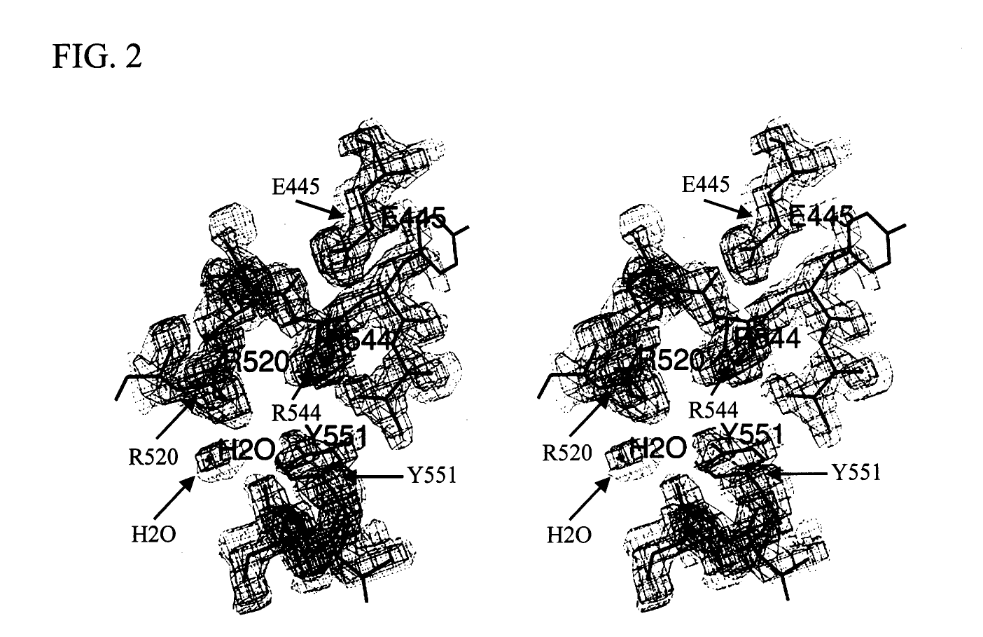

A heavy atom derivative search using a Patterson function calculation (in the RSPS program) followed by difference Fourier calculation (FFT) in the CCP4 suite (7) based on the heavy atom derivative data sets identified four Hg sites and six Au sites. These sites were used to phase all reflections at 4.0 Å and above by MLPHARE ((7), CCP4 suite). The correct handedness of the protein was determined by the sign of anomalous occupancies during the heavy atom site refinement. The map calculated from the combination of Hg and Au derivative phase information, including anomalous diffraction data of Hg atoms in the EMP derivative, was subject to several rounds of phase improvement using the DM program ((7), CCP4 suite) with a solvent flattening and histogram matching method. The improved maps demonstrated clear boundaries between the target protein and solvent region. The maps indicated two BTK-KD molecules in one asymmetric unit. Therefore, two polyglycine/polyalanine kinase search models were manually fit into the electron density and revealed how the two molecules are related by NCS. The matrix that relates the orientation of the two molecules was generated and refined into two different matrices corresponding to the N-lobe and C-lobe domains, respectively, using MAMA and 6D_IMP (26). The refined matrices were used in multi-domain averaging of the electron density map using 6D_AVE (26). The final map was considerably improved from the previous map with α-helix and β-strand structures, as well as many large amino acids clearly visible. The approximately five hundred amino acid residues (2×263 aa) and approximately 4000 atoms of the two BTK-KD molecules based on the BTK amino acid sequence were readily fit into the electron density map. The entire amino acid sequence of the BTK fragment starting from amino acid residue I397 to residue S659 were mostly traceable in the electron density map, except for a few disordered amino acid residues on the molecular surface (see FIG. 2).

Crystallographic Refinement

The kinase domain structures of the two BTK molecules were refined using simulated annealing in X-PLOR (6) and numerous structural adjustments were performed with help of CHAIN (53) and O programs (24). The refinement statistics are summarized in Table 4.

The final structure was refined at 2.1 Å resolution to an R-factor of 22% with all amino acid residues falling into favored or generously allowed regions in the Ramachandran plot (except for glycine residues), as indicated by PROCHECK (13). The average B-factor for all nonhydrogen atoms is 21 Å² and is below 20 Å² for more than half of the protein atoms. Two short regions that are disordered in the electron density map include part of the β1 strand (residues G409-Q412) and part of the activation (A)-loop (residues E550 and V555). Other regions that display a visible electron density in the original map and have high B-factors include the loop from residues V546 to D549, the loops around residue Q467, and the N-terminal end of helix αC that has B-factors mostly in the 20-30 Å² range.

Overall Architecture of the BTK Kinase Domain

A computer graphic of the BTK-KD crystal structure in its unphosphorylated state is shown in FIG. 1 as a ribbon representation. BTK-KD is packed in a dimeric form in the crystal lattice, but present mainly in monomeric form in solution (data not shown). The N-lobe and C-lobe of both BTK kinase molecules is shown. The secondary structure is labeled only on one molecule. The two BTK kinase domain molecules are related by a non-crystallographic two-fold axis that is approximately vertical in the center. This figure was prepared using MOLSCRIPT (28) and RASTER3D programs (36).

BTK-KD has a two-lobe fold reminiscent of the topology of other PTK kinase domain structures (14, 15). The secondary structure of the BTK-KD is labeled in FIG. 1 using the established nomenclature (27, 71). The N-terminal lobe (residues I397-E475) contains five strands of anti-parallel β sheets (β1-β5) and one α-helix (helix αC) (shown in FIG. 1). The C-terminal lobe (residues N479-S659) contains a 4-helix bundle (αD, αE, αF and αH) flanked by a short antiparallel β sheet (β6, β8 and β9) and four additional helices (αI, αDE, αEF, and αHI). The two helical structural segments that are too short to be labeled in the Figures, i.e. αDE and αHI, are located between alpha helices D and E and between H and I, respectively. The N— and C-lobes are connected by a linker region (residues E475-N479) and form a cleft at the ATP binding site.

TABLE 4
Statistics for Crystallographic Data Analysis
of the BTK Kinase Domain

| | Nat 1 | Nat 2 | EMP | Au |
|---|---|---|---|---|
| Crystal | | | | |
| X-ray source, wavelength | CHESS, | CHESS, | CHESS, | CHESS, |
| (Å) | λ = 1.009 | λ = 1.009 | λ = 1.009 | λ = 1.009 |
| Resolution limits (Å) | 2.1 | 3.0 | 3.0 | 3.0 |
| Unique reflections | 25,284 | 10,020 | 11,391 | 10,028 |
| Completeness (%) | 80.4 (79)* | 86.7 (81) | 96.5 (79) | 88.3 (68) |
| Rsym (%) | 5.4 (20) | 8.7 (21) | 8.0 (19) | 8.7 (23) |
| MIRAS analysis | | | | |
| Resolution range (Å) | | | 15-4 | 15-4 |
| Sites | | | 4 | 6 |
| Cullis R-factor (%) | | | 66 | 82 |
| Phasing Power | | | 1.21 | 0.73 |
| Overall FOM | | | 0.6 | |
| Refinement | | | | |
| Resolution range (Å) | 5-2.1 | | | |
| Reflections used | 20370 | | | |
| (F > 2σ) | | | | |
| Total nonhydrogen atoms | 4180 | | | |
| (protein) | | | | |
| Water molecules | 180 | | | |
| R_{cryst} (%) | 22.1 | | | |
| R_{free} (%) | 28.7 | | | |
| Rmsd from ideal | 0.006 | | | |
| bond length | | | | |
| Rmsd from ideal | 1.5 | | | |
| bond angle | | | | |
| Average B (Å²) | 19.8 | | | |
| (for 95% of protein | | | | |
| atoms with a B | | | | |
| factor < 40.0 Å²) | | | | |

Data for the outermost resolution shell are given in parentheses.
${R}_{sym} = {\sum }_{h} {\sum }_{i = 1}^{N} \vert \overline{I} ( h ) - {I}_{i} ( h ) \vert / {\sum }_{h} {\sum }_{i = 1}^{N} {I}_{i} ( h ) * 100 ,$
where Iᵢ(h) is the ith measurement of reflection h and \overline{I} (h) is
the mean value of the N equivalent reflections.
${R}_{cullis} = \sum \vert \vert {F}_{PH} \pm {F}_{P} \vert - {F}_{H ( calc )} \vert / \sum \vert {F}_{PH} + {F}_{P} \vert$
for all centric reflections.
Phasing power = rms(|F_{H}|/E), where |F_{H}| is the heavy
atom structure factor amplitude and E is the residual lack of closure.
R_{cryst} = Σ||F_{obs}| − |F_{calc}||/Σ|F_{obs}|, where
summation is over data used in the refinement.
R_{free} is the same as R_{cryst} but only include 5% of data excluded
from refinement.
*Reflections were not used in the resolution bins near or on ice rings.

### Example 7

### Structural Comparison

The BTK-KD crystal structure was compared with the crystal structures of other kinase domains including those of c-APK (PDB access code: 1ATP), LCK (3LCK), c-SRC (2SRC), HCK (1QCF), FGFRK (1AGW), IRK (1IRK for the apo structure; and 1IR3 for the ternary complex) using CHAIN and O (24, 53).

c-AMP-Dependent Protein Kinase (E.C.2.7.1.37) (cAPK) (Catalytic Subunit) Complex With The Peptide Inhibitor PKI (5-24) And MnATP (A Ternary Complex Of cAPK)

Phosphorylated Insulin Receptor Tyrosine Kinase In Complex With Peptide Substrate And Atp Analog

FIG. 3 shows the backbone positions of the A-loop and helix αC for BTK-KD, phospho-LCK, and c-SRC, superimposed to illustrate their conformational differences and similarities. An AMP-PNP molecule is present in the c-SRC crystal structure and was used to mark the location of active site. The side chains of R544 and Y551 in BTK and their equivalent residues in LCK and c-SRC on the A-loop are shown. All coordinates were superimposed in CHAIN (53). This figure was prepared using the Insight II program suite (1997, Molecular Simulations, Inc., San Diego, Calif.).

BTK-KD has a two-lobe fold with some similarity to that of other kinase domains and some differences. The rotation of the N-lobe relative to the C-lobe varies among the different KD structures and the ATP-binding cleft between the two lobes is closed when substrates or analogs are bound (18, 39). Therefore, both lobes of a KD need to adopt a mandatory closed conformation for the kinase domain to achieve a catalytically active state. The crystal structure of the unphosphorylated BTK kinase domain revealed that its N— and C-terminal lobes adopt a closed conformation very similar to the reported conformation of the lobes of the phosphorylated LCK-KD (root-mean-square deviation between the backbones of the central portion of β3 and β5<1 Å (71), and the central regions of the β3 and β5 strands are almost superimposable when the C-lobes are overlaid. See FIG. 3. However, the β1 and β2 strands, very much like the corresponding β strands of the cAK-KD, adopt a more “closed” conformation than their counterparts in LCK-KD. The rotation needed to superimpose the N-lobe of the BTK-KD onto the N-lobe the cAPK-KD is only 5.2°, whereas an 11° rotation is required to overlay the N-lobe of the LCK-KD with the N-lobe of the cAPK-KD (39). By comparison, the magnitudes of the rotation needed to open the BTK-KD and LCK-KD N-lobes to match the conformation observed in the apo-IRK structure are 22° and 17° (39), respectively.

In contrast to the similarity of the closed conformation of their N— and C-terminal lobes, the C-terminal ends of the helix αC in unphosphorylated BTK-KD versus phosphorylated LCK-KD are markedly different (see FIG. 3) where black lines indicate hydrogen bonds. The distance between the Cα position of residue D440 in BTK-KD and its counterpart in LCK is 6.7 Å. From the pivot point at residue F452, helix αC of BTK-KD needs to be rotated towards the ATP-binding cleft by 20° to be superimposed onto the helix αC of the LCK-KD. The helix αC in phosphorylated LCK-KD adopts a closed conformation consistent with a catalytically active state (71). By comparison, the helix αC of the unphosphorylated BTK-KD adopts a more open conformation than that of the LCK-KD. The conformation of helix αC of the BTK-KD is different from the open conformation of helix αC in c-SRC as well (see FIG. 3), in accordance with a unique conformation of the A-loop in BTK-KD. Significant structural differences between BTK-KD and LCK-KD were also found in helices αDE, αEF, αG and αI (which differed in location by approximately 2 Å), and in the glycine loop (β1↑β2). The β1 strand of the glycine loop is highly flexible and was observed in two distinct alternative conformations in the BTK-KD crystal structure. One conformation of the β1 strand is similar to the conformation of the corresponding β1 strand in cAPK-KD, whereas the other conformation places residues T410-N412 in a position that allows the triphosphate of ATP to bind BTK molecule A. A portion of the glycine loop is disordered in BTK molecule B that is related to molecule A by a non-crystallographic two-fold axis. The adopted conformation of the invariant PTK residues D439-G441 (DFG) in BTK-KD is consistent with the conformation of the same residues in the apo-IRK structure (19).

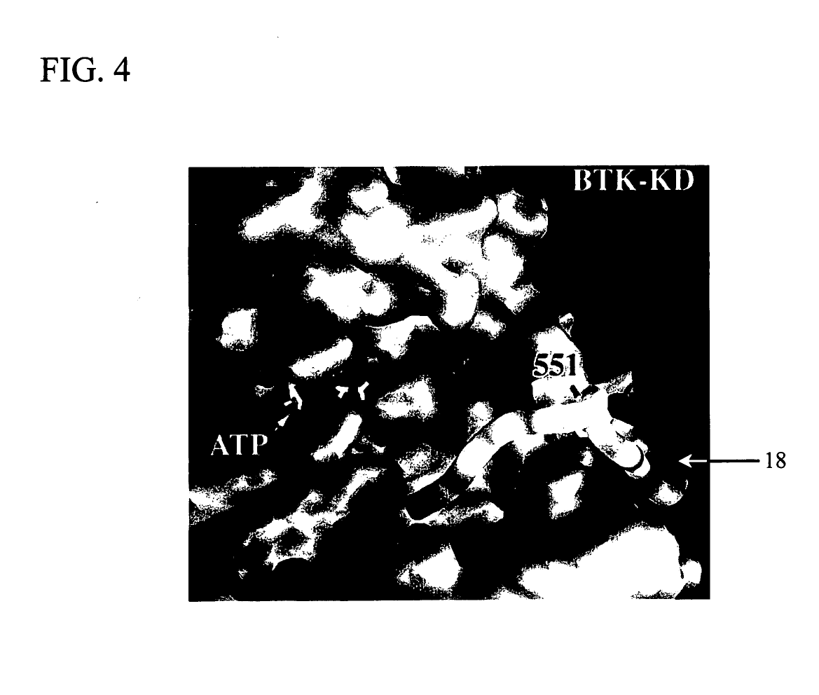

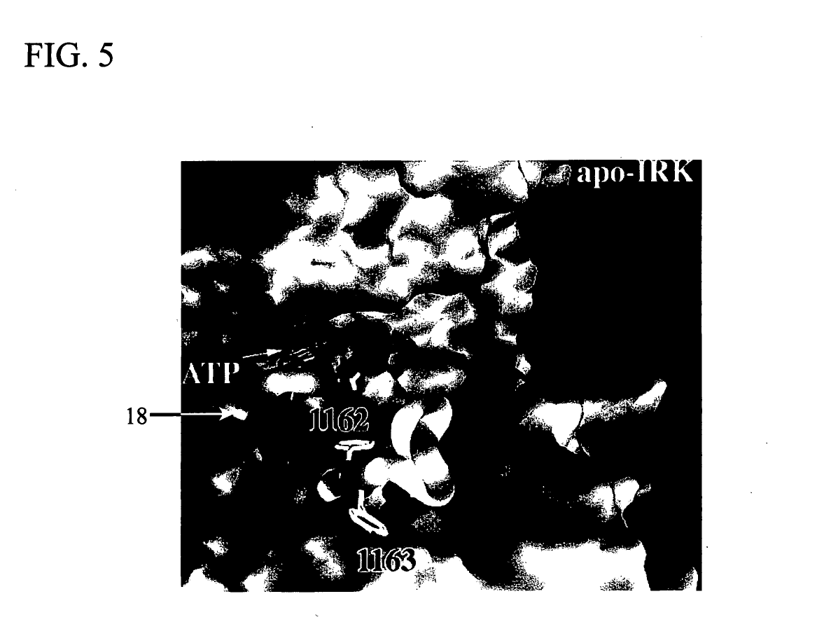

FIG. 4 and FIG. 5 are computer images showing the non-inhibitory (BTK) (FIG. 4) and inhibitory (IRK) (FIG. 5) conformations of the A-loop.

Unique Non-Inhibitory Conformation of the Activation Loop in BTK Kinase Domain

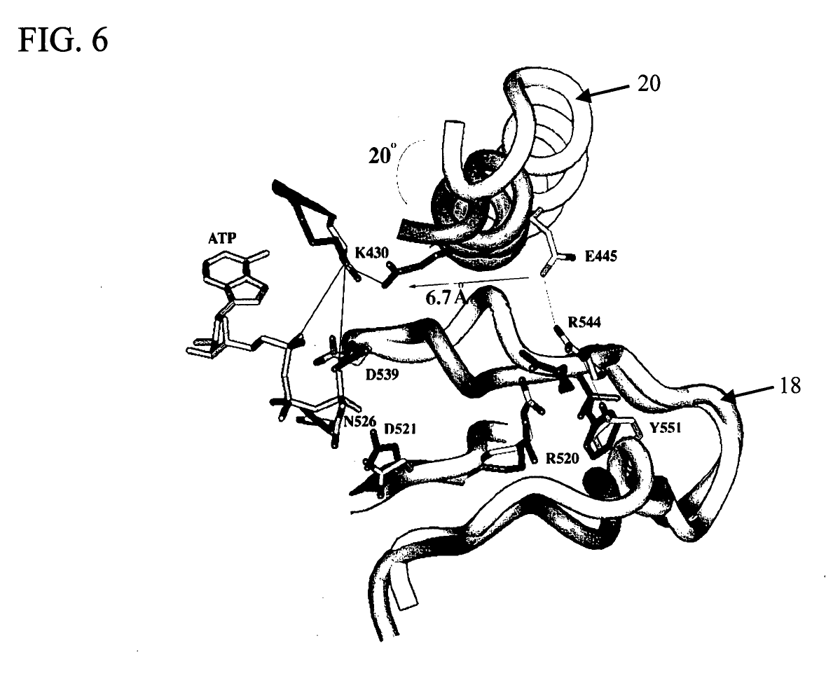

Although it is highly mobile (B-factors of 30-40 Å²), the “activation loop” (A-loop) of BTK-KD, except for residues S553-G556, is visible within the electron density map. The A-loop of the BTK-KD is structurally very similar to the A-loop in the phosphorylated LCK-KD and the peptide substrate-bound IRK-KD structures, which contain a phosphorylated tyrosine residue (see FIG. 3, IRK is not shown). R544, E445 and Y551 are well defined in electron densities, as shown in the 2Fo-Fc electron density map (FIG. 2, contoured at 1.0 σ and shown in stereo view). Based on the distance and geometry in the refined structure, the hydroxyl group of Y551 interacts with R544, S553, and a water molecule via hydrogen bonds, and this group possibly interacts electrostatically with the nearby R520 residue. The aromatic ring of Y551 has van der Waals contacts with V546 and F574. Y551 of the A-loop of the BTK-KD is not phosphorylated but it interacts with R544 as is the case for the phospho-IRK and phospho-LCK structures. The structural difference is that the unphosphorylated Y551 in the A-loop of the BTK-KD interacts with R544 via a hydroxyl group rather than through a phosphate (see FIG. 6). The crystal structure of the BTK-KD indicates that the A-loop is essentially in a closed noninhibitory conformation (FIGS. 3 and 4). Both crystal structures were first superimposed and shown separately in the same orientation with the A-loops highlighted as tubes. Neither of the activation tyrosines is phosphorylated in crystal structures. These figures were prepared with GRASP (41). Hence, only minor structural adjustments would be expected for Y551 and the surrounding residues upon Y551 phosphorylation. Similarly, only minor structural changes in the orientation of the loop residues G556-P560 would be expected upon substrate binding. A similar active conformation of the A-loop has also been observed in the crystal structures of the recently reported checkpoint (CHK1) kinase (19), and CHK1-like phosphorylase kinase (PHK) (32, 55), in which the catalytic core is constitutively active. This unique conformation of the A-loop of the unphosphorylated BTK-KD Supports a novel molecular mechanism for its initial activation (FIGS. 6 and 7).

The A-loop of kinase domains of PTKs and protein serine kinases (PSKs) usually serves as a negative regulator of kinase activity by blocking ATP binding and/or substrate peptide binding (20). Several protein kinases, including IRK, calmodulin-dependent protein kinase II, myosin light-chain kinase and protein kinase C, have a pseudosubstrate sequence within the A-loop that sterically blocks the access to the catalytic cleft by a substrate peptide (see review (23)). In the apo-IRK structure, the A-loop involving the Y551-equivalent tyrosine residue behaves as a substrate peptide mimic and sterically blocks access to the active site (FIG. 5) (18, 19). Similarly, the A-loop in the inactive c-SRC, although different from that in apo-IRK, also hinders peptide binding and blocks access to the active site (FIG. 3) (70, 72). The auto-inhibition mechanism illustrated in the apo-IRK structure was thought to be applicable to BTK as well (52). However, a close examination of the BTK-KD crystal structure reveals that Y551 is not near the active site residue D521 (FIGS. 3 and 4). Contrary to the expectations, the catalytic cleft of the BTK-KD is not occluded by the A-loop or by any other portion of the KD. The conformation of A-loop in unphosphorylated BTK-KD structure is very similar to the active conformation of phosphorylated LCK-KD and hence does not limit substrate access to the active site.

Activation of BTK by Transphosphorylation of the Activation Loop Tyrosine Residue Y551

In a typical protein kinase structure, the beginning and end of the loop from helix αC to β4 and the linker loop between the lobes (N and C) act as hinge points (22). Correspondingly, two critical structural components that are associated with the active state conformation include the closure of the two lobes and the position of helix αC relative to the N-lobe. The two lobes (N and C) in the BTK structure adopt a closed conformation. A nearly identical conformation was observed for the two BTK-KD molecules that are related by a 2-fold non-crystallographic symmetry and have different molecular packing. This suggests that the apo-BTK kinase domain favors a closed/active conformation. However, the distance of helix αC from the active site is larger in BTK-KD than it is in IRK and c-APK ternary complex structures. The distance between E445 and K430 is 10.2 Å in BTK-KD, and the corresponding distances in IRK and c-APK ternary complexes are approximately 3 Å.

K430 and E445 are two invariant residues in the structural super family of protein kinases (15). Even very conservative mutations of these residues in BTK-KD such as K430R and E445D have been associated with severe XLA (66). Mutations of the less-conserved R544 residue are also associated with severe XLA (47). The location of the E445-equivalent residue relative to the K430-equivalent residue and the location of the E445-equivalent residue relative to the ATP triphosphate serve as indicators of whether helix αC is in a favorable position for catalysis (70) (FIGS. 6 and 7). In the active KD structures such as those of the peptide-bound cAPK-KD and IRK-KD or phosphorylated LCK-KD (FIG. 6), the E445-equivalent residues are associated with the K430-equivalent residue and the ATP triphosphate either directly by hydrogen bonding, or indirectly through a medium of molecules such as water or Mg (FIG. 6). The C-terminal oxygen atoms of E445 in the BTK structure are 10.2 Å away from the K430 terminal atom. E445 is hydrogen bonded to R544, suggesting that R544 may play a regulatory role in preventing E445 from relocating to the active site and may hinder hydrogen bond formation with K430.

This unique structural arrangement strongly suggests a novel molecular mechanism of activation by Y551 phosphorylation (FIG. 7). Upon phosphorylation of Y551, R544 may be more engaged in the interaction with the phosphate group of P*Y551 and as a consequence, E445 could be released to relocate to the active site and form a hydrogen bond with K430. Helix αC in the BTK-KD structure has to be rotated by 20° from the pivot point at residue L452 (FIG. 6). Therefore, the phosphorylation of Y551 may activate BTK by disengaging R544 and E445, which consequently enables E445 to take part in the catalytic reaction, as illustrated in FIG. 7.

This unique regulatory inhibition of BTK by R544 differs from the regulatory inhibition of c-SRC by SH3, in which the salt bridge formation between E310 (equivalent to E445) and K295 (equivalent to K430) is prevented by the binding of the c-SRC SH3 domain to the proline-rich linker region between SH2 and catalytic domains (17, 69); it also differs from the mechanism of CDK2, in which the relocation of helix αC is stabilized with the help of binding with cyclin. In summary, the activation of BTK by Y551 phosphorylation likely involves an exchange of hydrogen-bonded pairs from E445/R544 to E445/K430, which can occur in concert with the phosphorylation of Y551 and subsequent relocation of helix αC.

The BTK Y551F mutant was reported to abrogate BTK autophosphorylation (33). Others observed that Y551F mutation causes a 90% reduction of LYN-mediated enhancement of both BTK tyrosine phosphorylation and kinase activity (52). A phenylalanine residue cannot engage in hydrogen-bonding interactions that link Tyr 551 to R544 and thus the conformation of the activation loop bearing this mutation may only partially resemble the internally bound inhibitory configuration. On the other hand, the Y551 F mutant loses the ability to be phosphorylated and based on our proposed mechanism cannot release E445 to the active site. In contrast, the Y551-equivalent residue of c-SRC when mutated to phenylalanine was predicted to unlock the inhibitory “A helix” and actually activate the kinase activity (70). These observations point to an interesting difference between BTK and the kinase proteins of the SRC family. The X-ray crystal structure of the BTK-KD revealed that neither Y551 nor Y545, the other tyrosine residue of the A-loop, reside in the catalytic site. This finding strongly supports the notion that BTK is activated by a trans-phosphorylation mechanism (via intermolecular interaction) at Y551 as suggested in previous experiments with LYN (43, 52, 68), rather than a cis-phosphorylation mechanism.

### Example 8

### BTK-KD Substrate Binding Model

To further elucidate the structural basis of the BTK activation, a P*Y551-PTK/ATP+Mg/peptide ternary complex model was constructed (FIGS. 8 and 9). The model was obtained by adjusting the coordinates of the BTK-KD structure by first rotating helix αC of BTK-KD to emulate the phospho-LCK structure and then adjusting the glycine loop to accommodate the substrates, based on the IRK ternary complex structure. An analysis of this new BTK-KD model and the BTK-KD crystal structure revealed no major steric clashes in the path of the 20° rotation between helix αC and the rest of the BTK-KD molecule.

FIG. 8 shows a computer image of a backbone model of phosphorylated BTK-KD associated with ATP, Mg⁺⁺ and the substrate Igα peptide. Specifically, Mg⁺⁺ ions (spheres), ATP triphosphate and peptide substrate are shown in contact via hydrogen bond or electrostatic interaction (thin lines). The in-line phosphoryl transfer mechanism for BTK is proposed (indicated by arrows). FIG. 9 shows a space-filling model of this phospho-BTK-KD/ATP/Mg⁺⁺/Igα peptide complex.

Structural Features of the Substrate Binding Site of BTK

The peptide binding loop (S557-P560) of BTK-KD adopts a β strand conformation and presumably interacts with the substrate peptide in an anti-parallel manner, as observed in the IRK, PHK and cAPK ternary structures. A peptide hexamer derived from an ITAM motif of Igα (NLY*EGL), a known physiologic substrate of BTK (46), has been modeled into the peptide binding site of BTK-KD using the IRK ternary structure as a template (shown in FIGS. 8 and 9).

The peptide substrate-binding site of BTK-KD is a shallow groove 16 Å long on the protein surface and can accommodate the binding of a portion of the target peptide substrate between the P−2 to P+3 positions. The first half of the binding site is a circular region of 5.8 Å in radius, centered around the P−1 carbonyl group. This region can bind the residues from P−2 to P, with the side chain groups of the P tyrosine and the P−1 residue being surrounded by the BTK-KD residues (FIGS. 8 and 9). The remaining atoms of the residues from P−2 to P are mostly exposed to the solvent environment.

An “enclosed” binding site is generally highly selective for peptide substrates whereas an “open” binding site is not. The cAPK crystal structure revealed an enclosed and negatively charged binding site for the Arg (P−1) residue (39). The PHK crystal structure demonstrated an enclosed polar binding site for Gln (P−2) formed by the P+1 loop which contains a Ser, Thr and Pro (32). In contrast, the BTK complex model suggests a half-buried and spacious P−1 binding site like the substrate binding site in IRK (18) (FIG. 9). However, L483 of BTK contributes to a preference for a hydrophobic P−1 residue whereas K1085 in IRK can be associated with a preference for a negatively charged P−1 residue.

SYK has an asparagine residue corresponding to L483 and preferentially selects an aspartic residue for the P−1 residue over other types of residues (e. g. SYK selects a DYE motif (54)). Overall, the aliphatic portion of R525 and the side chain groups of L483, C481, R487 and M596 likely define the P−1 binding subsite. The sequence alignment of these residues with those of SRC family PTKs indicates a similar binding environment and therefore a similar recognition pattern for the P−1 position. c-SRC, BLK and LYN all preferentially select a leucine or isoleucine as the P−1 residue (54). Similarly, BTK is also likely to preferentially select a leucine or isoleucine as the P−1 residue, which is consistent with the notion that LY(223)D is a more favored BTK autophosphorylation site than EY(551)TSS (52).

The side chain group of the tyrosine targeted for phosphorylation on the substrate peptide is in contact with P560, R525, D521, γ-phosphate and possibly with the side chain groups of Q412 and K558. The enclosed binding environment is consistent with a highly discriminating binding pocket for the P tyrosine.

The Glu (P+1) residue in BTK is close to F559 and interacts with N603. Residues larger than Glu can potentially interact with residue S604. Previous crystal structures for PTK kinase domains have not shown a specific and enclosed binding site for the P+1 residue, as well as in this current BTK-KD crystal structure. Therefore the selection for the P+1 residue is unlikely to be strict. However, in light of the differences for these three residues at the P+1 position, BTK is more similar to SRC family PTKs than it is to IRK or SYK. Residues that correspond to F559, N603 and S604 of BTK are specified in parentheses: IRK (L,N,E), SYK (K,G,S), SRC (F,N,R), LYN (F,N,A), BLK (F,N,P).

Our model is consistent with the phosphoryl transfer mechanism of an in-line nucleophilic attack. Specifically, D445, K430, a Mg ion, and α- and β-phosphate groups form a network of electrostatic interactions and hydrogen bonds presumably to align the ATP molecule in a conformation suitable for reaction (FIG. 8). D539 and the second Mg ion orient the γ-phosphate group for the in-line attack. The model indicates that the hydroxyl group of the P tyrosine forms a hydrogen bond with D521, consistent with the activation mechanism in which the attacking group is deprotonated by an aspartic acid residue.

### Example 9

### Design of Modulators of BTK

To design specific modulators of BTK, the crystal structure coordinates for the kinase domain of BTK are used to model, predict, and identify specific ligands for modulating (inhibiting or stimulating) BTK activity.

Kᵢ values that quantitate predicted binding interactions between the inhibitor and residues in the kinase domain of BTK are estimated, for example as described in Mahajan, et al. 1999 (34). Each ligand is individually modeled into the catalytic site of the BTK kinase domain using an advanced docking procedure (34, U.S. Pat. No. 6,294,575, issued Sep. 25, 2001 and U.S. Pat. No. 6,303,652, issued Oct. 16, 2001). The various docked positions of each ligand are qualitatively evaluated using a scoring procedure and consequently compared with the IC₅₀ values of the ligands in cell-free BTK inhibition assays. The interaction scores, calculated Kᵢ values, and measured IC₅₀ values for each ligand complexed with BTK is evaluated.

Apoptosis Assays

BTK inhibition assays include assays of cellular apoptosis induced by BTK. To induce apoptosis, cells were treated with an agonistic anti-Fas/APO-1 antibody (Biosource International, Camarillo, Calif., lot. 04/1295) at 0.1 μg/ml, 0.5 μg/ml, or 1.0 μg/ml final concentrations. MC540 binding (as an early marker of apoptosis) and PI permeability (as a marker of advanced stage apoptosis) were simultaneously measured in DT-40 cells 24 hours after exposure to anti-Fas antibody as previously described (59). Whole cells were analyzed with a FACStar Plus flow cytometer (Becton Dickinson, San Jose, Calif.). All analyses were done using 488 nm excitation from an argon laser. MC540 and PI emissions were split with a 600 nm short pass dichroic mirror and a 575 nm and pass filter was placed in front of one photomultiplier tube to measure MC540 emission and a 635 nm band pass filter was used for PI emission.

To detect apoptotic fragmentation of DNA, DT-40, NALM-6-UM 1, and RAMOS-1 cells were harvested 24 hours after exposure to anti-Fas. DNA was prepared from Triton-X-100 lysates for analysis of fragmentation (59, 60). Cells were lysed in hypotonic 10 mmol/L Tris-HCl (pH 7.4), 1 mmol/L EDTA, 0.2% Triton-X-100 detergent; and subsequently centrifuged at 11,000×g. To detect apoptosis-associated DNA fragmentation, supernatants were electrophoresed on a 1.2% agarose gel, and the DNA fragments were visualized by ultraviolet light after staining with ethidium bromide.

### Example 9

### Mapping XLA Mutations

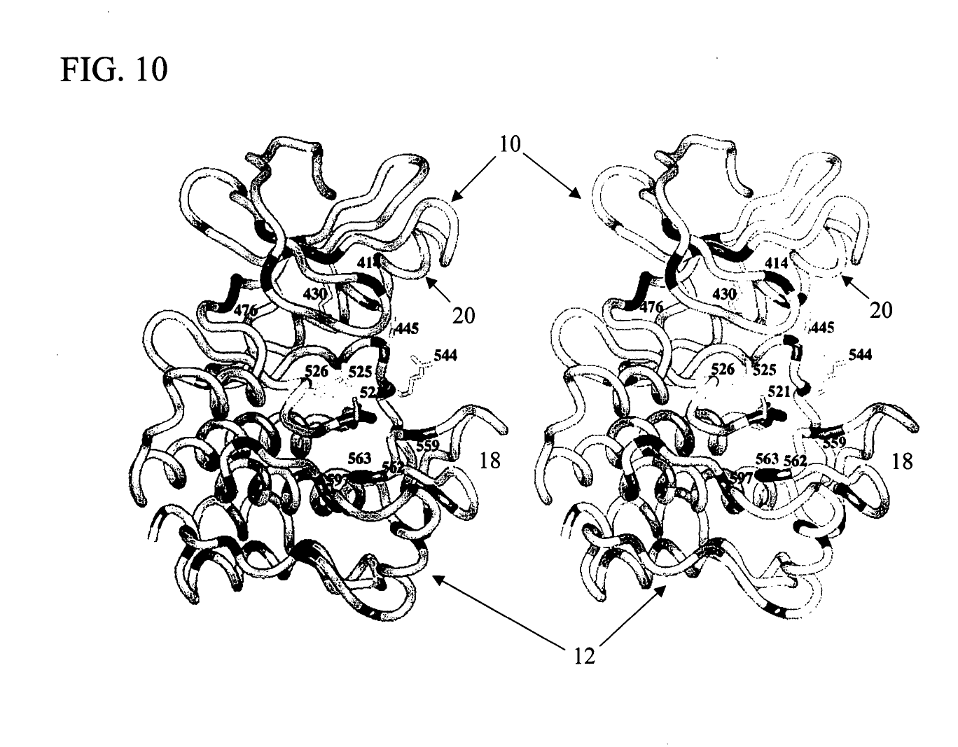

Although a homology model of BTK-KD is useful for providing the structural basis of XLA-causing mutations, a BTK-KD crystal structure provides a more accurate interpretation, especially considering that the two-lobe conformation among kinases and the geometry of active site could vary significantly. Most of known XLA-causing missense mutations are listed in Table 5, together with structural consequences of these mutations. Many of the XLA mutations map to the sites known to be important for the mechanism of BTK-KD activation described above (see FIG. 10). FIG. 10 maps X-linked agammaglobulinaemia (XLA) related mutations on the crystal structure of the BTK kinase domain, shown in stereo view. Several XLA mutations involve residues that are highly conserved and may be part of the catalysis machinery. Some XLA mutations involve other important active site residues. Many XLA mutations involve the residues, which stabilize the hydrophobic core structure of the C-lobe domain. Other XLA mutations involve the residues of the lobe linker region and the peptide substrate-binding region. This figure was prepared with the Insight II program suite (1997, Molecular Simulations, Inc., San Diego, Calif.).

TABLE 5
XLA associated BTK missense mutations

| BTK mutation | | | |
|---|---|---|---|
| associated with | | | |
| XLA | | | |
| | Amino | Structural Position and Function of Involved | Structural |
| Residue # | acid | Residue | Consequences |
| 408 | L→P | maintain the beta conformation of the α1 strand | A, indirectly |
| 414 | G→R | highly conserved residue, backbone forms a | A |
| | | hydrogen bond with the β phosphate of ATP | |
| 418 | Y→H | maintain the local hydrophobic structural | A, indirectly |
| | | stability near the domain hinge region | |
| 429 | I→N | in a hydrophobic cluster and maintain the natural | B |
| | | curvature of the N-lobe β sheet | |
| 430 | K→E | invariant residue of PTK family, align ATP via | C |
| | K→R | interaction with the α phosphate | |
| 445 | E→D | invariant residue of PTK family, align ATP via | C |
| | | interaction with the β phosphate | |
| 462 | G→D | on the β4 strand and the interface with β-sheet | E |
| | G→V | and helix C | |
| 476 | Y→D | close to adenine base of ATP | D |
| 477 | M→R | close to adenine base of ATP | D |
| 502 | C→F | on helix E, interact with F583 and M587 in a | B |
| | C→W | compact hydrophobic cluster | |
| 504 | D→V | ion pair with K456, near the hinge region of | D |
| | | helix C | |
| 506 | C→R | on helix E, interact with F583 (helix F) and the | B |
| | C→Y | carbonyl groups of 644 and 502 | |
| 508 | A→D | on helix E, near the hinge region of helix C | D |
| 509 | M→I | interact with I522 and V537 in a compact | B |
| | M→V | hydrophobic cluster | |
| 512 | L→P | near the side chains of L457, L452 and F517, | B |
| | L→Q | M509 in a compact hydrophobic cluster | |
| 518 | L→R | close to V546, F574 and Y551 in a hydrophobic | B + E |
| | | cluster | |
| 520 | R→Q | conserved in all “RD” kinases, interacts with the | B + E |
| | | nearby activation Y551, stabilizing the phosphotyrosine | |
| 521 | D→G | invariant residue of PTK family, activation of | C |
| | D→H | the nucleophilic attacking hydroxyl group of the | |
| | D→N | target residue (tyrosine in BTK) by | |
| | | deprotonation | |
| 525 | R→P | alignment and stabilization of the γ-phosphate | A |
| | R→Q | during phosphoryl transfer process | |
| 526 | N→K | invariant residue of PTK family, coupled with | A |
| | | D539 to align the γ-phosphate of ATP | |
| 535 | V→F | interact with L665 | D |
| 542 | L→P | near the highly conserved DFG motif which | D |
| | | adopts two different conformations during | |
| | | catalytic process | |
| 544 | R→K | form a hydrogen bond with E445 in a | see text |
| | R→G | nonproductive conformation | |
| 559 | F→S | in the P + 1 substrate binding pocket | F, P + 1 |
| 562 | R→P | on a helix turn starting from P560 to S564 in the | F |
| | R→W | protein substrate binding site | |
| 563 | W→L | hydrophobic interaction with A523 and is part of | F |
| | | protein substrate binding pocket | |
| 567 | E→K | ion pair with R641 and thus contribute to the | B |
| | | interaction between helix EF and helix H or I. | |
| 569 | L→P | on helix EF | B |
| 578 | S→Y | on helix F, hydrogen bonded with the 575 amide | B |
| | | near the activation loop | |
| 581 | W→R | on helix F, hydrophobic interaction with W634 | B |
| | | in a compact hydrophobic cluster | |
| 582 | A→V | near the 522 carbonyl group and D521 | A + E′ |
| 583 | F→S | see 506 | B |
| 587 | M→L | on helix F, see 502 | B |
| 589 | E→D | on helix F, see 506 | B |
| | E→G | | |
| 592 | S→P | forms a hydrogen bond with the 595 amide | B |
| | S→Y | | |
| 594 | G→E | near R487 which forms part of the P − 1 binding | F, P − 1 |
| | G→R | pocket | |
| 597 | P→T | buried near M596 which forms part of the P − 1 | F, P − 1 |
| | | binding pocket | |
| 598 | Y→C | interact with F601, L616 and T606 in a compact | B |
| | | hydrophobic cluster | |
| 606 | T→P | on the inward face of helix G, see 598 | B |
| 607 | A→D | on helix G, mostly exposed | X |
| 613 | G→D | near R615 | X |
| 616 | L→F | on helix G, interact with W634, Y598 and W588 | B |
| | | in a compact hydrophobic cluster | |
| 619 | P→A | on the inward face of helix G and close to | B |
| | P→/S/T | W588, S592 and A622 | |
| 622 | A→P | interact with 619, see 619 | B |
| 626 | V→G | form a hydrophobic cluster on the inward face of | B |
| | | helix H and close to Y591 | |
| 630 | M→K | on the inward face of helix H, interacts with | B |
| | M→T | W634 and W588 | |
| 633 | C→Y | on the inward face of helix H in a compact | B |
| | | hydrophobic cluster | |
| 641 | R→C | on helix H and pair with E567, see 567 | B |
| | R→H | | |
| 644 | F→L | on the inward face of helix I and in a | B |
| | F→S | hydrophobic cluster | |
| 647 | L→R | on the inward face of helix I and close to C633 | B |
| | | and F583 | |
| 652 | L→P | exposed | X |
| * Missense mutations were obtained from BTKbase, a mutation database for X-linked agammaglobulinemia (http://protein.uta.fi/BTKbase/btkpub.html); A: affect the ATP binding; B: affect local structural stability; C: disrupt interaction with E445 and affect the catalytic reaction; D: interfere with domain “breathing”; E: | | | |
| # interfere with the activation process; E′: affect helix C and activation; F: change sequence selection preference; X: no foreseeable effect on kinase activity: P − 1: affect the P − 1 binding pocket; P + 1: affect the P + 1 binding pocket. | | | |

Observations involving R544K/G and K430R mutations are intriguing. An arginine residue is different from a lysine residue in side chain length as well as hydrogen bonding capability and interactions with phosphate and/or glutamic acid residues. These differences may adversely affect the binding of ATP to the catalytic domain of BTK R544K/G. The R544G mutant may not be able to stabilize the phosphorylated Y551 and may consequently destabilize the A-loop, the position of which is sensitive for correct alignment of peptide substrate binding. On the other hand, R544G would be expected to unlock E445 and would probably trigger part of the activation process as discussed earlier in this paper. The effect of R544K mutation on BTK kinase activity is less certain because the R544-equivalent residues vary in different PTKs. The BTK structure suggests that R544K is unlikely to abolish the kinase activity entirely.

R520 is not entirely conserved in the protein kinase super family but is present in all “RD” kinases that require activation by phosphorylation (23). Notably, the R520-equivalent residue in IRK was found to be mutated (R1131N) in patients with non-insulin-dependent diabetes mellitus (NIDDM) (1). The side chain of R520 is close to Y551 in the BTK-KD crystal structure. A survey of the equivalent residue in the “RD” kinase structures revealed that the R520-equivalent residue is close to a phosphate or a carboxylate group and apparently plays a role in stabilizing the phospho-tyrosine/Ser/Thr. The hot-spot mutation R520Q certainly changes the interaction pattern with P*Y551 and a glutamine residue is much less likely to be associated with a phosphate group than an arginine residue (8). Thus, the R520Q mutant probably would have a destabilized activation loop.

XLA-associated BTK mutations involving the N-lobe of the kinase domain are less frequent than those involving the C-lobe (FIG. 10 and Table 5). G414, L408, Y418, and I429 were identified as “mutation hot spots” in XLA patients. Notably, residue G414 which is highly conserved as a glycine (or less likely, small residues like alanine) is located at the beginning of the β2 strand and is right on top of the triphosphate group of ATP where its backbone forms a hydrogen bond with the oxygen atom of the γ-phosphate. At this position, a large side chain substitution such as the G414R mutation would dramatically limit the loop flexibility that may be required to accommodate ATP and subsequently release ADP. With the arginine substitution, neither the hydrogen bonding nor the ATP binding conformation would be optimal.

In the crystal structure of the IRK ternary complex, the β1-β2 loop adopts a common β turn type III, in which the i+3 position, which corresponds to G414 in BTK, is predominantly occupied by a flexible glycine residue G1008 which allows the defined conformation and is presumably necessary for correctly placing the ATP phosphate in the BTK catalytic site. A G1008V mutation in IRK has been found in patients with NIDDM (1). A more dramatic G414R substitution in BTK is likely to alter the conformation to become incompatible with the correct alignment of ATP for catalysis.

Modeling studies indicate that the XLA-associated mutations W563L, P597T, F559S and R562 can be directly or indirectly involved in the peptide substrate binding. W563 is situated between P597 and A523, the latter of which is near the center of the active site. W563L mutation may alter the conformation of the peptide subsite and has been identified in patients with XLA. P597 is relatively distant from the central region of the active site but the side chain of P597 is totally buried behind the nearby residues including M596, which forms part of the P-1 binding pocket (see FIG. 9). The nearest atom pairs between W563 and P597 or A523 are 3.7 Å away. The three residues are packed against each other as the core part of the substrate peptide-binding site. Clearly, the P597T mutation would impair substrate binding. By comparison, the F559S mutation may change the selection preference of the binding region for the P+1 position (see FIG. 9). The side chain of R562 forms a network of hydrogen bonds with N603, which is a part of the P+1 binding pocket, and T606, which is connected to the main chain carbonyl group. The R562P mutation can be expected to alter the helical turn due to the rigid proline residue and thereby change the local conformation including that of the important PTK invariant P-site residue P560. The R1174N mutant (corresponds to a mutation of R562) in IRK has been identified in NIDDM patients.

Remarkable insights were gained by mapping the XLA-associated missense mutations of the C-lobe onto the BTK-KD crystal structure. Most of these mutations on the C-lobe are concentrated on the inward face of helices and loops (see residues in FIG. 10). According to our analysis, the majority of the side chains are at least half-buried, suggesting that these mutations may destabilize the C-lobe of the kinase domain by altering its interactions with neighboring residues (see also Table 5). The mutations include S578Y, W581R, A582V, F583S, M587L, E589D/G and S592P on both sides of helix αF that is flanked by other helices. The inward sides of helices αE, αC, and αI, appear to be most susceptible to XLA-causing mutations (see FIG. 10). The inward face of loops located between the helices of the C-lobe, although lying exposed on the protein surface, are also susceptible to mutation. L652P is an exception in that its side chain occurs on the protein surface. Therefore, the functional implications of this mutation should be interpreted with caution.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

References

1. Accili, D., Cama, A., Barbetti, F., Kadowaki, H., Kadowaki, T., and Taylor, S. 1. 1992. J. Endocrinol. Invest. 15:857-64.

2. Afar, D., Park, H., Howell, B., Rawlings, D., Cooper, J., and Witte, 0.1996. Mol. Cell. Biol. 16:3465-71.

3. Blundell, et al. 1976. Protein Crystallography, Academic Press, San Diego, Calif.

4. Bohm, 1992. J. Comput. Aided Molec. Design 6:61-78.

5. Bork, P., Schultz, J., and Ponting, C. 1997. Trends Biochem. Sci. 22:296-8.

6. Brünger, A. 1992. XPLOR version 3.1, Yale University Press, New Haven, Conn.

7. Collaborative Computational Project Number 4. 1994. Acta Crystallogr. D50:760-76.

8. Copley, R., and Barton, G. 1994. J Mol. Biol. 242:321-9.

9. Eisen, et al. 1994. Proteins 19:199-221.

10. Gillet, et al. 1993. J. Comput. Aided Mol. Design 7:127-53.

11. Goodford. 1985. J. Med. Chem. 28:849-57; available from Oxford University, Oxford, UK.

12. Goodman, et al. 1998. J Biol. Chem. 273:17742-48.

13. Goodsell, et al. 1990. Proteins 8:195-202.

14. Hanks, S., and Hunter, T. 1995. FASEB J. 9:576-96.

15. Hanks, S., and Quinn, A. M. 1991. Methods in Enzymology 200:38-62.

16. Hsu, et al. 1996. Biochiemistry 35:13871-77.

17. Hubbard, S. 1999. Nature Struct. Biol. 6:711-4.

18. S. Hubbard, S. 1997. EMBO J. 16:5572-81.

19. Hubbard, S., Wei, L., Ellis, L., and Hendrickson, W. 1994. Nature 372:746-54.

20. Hunter, T. 1998. Harvey Lect 94:81-119.

21. Hyvonen, M., and Saraste, M. 1997. EMBO J. 16:3396-404.

22. Johnson, L., Lowe, E., Noble, M., and Owen, D. 1998. FEBS Letters 430:1-11.

23. Johnson, L., Noble, M., and Owen, D. 1996. Cell 85:149-58.

24. Jones, T., Bergdoll, M., and Kjeldgaard, M. 1989. Crystallographic Computing and Modeling Methods in Molecular Design (Bugg, C., and Ealick, S., Eds.), Springer, New York, N.Y.

25. Kerner, J., Appleby, M., Mohr, R., Chien, S., Rawlings, D., Maliszewski, C., Witte, O., and Perlmutter, R. 1995. Immunity 3:301-12.

26. Kleywegt, et al. 1994. Halloween . . . Masks and Bones, in First Map to Final Model (Bailey, et al., Eds.), SERC Daresbury Laboratory, Warrington, UK.

27. Knighton, D., Zheng, J., Ten Eyck, L., Ashford, V., Xuong, N., Taylor, S., and Sowadski, J. 1991. Science 253:407-14.

28. Kraulis, P. 1991. J. Appl. Cryst. 24:946-50.

29. Kuntz, et al. 1982. J. Mol. Biol. 161:269-88.

30. Kulosaki, T., and Kurosaki, M. 1997. J. Biol. Chem. 272:15595-8.

31. Lattman. 1985. Methods in Enzymology 115:55-77.

32. Lowe, E. D., Noble, M. E., Skamnaki, V. T., Oikonomakos, N. G., Owen, D. J., and Johnson, L. 1997. EMBO J. 16:6646-58.

33. Mahajan, S., Fargnoli, J., Burkhardt, A. L., Kut, S. A., Saouaf, S. J., and Bolen, J. B., 1995. Mol. Cell. Biol. 15:5304-11.

34. Mahajan, S., Ghosh, S., Sudbeck, E., Zheng, Y., Downs, S., Hupke, M., and Uckun, F. 1999. J. Biol. Chem. 274:9587-99.

35. Meng, et al. 1992. J. Comp. Chem. 13:505-24.

36. Merritt, E., and Murphy, M. 1994. Acta Crystallogr. D50:869-73

37. Methods in Enzymology, Vol. 114 & 115, H. W. Wyckoff et al., eds., Academic Press (1985).

38. S. Miranker, et al. 1991. Proteins 11:29-34.

39. Narayana, N., Cox, S., Shaltiel, S., Taylor, S., and Xuong, N. 1997. Biochemistry 36:4438-48.

40. Navaza, J. 1994. Acta Crystallogr. A50:157-63, “AMoRe: an Automated Package for Molecular Replacement”.

41. Nicholls, A. 1991. Proteins 11:281-96

42. Nishibata, Y., and Itai, A. 1993. J. Med. Chem. 36:2921-8.

43. Nisitani, S., Kato, R. M., Rawlings, D. J., Witte, O. N., and Wahl, M. I. 1999. Proc. Natl. Acad. Sci. USA 96:2221-6.

44. Nisitani, S., Satterthwaite, A., Akashi, K., Weissman, I., Witte, O., and Wahl, M. 2000. Proc. Natl. Acad. Sci. USA 97:2737-42.

45. Noren, et al. 1989. Science 244:182-88.

46. Oda, A., Ikeda, Y., Ochs, H., Druker, B., Ozaki, K., Handa, M., Ariga, T., Sakiyama, Y., Witte, O., and Wahl, M. 2000. Blood 95:1663-70.

47. Orlandi, P., Ritis, K., Moschese, V., Angelini, F., Arvanitidis, K., Speletas, M., Sideras, P., Plebani, A., and Rossi, P. 2000. Hum. Mutat. 15:117 (Online Citation #285, 6 pp.).

48. Otwinowski, Z., and Minor, W. 1998. Methods in Enzymology 276:307-25.

49. Park, H., Wahl, M., Afar, D., Turck, C., Rawlings, D., Tam, C., Scharenberg, A., Kinet, J., and Witte, O. 1996. Immunity 4:515-25.

50. Rawlings, D., and Witte, O. 1994. Immunol. Reviews 138:105-19.

51. Rawlings, D., Saffran, D., Tsukada, S., Largaespada, D., Grimaldi, J., Cohen, L., Mohr, R., Bazan, J., Howard, M., Copeland, N., and et al. 1993. Science 261:358-61.

52. Rawlings, D., Scharenberg, A., Park, H., Wahl, M., Lin,.S., Kato, R., Fluckiger, A., Witte, O., and Kinet, J. 1996. Science 271:822-5.

53. Sack, J. 1988. J. Mol. Graphics 6:244-45.

54. Schmitz, R., Baumann, G., and Gram, H. 1996. J. Mol. Biol. 260:664-77.

55. Skamnaki, V., Owen, D., Noble, M., Lowe, E., Lowe, G., Oikonomakos, N., and Johnson, L. 1999. Biochemistry 38:14718-30.

56. Tatusova, et al. 1999 FEMS Microbiol. Lett. 174:247-50.

57. Travis. 1993. Science 262:1374.

58. Tsukada, S., and Witte, O. 1994. Adv. Exp. Med. & Biol. 365:233-8.

59. Uckun, et al. 1996. Science 273:1096-100.

60. Uckun, et al 1995. Science 267:886-91.

61. Uckun, F. 1998. Biochem. Pharm. 56:683-91.

62. Uckun, F., et al. 1999. J. Biol. Chem. 274:9587-99.

63. Van Duyne, et al. 1993. J. Mol. Biol. 229:105-24.

64. Vassilev, A., Ozer, Z., Navara, C., Mahajan, S., and Uckun, F. 1999. J. Biol. Chem. 274:1646-56.

65. Vetrie, D., Vorechovsky, I., Sideras, P., Holland, J., Davies, A., Flinter, F., Hammarstrom, L., Kinnon, C., Levinsky, R., Bobrow, M., and et al., 1993. Nature 361:226-33.

66. Vihinen, M., Brandau, O., Branden, L., Kwan, S., Lappalainen, I., Lester, T., Noordzij, J., Ochs, H., Ollila, J., Pienaar, S., Riikonen, P., Saha, B., and Smith, C. 1998. Nucl. Acids Res. 26:242-7.

67. Vihinen, M., Cooper, M. D., de Saint Basile, G., Fischer, A., Good, R. A., Hendriks, R. W., Kinnon, C., Kwan, S. P., Litman, G. W., Notarangelo, L. D., and et al. 1995. Immunology Today 16:460-5.

68. Wahl, M., Fluckiger, A., Kato, R., Park, H., Witte, O., and Rawlings, D. 1997. Proc. Natl. Acad. Sci. USA 94:11526-33.

69. Xu, W., Harrison, S., and Eck, M. 1997. Nature 385:595-602.

70. Xu, W., Doshi, A., Lei, M., Eck, M., and Harrison, S.1999. Mol. Cell 3:629-38.

71. Yamaguchi, H. and Hendrickson, W. 1996. Nature 384:484-9.

72. Schindler, T., Sicheri, F., Pico, A., Gazit, A., Levitzki, A., and Kuriyan, J. 1999. Mol. Cell 3:639-48.

TABLE 1

| | | | Amino | | | | | | | |
|---|---|---|---|---|---|---|---|---|---|---|
| | Atom | Atom | Acid | | | | | | Temp | |
| | Number | Type | Residue | | X | Y | Z | Occ. | Factor | |
| ATOM | 1 | CB | ILE | 397 | 39.984 | 4.826 | 17.892 | 1.00 | 26.05 | BTKA |
| ATOM | 2 | CG2 | ILE | 397 | 41.212 | 3.952 | 17.664 | 1.00 | 28.70 | BTKA |
| ATOM | 3 | CG1 | ILE | 397 | 39.927 | 5.294 | 19.346 | 1.00 | 21.76 | BTKA |
| ATOM | 4 | CD | ILE | 397 | 38.668 | 6.044 | 19.704 | 1.00 | 19.11 | BTKA |
| ATOM | 5 | C | ILE | 397 | 41.382 | 6.663 | 16.890 | 1.00 | 28.45 | BTKA |
| ATOM | 6 | O | ILE | 397 | 42.042 | 6.591 | 15.857 | 1.00 | 27.84 | BTKA |
| ATOM | 7 | N | ILE | 397 | 39.699 | 5.634 | 15.537 | 1.00 | 31.08 | BTKA |
| ATOM | 8 | CA | ILE | 397 | 39.996 | 6.043 | 16.938 | 1.00 | 27.95 | BTKA |
| ATOM | 9 | N | ASP | 398 | 41.833 | 7.254 | 17.988 | 1.00 | 29.89 | BTKA |
| ATOM | 10 | CA | ASP | 398 | 43.142 | 7.879 | 17.991 | 1.00 | 30.58 | BTKA |
| ATOM | 11 | CB | ASP | 398 | 42.982 | 9.399 | 17.868 | 1.00 | 27.36 | BTKA |
| ATOM | 12 | CG | ASP | 398 | 44.227 | 10.080 | 17.336 | 1.00 | 26.98 | BTKA |
| ATOM | 13 | OD1 | ASP | 398 | 45.346 | 9.607 | 17.635 | 1.00 | 23.87 | BTKA |
| ATOM | 14 | OD2 | ASP | 398 | 44.078 | 11.087 | 16.601 | 1.00 | 23.63 | BTKA |
| ATOM | 15 | C | ASP | 398 | 43.966 | 7.548 | 19.230 | 1.00 | 33.13 | BTKA |
| ATOM | 16 | O | ASP | 398 | 43.564 | 7.860 | 20.350 | 1.00 | 34.18 | BTKA |
| ATOM | 17 | N | PRO | 399 | 45.080 | 6.820 | 19.050 | 1.00 | 36.49 | BTKA |
| ATOM | 18 | CD | PRO | 399 | 45.461 | 6.040 | 17.861 | 1.00 | 38.51 | BTKA |
| ATOM | 19 | CA | PRO | 399 | 45.938 | 6.472 | 20.187 | 1.00 | 36.60 | BTKA |
| ATOM | 20 | CB | PRO | 399 | 46.941 | 5.494 | 19.573 | 1.00 | 34.91 | BTKA |
| ATOM | 21 | CG | PRO | 399 | 46.157 | 4.851 | 18.479 | 1.00 | 38.91 | BTKA |
| ATOM | 22 | C | PRO | 399 | 46.625 | 7.765 | 20.581 | 1.00 | 35.91 | BTKA |
| ATOM | 23 | O | PRO | 399 | 47.165 | 8.454 | 19.718 | 1.00 | 35.42 | BTKA |
| ATOM | 24 | N | LYS | 400 | 46.574 | 8.114 | 21.861 | 1.00 | 36.75 | BTKA |
| ATOM | 25 | CA | LYS | 400 | 47.189 | 9.349 | 22.325 | 1.00 | 33.61 | BTKA |
| ATOM | 26 | CB | LYS | 400 | 46.860 | 9.604 | 23.800 | 1.00 | 34.79 | BTKA |
| ATOM | 27 | CG | LYS | 400 | 47.406 | 10.924 | 24.332 | 1.00 | 32.92 | BTKA |
| ATOM | 28 | CD | LYS | 400 | 47.052 | 11.127 | 25.795 | 1.00 | 32.83 | BTKA |
| ATOM | 29 | CE | LYS | 400 | 45.589 | 11.506 | 25.982 | 1.00 | 33.63 | BTKA |
| ATOM | 30 | NZ | LYS | 400 | 45.298 | 12.915 | 25.566 | 1.00 | 32.25 | BTKA |
| ATOM | 31 | C | LYS | 400 | 48.698 | 9.448 | 22.083 | 1.00 | 31.96 | BTKA |
| ATOM | 32 | O | LYS | 400 | 49.510 | 9.153 | 22.958 | 1.00 | 27.95 | BTKA |
| ATOM | 33 | N | ASP | 401 | 49.059 | 9.828 | 20.863 | 1.00 | 32.35 | BTKA |
| ATOM | 34 | CA | ASP | 401 | 50.453 | 10.014 | 20.493 | 1.00 | 31.87 | BTKA |
| ATOM | 35 | CB | ASP | 401 | 50.805 | 9.258 | 19.196 | 1.00 | 32.78 | BTKA |
| ATOM | 36 | CG | ASP | 401 | 49.823 | 9.521 | 18.057 | 1.00 | 34.30 | BTKA |
| ATOM | 37 | OD1 | ASP | 401 | 49.422 | 8.542 | 17.390 | 1.00 | 31.33 | BTKA |
| ATOM | 38 | OD2 | ASP | 401 | 49.466 | 10.692 | 17.811 | 1.00 | 34.83 | BTKA |
| ATOM | 39 | C | ASP | 401 | 50.645 | 11.520 | 20.347 | 1.00 | 29.99 | BTKA |
| ATOM | 40 | O | ASP | 401 | 51.112 | 12.016 | 19.321 | 1.00 | 28.60 | BTKA |
| ATOM | 41 | N | LEU | 402 | 50.212 | 12.238 | 21.378 | 1.00 | 30.44 | BTKA |
| ATOM | 42 | CA | LEU | 402 | 50.307 | 13.686 | 21.416 | 1.00 | 32.58 | BTKA |
| ATOM | 43 | CB | LEU | 402 | 48.911 | 14.310 | 21.538 | 1.00 | 29.13 | BTKA |
| ATOM | 44 | CG | LEU | 402 | 47.769 | 13.911 | 20.596 | 1.00 | 28.32 | BTKA |
| ATOM | 45 | CD1 | LEU | 402 | 47.220 | 12.552 | 20.969 | 1.00 | 26.12 | BTKA |
| ATOM | 46 | CD2 | LEU | 402 | 46.662 | 14.931 | 20.708 | 1.00 | 26.92 | BTKA |
| ATOM | 47 | C | LEU | 402 | 51.150 | 14.087 | 22.624 | 1.00 | 35.40 | BTKA |
| ATOM | 48 | O | LEU | 402 | 51.460 | 13.254 | 23.476 | 1.00 | 36.97 | BTKA |
| ATOM | 49 | N | THR | 403 | 51.538 | 15.356 | 22.681 | 1.00 | 36.15 | BTKA |
| ATOM | 50 | CA | THR | 403 | 52.330 | 15.864 | 23.793 | 1.00 | 35.21 | BTKA |
| ATOM | 51 | CB | THR | 403 | 53.584 | 16.641 | 23.296 | 1.00 | 35.16 | BTKA |
| ATOM | 52 | OG1 | THR | 403 | 53.287 | 17.328 | 22.073 | 1.00 | 36.34 | BTKA |
| ATOM | 53 | CG2 | THR | 403 | 54.745 | 15.688 | 23.072 | 1.00 | 33.51 | BTKA |
| ATOM | 54 | C | THR | 403 | 51.448 | 16.724 | 24.705 | 1.00 | 35.29 | BTKA |
| ATOM | 55 | O | THR | 403 | 50.473 | 16.214 | 25.266 | 1.00 | 34.77 | BTKA |
| ATOM | 56 | N | PHE | 404 | 51.761 | 18.014 | 24.834 | 1.00 | 32.58 | BTKA |
| ATOM | 57 | CA | PHE | 404 | 50.985 | 18.918 | 25.683 | 1.00 | 32.99 | BTKA |
| ATOM | 58 | CB | PHE | 404 | 51.060 | 18.457 | 27.151 | 1.00 | 33.57 | BTKA |
| ATOM | 59 | CG | PHE | 404 | 49.973 | 19.023 | 28.034 | 1.00 | 36.18 | BTKA |
| ATOM | 60 | CD1 | PHE | 404 | 50.063 | 20.317 | 28.541 | 1.00 | 36.37 | BTKA |
| ATOM | 61 | CD2 | PHE | 404 | 48.873 | 18.248 | 28.383 | 1.00 | 35.88 | BTKA |
| ATOM | 62 | CE1 | PHE | 404 | 49.076 | 20.828 | 29.383 | 1.00 | 36.13 | BTKA |
| ATOM | 63 | CE2 | PHE | 404 | 47.881 | 18.750 | 29.224 | 1.00 | 36.02 | BTKA |
| ATOM | 64 | CZ | PHE | 404 | 47.982 | 20.041 | 29.726 | 1.00 | 36.27 | BTKA |
| ATOM | 65 | C | PHE | 404 | 51.559 | 20.327 | 25.551 | 1.00 | 30.92 | BTKA |
| ATOM | 66 | O | PHE | 404 | 52.748 | 20.484 | 25.275 | 1.00 | 28.05 | BTKA |
| ATOM | 67 | N | LEU | 405 | 50.710 | 21.341 | 25.706 | 1.00 | 31.51 | BTKA |
| ATOM | 68 | CA | LEU | 405 | 51.156 | 22.730 | 25.627 | 1.00 | 31.53 | BTKA |
| ATOM | 89 | CB | LEU | 405 | 51.175 | 23.239 | 24.183 | 1.00 | 32.59 | BTKA |
| ATOM | 70 | CG | LEU | 405 | 51.990 | 24.521 | 23.961 | 1.00 | 33.86 | BTKA |
| ATOM | 71 | CD1 | LEU | 405 | 53.466 | 24.150 | 23.845 | 1.00 | 34.64 | BTKA |
| ATOM | 72 | CD2 | LEU | 405 | 51.533 | 25.263 | 22.706 | 1.00 | 37.27 | BTKA |
| ATOM | 73 | C | LEU | 405 | 50.343 | 23.672 | 26.521 | 1.00 | 32.49 | BTKA |
| ATOM | 74 | O | LEU | 405 | 50.538 | 23.676 | 27.735 | 1.00 | 33.98 | BTKA |
| ATOM | 75 | N | LYS | 406 | 49.411 | 24.434 | 25.948 | 1.00 | 35.17 | BTKA |
| ATOM | 76 | CA | LYS | 406 | 48.630 | 25.387 | 26.742 | 1.00 | 36.21 | BTKA |
| ATOM | 77 | CB | LYS | 406 | 49.411 | 26.702 | 26.877 | 1.00 | 33.84 | BTKA |
| ATOM | 78 | CG | LYS | 406 | 50.477 | 26.685 | 27.960 | 1.00 | 33.94 | BTKA |
| ATOM | 79 | CD | LYS | 406 | 51.410 | 27.884 | 27.867 | 1.00 | 35.43 | BTKA |
| ATOM | 80 | CE | LYS | 406 | 52.445 | 27.713 | 26.760 | 1.00 | 37.40 | BTKA |
| ATOM | 81 | NZ | LYS | 406 | 53.438 | 26.632 | 27.055 | 1.00 | 35.32 | BTKA |
| ATOM | 82 | C | LYS | 406 | 47.182 | 25.679 | 26.303 | 1.00 | 37.77 | BTKA |
| ATOM | 83 | O | LYS | 406 | 46.588 | 24.939 | 25.512 | 1.00 | 39.12 | BTKA |
| ATOM | 84 | N | GLU | 407 | 46.629 | 26.752 | 26.873 | 1.00 | 35.20 | BTKA |
| ATOM | 85 | CA | GLU | 407 | 45.263 | 27.221 | 26.633 | 1.00 | 34.68 | BTKA |
| ATOM | 86 | CB | GLU | 407 | 45.105 | 28.626 | 27.220 | 1.00 | 34.04 | BTKA |
| ATOM | 87 | CG | GLU | 407 | 43.747 | 29.277 | 26.974 | 1.00 | 28.63 | BTKA |
| ATOM | 88 | CD | GLU | 407 | 43.628 | 30.632 | 27.624 | 1.00 | 23.09 | BTKA |
| ATOM | 89 | OE1 | GLU | 407 | 44.594 | 31.415 | 27.557 | 1.00 | 19.69 | BTKA |
| ATOM | 90 | OE2 | GLU | 407 | 42.570 | 30.912 | 28.213 | 1.00 | 19.52 | BTKA |
| ATOM | 91 | C | GLU | 407 | 44.833 | 27.253 | 25.178 | 1.00 | 34.30 | BTKA |
| ATOM | 92 | O | GLU | 407 | 45.664 | 27.386 | 24.283 | 1.00 | 38.04 | BTKA |
| ATOM | 93 | N | LEU | 408 | 43.523 | 27.209 | 24.957 | 1.00 | 33.77 | BTKA |
| ATOM | 94 | CA | LEU | 408 | 42.980 | 27.248 | 23.608 | 1.00 | 35.95 | BTKA |
| ATOM | 95 | CB | LEU | 408 | 43.335 | 25.958 | 22.888 | 1.00 | 35.53 | BTKA |
| ATOM | 96 | CG | LEU | 408 | 44.349 | 26.065 | 21.763 | 1.00 | 33.73 | BTKA |
| ATOM | 97 | CD1 | LEU | 408 | 45.091 | 24.751 | 21.657 | 1.00 | 32.87 | BTKA |
| ATOM | 98 | CD2 | LEU | 408 | 43.649 | 26.440 | 20.453 | 1.00 | 33.61 | BTKA |
| ATOM | 99 | C | LEU | 408 | 41.473 | 27.516 | 23.537 | 1.00 | 36.07 | BTKA |
| ATOM | 100 | O | LEU | 408 | 40.836 | 27.827 | 24.549 | 1.00 | 30.55 | BTKA |
| ATOM | 101 | N | GLY | 409 | 40.923 | 27.400 | 22.328 | 1.00 | 37.82 | BTKA |
| ATOM | 102 | CA | GLY | 409 | 39.509 | 27.647 | 22.086 | 1.00 | 42.67 | BTKA |
| ATOM | 103 | C | GLY | 409 | 38.531 | 26.929 | 23.000 | 1.00 | 43.77 | BTKA |
| ATOM | 104 | O | GLY | 409 | 38.872 | 25.926 | 23.621 | 1.00 | 44.19 | BTKA |
| ATOM | 105 | N | TER | 410 | 37.300 | 27.426 | 23.053 | 1.00 | 44.64 | BTKA |
| ATOM | 106 | CA | TER | 410 | 36.269 | 26.844 | 23.903 | 1.00 | 44.12 | BTKA |
| ATOM | 107 | CB | TER | 410 | 35.790 | 27.859 | 24.963 | 1.00 | 42.36 | BTKA |
| ATOM | 108 | OG1 | TER | 410 | 36.859 | 28.760 | 25.285 | 1.00 | 38.07 | BTKA |
| ATOM | 109 | CG2 | TER | 410 | 35.353 | 27.141 | 26.225 | 1.00 | 42.56 | BTKA |
| ATOM | 110 | C | THR | 410 | 35.056 | 26.429 | 23.076 | 1.00 | 45.63 | BTKA |
| ATOM | 111 | O | THR | 410 | 34.984 | 26.700 | 21.874 | 1.00 | 46.48 | BTKA |
| ATOM | 112 | N | GLY | 411 | 34.106 | 25.776 | 23.739 | 1.00 | 45.00 | BTKA |
| ATOM | 113 | CA | GLY | 411 | 32.882 | 25.326 | 23.104 | 1.00 | 43.61 | BTKA |
| ATOM | 114 | C | GLY | 411 | 31.968 | 24.888 | 24.228 | 1.00 | 42.95 | BTKA |
| ATOM | 115 | O | GLY | 411 | 32.438 | 24.681 | 25.347 | 1.00 | 42.15 | BTKA |
| ATOM | 116 | N | GLN | 412 | 30.682 | 24.697 | 23.946 | 1.00 | 41.91 | BTKA |
| ATOM | 117 | CA | GLN | 412 | 29.736 | 24.291 | 24.984 | 1.00 | 40.64 | BTKA |
| ATOM | 118 | CB | GLN | 412 | 28.300 | 24.300 | 24.452 | 1.00 | 40.22 | BTKA |
| ATOM | 119 | CG | GLN | 412 | 27.748 | 25.702 | 24.225 | 1.00 | 38.55 | BTKA |
| ATOM | 120 | CD | GLN | 412 | 26.335 | 25.717 | 23.663 | 1.00 | 34.81 | BTKA |
| ATOM | 121 | OE1 | GLN | 412 | 25.732 | 26.780 | 23.514 | 1.00 | 33.50 | BTKA |
| ATOM | 122 | NE2 | OLN | 412 | 25.799 | 24.545 | 23.354 | 1.00 | 28.18 | BTKA |
| ATOM | 123 | C | CLN | 412 | 30.050 | 22.969 | 25.692 | 1.00 | 37.36 | BTKA |
| ATOM | 124 | O | GLN | 412 | 29.342 | 22.585 | 26.627 | 1.00 | 36.95 | BTKA |
| ATOM | 125 | N | PHE | 413 | 31.060 | 22.248 | 25.214 | 1.00 | 34.84 | BTKA |
| ATOM | 126 | CA | PHE | 413 | 31.468 | 21.000 | 25.853 | 1.00 | 32.28 | BTKA |
| ATOM | 127 | CE | PEE | 413 | 31.890 | 19.942 | 24.825 | 1.00 | 33.34 | BTKA |
| ATOM | 128 | CG | PHE | 413 | 30.742 | 19.163 | 24.226 | 1.00 | 30.57 | BTKA |
| ATOM | 129 | CD1 | PEE | 413 | 29.490 | 19.749 | 24.032 | 1.00 | 30.22 | BTKA |
| ATOM | 130 | CD2 | PHE | 413 | 30.934 | 17.850 | 23.800 | 1.00 | 29.00 | BTKA |
| ATOM | 131 | CE1 | PEE | 413 | 28.451 | 19.039 | 23.417 | 1.00 | 29.05 | BTKA |
| ATOM | 132 | CE2 | PEE | 413 | 29.899 | 17.133 | 23.183 | 1.00 | 27.19 | BTKA |
| ATOM | 133 | CZ | PEE | 413 | 28.659 | 17.728 | 22.990 | 1.00 | 25.29 | BTKA |
| ATOM | 134 | C | PHE | 413 | 32.649 | 21.334 | 26.753 | 1.00 | 31.49 | BTKA |
| ATOM | 135 | O | PHE | 413 | 32.865 | 20.701 | 27.783 | 1.00 | 28.50 | BTKA |
| ATOM | 136 | N | GLY | 414 | 33.413 | 22.346 | 26.353 | 1.00 | 33.87 | BTKA |
| ATOM | 137 | CA | GLY | 414 | 34.562 | 22.763 | 27.134 | 1.00 | 36.54 | BTKA |
| ATOM | 138 | C | GLY | 414 | 35.623 | 23.439 | 26.288 | 1.00 | 38.54 | BTKA |
| ATOM | 139 | O | GLY | 414 | 35.372 | 23.843 | 25.144 | 1.00 | 37.26 | BTKA |
| ATOM | 140 | N | VAL | 415 | 36.800 | 23.604 | 26.883 | 1.00 | 38.40 | BTKA |
| ATOM | 141 | CA | VAL | 415 | 37.944 | 24.225 | 26.228 | 1.00 | 35.19 | BTKA |
| ATOM | 142 | CB | VAL | 415 | 38.859 | 24.900 | 27.286 | 1.00 | 33.66 | BTKA |
| ATOM | 143 | CG1 | VAL | 415 | 39.567 | 23.841 | 28.118 | 1.00 | 33.58 | BTKA |
| ATOM | 144 | CG2 | VAL | 415 | 39.851 | 25.858 | 26.639 | 1.00 | 29.68 | BTKA |
| ATOM | 145 | C | VAL | 415 | 38.735 | 23.136 | 25.499 | 1.00 | 33.93 | BTKA |
| ATOM | 146 | O | VAL | 415 | 38.348 | 21.961 | 25.515 | 1.00 | 33.38 | BTKA |
| ATOM | 147 | N | VAL | 416 | 39.821 | 23.544 | 24.845 | 1.00 | 33.13 | BTKA |
| ATOM | 148 | CA | VAL | 416 | 40.716 | 22.648 | 24.124 | 1.00 | 28.19 | BTKA |
| ATOM | 149 | CB | VAL | 416 | 40.608 | 22.849 | 22.570 | 1.00 | 27.16 | BTKA |
| ATOM | 150 | CG1 | VAL | 416 | 39.236 | 22.466 | 22.080 | 1.00 | 24.50 | BTKA |
| ATOM | 151 | CG2 | VAL | 416 | 40.838 | 24.282 | 22.184 | 1.00 | 21.15 | BTKA |
| ATOM | 152 | C | VAL | 416 | 42.138 | 22.937 | 24.628 | 1.00 | 25.73 | BTKA |
| ATOM | 153 | O | VAL | 416 | 42.344 | 23.904 | 25.354 | 1.00 | 25.11 | BTKA |
| ATOM | 154 | N | LYS | 417 | 43.100 | 22.079 | 24.298 | 1.00 | 27.45 | BTKA |
| ATOM | 155 | CA | LYS | 417 | 44.505 | 22.255 | 24.723 | 1.00 | 28.38 | BTKA |
| ATOM | 156 | CB | LYS | 417 | 44.875 | 21.266 | 25.838 | 1.00 | 30.82 | BTKA |
| ATOM | 157 | CG | LYS | 417 | 44.299 | 21.627 | 27.207 | 1.00 | 32.85 | BTKA |
| ATOM | 158 | CD | LYS | 417 | 44.889 | 22.936 | 27.747 | 1.00 | 32.78 | BTKA |
| ATOM | 159 | CE | LYS | 417 | 44.092 | 23.465 | 28.933 | 1.00 | 30.38 | BTKA |
| ATOM | 160 | NZ | LYS | 417 | 42.671 | 23.743 | 28.566 | 1.00 | 24.07 | BTKA |
| ATOM | 161 | C | LYE | 417 | 45.429 | 22.050 | 23.531 | 1.00 | 22.74 | BTKA |
| ATOM | 162 | O | LYS | 417 | 44.971 | 21.628 | 22.482 | 1.00 | 23.20 | BTKA |
| ATOM | 163 | N | ALA | 418 | 46.717 | 22.344 | 23.670 | 1.00 | 19.60 | BTKA |
| ATOM | 164 | CA | ALA | 418 | 47.625 | 22.172 | 22.532 | 1.00 | 19.78 | BTKA |
| ATOM | 165 | CB | ALA | 418 | 48.484 | 23.410 | 22.334 | 1.00 | 19.67 | BTKA |
| ATOM | 166 | C | ALA | 418 | 48.493 | 20.924 | 22.550 | 1.00 | 19.25 | BTKA |
| ATOM | 167 | O | ALA | 418 | 48.892 | 20.446 | 23.619 | 1.00 | 19.18 | BTKA |
| ATOM | 168 | N | GLY | 419 | 48.751 | 20.398 | 21.352 | 1.00 | 16.40 | BTKA |
| ATOM | 169 | CA | GLY | 419 | 49.570 | 19.210 | 21.176 | 1.00 | 14.78 | BTKA |
| ATOM | 170 | C | GLY | 419 | 49.886 | 18.994 | 19.704 | 1.00 | 14.35 | BTKA |
| ATOM | 171 | O | GLY | 419 | 49.350 | 19.692 | 18.841 | 1.00 | 15.21 | BTKA |
| ATOM | 172 | N | ALA | 420 | 50.787 | 18.063 | 19.411 | 1.00 | 12.74 | BTKA |
| ATOM | 173 | CA | ALA | 420 | 51.152 | 17.747 | 18.034 | 1.00 | 10.26 | BTKA |
| ATOM | 174 | CB | ALA | 420 | 52.660 | 17.800 | 17.855 | 1.00 | 10.16 | BTKA |
| ATOM | 175 | C | ALA | 420 | 50.631 | 16.345 | 17.776 | 1.00 | 9.89 | BTKA |
| ATOM | 176 | O | ALA | 420 | 50.619 | 15.520 | 18.685 | 1.00 | 8.22 | BTKA |
| ATOM | 177 | N | TRP | 421 | 50.185 | 16.073 | 16.557 | 1.00 | 10.66 | BTKA |
| ATOM | 178 | CA | TRP | 421 | 49.637 | 14.759 | 16.236 | 1.00 | 14.79 | BTKA |
| ATOM | 179 | CB | TRP | 421 | 48.117 | 14.878 | 16.060 | 1.00 | 13.99 | BTKA |
| ATOM | 180 | CG | TRP | 421 | 47.433 | 13.605 | 15.683 | 1.00 | 15.77 | BTKA |
| ATOM | 181 | CD2 | TRP | 421 | 46.685 | 13.355 | 14.486 | 1.00 | 17.12 | BTKA |
| ATOM | 182 | CE2 | TRP | 421 | 46.203 | 12.027 | 14.563 | 1.00 | 19.95 | BTKA |
| ATOM | 183 | CE3 | TRP | 421 | 46.373 | 14.123 | 13.357 | 1.00 | 13.78 | BTKA |
| ATOM | 184 | CD1 | TRP | 421 | 47.380 | 12.452 | 16.414 | 1.00 | 18.99 | BTKA |
| ATOM | 185 | NE1 | TRP | 421 | 46.646 | 11.498 | 15.746 | 1.00 | 21.46 | BTKA |
| ATOM | 186 | CZ2 | TRP | 421 | 45.419 | 11.454 | 13.552 | 1.00 | 19.23 | BTKA |
| ATOM | 187 | CZ3 | TRP | 421 | 45.595 | 13.552 | 12.354 | 1.00 | 9.53 | BTKA |
| ATOM | 188 | CH2 | TRP | 421 | 45.127 | 12.233 | 12.460 | 1.00 | 12.55 | BTKA |
| ATOM | 189 | C | TRP | 421 | 50.270 | 14.085 | 15.011 | 1.00 | 16.84 | BTKA |
| ATOM | 190 | O | TRP | 421 | 50.448 | 14.721 | 13.969 | 1.00 | 17.90 | BTKA |
| ATOM | 191 | N | ARG | 422 | 50.600 | 12.799 | 15.158 | 1.00 | 18.95 | BTKA |
| ATOM | 192 | CA | ARG | 422 | 51.194 | 11.983 | 14.091 | 1.00 | 24.07 | BTKA |
| ATOM | 193 | CB | ARG | 422 | 50.111 | 11.437 | 13.144 | 1.00 | 24.28 | BTKA |
| ATOM | 194 | CG | ARG | 422 | 49.243 | 10.342 | 13.724 | 1.00 | 21.64 | BTKA |
| ATOM | 195 | CD | ARG | 422 | 48.551 | 9.548 | 12.621 | 1.00 | 22.23 | BTKA |
| ATOM | 196 | NE | ARG | 422 | 47.770 | 10.392 | 11.720 | 1.00 | 25.72 | BTKA |
| ATOM | 197 | CZ | ARG | 422 | 46.956 | 9.936 | 10.769 | 1.00 | 26.16 | BTKA |
| ATOM | 198 | NH1 | ARG | 422 | 46.292 | 10.799 | 10.011 | 1.00 | 24.22 | BTKA |
| ATOM | 199 | NH2 | ARG | 422 | 46.799 | 8.630 | 10.568 | 1.00 | 25.98 | BTKA |
| ATOM | 200 | C | ARG | 422 | 52.263 | 12.701 | 13.267 | 1.00 | 26.23 | BTKA |
| ATOM | 201 | O | ARG | 422 | 52.163 | 12.802 | 12.043 | 1.00 | 25.36 | BTKA |
| ATOM | 202 | N | GLY | 423 | 53.308 | 13.160 | 13.935 | 1.00 | 28.03 | BTKA |
| ATOM | 203 | CA | GLY | 423 | 54.347 | 13.876 | 13.234 | 1.00 | 28.68 | BTKA |
| ATOM | 204 | C | GLY | 423 | 54.117 | 15.347 | 13.502 | 1.00 | 31.49 | BTKA |
| ATOM | 205 | O | GLY | 423 | 54.093 | 15.758 | 14.666 | 1.00 | 33.41 | BTKA |
| ATOM | 206 | N | ALA | 424 | 53.831 | 16.124 | 12.460 | 1.00 | 30.81 | BTKA |
| ATOM | 207 | CA | ALA | 424 | 53.635 | 17.553 | 12.660 | 1.00 | 29.14 | BTKA |
| ATOM | 208 | CB | ALA | 424 | 54.793 | 18.322 | 12.067 | 1.00 | 32.15 | BTKA |
| ATOM | 209 | C | ALA | 424 | 52.301 | 18.185 | 12.256 | 1.00 | 25.28 | BTKA |
| ATOM | 210 | O | ALA | 424 | 52.227 | 18.989 | 11.324 | 1.00 | 22.96 | BTKA |
| ATOM | 211 | N | ALA | 425 | 51.249 | 17.822 | 12.978 | 1.00 | 21.73 | BTKA |
| ATOM | 212 | CA | ALA | 425 | 49.936 | 18.390 | 12.753 | 1.00 | 17.25 | BTKA |
| ATOM | 213 | CB | ALA | 425 | 48.910 | 17.300 | 12.468 | 1.00 | 14.79 | BTKA |
| ATOM | 214 | C | ALA | 425 | 49.623 | 19.102 | 14.062 | 1.00 | 17.01 | BTKA |
| ATOM | 215 | O | ALA | 425 | 49.656 | 18.490 | 15.128 | 1.00 | 16.50 | BTKA |
| ATOM | 216 | N | ASP | 426 | 49.445 | 20.415 | 13.996 | 1.00 | 16.77 | BTKA |
| ATOM | 217 | CA | ASP | 426 | 49.137 | 21.200 | 15.179 | 1.00 | 14.51 | BTKA |
| ATOM | 218 | CB | ASP | 426 | 49.418 | 22.681 | 14.926 | 1.00 | 16.45 | BTKA |
| ATOM | 219 | CG | ASP | 426 | 50.882 | 22.956 | 14.643 | 1.00 | 20.73 | BTKA |
| ATOM | 220 | OD1 | ASP | 426 | 51.185 | 23.493 | 13.558 | 1.00 | 19.46 | BTKA |
| ATOM | 221 | OD2 | ASP | 426 | 51.732 | 22.637 | 15.504 | 1.00 | 22.08 | BTKA |
| ATOM | 222 | C | ASP | 426 | 47.672 | 20.983 | 15.507 | 1.00 | 15.14 | BTKA |
| ATOM | 223 | O | ASP | 426 | 46.796 | 21.258 | 14.682 | 1.00 | 15.14 | BTKA |
| ATOM | 224 | N | VAL | 427 | 47.408 | 20.473 | 16.706 | 1.00 | 15.54 | BTKA |
| ATOM | 225 | CA | VAL | 427 | 46.046 | 20.196 | 17.125 | 1.00 | 12.56 | BTKA |
| ATOM | 226 | CB | VAL | 427 | 45.814 | 18.664 | 17.291 | 1.00 | 13.80 | BTKA |
| ATOM | 227 | CG1 | VAL | 427 | 46.243 | 17.926 | 16.041 | 1.00 | 7.25 | BTKA |
| ATOM | 228 | CG2 | VAL | 427 | 46.557 | 18.122 | 18.511 | 1.00 | 13.29 | BTKA |
| ATOM | 229 | C | VAL | 427 | 45.659 | 20.908 | 18.421 | 1.00 | 12.59 | BTKA |
| ATOM | 230 | O | VAL | 427 | 46.516 | 21.391 | 19.166 | 1.00 | 15.43 | BTKA |
| ATOM | 231 | N | ALA | 428 | 44.354 | 21.023 | 18.634 | 1.00 | 11.11 | BTKA |
| ATOM | 232 | CA | ALA | 428 | 43.785 | 21.637 | 19.822 | 1.00 | 12.87 | BTKA |
| ATOM | 233 | CB | ALA | 428 | 42.995 | 22.872 | 19.449 | 1.00 | 9.07 | BTKA |
| ATOM | 234 | C | ALA | 428 | 42.860 | 20.565 | 20.380 | 1.00 | 12.26 | BTKA |
| ATOM | 235 | O | ALA | 428 | 41.833 | 20.263 | 19.775 | 1.00 | 17.33 | BTKA |
| ATOM | 236 | N | ILE | 429 | 43.246 | 19.957 | 21.501 | 1.00 | 16.62 | BTKA |
| ATOM | 237 | CA | ILE | 429 | 42.461 | 18.889 | 22.117 | 1.00 | 15.64 | BTKA |
| ATOM | 238 | CB | ILE | 429 | 43.383 | 17.773 | 22.733 | 1.00 | 14.98 | BTKA |
| ATOM | 239 | CG2 | ILE | 429 | 44.844 | 18.223 | 22.770 | 1.00 | 13.61 | BTKA |
| ATOM | 240 | CG1 | ILE | 429 | 42.855 | 17.290 | 24.088 | 1.00 | 15.92 | BTKA |
| ATOM | 241 | CD | ILE | 429 | 43.679 | 16.183 | 24.712 | 1.00 | 20.29 | BTKA |
| ATOM | 242 | C | ILE | 429 | 41.342 | 19.321 | 23.067 | 1.00 | 16.00 | BTKA |
| ATOM | 243 | O | ILE | 429 | 41.583 | 19.938 | 24.114 | 1.00 | 12.97 | BTKA |
| ATOM | 244 | N | LYS | 430 | 40.112 | 19.013 | 22.657 | 1.00 | 20.23 | BTKA |
| ATOM | 245 | CA | LYS | 430 | 38.892 | 19.327 | 23.403 | 1.00 | 22.33 | BTKA |
| ATOM | 246 | CB | LYS | 430 | 37.769 | 19.692 | 22.422 | 1.00 | 25.59 | BTKA |
| ATOM | 247 | CG | LYS | 430 | 36.474 | 20.199 | 23.049 | 1.00 | 29.20 | BTKA |
| ATOM | 248 | CD | LYS | 430 | 35.457 | 20.560 | 21.963 | 1.00 | 31.15 | BTKA |
| ATOM | 249 | CE | LYS | 430 | 34.196 | 21.193 | 22.544 | 1.00 | 32.32 | BTKA |
| ATOM | 250 | NZ | LYS | 430 | 33.185 | 21.522 | 21.491 | 1.00 | 25.96 | BTKA |
| ATOM | 251 | C | LYS | 430 | 38.454 | 18.119 | 24.208 | 1.00 | 21.53 | BTKA |
| ATOM | 252 | O | LYS | 430 | 38.614 | 16.984 | 23.767 | 1.00 | 23.61 | BTKA |
| ATOM | 253 | N | MET | 431 | 37.899 | 18.364 | 25.385 | 1.00 | 22.32 | BTKA |
| ATOM | 254 | CA | MET | 431 | 37.420 | 17.281 | 26.232 | 1.00 | 25.57 | BTKA |
| ATOM | 255 | CB | MET | 431 | 37.831 | 17.490 | 27.696 | 1.00 | 23.66 | BTKA |
| ATOM | 256 | CG | MET | 431 | 39.313 | 17.321 | 27.980 | 1.00 | 28.55 | BTKA |
| ATOM | 257 | SD | MET | 431 | 39.644 | 17.035 | 29.737 | 1.00 | 29.62 | BTKA |
| ATOM | 258 | CE | MET | 431 | 39.350 | 18.692 | 30.411 | 1.00 | 25.27 | BTKA |
| ATOM | 259 | C | MET | 431 | 35.906 | 17.271 | 26.143 | 1.00 | 24.67 | BTKA |
| ATOM | 260 | O | MET | 431 | 35.288 | 18.316 | 25.941 | 1.00 | 27.00 | BTKA |
| ATOM | 261 | N | ILE | 432 | 35.320 | 16.084 | 26.199 | 1.00 | 26.73 | BTKA |
| ATOM | 262 | CA | ILE | 432 | 33.871 | 15.969 | 26.178 | 1.00 | 30.90 | BTKA |
| ATOM | 263 | CB | ILE | 432 | 33.392 | 14.792 | 25.284 | 1.00 | 32.72 | BTKA |
| ATOM | 264 | CG2 | ILE | 432 | 31.937 | 14.456 | 25.569 | 1.00 | 29.29 | BTKA |
| ATOM | 265 | CG1 | ILE | 432 | 33.547 | 15.149 | 23.802 | 1.00 | 31.51 | BTKA |
| ATOM | 266 | CD | ILE | 432 | 34.977 | 15.269 | 23.324 | 1.00 | 30.77 | BTKA |
| ATOM | 267 | C | ILE | 432 | 33.450 | 15.767 | 27.638 | 1.00 | 32.06 | BTKA |
| ATOM | 268 | O | ILE | 432 | 32.315 | 16.075 | 28.011 | 1.00 | 33.24 | BTKA |
| ATOM | 269 | N | LYS | 433 | 34.419 | 15.307 | 28.440 | 1.00 | 33.53 | BTKA |
| ATOM | 270 | CA | LYS | 433 | 34.341 | 15.031 | 29.885 | 1.00 | 34.00 | BTKA |
| ATOM | 271 | CB | LYS | 433 | 33.097 | 15.629 | 30.549 | 1.00 | 31.21 | BTKA |
| ATOM | 272 | CG | LYS | 433 | 33.169 | 17.155 | 30.714 | 1.00 | 30.57 | BTKA |
| ATOM | 273 | CD | LYS | 433 | 33.736 | 17.553 | 32.070 | 1.00 | 25.39 | BTKA |
| ATOM | 274 | CE | LYS | 433 | 32.746 | 17.249 | 33.191 | 1.00 | 24.00 | BTKA |
| ATOM | 275 | NZ | LYS | 433 | 31.467 | 17.997 | 33.006 | 1.00 | 15.65 | BTKA |
| ATOM | 276 | C | LYS | 433 | 34.519 | 13.555 | 30.235 | 1.00 | 35.56 | BTKA |
| ATOM | 277 | O | LYS | 433 | 34.910 | 12.755 | 29.384 | 1.00 | 36.53 | BTKA |
| ATOM | 278 | N | GLU | 434 | 34.292 | 13.210 | 31.500 | 1.00 | 35.04 | BTKA |
| ATOM | 279 | CA | GLU | 434 | 34.464 | 11.841 | 31.979 | 1.00 | 31.68 | BTKA |
| ATOM | 280 | CB | CLU | 434 | 35.236 | 11.859 | 33.299 | 1.00 | 31.76 | BTKA |
| ATOM | 281 | CG | GLU | 434 | 35.525 | 10.480 | 33.868 | 1.00 | 25.49 | BTKA |
| ATOM | 282 | CD | GLU | 434 | 36.019 | 10.534 | 35.283 | 1.00 | 25.78 | BTKA |
| ATOM | 283 | OE1 | GLU | 434 | 35.196 | 10.333 | 36.191 | 1.00 | 30.32 | BTKA |
| ATOM | 284 | OE2 | GLU | 434 | 37.222 | 10.776 | 35.491 | 1.00 | 30.52 | BTKA |
| ATOM | 285 | C | GLU | 434 | 33.159 | 11.074 | 32.177 | 1.00 | 31.82 | BTKA |
| ATOM | 286 | O | GLU | 434 | 32.313 | 11.481 | 32.969 | 1.00 | 28.09 | BTKA |
| ATOM | 287 | N | GLY | 435 | 33.031 | 9.938 | 31.495 | 1.00 | 34.12 | BTKA |
| ATOM | 288 | CA | GLY | 435 | 31.844 | 9.110 | 31.610 | 1.00 | 35.84 | BTKA |
| ATOM | 289 | C | GLY | 435 | 30.583 | 9.721 | 31.023 | 1.00 | 37.43 | BTKA |
| ATOM | 290 | O | GLY | 435 | 29.953 | 9.120 | 30.144 | 1.00 | 36.18 | BTKA |
| ATOM | 291 | N | SER | 436 | 30.200 | 10.880 | 31.562 | 1.00 | 37.72 | BTKA |
| ATOM | 292 | CA | SER | 436 | 29.024 | 11.656 | 31.157 | 1.00 | 35.78 | BTKA |
| ATOM | 293 | CB | SER | 436 | 29.380 | 13.149 | 31.108 | 1.00 | 35.22 | BTKA |
| ATOM | 294 | OG | SER | 436 | 30.577 | 13.383 | 30.374 | 1.00 | 28.66 | BTKA |
| ATOM | 295 | C | SER | 436 | 28.442 | 11.227 | 29.825 | 1.00 | 34.88 | BTKA |
| ATOM | 296 | O | SER | 436 | 27.311 | 10.745 | 29.752 | 1.00 | 33.75 | BTKA |
| ATOM | 297 | N | MET | 437 | 29.236 | 11.407 | 28.778 | 1.00 | 35.33 | BTKA |
| ATOM | 298 | CA | MET | 437 | 28.848 | 11.037 | 27.432 | 1.00 | 36.32 | BTKA |
| ATOM | 299 | CB | MET | 437 | 29.146 | 12.191 | 26.467 | 1.00 | 35.52 | BTKA |
| ATOM | 300 | CG | MET | 437 | 28.879 | 13.578 | 27.063 | 1.00 | 36.44 | BTKA |
| ATOM | 301 | SD | MET | 437 | 27.262 | 13.773 | 27.876 | 1.00 | 30.54 | BTKA |
| ATOM | 302 | CE | MET | 437 | 26.468 | 14.965 | 26.768 | 1.00 | 30.24 | BTKA |
| ATOM | 303 | C | MET | 437 | 29.702 | 9.815 | 27.112 | 1.00 | 35.84 | BTKA |
| ATOM | 304 | O | MET | 437 | 30.916 | 9.829 | 27.323 | 1.00 | 33.00 | BTKA |
| ATOM | 305 | N | SER | 438 | 29.060 | 8.741 | 26.668 | 1.00 | 38.82 | BTKA |
| ATOM | 306 | CA | SER | 438 | 29.770 | 7.507 | 26.353 | 1.00 | 39.46 | BTKA |
| ATOM | 307 | CB | SER | 438 | 28.923 | 6.288 | 26.739 | 1.00 | 39.31 | BTKA |
| ATOM | 308 | OG | SER | 438 | 28.946 | 6.080 | 28.141 | 1.00 | 42.46 | BTKA |
| ATOM | 309 | C | SER | 438 | 30.291 | 7.349 | 24.928 | 1.00 | 37.92 | BTKA |
| ATOM | 310 | O | SER | 438 | 29.699 | 7.844 | 23.964 | 1.00 | 35.58 | BTKA |
| ATOM | 311 | N | GLU | 439 | 31.387 | 6.598 | 24.830 | 1.00 | 38.56 | BTKA |
| ATOM | 312 | CA | GLU | 439 | 32.072 | 6.281 | 23.579 | 1.00 | 37.22 | BTKA |
| ATOM | 313 | CB | GLU | 439 | 33.015 | 5.098 | 23.820 | 1.00 | 38.03 | BTKA |
| ATOM | 314 | CG | GLU | 439 | 33.578 | 4.444 | 22.567 | 1.00 | 39.09 | BTKA |
| ATOM | 315 | CD | GLU | 439 | 34.595 | 3.357 | 22.887 | 1.00 | 36.27 | BTKA |
| ATOM | 316 | OE1 | GLU | 439 | 34.313 | 2.514 | 23.771 | 1.00 | 36.28 | BTKA |
| ATOM | 317 | OE2 | GLU | 439 | 35.677 | 3.350 | 22.257 | 1.00 | 32.61 | BTKA |
| ATOM | 318 | C | GLU | 439 | 31.122 | 5.968 | 22.428 | 1.00 | 36.18 | BTKA |
| ATOM | 319 | O | GLU | 439 | 31.254 | 6.535 | 21.347 | 1.00 | 37.11 | BTKA |
| ATOM | 320 | N | ASP | 440 | 30.154 | 5.090 | 22.678 | 1.00 | 35.64 | BTKA |
| ATOM | 321 | CA | ASP | 440 | 29.172 | 4.692 | 21.665 | 1.00 | 36.29 | BTKA |
| ATOM | 322 | CB | ASP | 440 | 28.228 | 3.619 | 22.223 | 1.00 | 35.13 | BTKA |
| ATOM | 323 | CG | ASP | 440 | 28.822 | 2.223 | 22.157 | 1.00 | 33.91 | BTKA |
| ATOM | 324 | OD1 | ASP | 440 | 29.754 | 1.921 | 22.933 | 1.00 | 33.93 | BTKA |
| ATOM | 325 | OD2 | ASP | 440 | 28.342 | 1.425 | 21.328 | 1.00 | 33.14 | BTKA |
| ATOM | 326 | C | ASP | 440 | 28.343 | 5.838 | 21.089 | 1.00 | 35.32 | BTKA |
| ATOM | 327 | O | ASP | 440 | 28.079 | 5.876 | 19.884 | 1.00 | 31.62 | BTKA |
| ATOM | 328 | N | GLU | 441 | 27.939 | 6.764 | 21.954 | 1.00 | 34.63 | BTKA |
| ATOM | 329 | CA | GLU | 441 | 27.120 | 7.905 | 21.546 | 1.00 | 33.66 | BTKA |
| ATOM | 330 | CB | GLU | 441 | 26.845 | 8.819 | 22.742 | 1.00 | 35.94 | BTKA |
| ATOM | 331 | CG | GLU | 441 | 26.422 | 8.150 | 24.039 | 1.00 | 35.11 | BTKA |
| ATOM | 332 | CD | GLU | 441 | 26.394 | 9.138 | 25.203 | 1.00 | 35.16 | BTKA |
| ATOM | 333 | OE1 | GLU | 441 | 26.838 | 10.292 | 25.024 | 1.00 | 36.46 | BTKA |
| ATOM | 334 | OE2 | GLU | 441 | 25.946 | 8.765 | 26.306 | 1.00 | 35.56 | BTKA |
| ATOM | 335 | C | GLU | 441 | 27.786 | 8.756 | 20.462 | 1.00 | 32.20 | BTKA |
| ATOM | 336 | O | GLU | 441 | 27.301 | 8.861 | 19.333 | 1.00 | 29.93 | BTKA |
| ATOM | 337 | N | PHE | 442 | 28.908 | 9.361 | 20.841 | 1.00 | 31.11 | BTKA |
| ATOM | 338 | CA | PHE | 442 | 29.683 | 10.253 | 19.987 | 1.00 | 29.28 | BTKA |
| ATOM | 339 | CB | PHE | 442 | 30.854 | 10.837 | 20.804 | 1.00 | 29.09 | BTKA |
| ATOM | 340 | CG | PHE | 442 | 31.563 | 11.990 | 20.135 | 1.00 | 31.98 | BTKA |
| ATOM | 341 | CD1 | PHE | 442 | 30.865 | 12.888 | 19.325 | 1.00 | 32.20 | BTKA |
| ATOM | 342 | CD2 | PHE | 442 | 32.932 | 12.173 | 20.308 | 1.00 | 30.79 | BTKA |
| ATOM | 343 | CE1 | PHE | 442 | 31.517 | 13.947 | 18.697 | 1.00 | 27.29 | BTKA |
| ATOM | 344 | CE2 | PHE | 442 | 33.592 | 13.229 | 19.684 | 1.00 | 29.97 | BTKA |
| ATOM | 345 | CZ | PHE | 442 | 32.879 | 14.117 | 18.876 | 1.00 | 27.41 | BTKA |
| ATOM | 346 | C | PHE | 442 | 30.182 | 9.638 | 18.671 | 1.00 | 27.25 | BTKA |
| ATOM | 347 | O | PHE | 442 | 30.363 | 10.351 | 17.688 | 1.00 | 26.62 | BTKA |
| ATOM | 348 | N | ILE | 443 | 30.377 | 8.325 | 18.646 | 1.00 | 26.46 | BTKA |
| ATOM | 349 | CA | ILE | 443 | 30.866 | 7.639 | 17.450 | 1.00 | 26.92 | BTKA |
| ATOM | 350 | CB | ILE | 443 | 30.753 | 6.097 | 17.608 | 1.00 | 27.02 | BTKA |
| ATOM | 351 | CG2 | ILE | 443 | 30.893 | 5.399 | 16.262 | 1.00 | 29.28 | BTKA |
| ATOM | 352 | CG1 | ILE | 443 | 31.808 | 5.586 | 18.602 | 1.00 | 26.98 | BTKA |
| ATOM | 353 | CD | ILE | 443 | 33.253 | 5.879 | 18.207 | 1.00 | 23.34 | BTKA |
| ATOM | 354 | C | ILE | 443 | 30.173 | 8.079 | 16.160 | 1.00 | 27.64 | BTKA |
| ATOM | 355 | O | ILE | 443 | 30.834 | 8.451 | 15.189 | 1.00 | 27.13 | BTKA |
| ATOM | 356 | N | GLU | 444 | 28.843 | 8.057 | 16.163 | 1.00 | 29.25 | BTKA |
| ATOM | 357 | CA | GLU | 444 | 28.061 | 8.449 | 14.992 | 1.00 | 30.51 | BTKA |
| ATOM | 358 | CB | GLU | 444 | 26.552 | 8.274 | 15.256 | 1.00 | 32.35 | BTKA |
| ATOM | 359 | CG | GLU | 444 | 26.053 | 8.917 | 16.553 | 1.00 | 35.88 | BTKA |
| ATOM | 360 | CD | GLU | 444 | 24.549 | 9.144 | 16.582 | 1.00 | 35.26 | BTKA |
| ATOM | 361 | OE1 | GLU | 444 | 24.116 | 10.292 | 16.319 | 1.00 | 33.49 | BTKA |
| ATOM | 362 | OE2 | GLU | 444 | 23.802 | 8.186 | 16.886 | 1.00 | 36.30 | BTKA |
| ATOM | 363 | C | GLU | 444 | 28.353 | 9.886 | 14.571 | 1.00 | 29.10 | BTKA |
| ATOM | 364 | O | GLU | 444 | 28.781 | 10.139 | 13.445 | 1.00 | 29.32 | BTKA |
| ATOM | 365 | N | GLU | 445 | 28.173 | 10.816 | 15.502 | 1.00 | 26.25 | BTKA |
| ATOM | 366 | CA | GLU | 445 | 28.384 | 12.226 | 15.229 | 1.00 | 25.93 | BTKA |
| ATOM | 367 | CB | GLU | 445 | 27.935 | 13.073 | 16.420 | 1.00 | 24.15 | BTKA |
| ATOM | 368 | CG | GLU | 445 | 26.435 | 12.964 | 16.694 | 1.00 | 25.82 | BTKA |
| ATOM | 369 | CD | GLU | 445 | 25.860 | 14.206 | 17.357 | 1.00 | 26.92 | BTKA |
| ATOM | 370 | OE1 | GLU | 445 | 25.282 | 14.077 | 18.454 | 1.00 | 21.98 | BTKA |
| ATOM | 371 | OE2 | GLU | 445 | 25.973 | 15.308 | 16.775 | 1.00 | 27.91 | BTKA |
| ATOM | 372 | C | GLU | 445 | 29.817 | 12.534 | 14.835 | 1.00 | 26.08 | BTKA |
| ATOM | 373 | O | GLU | 445 | 30.075 | 13.505 | 14.117 | 1.00 | 26.43 | BTKA |
| ATOM | 374 | N | ALA | 446 | 30.739 | 11.679 | 15.272 | 1.00 | 26.04 | BTKA |
| ATOM | 375 | CA | ALA | 446 | 32.152 | 11.831 | 14.949 | 1.00 | 24.80 | BTKA |
| ATOM | 376 | CB | ALA | 446 | 32.998 | 10.909 | 15.813 | 1.00 | 24.38 | BTKA |
| ATOM | 377 | C | ALA | 446 | 32.346 | 11.504 | 13.468 | 1.00 | 24.74 | BTKA |
| ATOM | 378 | O | ALA | 446 | 33.003 | 12.264 | 12.746 | 1.00 | 23.97 | BTKA |
| ATOM | 379 | N | LYS | 447 | 31.755 | 10.391 | 13.020 | 1.00 | 22.24 | BTKA |
| ATOM | 380 | CA | LYS | 447 | 31.841 | 9.971 | 11.620 | 1.00 | 20.97 | BTKA |
| ATOM | 381 | CB | LYS | 447 | 31.065 | 8.669 | 11.380 | 1.00 | 20.58 | BTKA |
| ATOM | 382 | CG | LYS | 447 | 31.559 | 7.450 | 12.143 | 1.00 | 22.18 | BTKA |
| ATOM | 383 | CD | LYS | 447 | 30.767 | 6.201 | 11.752 | 1.00 | 22.45 | BTKA |
| ATOM | 384 | CE | LYS | 447 | 30.957 | 5.063 | 12.752 | 1.00 | 23.27 | BTKA |
| ATOM | 385 | NZ | LYS | 447 | 32.367 | 4.595 | 12.843 | 1.00 | 25.50 | BTKA |
| ATOM | 386 | C | LYS | 447 | 31.223 | 11.066 | 10.762 | 1.00 | 20.28 | BTKA |
| ATOM | 387 | O | LYS | 447 | 31.770 | 11.457 | 9.727 | 1.00 | 18.27 | BTKA |
| ATOM | 388 | N | VAL | 448 | 30.068 | 11.544 | 11.210 | 1.00 | 22.03 | BTKA |
| ATOM | 389 | CA | VAL | 448 | 29.332 | 12.599 | 10.532 | 1.00 | 23.98 | BTKA |
| ATOM | 390 | CB | VAL | 448 | 27.996 | 12.881 | 11.266 | 1.00 | 26.86 | BTKA |
| ATOM | 391 | CG1 | VAL | 448 | 27.403 | 14.218 | 10.835 | 1.00 | 26.50 | BTKA |
| ATOM | 392 | CG2 | VAL | 448 | 27.013 | 11.760 | 10.976 | 1.00 | 24.51 | BTKA |
| ATOM | 393 | C | VAL | 448 | 30.154 | 13.886 | 10.382 | 1.00 | 23.84 | BTKA |
| ATOM | 394 | O | VAL | 448 | 30.177 | 14.479 | 9.304 | 1.00 | 22.58 | BTKA |
| ATOM | 395 | N | MET | 449 | 30.848 | 14.300 | 11.439 | 1.00 | 25.01 | BTKA |
| ATOM | 396 | CA | MET | 449 | 31.659 | 15.514 | 11.375 | 1.00 | 26.00 | BTKA |
| ATOM | 397 | CB | MET | 449 | 32.036 | 15.992 | 12.775 | 1.00 | 28.34 | BTKA |
| ATOM | 398 | CG | MET | 449 | 30.941 | 16.778 | 13.471 | 1.00 | 29.82 | BTKA |
| ATOM | 399 | SD | MET | 449 | 31.446 | 17.269 | 15.116 | 1.00 | 32.37 | BTKA |
| ATOM | 400 | CE | MET | 449 | 32.786 | 18.412 | 14.721 | 1.00 | 32.43 | BTKA |
| ATOM | 401 | C | MET | 449 | 32.910 | 15.374 | 10.514 | 1.00 | 26.88 | BTKA |
| ATOM | 402 | O | MET | 449 | 33.239 | 16.271 | 9.742 | 1.00 | 24.06 | BTKA |
| ATOM | 403 | N | MET | 450 | 33.610 | 14.254 | 10.645 | 1.00 | 29.26 | BTKA |
| ATOM | 404 | CA | MET | 450 | 34.811 | 14.023 | 9.849 | 1.00 | 31.06 | BTKA |
| ATOM | 405 | CB | MET | 450 | 35.558 | 12.780 | 10.336 | 1.00 | 34.52 | BTKA |
| ATOM | 406 | CG | MET | 450 | 36.186 | 12.976 | 11.709 | 1.00 | 35.20 | BTKA |
| ATOM | 407 | SD | MET | 450 | 37.355 | 11.695 | 12.173 | 1.00 | 38.54 | BTKA |
| ATOM | 408 | CE | MET | 450 | 36.287 | 10.551 | 13.085 | 1.00 | 33.93 | BTKA |
| ATOM | 409 | C | MET | 450 | 34.502 | 13.938 | 8.351 | 1.00 | 28.32 | BTKA |
| ATOM | 410 | O | MET | 450 | 35.390 | 14.091 | 7.517 | 1.00 | 27.65 | BTKA |
| ATOM | 411 | N | ASN | 451 | 33.244 | 13.680 | 8.007 | 1.00 | 27.43 | BTKA |
| ATOM | 412 | CA | ASN | 451 | 32.853 | 13.636 | 6.603 | 1.00 | 25.78 | BTKA |
| ATOM | 413 | CB | ASN | 451 | 31.515 | 12.912 | 6.417 | 1.00 | 25.17 | BTKA |
| ATOM | 414 | CG | ASN | 451 | 31.681 | 11.499 | 5.858 | 1.00 | 24.38 | BTKA |
| ATOM | 415 | OD1 | ASN | 451 | 30.703 | 10.778 | 5.673 | 1.00 | 24.24 | BTKA |
| ATOM | 416 | ND2 | ASN | 451 | 32.919 | 11.104 | 5.576 | 1.00 | 27.13 | BTKA |
| ATOM | 417 | C | ASN | 451 | 32.781 | 15.071 | 6.073 | 1.00 | 24.17 | BTKA |
| ATOM | 418 | O | ASN | 451 | 32.831 | 15.308 | 4.862 | 1.00 | 23.10 | BTKA |
| ATOM | 419 | N | LEU | 452 | 32.656 | 16.025 | 6.989 | 1.00 | 23.71 | BTKA |
| ATOM | 420 | CA | LEU | 452 | 32.616 | 17.436 | 6.627 | 1.00 | 21.99 | BTKA |
| ATOM | 421 | CB | LEU | 452 | 32.023 | 18.279 | 7.767 | 1.00 | 18.83 | BTKA |
| ATOM | 422 | CG | LEU | 452 | 30.602 | 18.795 | 7.552 | 1.00 | 14.55 | BTKA |
| ATOM | 423 | CD1 | LEU | 452 | 29.724 | 17.669 | 7.063 | 1.00 | 13.88 | BTKA |
| ATOM | 424 | CD2 | LEU | 452 | 30.064 | 19.402 | 8.828 | 1.00 | 16.37 | BTKA |
| ATOM | 425 | C | LEU | 452 | 34.055 | 17.854 | 6.387 | 1.00 | 22.15 | BTKA |
| ATOM | 426 | O | LEU | 452 | 34.966 | 17.356 | 7.041 | 1.00 | 26.28 | BTKA |
| ATOM | 427 | N | SER | 453 | 34.267 | 18.720 | 5.414 | 1.00 | 18.69 | BTKA |
| ATOM | 428 | CA | SER | 453 | 35.598 | 19.194 | 5.117 | 1.00 | 17.44 | BTKA |
| ATOM | 429 | CB | SER | 453 | 36.468 | 18.074 | 4.552 | 1.00 | 20.39 | BTKA |
| ATOM | 430 | OG | SER | 453 | 37.270 | 17.498 | 5.569 | 1.00 | 32.76 | BTKA |
| ATOM | 431 | C | SER | 453 | 35.551 | 20.344 | 4.147 | 1.00 | 15.35 | BTKA |
| ATOM | 432 | O | SER | 453 | 34.960 | 20.253 | 3.083 | 1.00 | 16.35 | BTKA |
| ATOM | 433 | N | HIS | 454 | 36.153 | 21.445 | 4.554 | 1.00 | 11.81 | BTKA |
| ATOM | 434 | CA | HIS | 454 | 36.240 | 22.647 | 3.753 | 1.00 | 9.84 | BTKA |
| ATOM | 435 | CB | HIS | 454 | 34.942 | 23.469 | 3.833 | 1.00 | 11.56 | BTKA |
| ATOM | 436 | CG | HIS | 454 | 34.869 | 24.572 | 2.821 | 1.00 | 5.59 | BTKA |
| ATOM | 437 | CD2 | HIS | 454 | 34.345 | 24.601 | 1.574 | 1.00 | 3.51 | BTKA |
| ATOM | 438 | ND1 | HIS | 454 | 35.448 | 25.806 | 3.020 | 1.00 | 2.00 | BTKA |
| ATOM | 439 | CE1 | HIS | 454 | 35.292 | 26.545 | 1.935 | 1.00 | 3.01 | BTKA |
| ATOM | 440 | NE2 | HIS | 454 | 34.625 | 25.837 | 1.044 | 1.00 | 4.91 | BTKA |
| ATOM | 441 | C | HIS | 454 | 37.361 | 23.382 | 4.449 | 1.00 | 9.19 | BTKA |
| ATOM | 442 | O | HIS | 454 | 37.456 | 23.331 | 5.676 | 1.00 | 8.32 | BTKA |
| ATOM | 443 | N | GLU | 455 | 38.232 | 24.028 | 3.682 | 1.00 | 10.17 | BTKA |
| ATOM | 444 | CA | GLU | 455 | 39.351 | 24.770 | 4.262 | 1.00 | 10.17 | BTKA |
| ATOM | 445 | CB | GLU | 455 | 40.280 | 25.267 | 3.154 | 1.00 | 13.02 | BTKA |
| ATOM | 446 | CG | GLU | 455 | 39.603 | 26.176 | 2.162 | 1.00 | 15.79 | BTKA |
| ATOM | 447 | CD | GLU | 455 | 40.544 | 26.677 | 1.091 | 1.00 | 20.82 | BTKA |
| ATOM | 448 | OE1 | GLU | 455 | 40.670 | 27.909 | 0.940 | 1.00 | 24.47 | BTKA |
| ATOM | 449 | OE2 | GLU | 455 | 41.145 | 25.840 | 0.387 | 1.00 | 24.08 | BTKA |
| ATOM | 450 | C | GLU | 455 | 38.913 | 25.952 | 5.141 | 1.00 | 12.87 | BTKA |
| ATOM | 451 | O | GLU | 455 | 39.748 | 26.617 | 5.762 | 1.00 | 14.07 | BTKA |
| ATOM | 452 | N | LYS | 456 | 37.611 | 26.234 | 5.163 | 1.00 | 8.19 | BTKA |
| ATOM | 453 | CA | LYS | 456 | 37.084 | 27.334 | 5.963 | 1.00 | 7.61 | BTKA |
| ATOM | 454 | CB | LYS | 456 | 36.272 | 28.282 | 5.078 | 1.00 | 6.39 | BTKA |
| ATOM | 455 | CG | LYS | 456 | 37.102 | 28.927 | 3.974 | 1.00 | 9.24 | BTKA |
| ATOM | 456 | CD | LYS | 456 | 38.167 | 29.879 | 4.537 | 1.00 | 11.93 | BTKA |
| ATOM | 457 | CE | LYS | 456 | 39.236 | 30.217 | 3.493 | 1.00 | 13.97 | BTKA |
| ATOM | 458 | NZ | LYS | 456 | 38.693 | 30.793 | 2.222 | 1.00 | 11.85 | BTKA |
| ATOM | 459 | C | LYS | 456 | 36.266 | 26.821 | 7.152 | 1.00 | 5.26 | BTKA |
| ATOM | 460 | O | LYS | 456 | 35.500 | 27.555 | 7.770 | 1.00 | 3.31 | BTKA |
| ATOM | 461 | N | LEU | 457 | 36.419 | 25.533 | 7.429 | 1.00 | 7.43 | BTKA |
| ATOM | 462 | CA | LEU | 457 | 35.747 | 24.870 | 8.542 | 1.00 | 7.04 | BTKA |
| ATOM | 463 | CB | LEU | 457 | 34.833 | 23.732 | 8.051 | 1.00 | 5.48 | BTKA |
| ATOM | 464 | CG | LEU | 457 | 33.423 | 24.015 | 7.512 | 1.00 | 12.38 | BTKA |
| ATOM | 465 | CD1 | LEU | 457 | 32.887 | 22.821 | 6.734 | 1.00 | 6.71 | BTKA |
| ATOM | 466 | CD2 | LEU | 457 | 32.491 | 24.355 | 8.648 | 1.00 | 2.00 | BTKA |
| ATOM | 467 | C | LEU | 457 | 36.886 | 24.267 | 9.335 | 1.00 | 5.80 | BTKA |
| ATOM | 468 | O | LEU | 457 | 37.797 | 23.684 | 8.756 | 1.00 | 8.77 | BTKA |
| ATOM | 469 | N | VAL | 458 | 36.897 | 24.497 | 10.638 | 1.00 | 5.47 | BTKA |
| ATOM | 470 | CA | VAL | 458 | 37.928 | 23.925 | 11.478 | 1.00 | 5.34 | BTKA |
| ATOM | 471 | CB | VAL | 458 | 37.817 | 24.471 | 12.907 | 1.00 | 5.74 | BTKA |
| ATOM | 472 | CG1 | VAL | 458 | 38.738 | 23.703 | 13.862 | 1.00 | 4.86 | BTKA |
| ATOM | 473 | CG2 | VAL | 458 | 38.169 | 25.956 | 12.901 | 1.00 | 2.00 | BTKA |
| ATOM | 474 | C | VAL | 458 | 37.645 | 22.427 | 11.404 | 1.00 | 9.05 | BTKA |
| ATOM | 475 | O | VAL | 458 | 36.517 | 21.990 | 11.660 | 1.00 | 8.51 | BTKA |
| ATOM | 476 | N | GLN | 459 | 38.640 | 21.670 | 10.947 | 1.00 | 12.50 | BTKA |
| ATOM | 477 | CA | GLN | 459 | 38.532 | 20.223 | 10.769 | 1.00 | 11.11 | BTKA |
| ATOM | 478 | CB | GLN | 459 | 39.567 | 19.716 | 9.751 | 1.00 | 12.18 | BTKA |
| ATOM | 479 | CG | GLN | 459 | 39.885 | 20.639 | 8.588 | 1.00 | 14.96 | BTKA |
| ATOM | 480 | CD | GLN | 459 | 38.883 | 20.575 | 7.462 | 1.00 | 16.30 | BTKA |
| ATOM | 481 | OE1 | GLN | 459 | 37.686 | 20.837 | 7.644 | 1.00 | 18.40 | BTKA |
| ATOM | 482 | NE2 | GLN | 459 | 39.375 | 20.255 | 6.265 | 1.00 | 15.08 | BTKA |
| ATOM | 483 | C | GLN | 459 | 38.737 | 19.413 | 12.034 | 1.00 | 10.27 | BTKA |
| ATOM | 484 | O | GLN | 459 | 39.607 | 19.710 | 12.855 | 1.00 | 10.07 | BTKA |
| ATOM | 485 | N | LEU | 460 | 37.964 | 18.345 | 12.143 | 1.00 | 11.55 | BTKA |
| ATOM | 486 | CA | LEU | 460 | 38.069 | 17.422 | 13.253 | 1.00 | 12.61 | BTKA |
| ATOM | 487 | CB | LEU | 460 | 36.702 | 16.791 | 13.552 | 1.00 | 10.46 | BTKA |
| ATOM | 488 | CG | LEU | 460 | 36.480 | 16.009 | 14.854 | 1.00 | 9.21 | BTKA |
| ATOM | 489 | CD1 | LEU | 460 | 35.616 | 14.793 | 14.552 | 1.00 | 10.03 | BTKA |
| ATOM | 490 | CD2 | LEU | 460 | 37.789 | 15.552 | 15.490 | 1.00 | 10.95 | BTKA |
| ATOM | 491 | C | LEU | 460 | 39.012 | 16.356 | 12.712 | 1.00 | 12.22 | BTKA |
| ATOM | 492 | O | LEU | 460 | 38.832 | 15.883 | 11.588 | 1.00 | 9.11 | BTKA |
| ATOM | 493 | N | TYR | 461 | 40.019 | 15.988 | 13.496 | 1.00 | 14.03 | BTKA |
| ATOM | 494 | CA | TYR | 461 | 40.970 | 14.972 | 13.063 | 1.00 | 17.98 | BTKA |
| ATOM | 495 | CB | TYR | 461 | 42.385 | 15.311 | 13.531 | 1.00 | 15.90 | BTKA |
| ATOM | 496 | CG | TYR | 461 | 42.994 | 16.477 | 12.797 | 1.00 | 14.99 | BTKA |
| ATOM | 497 | CD1 | TYR | 461 | 43.523 | 17.553 | 13.489 | 1.00 | 10.88 | BTKA |
| ATOM | 498 | CE1 | TYR | 461 | 44.047 | 18.645 | 12.821 | 1.00 | 18.55 | BTKA |
| ATOM | 499 | CD2 | TYR | 461 | 43.007 | 16.517 | 11.403 | 1.00 | 18.59 | BTKA |
| ATOM | 500 | CE2 | TYR | 461 | 43.532 | 17.611 | 10.720 | 1.00 | 18.77 | BTKA |
| ATOM | 501 | CZ | TYR | 461 | 44.046 | 18.673 | 11.439 | 1.00 | 18.48 | BTKA |
| ATOM | 502 | OH | TYR | 461 | 44.539 | 19.774 | 10.780 | 1.00 | 17.96 | BTKA |
| ATOM | 503 | C | TYR | 461 | 40.593 | 13.578 | 13.527 | 1.00 | 19.94 | BTKA |
| ATOM | 504 | O | TYR | 461 | 40.611 | 12.630 | 12.747 | 1.00 | 20.11 | BTKA |
| ATOM | 505 | N | GLY | 462 | 40.226 | 13.455 | 14.796 | 1.00 | 22.97 | BTKA |
| ATOM | 506 | CA | GLY | 462 | 39.872 | 12.155 | 15.319 | 1.00 | 21.20 | BTKA |
| ATOM | 507 | C | GLY | 462 | 39.280 | 12.224 | 16.705 | 1.00 | 19.72 | BTKA |
| ATOM | 508 | O | GLY | 462 | 39.205 | 13.291 | 17.320 | 1.00 | 15.82 | BTKA |
| ATOM | 509 | N | VAL | 463 | 38.841 | 11.070 | 17.186 | 1.00 | 17.64 | BTKA |
| ATOM | 510 | CA | VAL | 463 | 38.231 | 10.961 | 18.494 | 1.00 | 19.56 | BTKA |
| ATOM | 511 | CB | VAL | 463 | 36.778 | 10.448 | 18.397 | 1.00 | 14.65 | BTKA |
| ATOM | 512 | CG1 | VAL | 463 | 35.920 | 11.446 | 17.649 | 1.00 | 12.04 | BTKA |
| ATOM | 513 | CG2 | VAL | 463 | 36.737 | 9.090 | 17.715 | 1.00 | 16.09 | BTKA |
| ATOM | 514 | C | VAL | 463 | 39.036 | 10.004 | 19.345 | 1.00 | 22.19 | BTKA |
| ATOM | 515 | O | VAL | 463 | 39.563 | 9.008 | 18.851 | 1.00 | 26.83 | BTKA |
| ATOM | 516 | N | CYS | 464 | 39.170 | 10.340 | 20.617 | 1.00 | 26.12 | BTKA |
| ATOM | 517 | CA | CYS | 464 | 39.893 | 9.505 | 21.559 | 1.00 | 28.36 | BTKA |
| ATOM | 518 | CB | CYS | 464 | 41.077 | 10.279 | 22.153 | 1.00 | 28.93 | BTKA |
| ATOM | 519 | SG | CYS | 464 | 42.375 | 9.282 | 22.957 | 1.00 | 33.22 | BTKA |
| ATOM | 520 | C | CYS | 464 | 38.834 | 9.182 | 22.613 | 1.00 | 31.37 | BTKA |
| ATOM | 521 | O | CYS | 464 | 38.850 | 9.707 | 23.726 | 1.00 | 32.82 | BTKA |
| ATOM | 522 | N | THR | 465 | 37.859 | 8.377 | 22.204 | 1.00 | 32.79 | BTKA |
| ATOM | 523 | CA | THR | 465 | 36.759 | 7.968 | 23.071 | 1.00 | 33.01 | BTKA |
| ATOM | 524 | CB | THR | 465 | 35.521 | 7.569 | 22.227 | 1.00 | 31.07 | BTKA |
| ATOM | 525 | OG1 | THR | 465 | 35.870 | 6.490 | 21.350 | 1.00 | 30.45 | BTKA |
| ATOM | 526 | CG2 | THR | 465 | 35.026 | 8.741 | 21.397 | 1.00 | 28.93 | BTKA |
| ATOM | 527 | C | THR | 465 | 37.150 | 6.759 | 23.921 | 1.00 | 34.05 | BTKA |
| ATOM | 528 | O | THR | 465 | 36.283 | 6.010 | 24.378 | 1.00 | 31.83 | BTKA |
| ATOM | 529 | N | LYS | 466 | 38.447 | 6.571 | 24.138 | 1.00 | 35.70 | BTKA |
| ATOM | 530 | CA | LYS | 466 | 38.926 | 5.426 | 24.901 | 1.00 | 36.53 | BTKA |
| ATOM | 531 | CB | LYS | 466 | 40.447 | 5.308 | 24.793 | 1.00 | 35.48 | BTKA |
| ATOM | 532 | CG | LYS | 466 | 40.955 | 4.991 | 23.376 | 1.00 | 38.68 | BTKA |
| ATOM | 533 | CD | LYS | 466 | 40.542 | 3.589 | 22.894 | 1.00 | 39.29 | BTKA |
| ATOM | 534 | CE | LYS | 466 | 39.081 | 3.514 | 22.451 | 1.00 | 38.59 | BTKA |
| ATOM | 535 | NZ | LYS | 466 | 38.664 | 2.137 | 22.077 | 1.00 | 40.01 | BTKA |
| ATOM | 536 | C | LYS | 466 | 38.461 | 5.332 | 26.349 | 1.00 | 36.75 | BTKA |
| ATOM | 537 | O | LYS | 466 | 39.215 | 5.624 | 27.278 | 1.00 | 34.39 | BTKA |
| ATOM | 538 | N | GLN | 467 | 37.226 | 4.861 | 26.513 | 1.00 | 37.31 | BTKA |
| ATOM | 539 | CA | GLN | 467 | 36.588 | 4.671 | 27.809 | 1.00 | 38.68 | BTKA |
| ATOM | 540 | CB | GLN | 467 | 37.121 | 3.402 | 28.476 | 1.00 | 36.64 | BTKA |
| ATOM | 541 | CG | GLN | 467 | 36.249 | 2.166 | 28.246 | 1.00 | 35.25 | BTKA |
| ATOM | 542 | CD | GLN | 467 | 35.842 | 1.967 | 26.787 | 1.00 | 33.56 | BTKA |
| ATOM | 543 | OE1 | GLN | 467 | 36.529 | 2.412 | 25.864 | 1.00 | 32.22 | BTKA |
| ATOM | 544 | NE2 | GLN | 467 | 34.716 | 1.289 | 26.578 | 1.00 | 27.03 | BTKA |
| ATOM | 545 | C | GLN | 467 | 36.671 | 5.870 | 28.743 | 1.00 | 40.26 | BTKA |
| ATOM | 546 | O | GLN | 467 | 35.659 | 6.531 | 29.004 | 1.00 | 43.24 | BTKA |
| ATOM | 547 | N | ARG | 468 | 37.860 | 6.118 | 29.282 | 1.00 | 40.19 | BTKA |
| ATOM | 548 | CA | ARG | 468 | 38.082 | 7.248 | 30.173 | 1.00 | 38.09 | BTKA |
| ATOM | 549 | CB | ARG | 468 | 39.501 | 7.183 | 30.746 | 1.00 | 35.92 | BTKA |
| ATOM | 550 | CG | ARG | 468 | 39.801 | 5.921 | 31.551 | 1.00 | 30.86 | BTKA |
| ATOM | 551 | CD | ARG | 468 | 38.832 | 5.707 | 32.719 | 1.00 | 32.42 | BTKA |
| ATOM | 552 | NE | ARG | 468 | 37.508 | 5.243 | 32.293 | 1.00 | 33.04 | BTKA |
| ATOM | 553 | CZ | ARG | 468 | 37.243 | 4.020 | 31.837 | 1.00 | 35.40 | BTKA |
| ATOM | 554 | NH1 | ARG | 468 | 38.206 | 3.109 | 31.740 | 1.00 | 37.18 | BTKA |
| ATOM | 555 | NH2 | ARG | 468 | 36.010 | 3.707 | 31.465 | 1.00 | 35.12 | BTKA |
| ATOM | 556 | C | ARG | 468 | 37.861 | 8.506 | 29.328 | 1.00 | 37.39 | BTKA |
| ATOM | 557 | O | ARG | 468 | 37.805 | 8.408 | 28.105 | 1.00 | 37.02 | BTKA |
| ATOM | 558 | N | PRO | 469 | 37.755 | 9.695 | 29.961 | 1.00 | 37.24 | BTKA |
| ATOM | 559 | CD | PRO | 469 | 38.219 | 9.958 | 31.336 | 1.00 | 35.17 | BTKA |
| ATOM | 560 | CA | PRO | 469 | 37.532 | 10.971 | 29.266 | 1.00 | 36.80 | BTKA |
| ATOM | 561 | CB | PRO | 469 | 38.542 | 11.886 | 29.943 | 1.00 | 36.83 | BTKA |
| ATOM | 562 | CG | PRO | 469 | 38.390 | 11.487 | 31.363 | 1.00 | 35.94 | BTKA |
| ATOM | 563 | C | PRO | 469 | 37.672 | 10.973 | 27.742 | 1.00 | 34.06 | BTKA |
| ATOM | 564 | O | PRO | 469 | 38.733 | 10.653 | 27.196 | 1.00 | 32.99 | BTKA |
| ATOM | 565 | N | ILE | 470 | 36.569 | 11.286 | 27.066 | 1.00 | 33.42 | BTKA |
| ATOM | 566 | CA | ILE | 470 | 36.537 | 11.338 | 25.608 | 1.00 | 29.80 | BTKA |
| ATOM | 567 | CB | ILE | 470 | 35.096 | 11.277 | 25.068 | 1.00 | 28.92 | BTKA |
| ATOM | 568 | CG2 | ILE | 470 | 35.095 | 11.469 | 23.561 | 1.00 | 28.43 | BTKA |
| ATOM | 569 | CG1 | ILE | 470 | 34.434 | 9.950 | 25.447 | 1.00 | 32.53 | BTKA |
| ATOM | 570 | CD | ILE | 470 | 32.980 | 9.858 | 25.017 | 1.00 | 31.41 | BTKA |
| ATOM | 571 | C | ILE | 470 | 37.156 | 12.645 | 25.148 | 1.00 | 28.29 | BTKA |
| ATOM | 572 | O | ILE | 470 | 36.724 | 13.725 | 25.563 | 1.00 | 28.16 | BTKA |
| ATOM | 573 | N | PHE | 471 | 38.171 | 12.542 | 24.302 | 1.00 | 26.48 | BTKA |
| ATOM | 574 | CA | PHE | 471 | 38.848 | 13.718 | 23.782 | 1.00 | 23.24 | BTKA |
| ATOM | 575 | CB | PHE | 471 | 40.362 | 13.623 | 24.005 | 1.00 | 26.91 | BTKA |
| ATOM | 576 | CG | PHE | 471 | 40.753 | 12.964 | 25.294 | 1.00 | 33.25 | BTKA |
| ATOM | 577 | CD1 | PHE | 471 | 40.457 | 13.560 | 26.517 | 1.00 | 34.40 | BTKA |
| ATOM | 578 | CD2 | PHE | 471 | 41.419 | 11.742 | 25.286 | 1.00 | 33.15 | BTKA |
| ATOM | 579 | CE1 | PHE | 471 | 40.821 | 12.949 | 27.708 | 1.00 | 37.94 | BTKA |
| ATOM | 580 | CE2 | PHE | 471 | 41.787 | 11.120 | 26.474 | 1.00 | 37.98 | BTKA |
| ATOM | 581 | CZ | PHE | 471 | 41.489 | 11.723 | 27.687 | 1.00 | 38.55 | BTKA |
| ATOM | 582 | C | PHE | 471 | 38.589 | 13.837 | 22.289 | 1.00 | 19.98 | BTKA |
| ATOM | 583 | O | PHE | 471 | 38.237 | 12.862 | 21.614 | 1.00 | 18.06 | BTKA |
| ATOM | 584 | N | ILE | 472 | 38.796 | 15.038 | 21.776 | 1.00 | 15.89 | BTKA |
| ATOM | 585 | CA | ILE | 472 | 38.624 | 15.310 | 20.367 | 1.00 | 16.13 | BTKA |
| ATOM | 586 | CB | ILE | 472 | 37.214 | 15.928 | 20.068 | 1.00 | 19.20 | BTKA |
| ATOM | 587 | CG2 | ILE | 472 | 37.237 | 17.445 | 20.155 | 1.00 | 21.36 | BTKA |
| ATOM | 588 | CG1 | ILE | 472 | 36.746 | 15.519 | 18.672 | 1.00 | 22.75 | BTKA |
| ATOM | 589 | CD | ILE | 472 | 35.491 | 16.217 | 18.204 | 1.00 | 19.43 | BTKA |
| ATOM | 590 | C | ILE | 472 | 39.763 | 16.270 | 20.030 | 1.00 | 15.65 | BTKA |
| ATOM | 591 | O | ILE | 472 | 40.164 | 17.076 | 20.876 | 1.00 | 14.58 | BTKA |
| ATOM | 592 | N | ILE | 473 | 40.374 | 16.081 | 18.861 | 1.00 | 12.55 | BTKA |
| ATOM | 593 | CA | ILE | 473 | 41.460 | 16.941 | 18.410 | 1.00 | 8.29 | BTKA |
| ATOM | 594 | CB | ILE | 473 | 42.760 | 16.179 | 18.121 | 1.00 | 6.11 | BTKA |
| ATOM | 595 | CG2 | ILE | 473 | 43.512 | 15.930 | 19.394 | 1.00 | 8.19 | BTKA |
| ATOM | 596 | CG1 | ILE | 473 | 42.469 | 14.896 | 17.351 | 1.00 | 12.26 | BTKA |
| ATOM | 597 | CD | ILE | 473 | 43.722 | 14.129 | 16.949 | 1.00 | 12.28 | BTKA |
| ATOM | 598 | C | ILE | 473 | 41.023 | 17.624 | 17.140 | 1.00 | 10.43 | BTKA |
| ATOM | 599 | O | ILE | 473 | 40.655 | 16.974 | 16.163 | 1.00 | 10.61 | BTKA |
| ATOM | 600 | N | THR | 474 | 41.039 | 18.943 | 17.169 | 1.00 | 10.11 | BTKA |
| ATOM | 601 | CA | THR | 474 | 40.646 | 19.740 | 16.031 | 1.00 | 12.13 | BTKA |
| ATOM | 602 | CB | THR | 474 | 39.527 | 20.724 | 16.452 | 1.00 | 13.64 | BTKA |
| ATOM | 603 | OG1 | THR | 474 | 39.496 | 20.831 | 17.885 | 1.00 | 11.89 | BTKA |
| ATOM | 604 | CG2 | THR | 474 | 38.169 | 20.231 | 15.967 | 1.00 | 14.36 | BTKA |
| ATOM | 605 | C | THR | 474 | 41.897 | 20.487 | 15.586 | 1.00 | 12.41 | BTKA |
| ATOM | 606 | O | THR | 474 | 42.914 | 20.444 | 16.278 | 1.00 | 12.99 | BTKA |
| ATOM | 607 | N | GLU | 475 | 41.862 | 21.114 | 14.413 | 1.00 | 14.37 | BTKA |
| ATOM | 608 | CA | GLU | 475 | 43.032 | 21.851 | 13.951 | 1.00 | 14.93 | BTKA |
| ATOM | 609 | CB | GLU | 475 | 42.927 | 22.210 | 12.462 | 1.00 | 14.14 | BTKA |
| ATOM | 610 | CG | GLU | 475 | 41.841 | 23.163 | 12.075 | 1.00 | 18.34 | BTKA |
| ATOM | 611 | CD | GLU | 475 | 41.887 | 23.516 | 10.590 | 1.00 | 18.18 | BTKA |
| ATOM | 612 | OE1 | GLU | 475 | 42.827 | 24.212 | 10.143 | 1.00 | 19.64 | BTKA |
| ATOM | 613 | OE2 | GLU | 475 | 40.976 | 23.102 | 9.860 | 1.00 | 17.68 | BTKA |
| ATOM | 614 | C | GLU | 475 | 43.267 | 23.084 | 14.813 | 1.00 | 15.03 | BTKA |
| ATOM | 615 | O | GLU | 475 | 42.311 | 23.751 | 15.216 | 1.00 | 16.87 | BTKA |
| ATOM | 616 | N | TYR | 476 | 44.533 | 23.327 | 15.156 | 1.00 | 16.03 | BTKA |
| ATOM | 617 | CA | TYR | 476 | 44.938 | 24.462 | 15.985 | 1.00 | 12.78 | BTKA |
| ATOM | 618 | CB | TYR | 476 | 46.367 | 24.263 | 16.516 | 1.00 | 14.76 | BTKA |
| ATOM | 619 | CG | TYR | 476 | 46.849 | 25.389 | 17.397 | 1.00 | 17.47 | BTKA |
| ATOM | 620 | CD1 | TYR | 476 | 46.426 | 25.491 | 18.719 | 1.00 | 21.65 | BTKA |
| ATOM | 621 | CE1 | TYR | 476 | 46.806 | 26.566 | 19.519 | 1.00 | 18.11 | BTKA |
| ATOM | 622 | CD2 | TYR | 476 | 47.676 | 26.394 | 16.895 | 1.00 | 16.67 | BTKA |
| ATOM | 623 | CE2 | TYR | 476 | 48.059 | 27.477 | 17.690 | 1.00 | 15.07 | BTKA |
| ATOM | 624 | CZ | TYR | 476 | 47.617 | 27.555 | 19.000 | 1.00 | 20.25 | BTKA |
| ATOM | 625 | OH | TYR | 476 | 47.963 | 28.621 | 19.801 | 1.00 | 22.07 | BTKA |
| ATOM | 626 | C | TYR | 476 | 44.864 | 25.760 | 15.202 | 1.00 | 9.86 | BTKA |
| ATOM | 627 | O | TYR | 476 | 45.291 | 25.822 | 14.053 | 1.00 | 12.02 | BTKA |
| ATOM | 628 | N | MET | 477 | 44.341 | 26.798 | 15.850 | 1.00 | 12.43 | BTKA |
| ATOM | 629 | CA | MET | 477 | 44.180 | 28.122 | 15.248 | 1.00 | 10.82 | BTKA |
| ATOM | 630 | CB | MET | 477 | 42.690 | 28.470 | 15.173 | 1.00 | 13.68 | BTKA |
| ATOM | 631 | CG | MET | 477 | 41.854 | 27.466 | 14.373 | 1.00 | 12.55 | BTKA |
| ATOM | 632 | SD | MET | 477 | 42.224 | 27.500 | 12.622 | 1.00 | 8.47 | BTKA |
| ATOM | 633 | CE | MET | 477 | 41.240 | 28.862 | 12.158 | 1.00 | 8.85 | BTKA |
| ATOM | 634 | C | MET | 477 | 44.923 | 29.171 | 16.076 | 1.00 | 8.89 | BTKA |
| ATOM | 635 | O | MET | 477 | 44.402 | 29.665 | 17.076 | 1.00 | 10.46 | BTKA |
| ATOM | 636 | N | ALA | 478 | 46.112 | 29.547 | 15.613 | 1.00 | 8.05 | BTKA |
| ATOM | 637 | CA | ALA | 478 | 46.980 | 30.503 | 16.300 | 1.00 | 6.04 | BTKA |
| ATOM | 638 | CB | ALA | 478 | 48.218 | 30.762 | 15.478 | 1.00 | 6.02 | BTKA |
| ATOM | 639 | C | ALA | 478 | 46.437 | 31.822 | 16.827 | 1.00 | 7.42 | BTKA |
| ATOM | 640 | O | ALA | 478 | 46.895 | 32.297 | 17.862 | 1.00 | 9.51 | BTKA |
| ATOM | 641 | N | ASN | 479 | 45.508 | 32.452 | 16.127 | 1.00 | 8.70 | BTKA |
| ATOM | 642 | CA | ASN | 479 | 44.997 | 33.721 | 16.625 | 1.00 | 11.12 | BTKA |
| ATOM | 643 | CB | ASN | 479 | 44.736 | 34.705 | 15.482 | 1.00 | 10.56 | BTKA |
| ATOM | 644 | CG | ASN | 479 | 46.014 | 35.189 | 14.823 | 1.00 | 10.54 | BTKA |
| ATOM | 645 | OD1 | ASN | 479 | 46.162 | 35.116 | 13.607 | 1.00 | 14.22 | BTKA |
| ATOM | 646 | ND2 | ASN | 479 | 46.944 | 35.693 | 15.623 | 1.00 | 5.93 | BTKA |
| ATOM | 647 | C | ASN | 479 | 43.778 | 33.594 | 17.527 | 1.00 | 10.80 | BTKA |
| ATOM | 648 | O | ASN | 479 | 43.300 | 34.593 | 18.066 | 1.00 | 13.56 | BTKA |
| ATOM | 649 | N | GLY | 480 | 43.309 | 32.371 | 17.742 | 1.00 | 13.64 | BTKA |
| ATOM | 650 | CA | GLY | 480 | 42.153 | 32.165 | 18.600 | 1.00 | 18.12 | BTKA |
| ATOM | 651 | C | GLY | 480 | 40.822 | 32.436 | 17.919 | 1.00 | 18.79 | BTKA |
| ATOM | 652 | O | GLY | 480 | 40.670 | 32.194 | 16.723 | 1.00 | 18.15 | BTKA |
| ATOM | 653 | N | CYS | 481 | 39.844 | 32.921 | 18.679 | 1.00 | 20.27 | BTKA |
| ATOM | 654 | CA | CYS | 481 | 38.534 | 33.210 | 18.108 | 1.00 | 16.03 | BTKA |
| ATOM | 655 | CB | CYS | 481 | 37.429 | 33.058 | 19.155 | 1.00 | 12.24 | BTKA |
| ATOM | 656 | SG | CYS | 481 | 37.336 | 34.396 | 20.332 | 1.00 | 19.49 | BTKA |
| ATOM | 657 | C | CYS | 481 | 38.471 | 34.591 | 17.466 | 1.00 | 14.17 | BTKA |
| ATOM | 658 | O | CYS | 481 | 39.155 | 35.525 | 17.895 | 1.00 | 12.70 | BTKA |
| ATOM | 659 | N | LEU | 482 | 37.617 | 34.710 | 16.457 | 1.00 | 13.70 | BTKA |
| ATOM | 660 | CA | LEU | 482 | 37.419 | 35.940 | 15.711 | 1.00 | 14.10 | BTKA |
| ATOM | 661 | CB | LEU | 482 | 36.332 | 35.727 | 14.658 | 1.00 | 15.16 | BTKA |
| ATOM | 662 | CG | LEU | 482 | 35.978 | 36.880 | 13.718 | 1.00 | 15.54 | BTKA |
| ATOM | 663 | CD1 | LEU | 482 | 37.163 | 37.237 | 12.839 | 1.00 | 13.49 | BTKA |
| ATOM | 664 | CD2 | LEU | 482 | 34.792 | 36.478 | 12.866 | 1.00 | 11.61 | BTKA |
| ATOM | 665 | C | LEU | 482 | 37.042 | 37.100 | 16.620 | 1.00 | 13.30 | BTKA |
| ATOM | 666 | O | LEU | 482 | 37.436 | 38.235 | 16.371 | 1.00 | 11.50 | BTKA |
| ATOM | 667 | N | LEU | 483 | 36.283 | 36.816 | 17.674 | 1.00 | 14.77 | BTKA |
| ATOM | 668 | CA | LEU | 483 | 35.861 | 37.856 | 18.604 | 1.00 | 18.16 | BTKA |
| ATOM | 669 | CB | LEU | 483 | 34.949 | 37.266 | 19.682 | 1.00 | 16.94 | BTKA |
| ATOM | 670 | CG | LEU | 483 | 34.146 | 38.245 | 20.538 | 1.00 | 14.08 | BTKA |
| ATOM | 671 | CD1 | LEU | 483 | 33.408 | 39.261 | 19.667 | 1.00 | 16.88 | BTKA |
| ATOM | 672 | CD2 | LEU | 483 | 33.162 | 37.464 | 21.361 | 1.00 | 14.16 | BTKA |
| ATOM | 673 | C | LEU | 483 | 37.079 | 38.543 | 19.224 | 1.00 | 18.15 | BTKA |
| ATOM | 674 | O | LEU | 483 | 37.198 | 39.770 | 19.181 | 1.00 | 17.24 | BTKA |
| ATOM | 675 | N | ASN | 484 | 38.009 | 37.739 | 19.729 | 1.00 | 18.40 | BTKA |
| ATOM | 676 | CA | ASN | 484 | 39.238 | 38.239 | 20.340 | 1.00 | 17.40 | BTKA |
| ATOM | 677 | CB | ASN | 484 | 40.024 | 37.078 | 20.957 | 1.00 | 20.05 | BTKA |
| ATOM | 678 | CG | ASN | 484 | 41.426 | 37.474 | 21.390 | 1.00 | 22.96 | BTKA |
| ATOM | 679 | OD1 | ASN | 484 | 41.687 | 38.628 | 21.741 | 1.00 | 27.75 | BTKA |
| ATOM | 680 | ND2 | ASN | 484 | 42.338 | 36.511 | 21.374 | 1.00 | 26.58 | BTKA |
| ATOM | 681 | C | ASN | 484 | 40.086 | 38.925 | 19.286 | 1.00 | 16.06 | BTKA |
| ATOM | 682 | O | ASN | 484 | 40.682 | 39.971 | 19.535 | 1.00 | 22.07 | BTKA |
| ATOM | 683 | N | TYR | 485 | 40.101 | 38.350 | 18.092 | 1.00 | 13.87 | BTKA |
| ATOM | 684 | CA | TYR | 485 | 40.880 | 38.889 | 16.985 | 1.00 | 8.60 | BTKA |
| ATOM | 685 | CB | TYR | 485 | 40.775 | 37.944 | 15.791 | 1.00 | 5.86 | BTKA |
| ATOM | 686 | CG | TYR | 485 | 41.861 | 38.126 | 14.770 | 1.00 | 6.11 | BTKA |
| ATOM | 687 | CD1 | TYR | 485 | 43.186 | 37.815 | 15.073 | 1.00 | 8.26 | BTKA |
| ATOM | 688 | CE1 | TYR | 485 | 44.193 | 37.969 | 14.128 | 1.00 | 12.73 | BTKA |
| ATOM | 689 | CD2 | TYR | 485 | 41.568 | 38.600 | 13.492 | 1.00 | 11.03 | BTKA |
| ATOM | 690 | CE2 | TYR | 485 | 42.575 | 38.763 | 12.535 | 1.00 | 12.88 | BTKA |
| ATOM | 691 | CZ | TYR | 485 | 43.881 | 38.445 | 12.861 | 1.00 | 13.61 | BTKA |
| ATOM | 692 | OH | TYR | 485 | 44.873 | 38.605 | 11.920 | 1.00 | 19.94 | BTKA |
| ATOM | 693 | C | TYR | 485 | 40.432 | 40.296 | 16.587 | 1.00 | 9.48 | BTKA |
| ATOM | 694 | O | TYR | 485 | 41.250 | 41.203 | 16.394 | 1.00 | 8.68 | BTKA |
| ATOM | 695 | N | LEU | 486 | 39.121 | 40.472 | 16.516 | 1.00 | 8.14 | BTKA |
| ATOM | 696 | CA | LEU | 486 | 38.504 | 41.729 | 16.137 | 1.00 | 10.39 | BTKA |
| ATOM | 697 | CB | LEU | 486 | 36.996 | 41.521 | 15.971 | 1.00 | 7.77 | BTKA |
| ATOM | 698 | CG | LEU | 486 | 36.398 | 41.527 | 14.560 | 1.00 | 10.49 | BTKA |
| ATOM | 699 | CD1 | LEU | 486 | 37.380 | 41.001 | 13.537 | 1.00 | 13.85 | BTKA |
| ATOM | 700 | CD2 | LEU | 486 | 35.114 | 40.699 | 14.561 | 1.00 | 13.88 | BTKA |
| ATOM | 701 | C | LEU | 486 | 38.770 | 42.885 | 17.088 | 1.00 | 9.35 | BTKA |
| ATOM | 702 | O | LEU | 486 | 38.730 | 44.047 | 16.679 | 1.00 | 10.01 | BTKA |
| ATOM | 703 | N | ARG | 487 | 39.022 | 42.585 | 18.353 | 1.00 | 11.39 | BTKA |
| ATOM | 704 | CA | ARG | 487 | 39.269 | 43.634 | 19.337 | 1.00 | 13.42 | BTKA |
| ATOM | 705 | CB | ARG | 487 | 38.530 | 43.331 | 20.623 | 1.00 | 12.34 | BTKA |
| ATOM | 706 | CG | ARG | 487 | 37.048 | 43.342 | 20.498 | 1.00 | 6.56 | BTKA |
| ATOM | 707 | CD | ARG | 487 | 36.480 | 42.775 | 21.757 | 1.00 | 9.94 | BTKA |
| ATOM | 708 | NE | ARG | 487 | 35.036 | 42.696 | 21.683 | 1.00 | 10.94 | BTKA |
| ATOM | 709 | CZ | ARG | 487 | 34.323 | 41.676 | 22.134 | 1.00 | 8.89 | BTKA |
| ATOM | 710 | NH1 | ARG | 487 | 34.917 | 40.631 | 22.700 | 1.00 | 8.34 | BTKA |
| ATOM | 711 | NH2 | ARG | 487 | 33.004 | 41.715 | 22.036 | 1.00 | 6.96 | BTKA |
| ATOM | 712 | C | ARG | 487 | 40.718 | 43.882 | 19.691 | 1.00 | 15.16 | BTKA |
| ATOM | 713 | O | ARG | 487 | 41.069 | 45.007 | 20.043 | 1.00 | 15.60 | BTKA |
| ATOM | 714 | N | GLU | 488 | 41.542 | 42.835 | 19.625 | 1.00 | 21.25 | BTKA |
| ATOM | 715 | CA | GLU | 488 | 42.958 | 42.918 | 19.980 | 1.00 | 22.43 | BTKA |
| ATOM | 716 | CB | GLU | 488 | 43.576 | 41.513 | 20.072 | 1.00 | 20.93 | BTKA |
| ATOM | 717 | CG | GLU | 488 | 43.928 | 40.849 | 18.742 | 1.00 | 22.66 | BTKA |
| ATOM | 718 | CD | GLU | 488 | 44.657 | 39.525 | 18.926 | 1.00 | 25.51 | BTKA |
| ATOM | 719 | OE1 | GLU | 488 | 45.809 | 39.523 | 19.417 | 1.00 | 25.84 | BTKA |
| ATOM | 720 | OE2 | GLU | 488 | 44.072 | 38.476 | 18.590 | 1.00 | 29.76 | BTKA |
| ATOM | 721 | C | GLU | 488 | 43.813 | 43.825 | 19.095 | 1.00 | 24.56 | BTKA |
| ATOM | 722 | O | GLU | 488 | 43.294 | 44.710 | 18.418 | 1.00 | 24.48 | BTKA |
| ATOM | 723 | N | MET | 489 | 45.130 | 43.609 | 19.127 | 1.00 | 28.65 | BTKA |
| ATOM | 724 | CA | MET | 489 | 46.093 | 44.396 | 18.351 | 1.00 | 31.30 | BTKA |
| ATOM | 725 | CB | MET | 489 | 47.494 | 43.788 | 18.466 | 1.00 | 31.45 | BTKA |
| ATOM | 726 | CG | MET | 489 | 48.348 | 44.318 | 19.616 | 1.00 | 29.69 | BTKA |
| ATOM | 727 | SD | MET | 489 | 47.736 | 43.928 | 21.263 | 1.00 | 34.34 | BTKA |
| ATOM | 728 | CE | MET | 489 | 46.980 | 45.509 | 21.726 | 1.00 | 31.43 | BTKA |
| ATOM | 729 | C | MET | 489 | 45.736 | 44.554 | 16.880 | 1.00 | 31.31 | BTKA |
| ATOM | 730 | O | MET | 489 | 46.058 | 45.571 | 16.263 | 1.00 | 28.54 | BTKA |
| ATOM | 731 | N | ARG | 490 | 45.066 | 43.553 | 16.319 | 1.00 | 32.07 | BTKA |
| ATOM | 732 | CA | ARG | 490 | 44.671 | 43.593 | 14.914 | 1.00 | 31.59 | BTKA |
| ATOM | 733 | CB | ARG | 490 | 44.288 | 42.197 | 14.419 | 1.00 | 28.74 | BTKA |
| ATOM | 734 | CG | ARG | 490 | 45.454 | 41.270 | 14.138 | 1.00 | 27.20 | BTKA |
| ATOM | 735 | CD | ARG | 490 | 45.992 | 40.612 | 15.406 | 1.00 | 29.57 | BTKA |
| ATOM | 736 | NE | ARG | 490 | 47.097 | 41.349 | 16.016 | 1.00 | 30.05 | BTKA |
| ATOM | 737 | CZ | ARG | 490 | 48.120 | 40.767 | 16.631 | 1.00 | 25.67 | BTKA |
| ATOM | 738 | NH1 | ARG | 490 | 48.180 | 39.446 | 16.723 | 1.00 | 24.51 | BTKA |
| ATOM | 739 | NH2 | ARG | 490 | 49.098 | 41.505 | 17.129 | 1.00 | 26.01 | BTKA |
| ATOM | 740 | C | ARG | 490 | 43.524 | 44.567 | 14.632 | 1.00 | 31.84 | BTKA |
| ATOM | 741 | O | ARG | 490 | 43.181 | 44.797 | 13.475 | 1.00 | 31.98 | BTKA |
| ATOM | 742 | N | HIS | 491 | 42.977 | 45.166 | 15.688 | 1.00 | 31.55 | BTKA |
| ATOM | 743 | CA | HIS | 491 | 41.863 | 46.110 | 15.596 | 1.00 | 33.20 | BTKA |
| ATOM | 744 | CB | HIS | 491 | 41.770 | 46.937 | 16.885 | 1.00 | 33.15 | BTKA |
| ATOM | 745 | CG | HIS | 491 | 40.366 | 47.220 | 17.328 | 1.00 | 34.60 | BTKA |
| ATOM | 746 | CD2 | HIS | 491 | 39.877 | 48.096 | 18.237 | 1.00 | 34.07 | BTKA |
| ATOM | 747 | ND1 | HIS | 491 | 39.277 | 46.525 | 16.847 | 1.00 | 33.41 | BTKA |
| ATOM | 748 | CE1 | HIS | 491 | 38.180 | 46.956 | 17.444 | 1.00 | 31.28 | BTKA |
| ATOM | 749 | NE2 | HIS | 491 | 38.517 | 47.909 | 18.292 | 1.00 | 32.75 | BTKA |
| ATOM | 750 | C | HIS | 491 | 41.927 | 47.055 | 14.401 | 1.00 | 35.09 | BTKA |
| ATOM | 751 | O | HIS | 491 | 40.892 | 47.404 | 13.828 | 1.00 | 37.68 | BTKA |
| ATOM | 752 | N | ARG | 492 | 43.136 | 47.484 | 14.046 | 1.00 | 34.42 | BTKA |
| ATOM | 753 | CA | ARG | 492 | 43.341 | 48.393 | 12.922 | 1.00 | 31.97 | BTKA |
| ATOM | 754 | CB | ARG | 492 | 44.726 | 49.047 | 13.013 | 1.00 | 33.81 | BTKA |
| ATOM | 755 | CG | ARG | 492 | 44.925 | 49.990 | 14.200 | 1.00 | 34.48 | BTKA |
| ATOM | 756 | CD | ARG | 492 | 44.943 | 49.258 | 15.537 | 1.00 | 35.35 | BTKA |
| ATOM | 757 | NE | ARG | 492 | 46.024 | 48.277 | 15.623 | 1.00 | 37.69 | BTKA |
| ATOM | 758 | CZ | ARG | 492 | 47.317 | 48.584 | 15.713 | 1.00 | 41.57 | BTKA |
| ATOM | 759 | NH1 | ARG | 492 | 47.711 | 49.853 | 15.725 | 1.00 | 42.87 | BTKA |
| ATOM | 760 | NH2 | ARG | 492 | 48.223 | 47.619 | 15.805 | 1.00 | 41.91 | BTKA |
| ATOM | 761 | C | ARG | 492 | 43.208 | 47.640 | 11.600 | 1.00 | 29.95 | BTKA |
| ATOM | 762 | O | ARG | 492 | 44.205 | 47.236 | 11.004 | 1.00 | 30.14 | BTKA |
| ATOM | 763 | N | PHE | 493 | 41.972 | 47.420 | 11.169 | 1.00 | 25.31 | BTKA |
| ATOM | 764 | CA | PHE | 493 | 41.722 | 46.704 | 9.927 | 1.00 | 25.42 | BTKA |
| ATOM | 765 | CB | PHE | 493 | 40.492 | 45.790 | 10.069 | 1.00 | 21.25 | BTKA |
| ATOM | 766 | CG | PHE | 493 | 40.713 | 44.585 | 10.943 | 1.00 | 20.61 | BTKA |
| ATOM | 767 | CD1 | PHE | 493 | 41.466 | 43.508 | 10.488 | 1.00 | 19.35 | BTKA |
| ATOM | 768 | CD2 | PHE | 493 | 40.151 | 44.518 | 12.215 | 1.00 | 18.30 | BTKA |
| ATOM | 769 | CE1 | PHE | 493 | 41.656 | 42.382 | 11.290 | 1.00 | 19.86 | BTKA |
| ATOM | 770 | CE2 | PHE | 493 | 40.335 | 43.396 | 13.024 | 1.00 | 18.27 | BTKA |
| ATOM | 771 | CZ | PHE | 493 | 41.084 | 42.328 | 12.565 | 1.00 | 14.30 | BTKA |
| ATOM | 772 | C | PHE | 493 | 41.532 | 47.663 | 8.751 | 1.00 | 28.44 | BTKA |
| ATOM | 773 | O | PHE | 493 | 42.235 | 48.669 | 8.625 | 1.00 | 28.08 | BTKA |
| ATOM | 774 | N | GLN | 494 | 40.585 | 47.315 | 7.884 | 1.00 | 28.76 | BTKA |
| ATOM | 775 | CA | GLN | 494 | 40.222 | 48.074 | 6.695 | 1.00 | 25.62 | BTKA |
| ATOM | 776 | CB | GLN | 494 | 41.401 | 48.139 | 5.723 | 1.00 | 26.48 | BTKA |
| ATOM | 777 | CG | GLN | 494 | 41.889 | 46.775 | 5.258 | 1.00 | 31.44 | BTKA |
| ATOM | 778 | CD | GLN | 494 | 43.164 | 46.832 | 4.437 | 1.00 | 31.26 | BTKA |
| ATOM | 779 | OE1 | GLN | 494 | 43.411 | 45.961 | 3.598 | 1.00 | 31.88 | BTKA |
| ATOM | 780 | NE2 | GLN | 494 | 43.989 | 47.846 | 4.679 | 1.00 | 31.84 | BTKA |
| ATOM | 781 | C | GLN | 494 | 39.082 | 47.251 | 6.098 | 1.00 | 23.81 | BTKA |
| ATOM | 782 | O | GLN | 494 | 39.051 | 46.032 | 6.270 | 1.00 | 22.09 | BTKA |
| ATOM | 783 | N | THR | 495 | 38.140 | 47.908 | 5.429 | 1.00 | 24.19 | BTKA |
| ATOM | 784 | CA | THR | 495 | 36.991 | 47.241 | 4.820 | 1.00 | 21.26 | BTKA |
| ATOM | 785 | CB | THR | 495 | 36.291 | 48.166 | 3.820 | 1.00 | 24.52 | BTKA |
| ATOM | 786 | OG1 | THR | 495 | 36.242 | 49.495 | 4.352 | 1.00 | 28.48 | BTKA |
| ATOM | 787 | CG2 | THR | 495 | 34.875 | 47.661 | 3.529 | 1.00 | 24.14 | BTKA |
| ATOM | 788 | C | THR | 495 | 37.376 | 45.978 | 4.061 | 1.00 | 18.91 | BTKA |
| ATOM | 789 | O | THR | 495 | 36.650 | 44.982 | 4.081 | 1.00 | 19.68 | BTKA |
| ATOM | 790 | N | GLN | 496 | 38.528 | 46.030 | 3.400 | 1.00 | 19.97 | BTKA |
| ATOM | 791 | CA | GLN | 496 | 39.026 | 44.915 | 2.612 | 1.00 | 19.83 | BTKA |
| ATOM | 792 | CB | GLN | 496 | 40.354 | 45.289 | 1.954 | 1.00 | 18.70 | BTKA |
| ATOM | 793 | CG | GLN | 496 | 40.198 | 46.264 | 0.789 | 1.00 | 23.77 | BTKA |
| ATOM | 794 | CD | GLN | 496 | 39.583 | 47.592 | 1.201 | 1.00 | 26.56 | BTKA |
| ATOM | 795 | OE1 | GLN | 496 | 39.831 | 48.089 | 2.301 | 1.00 | 27.00 | BTKA |
| ATOM | 796 | NE2 | GLN | 496 | 38.762 | 48.166 | 0.328 | 1.00 | 24.89 | BTKA |
| ATOM | 797 | C | GLN | 496 | 39.162 | 43.639 | 3.431 | 1.00 | 18.82 | BTKA |
| ATOM | 798 | O | GLN | 496 | 38.719 | 42.570 | 3.001 | 1.00 | 18.80 | BTKA |
| ATOM | 799 | N | GLN | 497 | 39.716 | 43.773 | 4.634 | 1.00 | 18.01 | BTKA |
| ATOM | 800 | CA | GLN | 497 | 39.910 | 42.644 | 5.542 | 1.00 | 16.28 | BTKA |
| ATOM | 801 | CB | GLN | 497 | 40.942 | 43.003 | 6.613 | 1.00 | 15.98 | BTKA |
| ATOM | 802 | CG | GLN | 497 | 42.329 | 43.311 | 6.059 | 1.00 | 16.87 | BTKA |
| ATOM | 803 | CD | GLN | 497 | 43.258 | 43.930 | 7.096 | 1.00 | 21.37 | BTKA |
| ATOM | 804 | OE1 | GLN | 497 | 43.364 | 45.153 | 7.202 | 1.00 | 21.47 | BTKA |
| ATOM | 805 | NE2 | GLN | 497 | 43.936 | 43.087 | 7.864 | 1.00 | 22.57 | BTKA |
| ATOM | 806 | C | GLN | 497 | 38.586 | 42.243 | 6.198 | 1.00 | 12.86 | BTKA |
| ATOM | 807 | O | GLN | 497 | 38.377 | 41.085 | 6.546 | 1.00 | 10.95 | BTKA |
| ATOM | 808 | N | LEU | 498 | 37.683 | 43.202 | 6.342 | 1.00 | 10.21 | BTKA |
| ATOM | 809 | CA | LEU | 498 | 36.388 | 42.924 | 6.944 | 1.00 | 9.62 | BTKA |
| ATOM | 810 | CB | LEU | 498 | 35.682 | 44.230 | 7.306 | 1.00 | 10.33 | BTKA |
| ATOM | 811 | CG | LEU | 498 | 35.894 | 44.842 | 8.700 | 1.00 | 8.10 | BTKA |
| ATOM | 812 | CD1 | LEU | 498 | 37.228 | 44.470 | 9.307 | 1.00 | 11.10 | BTKA |
| ATOM | 813 | CD2 | LEU | 498 | 35.750 | 46.335 | 8.611 | 1.00 | 6.67 | BTKA |
| ATOM | 814 | C | LEU | 498 | 35.535 | 42.078 | 6.004 | 1.00 | 10.86 | BTKA |
| ATOM | 815 | O | LEU | 498 | 34.959 | 41.070 | 6.420 | 1.00 | 12.75 | BTKA |
| ATOM | 816 | N | LEU | 499 | 35.514 | 42.429 | 4.722 | 1.00 | 9.42 | BTKA |
| ATOM | 817 | CA | LEU | 499 | 34.725 | 41.661 | 3.764 | 1.00 | 11.87 | BTKA |
| ATOM | 818 | CB | LEU | 499 | 34.588 | 42.401 | 2.424 | 1.00 | 8.90 | BTKA |
| ATOM | 819 | CG | LEU | 499 | 33.332 | 42.005 | 1.634 | 1.00 | 9.18 | BTKA |
| ATOM | 820 | CD1 | LEU | 499 | 32.100 | 42.507 | 2.347 | 1.00 | 4.94 | BTKA |
| ATOM | 821 | CD2 | LEU | 499 | 33.371 | 42.551 | 0.226 | 1.00 | 9.87 | BTKA |
| ATOM | 822 | C | LEU | 499 | 35.327 | 40.270 | 3.548 | 1.00 | 12.95 | BTKA |
| ATOM | 823 | O | LEU | 499 | 34.592 | 39.287 | 3.410 | 1.00 | 16.98 | BTKA |
| ATOM | 824 | N | GLU | 500 | 36.658 | 40.190 | 3.515 | 1.00 | 13.94 | BTKA |
| ATOM | 825 | CA | GLU | 500 | 37.369 | 38.922 | 3.327 | 1.00 | 13.24 | BTKA |
| ATOM | 826 | CB | GLU | 500 | 38.887 | 39.150 | 3.415 | 1.00 | 16.16 | BTKA |
| ATOM | 827 | CG | GLU | 500 | 39.771 | 37.887 | 3.353 | 1.00 | 20.06 | BTKA |
| ATOM | 828 | CD | GLU | 500 | 39.721 | 37.146 | 2.016 | 1.00 | 22.89 | BTKA |
| ATOM | 829 | OE1 | GLU | 500 | 39.841 | 35.901 | 2.032 | 1.00 | 26.56 | BTKA |
| ATOM | 830 | OE2 | GLU | 500 | 39.571 | 37.791 | 0.953 | 1.00 | 24.13 | BTKA |
| ATOM | 831 | C | GLU | 500 | 36.923 | 37.909 | 4.377 | 1.00 | 10.91 | BTKA |
| ATOM | 832 | O | GLU | 500 | 36.671 | 36.739 | 4.068 | 1.00 | 10.95 | BTKA |
| ATOM | 833 | N | MET | 501 | 36.795 | 38.377 | 5.614 | 1.00 | 11.96 | BTKA |
| ATOM | 834 | CA | MET | 501 | 36.365 | 37.533 | 6.722 | 1.00 | 11.06 | BTKA |
| ATOM | 835 | CB | MET | 501 | 36.449 | 38.289 | 8.047 | 1.00 | 10.47 | BTKA |
| ATOM | 836 | CG | MET | 501 | 37.854 | 38.714 | 8.415 | 1.00 | 13.77 | BTKA |
| ATOM | 837 | SD | MET | 501 | 37.954 | 39.544 | 10.013 | 1.00 | 20.98 | BTKA |
| ATOM | 838 | CE | MET | 501 | 39.741 | 39.784 | 10.118 | 1.00 | 14.07 | BTKA |
| ATOM | 839 | C | MET | 501 | 34.947 | 37.032 | 6.503 | 1.00 | 9.64 | BTKA |
| ATOM | 840 | O | MET | 501 | 34.661 | 35.876 | 6.772 | 1.00 | 8.63 | BTKA |
| ATOM | 841 | N | CYS | 502 | 34.057 | 37.897 | 6.027 | 1.00 | 11.41 | BTKA |
| ATOM | 842 | CA | CYS | 502 | 32.676 | 37.493 | 5.768 | 1.00 | 13.88 | BTKA |
| ATOM | 843 | CB | CYS | 502 | 31.827 | 38.688 | 5.321 | 1.00 | 16.90 | BTKA |
| ATOM | 844 | SG | CYS | 502 | 31.751 | 40.046 | 6.496 | 1.00 | 14.48 | BTKA |
| ATOM | 845 | C | CYS | 502 | 32.642 | 36.410 | 4.688 | 1.00 | 12.88 | BTKA |
| ATOM | 846 | O | CYS | 502 | 31.844 | 35.473 | 4.763 | 1.00 | 14.89 | BTKA |
| ATOM | 847 | N | LYS | 503 | 33.508 | 36.552 | 3.688 | 1.00 | 13.23 | BTKA |
| ATOM | 848 | CA | LYS | 503 | 33.622 | 35.597 | 2.586 | 1.00 | 10.87 | BTKA |
| ATOM | 849 | CB | LYS | 503 | 34.606 | 36.132 | 1.539 | 1.00 | 12.02 | BTKA |
| ATOM | 850 | CG | LYS | 503 | 34.948 | 35.161 | 0.415 | 1.00 | 11.71 | BTKA |
| ATOM | 851 | CD | LYS | 503 | 35.937 | 35.789 | −0.550 | 1.00 | 17.80 | BTKA |
| ATOM | 852 | CE | LYS | 503 | 36.351 | 34.811 | −1.638 | 1.00 | 18.39 | BTKA |
| ATOM | 853 | NZ | LYS | 503 | 37.022 | 35.506 | −2.763 | 1.00 | 19.21 | BTKA |
| ATOM | 854 | C | LYS | 503 | 34.082 | 34.224 | 3.095 | 1.00 | 10.24 | BTKA |
| ATOM | 855 | O | LYS | 503 | 33.471 | 33.203 | 2.765 | 1.00 | 9.05 | BTKA |
| ATOM | 856 | N | ASP | 504 | 35.131 | 34.204 | 3.921 | 1.00 | 10.68 | BTKA |
| ATOM | 857 | CA | ASP | 504 | 35.653 | 32.954 | 4.486 | 1.00 | 7.72 | BTKA |
| ATOM | 858 | CB | ASP | 504 | 36.748 | 33.229 | 5.530 | 1.00 | 8.01 | BTKA |
| ATOM | 859 | CG | ASP | 504 | 37.947 | 33.983 | 4.972 | 1.00 | 12.07 | BTKA |
| ATOM | 860 | OD1 | ASP | 504 | 38.633 | 34.654 | 5.773 | 1.00 | 7.24 | BTKA |
| ATOM | 861 | OD2 | ASP | 504 | 38.225 | 33.906 | 3.756 | 1.00 | 18.62 | BTKA |
| ATOM | 862 | C | ASP | 504 | 34.538 | 32.178 | 5.185 | 1.00 | 7.40 | BTKA |
| ATOM | 863 | O | ASP | 504 | 34.425 | 30.957 | 5.041 | 1.00 | 7.01 | BTKA |
| ATOM | 864 | N | VAL | 505 | 33.740 | 32.896 | 5.971 | 1.00 | 8.06 | BTKA |
| ATOM | 865 | CA | VAL | 505 | 32.640 | 32.302 | 6.726 | 1.00 | 6.77 | BTKA |
| ATOM | 866 | CB | VAL | 505 | 32.097 | 33.280 | 7.799 | 1.00 | 7.29 | BTKA |
| ATOM | 867 | CG1 | VAL | 505 | 31.036 | 32.590 | 8.662 | 1.00 | 6.82 | BTKA |
| ATOM | 868 | CG2 | VAL | 505 | 33.230 | 33.779 | 8.673 | 1.00 | 10.15 | BTKA |
| ATOM | 869 | C | VAL | 505 | 31.507 | 31.854 | 5.809 | 1.00 | 4.31 | BTKA |
| ATOM | 870 | O | VAL | 505 | 30.924 | 30.787 | 6.008 | 1.00 | 4.27 | BTKA |
| ATOM | 871 | N | CYS | 506 | 31.201 | 32.660 | 4.803 | 1.00 | 7.86 | BTKA |
| ATOM | 872 | CA | CYS | 506 | 30.144 | 32.331 | 3.847 | 1.00 | 8.57 | BTKA |
| ATOM | 873 | CB | CYS | 506 | 29.838 | 33.529 | 2.950 | 1.00 | 8.83 | BTKA |
| ATOM | 874 | SG | CYS | 506 | 28.173 | 33.501 | 2.267 | 1.00 | 9.16 | BTKA |
| ATOM | 875 | C | CYS | 506 | 30.531 | 31.120 | 2.987 | 1.00 | 8.76 | BTKA |
| ATOM | 876 | O | CYS | 506 | 29.668 | 30.406 | 2.479 | 1.00 | 8.42 | BTKA |
| ATOM | 877 | N | GLU | 507 | 31.831 | 30.904 | 2.810 | 1.00 | 9.14 | BTKA |
| ATOM | 878 | CA | GLU | 507 | 32.307 | 29.765 | 2.044 | 1.00 | 8.13 | BTKA |
| ATOM | 879 | CB | GLU | 507 | 33.786 | 29.915 | 1.707 | 1.00 | 7.62 | BTKA |
| ATOM | 880 | CG | GLU | 507 | 34.049 | 30.890 | 0.567 | 1.00 | 10.42 | BTKA |
| ATOM | 881 | CD | GLU | 507 | 35.508 | 30.972 | 0.200 | 1.00 | 8.86 | BTKA |
| ATOM | 882 | OE1 | GLU | 507 | 35.807 | 31.383 | −0.932 | 1.00 | 14.46 | BTKA |
| ATOM | 883 | OE2 | GLU | 507 | 36.364 | 30.625 | 1.039 | 1.00 | 11.94 | BTKA |
| ATOM | 884 | C | GLU | 507 | 32.079 | 28.493 | 2.836 | 1.00 | 4.79 | BTKA |
| ATOM | 885 | O | GLU | 507 | 31.679 | 27.467 | 2.290 | 1.00 | 5.50 | BTKA |
| ATOM | 886 | N | ALA | 508 | 32.331 | 28.560 | 4.133 | 1.00 | 4.69 | BTKA |
| ATOM | 887 | CA | ALA | 508 | 32.132 | 27.411 | 5.003 | 1.00 | 2.84 | BTKA |
| ATOM | 888 | CB | ALA | 508 | 32.819 | 27.647 | 6.328 | 1.00 | 3.35 | BTKA |
| ATOM | 889 | C | ALA | 508 | 30.640 | 27.148 | 5.217 | 1.00 | 2.47 | BTKA |
| ATOM | 890 | O | ALA | 508 | 30.208 | 26.006 | 5.312 | 1.00 | 3.01 | BTKA |
| ATOM | 891 | N | MET | 509 | 29.851 | 28.214 | 5.261 | 1.00 | 4.27 | BTKA |
| ATOM | 892 | CA | MET | 509 | 28.415 | 28.096 | 5.459 | 1.00 | 3.24 | BTKA |
| ATOM | 893 | CB | MET | 509 | 27.829 | 29.434 | 5.905 | 1.00 | 2.63 | BTKA |
| ATOM | 894 | CG | MET | 509 | 28.209 | 29.825 | 7.328 | 1.00 | 5.18 | BTKA |
| ATOM | 895 | SD | MET | 509 | 27.764 | 28.538 | 8.519 | 1.00 | 12.83 | BTKA |
| ATOM | 896 | CE | MET | 509 | 29.227 | 28.333 | 9.323 | 1.00 | 11.58 | BTKA |
| ATOM | 897 | C | MET | 509 | 27.699 | 27.571 | 4.224 | 1.00 | 4.52 | BTKA |
| ATOM | 898 | O | MET | 509 | 26.794 | 26.748 | 4.333 | 1.00 | 8.63 | BTKA |
| ATOM | 899 | N | GLU | 510 | 28.094 | 28.057 | 3.050 | 1.00 | 5.93 | BTKA |
| ATOM | 900 | CA | GLU | 510 | 27.504 | 27.606 | 1.788 | 1.00 | 7.82 | BTKA |
| ATOM | 901 | CB | GLU | 510 | 28.167 | 28.327 | 0.615 | 1.00 | 7.50 | BTKA |
| ATOM | 902 | CG | GLU | 510 | 27.557 | 28.018 | −0.737 | 1.00 | 18.01 | BTKA |
| ATOM | 903 | CD | GLU | 510 | 28.399 | 28.535 | −1.898 | 1.00 | 21.81 | BTKA |
| ATOM | 904 | OE1 | GLU | 510 | 27.811 | 28.919 | −2.933 | 1.00 | 27.32 | BTKA |
| ATOM | 905 | OE2 | GLU | 510 | 29.645 | 28.544 | −1.781 | 1.00 | 24.20 | BTKA |
| ATOM | 906 | C | GLU | 510 | 27.719 | 26.090 | 1.671 | 1.00 | 5.58 | BTKA |
| ATOM | 907 | O | GLU | 510 | 26.811 | 25.347 | 1.292 | 1.00 | 5.09 | BTKA |
| ATOM | 908 | N | TYR | 511 | 28.918 | 25.643 | 2.049 | 1.00 | 7.55 | BTKA |
| ATOM | 909 | CA | TYR | 511 | 29.269 | 24.229 | 2.031 | 1.00 | 6.74 | BTKA |
| ATOM | 910 | CB | TYR | 511 | 30.748 | 24.049 | 2.400 | 1.00 | 9.67 | BTKA |
| ATOM | 911 | CG | TYR | 511 | 31.172 | 22.598 | 2.511 | 1.00 | 14.31 | BTKA |
| ATOM | 912 | CD1 | TYR | 511 | 31.217 | 21.774 | 1.386 | 1.00 | 12.15 | BTKA |
| ATOM | 913 | CE1 | TYR | 511 | 31.526 | 20.423 | 1.498 | 1.00 | 11.02 | BTKA |
| ATOM | 914 | CD2 | TYR | 511 | 31.459 | 22.032 | 3.751 | 1.00 | 13.80 | BTKA |
| ATOM | 915 | CE2 | TYR | 511 | 31.768 | 20.685 | 3.868 | 1.00 | 12.93 | BTKA |
| ATOM | 916 | CZ | TYR | 511 | 31.796 | 19.886 | 2.739 | 1.00 | 11.33 | BTKA |
| ATOM | 917 | OH | TYR | 511 | 32.057 | 18.545 | 2.875 | 1.00 | 14.81 | BTKA |
| ATOM | 918 | C | TYR | 511 | 28.368 | 23.405 | 2.975 | 1.00 | 8.27 | BTKA |
| ATOM | 919 | O | TYR | 511 | 27.916 | 22.316 | 2.611 | 1.00 | 8.84 | BTKA |
| ATOM | 920 | N | LEU | 512 | 28.121 | 23.907 | 4.184 | 1.00 | 8.72 | BTKA |
| ATOM | 921 | CA | LEU | 512 | 27.267 | 23.188 | 5.131 | 1.00 | 10.18 | BTKA |
| ATOM | 922 | CB | LEU | 512 | 27.232 | 23.878 | 6.503 | 1.00 | 4.04 | BTKA |
| ATOM | 923 | CG | LEU | 512 | 28.534 | 23.878 | 7.307 | 1.00 | 3.83 | BTKA |
| ATOM | 924 | CD1 | LEU | 512 | 28.300 | 24.439 | 8.703 | 1.00 | 2.00 | BTKA |
| ATOM | 925 | CD2 | LEU | 512 | 29.073 | 22.449 | 7.386 | 1.00 | 2.00 | BTKA |
| ATOM | 926 | C | LEU | 512 | 25.859 | 23.061 | 4.579 | 1.00 | 12.90 | BTKA |
| ATOM | 927 | O | LEU | 512 | 25.251 | 21.991 | 4.639 | 1.00 | 12.40 | BTKA |
| ATOM | 928 | N | GLU | 513 | 25.359 | 24.156 | 4.012 | 1.00 | 13.36 | BTKA |
| ATOM | 929 | CA | GLU | 513 | 24.028 | 24.197 | 3.428 | 1.00 | 11.85 | BTKA |
| ATOM | 930 | CB | GLU | 513 | 23.777 | 25.584 | 2.831 | 1.00 | 13.01 | BTKA |
| ATOM | 931 | CG | GLU | 513 | 22.368 | 25.822 | 2.284 | 1.00 | 10.82 | BTKA |
| ATOM | 932 | CD | GLU | 513 | 22.151 | 27.264 | 1.828 | 1.00 | 11.92 | BTKA |
| ATOM | 933 | OE1 | GLU | 513 | 22.923 | 27.742 | 0.960 | 1.00 | 14.37 | BTKA |
| ATOM | 934 | OE2 | GLU | 513 | 21.208 | 27.919 | 2.338 | 1.00 | 10.65 | BTKA |
| ATOM | 935 | C | GLU | 513 | 23.852 | 23.101 | 2.370 | 1.00 | 11.40 | BTKA |
| ATOM | 936 | O | GLU | 513 | 22.850 | 22.389 | 2.375 | 1.00 | 9.38 | BTKA |
| ATOM | 937 | N | SER | 514 | 24.848 | 22.932 | 1.502 | 1.00 | 13.91 | BTKA |
| ATOM | 938 | CA | SER | 514 | 24.785 | 21.915 | 0.453 | 1.00 | 15.43 | BTKA |
| ATOM | 939 | CB | SER | 514 | 25.916 | 22.109 | −0.561 | 1.00 | 15.20 | BTKA |
| ATOM | 940 | OG | SER | 514 | 27.179 | 22.170 | 0.076 | 1.00 | 13.45 | BTKA |
| ATOM | 941 | C | SER | 514 | 24.834 | 20.500 | 1.024 | 1.00 | 17.44 | BTKA |
| ATOM | 942 | O | SER | 514 | 24.500 | 19.528 | 0.343 | 1.00 | 15.53 | BTKA |
| ATOM | 943 | N | LYS | 515 | 25.278 | 20.391 | 2.271 | 1.00 | 17.30 | BTKA |
| ATOM | 944 | CA | LYS | 515 | 25.371 | 19.107 | 2.943 | 1.00 | 12.99 | BTKA |
| ATOM | 945 | CB | LYS | 515 | 26.740 | 18.968 | 3.618 | 1.00 | 13.16 | BTKA |
| ATOM | 946 | CG | LYS | 515 | 27.920 | 18.997 | 2.643 | 1.00 | 13.40 | BTKA |
| ATOM | 947 | CD | LYS | 515 | 27.911 | 17.783 | 1.725 | 1.00 | 10.90 | BTKA |
| ATOM | 948 | CE | LYS | 515 | 29.045 | 17.846 | 0.731 | 1.00 | 9.83 | BTKA |
| ATOM | 949 | NZ | LYS | 515 | 29.193 | 16.567 | −0.031 | 1.00 | 11.46 | BTKA |
| ATOM | 950 | C | LYS | 515 | 24.256 | 18.942 | 3.973 | 1.00 | 13.83 | BTKA |
| ATOM | 951 | O | LYS | 515 | 24.292 | 18.024 | 4.780 | 1.00 | 15.25 | BTKA |
| ATOM | 952 | N | GLN | 516 | 23.265 | 19.831 | 3.928 | 1.00 | 13.54 | BTKA |
| ATOM | 953 | CA | GLN | 516 | 22.120 | 19.814 | 4.848 | 1.00 | 15.50 | BTKA |
| ATOM | 954 | CB | GLN | 516 | 21.149 | 18.679 | 4.498 | 1.00 | 20.10 | BTKA |
| ATOM | 955 | CG | GLN | 516 | 20.481 | 18.812 | 3.128 | 1.00 | 27.83 | BTKA |
| ATOM | 956 | CD | GLN | 516 | 21.461 | 18.701 | 1.976 | 1.00 | 30.31 | BTKA |
| ATOM | 957 | OE1 | GLN | 516 | 21.517 | 19.568 | 1.107 | 1.00 | 31.11 | BTKA |
| ATOM | 958 | NE2 | GLN | 516 | 22.246 | 17.634 | 1.968 | 1.00 | 32.70 | BTKA |
| ATOM | 959 | C | GLN | 516 | 22.547 | 19.738 | 6.313 | 1.00 | 14.29 | BTKA |
| ATOM | 960 | O | GLN | 516 | 21.888 | 19.123 | 7.155 | 1.00 | 10.17 | BTKA |
| ATOM | 961 | N | PHE | 517 | 23.674 | 20.362 | 6.608 | 1.00 | 15.64 | BTKA |
| ATOM | 962 | CA | PHE | 517 | 24.192 | 20.388 | 7.963 | 1.00 | 16.92 | BTKA |
| ATOM | 963 | CB | PHE | 517 | 25.668 | 20.004 | 7.974 | 1.00 | 13.86 | BTKA |
| ATOM | 964 | CG | PHE | 517 | 26.230 | 19.814 | 9.349 | 1.00 | 14.42 | BTKA |
| ATOM | 965 | CD1 | PHE | 517 | 26.109 | 18.589 | 9.996 | 1.00 | 20.02 | BTKA |
| ATOM | 966 | CD2 | PHE | 517 | 26.888 | 20.851 | 9.994 | 1.00 | 13.85 | BTKA |
| ATOM | 967 | CE1 | PHE | 517 | 26.639 | 18.398 | 11.267 | 1.00 | 22.95 | BTKA |
| ATOM | 968 | CE2 | PHE | 517 | 27.421 | 20.673 | 11.262 | 1.00 | 18.84 | BTKA |
| ATOM | 969 | CZ | PHE | 517 | 27.297 | 19.442 | 11.901 | 1.00 | 23.12 | BTKA |
| ATOM | 970 | C | PHE | 517 | 24.022 | 21.814 | 8.458 | 1.00 | 19.46 | BTKA |
| ATOM | 971 | O | PHE | 517 | 24.510 | 22.758 | 7.830 | 1.00 | 20.11 | BTKA |
| ATOM | 972 | N | LEU | 518 | 23.293 | 21.986 | 9.551 | 1.00 | 18.01 | BTKA |
| ATOM | 973 | CA | LEU | 518 | 23.091 | 23.323 | 10.069 | 1.00 | 17.59 | BTKA |
| ATOM | 974 | CB | LEU | 518 | 21.604 | 23.598 | 10.328 | 1.00 | 15.17 | BTKA |
| ATOM | 975 | CG | LEU | 518 | 20.968 | 23.286 | 11.679 | 1.00 | 15.72 | BTKA |
| ATOM | 976 | CD1 | LEU | 518 | 20.619 | 24.599 | 12.368 | 1.00 | 18.80 | BTKA |
| ATOM | 977 | CD2 | LEU | 518 | 19.717 | 22.461 | 11.477 | 1.00 | 16.60 | BTKA |
| ATOM | 978 | C | LEU | 518 | 23.926 | 23.569 | 11.310 | 1.00 | 14.94 | BTKA |
| ATOM | 979 | O | LEU | 518 | 24.198 | 22.660 | 12.086 | 1.00 | 14.17 | BTKA |
| ATOM | 980 | N | HIS | 519 | 24.395 | 24.801 | 11.424 | 1.00 | 15.04 | BTKA |
| ATOM | 981 | CA | HIS | 519 | 25.205 | 25.286 | 12.538 | 1.00 | 15.75 | BTKA |
| ATOM | 982 | CB | HIS | 519 | 26.276 | 26.213 | 11.970 | 1.00 | 13.52 | BTKA |
| ATOM | 983 | CG | HIS | 519 | 27.416 | 26.469 | 12.894 | 1.00 | 8.55 | BTKA |
| ATOM | 984 | CD2 | HIS | 519 | 28.744 | 26.566 | 12.651 | 1.00 | 4.28 | BTKA |
| ATOM | 985 | ND1 | HIS | 519 | 27.261 | 26.635 | 14.250 | 1.00 | 2.10 | BTKA |
| ATOM | 986 | CE1 | HIS | 519 | 28.444 | 26.816 | 14.806 | 1.00 | 8.67 | BTKA |
| ATOM | 987 | NE2 | HIS | 519 | 29.360 | 26.779 | 13.858 | 1.00 | 4.20 | BTKA |
| ATOM | 988 | C | HIS | 519 | 24.226 | 26.090 | 13.405 | 1.00 | 17.47 | BTKA |
| ATOM | 989 | O | HIS | 519 | 23.929 | 27.239 | 13.093 | 1.00 | 21.50 | BTKA |
| ATOM | 990 | N | ARG | 520 | 23.774 | 25.510 | 14.511 | 1.00 | 15.16 | BTKA |
| ATOM | 991 | CA | ARG | 520 | 22.785 | 26.156 | 15.382 | 1.00 | 15.58 | BTKA |
| ATOM | 992 | CB | ARG | 520 | 22.065 | 25.114 | 16.245 | 1.00 | 15.27 | BTKA |
| ATOM | 993 | CG | ARG | 520 | 21.823 | 23.780 | 15.555 | 1.00 | 18.29 | BTKA |
| ATOM | 994 | CD | ARG | 520 | 20.966 | 22.857 | 16.392 | 1.00 | 20.10 | BTKA |
| ATOM | 995 | NE | ARG | 520 | 21.449 | 22.725 | 17.769 | 1.00 | 27.27 | BTKA |
| ATOM | 996 | CZ | ARG | 520 | 22.581 | 22.124 | 18.127 | 1.00 | 27.89 | BTKA |
| ATOM | 997 | NH1 | ARG | 520 | 23.378 | 21.592 | 17.215 | 1.00 | 27.36 | BTKA |
| ATOM | 998 | NH2 | ARG | 520 | 22.904 | 22.038 | 19.408 | 1.00 | 25.47 | BTKA |
| ATOM | 999 | C | ARG | 520 | 23.294 | 27.261 | 16.290 | 1.00 | 16.19 | BTKA |
| ATOM | 1000 | O | ARG | 520 | 22.536 | 27.798 | 17.095 | 1.00 | 16.18 | BTKA |
| ATOM | 1001 | N | ASP | 521 | 24.557 | 27.635 | 16.141 | 1.00 | 16.78 | BTKA |
| ATOM | 1002 | CA | ASP | 521 | 25.128 | 28.669 | 16.992 | 1.00 | 14.05 | BTKA |
| ATOM | 1003 | CB | ASP | 521 | 25.668 | 28.023 | 18.278 | 1.00 | 14.95 | BTKA |
| ATOM | 1004 | CG | ASP | 521 | 26.067 | 29.042 | 19.330 | 1.00 | 13.62 | BTKA |
| ATOM | 1005 | OD1 | ASP | 521 | 25.579 | 30.194 | 19.278 | 1.00 | 13.41 | BTKA |
| ATOM | 1006 | OD2 | ASP | 521 | 26.875 | 28.681 | 20.209 | 1.00 | 8.70 | BTKA |
| ATOM | 1007 | C | ASP | 521 | 26.235 | 29.425 | 16.269 | 1.00 | 11.05 | BTKA |
| ATOM | 1008 | O | ASP | 521 | 27.409 | 29.277 | 16.583 | 1.00 | 10.26 | BTKA |
| ATOM | 1009 | N | LEU | 522 | 25.871 | 30.188 | 15.251 | 1.00 | 12.24 | BTKA |
| ATOM | 1010 | CA | LEU | 522 | 26.868 | 30.949 | 14.511 | 1.00 | 11.62 | BTKA |
| ATOM | 1011 | CB | LEU | 522 | 26.451 | 31.112 | 13.049 | 1.00 | 11.48 | BTKA |
| ATOM | 1012 | CG | LEU | 522 | 27.453 | 30.814 | 11.932 | 1.00 | 8.77 | BTKA |
| ATOM | 1013 | CD1 | LEU | 522 | 26.970 | 31.571 | 10.728 | 1.00 | 8.36 | BTKA |
| ATOM | 1014 | CD2 | LEU | 522 | 28.891 | 31.232 | 12.269 | 1.00 | 10.90 | BTKA |
| ATOM | 1015 | C | LEU | 522 | 27.017 | 32.325 | 15.149 | 1.00 | 12.66 | BTKA |
| ATOM | 1016 | O | LEU | 522 | 26.043 | 33.058 | 15.286 | 1.00 | 14.12 | BTKA |
| ATOM | 1017 | N | ALA | 523 | 28.237 | 32.657 | 15.544 | 1.00 | 13.37 | BTKA |
| ATOM | 1018 | CA | ALA | 523 | 28.546 | 33.941 | 16.157 | 1.00 | 13.35 | BTKA |
| ATOM | 1019 | CB | ALA | 523 | 28.169 | 33.934 | 17.639 | 1.00 | 6.92 | BTKA |
| ATOM | 1020 | C | ALA | 523 | 30.050 | 34.109 | 15.989 | 1.00 | 13.67 | BTKA |
| ATOM | 1021 | O | ALA | 523 | 30.743 | 33.138 | 15.661 | 1.00 | 14.16 | BTKA |
| ATOM | 1022 | N | ALA | 524 | 30.552 | 35.326 | 16.193 | 1.00 | 11.77 | BTKA |
| ATOM | 1023 | CA | ALA | 524 | 31.980 | 35.597 | 16.062 | 1.00 | 12.32 | BTKA |
| ATOM | 1024 | CB | ALA | 524 | 32.262 | 37.083 | 16.210 | 1.00 | 10.35 | BTKA |
| ATOM | 1025 | C | ALA | 524 | 32.771 | 34.809 | 17.091 | 1.00 | 13.86 | BTKA |
| ATOM | 1026 | O | ALA | 524 | 33.936 | 34.468 | 16.856 | 1.00 | 17.70 | BTKA |
| ATOM | 1027 | N | ARG | 525 | 32.143 | 34.541 | 18.237 | 1.00 | 13.11 | BTKA |
| ATOM | 1028 | CA | ARG | 525 | 32.779 | 33.780 | 19.302 | 1.00 | 13.31 | BTKA |
| ATOM | 1029 | CB | ARG | 525 | 31.873 | 33.692 | 20.533 | 1.00 | 14.94 | BTKA |
| ATOM | 1030 | CG | ARG | 525 | 30.736 | 32.687 | 20.422 | 1.00 | 16.14 | BTKA |
| ATOM | 1031 | CD | ARG | 525 | 30.031 | 32.492 | 21.751 | 1.00 | 13.88 | BTKA |
| ATOM | 1032 | NE | ARG | 525 | 29.478 | 33.746 | 22.255 | 1.00 | 21.28 | BTKA |
| ATOM | 1033 | CZ | ARG | 525 | 28.257 | 34.205 | 21.989 | 1.00 | 22.82 | BTKA |
| ATOM | 1034 | NH1 | ARG | 525 | 27.425 | 33.522 | 21.210 | 1.00 | 24.36 | BTKA |
| ATOM | 1035 | NH2 | ARG | 525 | 27.859 | 35.343 | 22.536 | 1.00 | 21.29 | BTKA |
| ATOM | 1036 | C | ARG | 525 | 33.112 | 32.369 | 18.833 | 1.00 | 13.03 | BTKA |
| ATOM | 1037 | O | ARG | 525 | 34.027 | 31.750 | 19.360 | 1.00 | 12.07 | BTKA |
| ATOM | 1038 | N | ASN | 526 | 32.361 | 31.873 | 17.849 | 1.00 | 11.96 | BTKA |
| ATOM | 1039 | CA | ASN | 526 | 32.559 | 30.533 | 17.314 | 1.00 | 10.29 | BTKA |
| ATOM | 1040 | CB | ASN | 526 | 31.225 | 29.871 | 16.985 | 1.00 | 8.11 | BTKA |
| ATOM | 1041 | CG | ASN | 526 | 30.715 | 29.039 | 18.119 | 1.00 | 9.90 | BTKA |
| ATOM | 1042 | OD1 | ASN | 526 | 31.187 | 29.165 | 19.247 | 1.00 | 11.02 | BTKA |
| ATOM | 1043 | ND2 | ASN | 526 | 29.761 | 28.167 | 17.836 | 1.00 | 15.50 | BTKA |
| ATOM | 1044 | C | ASN | 526 | 33.475 | 30.422 | 16.111 | 1.00 | 8.66 | BTKA |
| ATOM | 1045 | O | ASN | 526 | 33.639 | 29.341 | 15.560 | 1.00 | 10.60 | BTKA |
| ATOM | 1046 | N | CYS | 527 | 34.056 | 31.526 | 15.678 | 1.00 | 6.87 | BTKA |
| ATOM | 1047 | CA | CYS | 527 | 34.963 | 31.476 | 14.541 | 1.00 | 8.50 | BTKA |
| ATOM | 1048 | CB | CYS | 527 | 34.656 | 32.610 | 13.558 | 1.00 | 10.13 | BTKA |
| ATOM | 1049 | SG | CYS | 527 | 33.099 | 32.432 | 12.694 | 1.00 | 7.24 | BTKA |
| ATOM | 1050 | C | CYS | 527 | 36.377 | 31.610 | 15.081 | 1.00 | 9.42 | BTKA |
| ATOM | 1051 | O | CYS | 527 | 36.583 | 32.269 | 16.093 | 1.00 | 10.96 | BTKA |
| ATOM | 1052 | N | LEU | 528 | 37.331 | 30.948 | 14.439 | 1.00 | 9.95 | BTKA |
| ATOM | 1053 | CA | LEU | 528 | 38.732 | 30.998 | 14.854 | 1.00 | 7.80 | BTKA |
| ATOM | 1054 | CB | LEU | 528 | 39.222 | 29.602 | 15.262 | 1.00 | 8.09 | BTKA |
| ATOM | 1055 | CG | LEU | 528 | 38.507 | 28.876 | 16.411 | 1.00 | 4.85 | BTKA |
| ATOM | 1056 | CD1 | LEU | 528 | 39.088 | 27.501 | 16.633 | 1.00 | 5.23 | BTKA |
| ATOM | 1057 | CD2 | LEU | 528 | 38.623 | 29.682 | 17.687 | 1.00 | 12.77 | BTKA |
| ATOM | 1058 | C | LEU | 528 | 39.560 | 31.539 | 13.693 | 1.00 | 7.58 | BTKA |
| ATOM | 1059 | O | LEU | 528 | 39.130 | 31.474 | 12.533 | 1.00 | 8.42 | BTKA |
| ATOM | 1060 | N | VAL | 529 | 40.725 | 32.100 | 14.010 | 1.00 | 9.03 | BTKA |
| ATOM | 1061 | CA | VAL | 529 | 41.631 | 32.674 | 13.013 | 1.00 | 9.97 | BTKA |
| ATOM | 1062 | CB | VAL | 529 | 41.836 | 34.180 | 13.268 | 1.00 | 4.59 | BTKA |
| ATOM | 1063 | CG1 | VAL | 529 | 42.609 | 34.802 | 12.122 | 1.00 | 3.83 | BTKA |
| ATOM | 1064 | CG2 | VAL | 529 | 40.513 | 34.871 | 13.483 | 1.00 | 3.81 | BTKA |
| ATOM | 1065 | C | VAL | 529 | 43.003 | 32.008 | 13.107 | 1.00 | 11.90 | BTKA |
| ATOM | 1066 | O | VAL | 529 | 43.581 | 31.952 | 14.193 | 1.00 | 16.82 | BTKA |
| ATOM | 1067 | N | ASN | 530 | 43.524 | 31.492 | 11.993 | 1.00 | 15.54 | BTKA |
| ATOM | 1068 | CA | ASN | 530 | 44.839 | 30.849 | 12.016 | 1.00 | 16.52 | BTKA |
| ATOM | 1069 | CB | ASN | 530 | 44.975 | 29.783 | 10.918 | 1.00 | 15.67 | BTKA |
| ATOM | 1070 | CG | ASN | 530 | 44.676 | 30.312 | 9.512 | 1.00 | 17.17 | BTKA |
| ATOM | 1071 | OD1 | ASN | 530 | 44.972 | 31.462 | 9.168 | 1.00 | 11.18 | BTKA |
| ATOM | 1072 | ND2 | ASN | 530 | 44.111 | 29.448 | 8.683 | 1.00 | 15.71 | BTKA |
| ATOM | 1073 | C | ASN | 530 | 45.959 | 31.886 | 11.945 | 1.00 | 18.49 | BTKA |
| ATOM | 1074 | O | ASN | 530 | 45.688 | 33.074 | 12.064 | 1.00 | 23.10 | BTKA |
| ATOM | 1075 | N | ASP | 531 | 47.208 | 31.454 | 11.768 | 1.00 | 20.52 | BTKA |
| ATOM | 1076 | CA | ASP | 531 | 48.329 | 32.397 | 11.708 | 1.00 | 22.28 | BTKA |
| ATOM | 1077 | CB | ASP | 531 | 49.662 | 31.695 | 12.018 | 1.00 | 24.91 | BTKA |
| ATOM | 1078 | CG | ASP | 531 | 50.626 | 32.581 | 12.834 | 1.00 | 28.03 | BTKA |
| ATOM | 1079 | OD1 | ASP | 531 | 50.883 | 32.262 | 14.019 | 1.00 | 25.89 | BTKA |
| ATOM | 1080 | OD2 | ASP | 531 | 51.130 | 33.595 | 12.298 | 1.00 | 27.88 | BTKA |
| ATOM | 1081 | C | ASP | 531 | 48.403 | 33.141 | 10.370 | 1.00 | 24.22 | BTKA |
| ATOM | 1082 | O | ASP | 531 | 49.089 | 34.162 | 10.253 | 1.00 | 26.63 | BTKA |
| ATOM | 1083 | N | GLN | 532 | 47.693 | 32.636 | 9.365 | 1.00 | 22.72 | BTKA |
| ATOM | 1084 | CA | GLN | 532 | 47.663 | 33.279 | 8.052 | 1.00 | 21.96 | BTKA |
| ATOM | 1085 | CB | GLN | 532 | 47.438 | 32.255 | 6.933 | 1.00 | 25.06 | BTKA |
| ATOM | 1086 | CG | GLN | 532 | 48.588 | 31.275 | 6.710 | 1.00 | 29.08 | BTKA |
| ATOM | 1087 | CD | GLN | 532 | 48.653 | 30.189 | 7.765 | 1.00 | 32.01 | BTKA |
| ATOM | 1088 | OE1 | GLN | 532 | 49.302 | 30.346 | 8.801 | 1.00 | 31.49 | BTKA |
| ATOM | 1089 | NE2 | GLN | 532 | 47.971 | 29.080 | 7.509 | 1.00 | 34.18 | BTKA |
| ATOM | 1090 | C | GLN | 532 | 46.556 | 34.333 | 8.009 | 1.00 | 20.47 | BTKA |
| ATOM | 1091 | O | GLN | 532 | 46.376 | 35.018 | 6.998 | 1.00 | 19.50 | BTKA |
| ATOM | 1092 | N | GLY | 533 | 45.814 | 34.448 | 9.109 | 1.00 | 19.48 | BTKA |
| ATOM | 1093 | CA | GLY | 533 | 44.734 | 35.409 | 9.181 | 1.00 | 17.27 | BTKA |
| ATOM | 1094 | C | GLY | 533 | 43.434 | 34.858 | 8.632 | 1.00 | 16.09 | BTKA |
| ATOM | 1095 | O | GLY | 533 | 42.411 | 35.549 | 8.624 | 1.00 | 15.63 | BTKA |
| ATOM | 1096 | N | VAL | 534 | 43.462 | 33.606 | 8.186 | 1.00 | 14.60 | BTKA |
| ATOM | 1097 | CA | VAL | 534 | 42.265 | 32.984 | 7.633 | 1.00 | 10.73 | BTKA |
| ATOM | 1098 | CB | VAL | 534 | 42.579 | 31.675 | 6.869 | 1.00 | 8.97 | BTKA |
| ATOM | 1099 | CG1 | VAL | 534 | 41.299 | 31.097 | 6.283 | 1.00 | 10.35 | BTKA |
| ATOM | 1100 | CG2 | VAL | 534 | 43.589 | 31.940 | 5.756 | 1.00 | 5.02 | BTKA |
| ATOM | 1101 | C | VAL | 534 | 41.257 | 32.719 | 8.744 | 1.00 | 6.19 | BTKA |
| ATOM | 1102 | O | VAL | 534 | 41.620 | 32.335 | 9.859 | 1.00 | 8.54 | BTKA |
| ATOM | 1103 | N | VAL | 535 | 39.998 | 32.994 | 8.440 | 1.00 | 7.49 | BTKA |
| ATOM | 1104 | CA | VAL | 535 | 38.915 | 32.820 | 9.385 | 1.00 | 6.02 | BTKA |
| ATOM | 1105 | CB | VAL | 535 | 37.918 | 34.028 | 9.338 | 1.00 | 5.47 | BTKA |
| ATOM | 1106 | CG1 | VAL | 535 | 36.737 | 33.808 | 10.281 | 1.00 | 2.00 | BTKA |
| ATOM | 1107 | CG2 | VAL | 535 | 38.632 | 35.308 | 9.725 | 1.00 | 4.76 | BTKA |
| ATOM | 1108 | C | VAL | 535 | 38.214 | 31.540 | 9.018 | 1.00 | 10.54 | BTKA |
| ATOM | 1109 | O | VAL | 535 | 37.929 | 31.283 | 7.840 | 1.00 | 11.45 | BTKA |
| ATOM | 1110 | N | LYS | 536 | 37.985 | 30.714 | 10.029 | 1.00 | 13.48 | BTKA |
| ATOM | 1111 | CA | LYS | 536 | 37.314 | 29.440 | 9.841 | 1.00 | 10.58 | BTKA |
| ATOM | 1112 | CB | LYS | 536 | 38.303 | 28.286 | 10.030 | 1.00 | 7.32 | BTKA |
| ATOM | 1113 | CG | LYS | 536 | 39.494 | 28.342 | 9.113 | 1.00 | 2.94 | BTKA |
| ATOM | 1114 | CD | LYS | 536 | 40.172 | 26.996 | 9.009 | 1.00 | 2.00 | BTKA |
| ATOM | 1115 | CE | LYS | 536 | 41.398 | 27.103 | 8.113 | 1.00 | 8.60 | BTKA |
| ATOM | 1116 | NZ | LYS | 536 | 41.911 | 25.779 | 7.679 | 1.00 | 8.95 | BTKA |
| ATOM | 1117 | C | LYS | 536 | 36.194 | 29.328 | 10.863 | 1.00 | 11.55 | BTKA |
| ATOM | 1118 | O | LYS | 536 | 36.291 | 29.859 | 11.972 | 1.00 | 13.24 | BTKA |
| ATOM | 1119 | N | VAL | 537 | 35.112 | 28.673 | 10.481 | 1.00 | 12.34 | BTKA |
| ATOM | 1120 | CA | VAL | 537 | 34.000 | 28.503 | 11.395 | 1.00 | 10.90 | BTKA |
| ATOM | 1121 | CB | VAL | 537 | 32.635 | 28.484 | 10.649 | 1.00 | 11.82 | BTKA |
| ATOM | 1122 | CG1 | VAL | 537 | 32.547 | 29.644 | 9.678 | 1.00 | 5.08 | BTKA |
| ATOM | 1123 | CG2 | VAL | 537 | 32.439 | 27.188 | 9.923 | 1.00 | 8.05 | BTKA |
| ATOM | 1124 | C | VAL | 537 | 34.216 | 27.200 | 12.165 | 1.00 | 8.75 | BTKA |
| ATOM | 1125 | O | VAL | 537 | 34.718 | 26.220 | 11.623 | 1.00 | 4.07 | BTKA |
| ATOM | 1126 | N | SER | 538 | 33.850 | 27.191 | 13.436 | 1.00 | 8.53 | BTKA |
| ATOM | 1127 | CA | SER | 538 | 34.023 | 26.004 | 14.244 | 1.00 | 9.45 | BTKA |
| ATOM | 1128 | CB | SER | 538 | 35.286 | 26.161 | 15.091 | 1.00 | 12.59 | BTKA |
| ATOM | 1129 | OG | SER | 538 | 35.492 | 27.515 | 15.458 | 1.00 | 17.47 | BTKA |
| ATOM | 1130 | C | SER | 538 | 32.810 | 25.733 | 15.121 | 1.00 | 10.31 | BTKA |
| ATOM | 1131 | O | SER | 538 | 31.716 | 26.215 | 14.844 | 1.00 | 6.08 | BTKA |
| ATOM | 1132 | N | ASP | 539 | 33.005 | 24.916 | 16.150 | 1.00 | 15.14 | BTKA |
| ATOM | 1133 | CA | ASP | 539 | 31.962 | 24.551 | 17.096 | 1.00 | 16.76 | BTKA |
| ATOM | 1134 | CB | ASP | 539 | 31.806 | 25.633 | 18.170 | 1.00 | 18.57 | BTKA |
| ATOM | 1135 | CG | ASP | 539 | 32.995 | 25.693 | 19.113 | 1.00 | 21.43 | BTKA |
| ATOM | 1136 | OD1 | ASP | 539 | 33.900 | 24.834 | 18.995 | 1.00 | 22.15 | BTKA |
| ATOM | 1137 | OD2 | ASP | 539 | 33.025 | 26.597 | 19.975 | 1.00 | 22.92 | BTKA |
| ATOM | 1138 | C | ASP | 539 | 30.614 | 24.162 | 16.488 | 1.00 | 17.48 | BTKA |
| ATOM | 1139 | O | ASP | 539 | 29.555 | 24.628 | 16.912 | 1.00 | 16.42 | BTKA |
| ATOM | 1140 | N | PHE | 540 | 30.674 | 23.321 | 15.462 | 1.00 | 16.99 | BTKA |
| ATOM | 1141 | CA | PHE | 540 | 29.475 | 22.810 | 14.814 | 1.00 | 16.89 | BTKA |
| ATOM | 1142 | CB | PHE | 540 | 29.500 | 23.037 | 13.294 | 1.00 | 16.17 | BTKA |
| ATOM | 1143 | CG | PHE | 540 | 30.740 | 22.539 | 12.615 | 1.00 | 13.71 | BTKA |
| ATOM | 1144 | CD1 | PHE | 540 | 31.805 | 23.397 | 12.371 | 1.00 | 16.51 | BTKA |
| ATOM | 1145 | CD2 | PHE | 540 | 30.845 | 21.216 | 12.206 | 1.00 | 15.68 | BTKA |
| ATOM | 1146 | CE1 | PHE | 540 | 32.961 | 22.945 | 11.729 | 1.00 | 16.66 | BTKA |
| ATOM | 1147 | CE2 | PHE | 540 | 31.992 | 20.757 | 11.565 | 1.00 | 17.77 | BTKA |
| ATOM | 1148 | CZ | PHE | 540 | 33.053 | 21.626 | 11.327 | 1.00 | 15.13 | BTKA |
| ATOM | 1149 | C | PHE | 540 | 29.408 | 21.326 | 15.143 | 1.00 | 21.34 | BTKA |
| ATOM | 1150 | O | PHE | 540 | 28.789 | 20.547 | 14.428 | 1.00 | 23.74 | BTKA |
| ATOM | 1151 | N | GLY | 541 | 30.058 | 20.948 | 16.240 | 1.00 | 26.85 | BTKA |
| ATOM | 1152 | CA | GLY | 541 | 30.077 | 19.561 | 16.665 | 1.00 | 29.31 | BTKA |
| ATOM | 1153 | C | GLY | 541 | 28.691 | 19.006 | 16.891 | 1.00 | 30.12 | BTKA |
| ATOM | 1154 | O | GLY | 541 | 28.263 | 18.096 | 16.195 | 1.00 | 34.04 | BTKA |
| ATOM | 1155 | N | LEU | 542 | 27.977 | 19.593 | 17.843 | 1.00 | 33.66 | BTKA |
| ATOM | 1156 | CA | LEU | 542 | 26.624 | 19.162 | 18.176 | 1.00 | 35.98 | BTKA |
| ATOM | 1157 | CB | LEU | 542 | 26.172 | 19.825 | 19.477 | 1.00 | 34.47 | BTKA |
| ATOM | 1158 | CG | LEU | 542 | 24.945 | 19.203 | 20.138 | 1.00 | 32.57 | BTKA |
| ATOM | 1159 | CD1 | LEU | 542 | 25.249 | 17.747 | 20.483 | 1.00 | 31.46 | BTKA |
| ATOM | 1160 | CD2 | LEU | 542 | 24.570 | 19.988 | 21.384 | 1.00 | 34.75 | BTKA |
| ATOM | 1161 | C | LEU | 542 | 25.611 | 19.465 | 17.073 | 1.00 | 37.74 | BTKA |
| ATOM | 1162 | O | LEU | 542 | 24.484 | 18.974 | 17.115 | 1.00 | 41.14 | BTKA |
| ATOM | 1163 | N | SER | 543 | 25.998 | 20.302 | 16.113 | 1.00 | 39.87 | BTKA |
| ATOM | 1164 | CA | SER | 543 | 25.119 | 20.670 | 15.006 | 1.00 | 37.74 | BTKA |
| ATOM | 1165 | CB | SER | 543 | 25.884 | 21.503 | 13.991 | 1.00 | 36.22 | BTKA |
| ATOM | 1166 | OG | SER | 543 | 26.286 | 22.741 | 14.549 | 1.00 | 39.50 | BTKA |
| ATOM | 1167 | C | SER | 543 | 24.500 | 19.438 | 14.353 | 1.00 | 36.64 | BTKA |
| ATOM | 1168 | O | SER | 543 | 25.122 | 18.378 | 14.295 | 1.00 | 39.71 | BTKA |
| ATOM | 1169 | N | ARG | 544 | 23.291 | 19.588 | 13.823 | 1.00 | 35.36 | BTKA |
| ATOM | 1170 | CA | ARG | 544 | 22.586 | 18.449 | 13.243 | 1.00 | 35.31 | BTKA |
| ATOM | 1171 | CB | ARG | 544 | 21.158 | 18.375 | 13.812 | 1.00 | 33.92 | BTKA |
| ATOM | 1172 | CG | ARG | 544 | 21.042 | 18.446 | 15.337 | 1.00 | 31.74 | BTKA |
| ATOM | 1173 | CD | ARG | 544 | 21.624 | 17.217 | 16.023 | 1.00 | 31.10 | BTKA |
| ATOM | 1174 | NE | ARG | 544 | 21.323 | 17.206 | 17.455 | 1.00 | 28.02 | BTKA |
| ATOM | 1175 | CZ | ARG | 544 | 22.167 | 16.789 | 18.394 | 1.00 | 27.42 | BTKA |
| ATOM | 1176 | NH1 | ARG | 544 | 23.374 | 16.347 | 18.063 | 1.00 | 29.84 | BTKA |
| ATOM | 1177 | NH2 | ARG | 544 | 21.803 | 16.809 | 19.669 | 1.00 | 24.16 | BTKA |
| ATOM | 1178 | C | ARG | 544 | 22.490 | 18.345 | 11.726 | 1.00 | 34.69 | BTKA |
| ATOM | 1179 | O | ARG | 544 | 22.040 | 19.275 | 11.050 | 1.00 | 34.23 | BTKA |
| ATOM | 1180 | N | TYR | 545 | 22.900 | 17.195 | 11.196 | 1.00 | 34.38 | BTKA |
| ATOM | 1181 | CA | TYR | 545 | 22.771 | 16.931 | 9.772 | 1.00 | 33.59 | BTKA |
| ATOM | 1182 | CB | TYR | 545 | 23.675 | 15.769 | 9.337 | 1.00 | 30.93 | BTKA |
| ATOM | 1183 | CG | TYR | 545 | 23.699 | 15.529 | 7.834 | 1.00 | 33.42 | BTKA |
| ATOM | 1184 | CD1 | TYR | 545 | 22.515 | 15.298 | 7.120 | 1.00 | 31.23 | BTKA |
| ATOM | 1185 | CE1 | TYR | 545 | 22.530 | 15.081 | 5.743 | 1.00 | 28.45 | BTKA |
| ATOM | 1186 | CD2 | TYR | 545 | 24.904 | 15.531 | 7.125 | 1.00 | 28.39 | BTKA |
| ATOM | 1187 | CE2 | TYR | 545 | 24.927 | 15.314 | 5.743 | 1.00 | 28.97 | BTKA |
| ATOM | 1188 | CZ | TYR | 545 | 23.735 | 15.089 | 5.059 | 1.00 | 28.78 | BTKA |
| ATOM | 1189 | OH | TYR | 545 | 23.743 | 14.896 | 3.694 | 1.00 | 25.38 | BTKA |
| ATOM | 1190 | C | TYR | 545 | 21.303 | 16.510 | 9.724 | 1.00 | 35.39 | BTKA |
| ATOM | 1191 | O | TYR | 545 | 20.970 | 15.333 | 9.879 | 1.00 | 37.41 | BTKA |
| ATOM | 1192 | N | VAL | 546 | 20.428 | 17.498 | 9.602 | 1.00 | 37.55 | BTKA |
| ATOM | 1193 | CA | VAL | 546 | 18.990 | 17.264 | 9.584 | 1.00 | 37.08 | BTKA |
| ATOM | 1194 | CB | VAL | 546 | 18.229 | 18.544 | 10.010 | 1.00 | 37.41 | BTKA |
| ATOM | 1195 | CG1 | VAL | 546 | 16.764 | 18.249 | 10.197 | 1.00 | 37.58 | BTKA |
| ATOM | 1196 | CG2 | VAL | 546 | 18.810 | 19.090 | 11.301 | 1.00 | 35.82 | BTKA |
| ATOM | 1197 | C | VAL | 546 | 18.493 | 16.787 | 8.224 | 1.00 | 37.83 | BTKA |
| ATOM | 1198 | O | VAL | 546 | 18.579 | 17.512 | 7.235 | 1.00 | 39.72 | BTKA |
| ATOM | 1199 | N | LEU | 547 | 17.979 | 15.561 | 8.183 | 1.00 | 38.29 | BTKA |
| ATOM | 1200 | CA | LEU | 547 | 17.464 | 14.983 | 6.942 | 1.00 | 38.05 | BTKA |
| ATOM | 1201 | CB | LEU | 547 | 17.040 | 13.525 | 7.145 | 1.00 | 36.02 | BTKA |
| ATOM | 1202 | CG | LEU | 547 | 18.080 | 12.537 | 7.681 | 1.00 | 37.29 | BTKA |
| ATOM | 1203 | CD1 | LEU | 547 | 18.006 | 12.487 | 9.203 | 1.00 | 36.45 | BTKA |
| ATOM | 1204 | CD2 | LEU | 547 | 17.816 | 11.151 | 7.105 | 1.00 | 41.49 | BTKA |
| ATOM | 1205 | C | LEU | 547 | 16.279 | 15.781 | 6.421 | 1.00 | 37.96 | BTKA |
| ATOM | 1206 | O | LEU | 547 | 16.163 | 16.022 | 5.226 | 1.00 | 39.78 | BTKA |
| ATOM | 1207 | N | ASP | 548 | 15.392 | 16.174 | 7.328 | 1.00 | 39.49 | BTKA |
| ATOM | 1208 | CA | ASP | 548 | 14.213 | 16.947 | 6.961 | 1.00 | 38.62 | BTKA |
| ATOM | 1209 | CB | ASP | 548 | 13.035 | 16.609 | 7.893 | 1.00 | 39.75 | BTKA |
| ATOM | 1210 | CG | ASP | 548 | 13.449 | 16.453 | 9.347 | 1.00 | 35.16 | BTKA |
| ATOM | 1211 | OD1 | ASP | 548 | 13.389 | 15.323 | 9.871 | 1.00 | 35.03 | BTKA |
| ATOM | 1212 | OD2 | ASP | 548 | 13.823 | 17.458 | 9.974 | 1.00 | 34.37 | BTKA |
| ATOM | 1213 | C | ASP | 548 | 14.523 | 18.444 | 6.963 | 1.00 | 37.92 | BTKA |
| ATOM | 1214 | O | ASP | 548 | 15.281 | 18.921 | 7.799 | 1.00 | 39.58 | BTKA |
| ATOM | 1215 | N | ASP | 549 | 13.945 | 19.177 | 6.017 | 1.00 | 37.16 | BTKA |
| ATOM | 1216 | CA | ASP | 549 | 14.182 | 20.621 | 5.904 | 1.00 | 37.60 | BTKA |
| ATOM | 1217 | CB | ASP | 549 | 13.639 | 21.165 | 4.571 | 1.00 | 29.74 | BTKA |
| ATOM | 1218 | CG | ASP | 549 | 12.463 | 20.366 | 4.046 | 1.00 | 24.70 | BTKA |
| ATOM | 1219 | OD1 | ASP | 549 | 11.311 | 20.721 | 4.369 | 1.00 | 21.17 | BTKA |
| ATOM | 1220 | OD2 | ASP | 549 | 12.696 | 19.376 | 3.316 | 1.00 | 21.37 | BTKA |
| ATOM | 1221 | C | ASP | 549 | 13.715 | 21.492 | 7.081 | 1.00 | 39.63 | BTKA |
| ATOM | 1222 | O | ASP | 549 | 14.267 | 22.570 | 7.318 | 1.00 | 38.62 | BTKA |
| ATOM | 1223 | N | GLU | 550 | 12.692 | 21.042 | 7.800 | 1.00 | 42.46 | BTKA |
| ATOM | 1224 | CA | GLU | 550 | 12.183 | 21.783 | 8.951 | 1.00 | 43.87 | BTKA |
| ATOM | 1225 | CB | GLU | 550 | 10.653 | 21.676 | 9.014 | 1.00 | 44.48 | BTKA |
| ATOM | 1226 | CG | GLU | 550 | 9.950 | 22.690 | 9.924 | 1.00 | 42.81 | BTKA |
| ATOM | 1227 | CD | GLU | 550 | 10.269 | 22.514 | 11.401 | 1.00 | 43.82 | BTKA |
| ATOM | 1228 | OE1 | GLU | 550 | 9.910 | 21.468 | 11.986 | 1.00 | 41.82 | BTKA |
| ATOM | 1229 | OE2 | GLU | 550 | 10.884 | 23.428 | 11.984 | 1.00 | 43.35 | BTKA |
| ATOM | 1230 | C | GLU | 550 | 12.811 | 21.061 | 10.132 | 1.00 | 44.39 | BTKA |
| ATOM | 1231 | O | GLU | 550 | 12.798 | 19.833 | 10.161 | 1.00 | 45.16 | BTKA |
| ATOM | 1232 | N | TYR | 551 | 13.383 | 21.803 | 11.080 | 1.00 | 44.30 | BTKA |
| ATOM | 1233 | CA | TYR | 551 | 14.026 | 21.184 | 12.239 | 1.00 | 42.30 | BTKA |
| ATOM | 1234 | CB | TYR | 551 | 15.516 | 21.535 | 12.282 | 1.00 | 41.57 | BTKA |
| ATOM | 1235 | CG | TYR | 551 | 16.221 | 21.112 | 13.559 | 1.00 | 42.35 | BTKA |
| ATOM | 1236 | CD1 | TYR | 551 | 16.573 | 19.780 | 13.786 | 1.00 | 43.14 | BTKA |
| ATOM | 1237 | CE1 | TYR | 551 | 17.241 | 19.402 | 14.952 | 1.00 | 40.79 | BTKA |
| ATOM | 1238 | CD2 | TYR | 551 | 16.550 | 22.051 | 14.533 | 1.00 | 40.74 | BTKA |
| ATOM | 1239 | CE2 | TYR | 551 | 17.214 | 21.683 | 15.697 | 1.00 | 38.38 | BTKA |
| ATOM | 1240 | CZ | TYR | 551 | 17.555 | 20.365 | 15.902 | 1.00 | 39.27 | BTKA |
| ATOM | 1241 | OH | TYR | 551 | 18.206 | 20.021 | 17.061 | 1.00 | 41.15 | BTKA |
| ATOM | 1242 | C | TYR | 551 | 13.384 | 21.501 | 13.586 | 1.00 | 43.53 | BTKA |
| ATOM | 1243 | O | TYR | 551 | 12.965 | 22.633 | 13.849 | 1.00 | 41.79 | BTKA |
| ATOM | 1244 | N | THR | 552 | 13.329 | 20.475 | 14.431 | 1.00 | 44.17 | BTKA |
| ATOM | 1245 | CA | THR | 552 | 12.777 | 20.559 | 15.777 | 1.00 | 42.48 | BTKA |
| ATOM | 1246 | CB | THR | 552 | 11.555 | 19.626 | 15.927 | 1.00 | 40.83 | BTKA |
| ATOM | 1247 | OG1 | THR | 552 | 11.850 | 18.351 | 15.345 | 1.00 | 39.32 | BTKA |
| ATOM | 1248 | CG2 | THR | 552 | 10.343 | 20.216 | 15.226 | 1.00 | 40.05 | BTKA |
| ATOM | 1249 | C | THR | 552 | 13.900 | 20.118 | 16.716 | 1.00 | 43.93 | BTKA |
| ATOM | 1250 | O | THR | 552 | 14.784 | 19.366 | 16.300 | 1.00 | 45.33 | BTKA |
| ATOM | 1251 | N | SER | 553 | 13.884 | 20.584 | 17.963 | 1.00 | 42.56 | BTKA |
| ATOM | 1252 | CA | SER | 553 | 14.940 | 20.231 | 18.909 | 1.00 | 41.52 | BTKA |
| ATOM | 1253 | CB | SER | 553 | 15.942 | 21.388 | 18.998 | 1.00 | 43.12 | BTKA |
| ATOM | 1254 | OG | SER | 553 | 17.207 | 20.961 | 19.479 | 1.00 | 42.78 | BTKA |
| ATOM | 1255 | C | SER | 553 | 14.384 | 19.902 | 20.299 | 1.00 | 41.75 | BTKA |
| ATOM | 1256 | O | SER | 553 | 13.185 | 19.638 | 20.446 | 1.00 | 41.54 | BTKA |
| ATOM | 1257 | N | SER | 554 | 15.269 | 19.863 | 21.296 | 1.00 | 40.52 | BTKA |
| ATOM | 1258 | CA | SER | 554 | 14.883 | 19.589 | 22.678 | 1.00 | 41.03 | BTKA |
| ATOM | 1259 | CB | SER | 554 | 15.895 | 18.655 | 23.349 | 1.00 | 41.04 | BTKA |
| ATOM | 1260 | OG | SER | 554 | 15.346 | 18.053 | 24.515 | 1.00 | 41.68 | BTKA |
| ATOM | 1261 | C | SER | 554 | 14.833 | 20.932 | 23.406 | 1.00 | 41.29 | BTKA |
| ATOM | 1262 | O | SER | 554 | 13.833 | 21.273 | 24.033 | 1.00 | 40.26 | BTKA |
| ATOM | 1263 | N | VAL | 555 | 15.928 | 21.681 | 23.287 | 1.00 | 42.31 | BTKA |
| ATOM | 1264 | CA | VAL | 555 | 16.104 | 23.024 | 23.858 | 1.00 | 44.31 | BTKA |
| ATOM | 1265 | CB | VAL | 555 | 15.284 | 24.090 | 23.082 | 1.00 | 46.09 | BTKA |
| ATOM | 1266 | CG1 | VAL | 555 | 15.359 | 23.808 | 21.607 | 1.00 | 45.26 | BTKA |
| ATOM | 1267 | CG2 | VAL | 555 | 13.840 | 24.159 | 23.568 | 1.00 | 46.08 | BTKA |
| ATOM | 1268 | C | VAL | 555 | 15.979 | 23.284 | 25.365 | 1.00 | 43.78 | BTKA |
| ATOM | 1269 | O | VAL | 555 | 15.291 | 22.570 | 26.101 | 1.00 | 43.91 | BTKA |
| ATOM | 1270 | N | GLY | 556 | 16.690 | 24.324 | 25.797 | 1.00 | 42.58 | BTKA |
| ATOM | 1271 | CA | GLY | 556 | 16.712 | 24.732 | 27.187 | 1.00 | 42.48 | BTKA |
| ATOM | 1272 | C | GLY | 556 | 18.047 | 25.357 | 27.556 | 1.00 | 41.57 | BTKA |
| ATOM | 1273 | O | GLY | 556 | 18.784 | 25.812 | 26.675 | 1.00 | 41.16 | BTKA |
| ATOM | 1274 | N | SER | 557 | 18.352 | 25.378 | 28.853 | 1.00 | 40.73 | BTKA |
| ATOM | 1275 | CA | SER | 557 | 19.603 | 25.934 | 29.383 | 1.00 | 38.88 | BTKA |
| ATOM | 1276 | CB | SER | 557 | 20.812 | 25.220 | 28.764 | 1.00 | 40.14 | BTKA |
| ATOM | 1277 | OG | SER | 557 | 20.724 | 23.812 | 28.944 | 1.00 | 36.05 | BTKA |
| ATOM | 1278 | C | SER | 557 | 19.721 | 27.453 | 29.214 | 1.00 | 38.15 | BTKA |
| ATOM | 1279 | O | SER | 557 | 19.729 | 28.193 | 30.197 | 1.00 | 37.76 | BTKA |
| ATOM | 1280 | N | LYS | 558 | 19.826 | 27.905 | 27.971 | 1.00 | 36.40 | BTKA |
| ATOM | 1281 | CA | LYS | 558 | 19.926 | 29.323 | 27.648 | 1.00 | 33.00 | BTKA |
| ATOM | 1282 | CB | LYS | 558 | 21.170 | 29.948 | 28.284 | 1.00 | 32.93 | BTKA |
| ATOM | 1283 | CG | LYS | 558 | 21.234 | 31.480 | 28.249 | 1.00 | 28.95 | BTKA |
| ATOM | 1284 | CD | LYS | 558 | 20.109 | 32.151 | 29.034 | 1.00 | 25.19 | BTKA |
| ATOM | 1285 | CE | LYS | 558 | 18.864 | 32.378 | 28.182 | 1.00 | 24.86 | BTKA |
| ATOM | 1286 | NZ | LYS | 558 | 19.140 | 33.238 | 26.987 | 1.00 | 27.50 | BTKA |
| ATOM | 1287 | C | LYS | 558 | 19.987 | 29.415 | 26.127 | 1.00 | 32.13 | BTKA |
| ATOM | 1288 | O | LYS | 558 | 20.184 | 28.403 | 25.448 | 1.00 | 31.90 | BTKA |
| ATOM | 1289 | N | PHE | 559 | 19.843 | 30.618 | 25.591 | 1.00 | 31.72 | BTKA |
| ATOM | 1290 | CA | PHE | 559 | 19.848 | 30.797 | 24.147 | 1.00 | 30.24 | BTKA |
| ATOM | 1291 | CB | PHE | 559 | 18.410 | 31.033 | 23.671 | 1.00 | 33.13 | BTKA |
| ATOM | 1292 | CG | PHE | 559 | 18.028 | 30.265 | 22.440 | 1.00 | 36.97 | BTKA |
| ATOM | 1293 | CD1 | PHE | 559 | 18.976 | 29.572 | 21.698 | 1.00 | 37.84 | BTKA |
| ATOM | 1294 | CD2 | PHE | 559 | 16.697 | 30.207 | 22.042 | 1.00 | 40.25 | BTKA |
| ATOM | 1295 | CE1 | PHE | 559 | 18.605 | 28.832 | 20.582 | 1.00 | 36.42 | BTKA |
| ATOM | 1296 | CE2 | PHE | 559 | 16.318 | 29.468 | 20.932 | 1.00 | 39.23 | BTKA |
| ATOM | 1297 | CZ | PHE | 559 | 17.275 | 28.779 | 20.203 | 1.00 | 39.57 | BTKA |
| ATOM | 1298 | C | PHE | 559 | 20.672 | 32.005 | 23.738 | 1.00 | 28.68 | BTKA |
| ATOM | 1299 | O | PHE | 559 | 20.647 | 33.036 | 24.418 | 1.00 | 32.43 | BTKA |
| ATOM | 1300 | N | PRO | 560 | 21.505 | 31.859 | 22.696 | 1.00 | 25.69 | BTKA |
| ATOM | 1301 | CD | PRO | 560 | 21.989 | 30.606 | 22.090 | 1.00 | 23.07 | BTKA |
| ATOM | 1302 | CA | PRO | 560 | 22.303 | 33.003 | 22.244 | 1.00 | 25.30 | BTKA |
| ATOM | 1303 | CB | PRO | 560 | 23.371 | 32.343 | 21.371 | 1.00 | 24.23 | BTKA |
| ATOM | 1304 | CG | PRO | 560 | 22.695 | 31.103 | 20.873 | 1.00 | 23.44 | BTKA |
| ATOM | 1305 | C | PRO | 560 | 21.322 | 33.869 | 21.439 | 1.00 | 22.00 | BTKA |
| ATOM | 1306 | O | PRO | 560 | 21.428 | 34.002 | 20.220 | 1.00 | 20.54 | BTKA |
| ATOM | 1307 | N | VAL | 561 | 20.370 | 34.447 | 22.170 | 1.00 | 18.60 | BTKA |
| ATOM | 1308 | CA | VAL | 561 | 19.271 | 35.271 | 21.653 | 1.00 | 15.90 | BTKA |
| ATOM | 1309 | CB | VAL | 561 | 18.475 | 35.908 | 22.840 | 1.00 | 13.27 | BTKA |
| ATOM | 1310 | CG1 | VAL | 561 | 17.202 | 36.587 | 22.348 | 1.00 | 12.23 | BTKA |
| ATOM | 1311 | CG2 | VAL | 561 | 18.126 | 34.848 | 23.858 | 1.00 | 10.66 | BTKA |
| ATOM | 1312 | C | VAL | 561 | 19.631 | 36.360 | 20.645 | 1.00 | 12.76 | BTKA |
| ATOM | 1313 | O | VAL | 561 | 18.900 | 36.595 | 19.686 | 1.00 | 14.28 | BTKA |
| ATOM | 1314 | N | ARG | 562 | 20.778 | 36.990 | 20.849 | 1.00 | 11.30 | BTKA |
| ATOM | 1315 | CA | ARG | 562 | 21.238 | 38.078 | 19.996 | 1.00 | 7.17 | BTKA |
| ATOM | 1316 | CB | ARG | 562 | 22.435 | 38.756 | 20.647 | 1.00 | 6.00 | BTKA |
| ATOM | 1317 | CG | ARG | 562 | 22.184 | 39.160 | 22.086 | 1.00 | 7.62 | BTKA |
| ATOM | 1318 | CD | ARG | 562 | 23.396 | 39.859 | 22.678 | 1.00 | 8.88 | BTKA |
| ATOM | 1319 | NE | ARG | 562 | 23.231 | 40.134 | 24.100 | 1.00 | 12.40 | BTKA |
| ATOM | 1320 | CZ | ARG | 562 | 22.311 | 40.944 | 24.614 | 1.00 | 6.84 | BTKA |
| ATOM | 1321 | NH1 | ARG | 562 | 22.250 | 41.118 | 25.921 | 1.00 | 15.72 | BTKA |
| ATOM | 1322 | NH2 | ARG | 562 | 21.458 | 41.583 | 23.835 | 1.00 | 7.47 | BTKA |
| ATOM | 1323 | C | ARG | 562 | 21.587 | 37.696 | 18.569 | 1.00 | 8.37 | BTKA |
| ATOM | 1324 | O | ARG | 562 | 21.885 | 38.566 | 17.763 | 1.00 | 7.58 | BTKA |
| ATOM | 1325 | N | TRP | 563 | 21.584 | 36.402 | 18.264 | 1.00 | 9.41 | BTKA |
| ATOM | 1326 | CA | TRP | 563 | 21.909 | 35.920 | 16.918 | 1.00 | 7.23 | BTKA |
| ATOM | 1327 | CB | TRP | 563 | 23.207 | 35.107 | 16.932 | 1.00 | 7.42 | BTKA |
| ATOM | 1328 | CG | TRP | 563 | 24.422 | 35.878 | 17.243 | 1.00 | 6.68 | BTKA |
| ATOM | 1329 | CD2 | TRP | 563 | 24.892 | 36.249 | 18.541 | 1.00 | 8.73 | BTKA |
| ATOM | 1330 | CE2 | TRP | 563 | 26.070 | 36.995 | 18.358 | 1.00 | 8.54 | BTKA |
| ATOM | 1331 | CE3 | TRP | 563 | 24.432 | 36.021 | 19.845 | 1.00 | 7.52 | BTKA |
| ATOM | 1332 | CD1 | TRP | 563 | 25.306 | 36.388 | 16.351 | 1.00 | 10.62 | BTKA |
| ATOM | 1333 | NE1 | TRP | 563 | 26.297 | 37.071 | 17.010 | 1.00 | 11.83 | BTKA |
| ATOM | 1334 | CZ2 | TRP | 563 | 26.796 | 37.525 | 19.423 | 1.00 | 10.18 | BTKA |
| ATOM | 1335 | CZ3 | TRP | 563 | 25.153 | 36.549 | 20.907 | 1.00 | 14.27 | BTKA |
| ATOM | 1336 | CH2 | TRP | 563 | 26.325 | 37.292 | 20.686 | 1.00 | 11.16 | BTKA |
| ATOM | 1337 | C | TRP | 563 | 20.806 | 35.000 | 16.434 | 1.00 | 3.39 | BTKA |
| ATOM | 1338 | O | TRP | 563 | 20.986 | 34.269 | 15.470 | 1.00 | 6.67 | BTKA |
| ATOM | 1339 | N | SER | 564 | 19.654 | 35.063 | 17.080 | 1.00 | 4.53 | BTKA |
| ATOM | 1340 | CA | SER | 564 | 18.568 | 34.179 | 16.724 | 1.00 | 7.96 | BTKA |
| ATOM | 1341 | CB | SER | 564 | 18.134 | 33.404 | 17.971 | 1.00 | 9.14 | BTKA |
| ATOM | 1342 | OG | SER | 564 | 19.237 | 32.702 | 18.549 | 1.00 | 15.71 | BTKA |
| ATOM | 1343 | C | SER | 564 | 17.364 | 34.853 | 16.088 | 1.00 | 9.34 | BTKA |
| ATOM | 1344 | O | SER | 564 | 16.952 | 35.938 | 16.501 | 1.00 | 8.42 | BTKA |
| ATOM | 1345 | N | PRO | 565 | 16.839 | 34.255 | 15.004 | 1.00 | 9.83 | BTKA |
| ATOM | 1346 | CD | PRO | 565 | 17.389 | 33.137 | 14.219 | 1.00 | 10.36 | BTKA |
| ATOM | 1347 | CA | PRO | 565 | 15.671 | 34.821 | 14.336 | 1.00 | 11.63 | BTKA |
| ATOM | 1348 | CB | PRO | 565 | 15.605 | 34.018 | 13.033 | 1.00 | 7.64 | BTKA |
| ATOM | 1349 | CG | PRO | 565 | 16.203 | 32.723 | 13.395 | 1.00 | 3.54 | BTKA |
| ATOM | 1350 | C | PRO | 565 | 14.451 | 34.575 | 15.225 | 1.00 | 11.02 | BTKA |
| ATOM | 1351 | O | PRO | 565 | 14.444 | 33.649 | 16.040 | 1.00 | 9.72 | BTKA |
| ATOM | 1352 | N | PRO | 566 | 13.397 | 35.382 | 15.064 | 1.00 | 14.93 | BTKA |
| ATOM | 1353 | CD | PRO | 566 | 13.208 | 36.466 | 14.085 | 1.00 | 16.14 | BTKA |
| ATOM | 1354 | CA | PRO | 566 | 12.194 | 35.207 | 15.881 | 1.00 | 14.83 | BTKA |
| ATOM | 1355 | CB | PRO | 566 | 11.217 | 36.217 | 15.272 | 1.00 | 14.50 | BTKA |
| ATOM | 1356 | CG | PRO | 566 | 11.729 | 36.426 | 13.876 | 1.00 | 18.85 | BTKA |
| ATOM | 1357 | C | PRO | 566 | 11.617 | 33.781 | 15.963 | 1.00 | 12.73 | BTKA |
| ATOM | 1358 | O | PRO | 566 | 11.195 | 33.361 | 17.038 | 1.00 | 13.91 | BTKA |
| ATOM | 1359 | N | GLU | 567 | 11.636 | 33.012 | 14.874 | 1.00 | 13.43 | BTKA |
| ATOM | 1360 | CA | GLU | 567 | 11.075 | 31.652 | 14.932 | 1.00 | 13.41 | BTKA |
| ATOM | 1361 | CB | GLU | 567 | 10.789 | 31.072 | 13.541 | 1.00 | 15.94 | BTKA |
| ATOM | 1362 | CG | GLU | 567 | 12.010 | 30.662 | 12.730 | 1.00 | 17.33 | BTKA |
| ATOM | 1363 | CD | GLU | 567 | 12.733 | 31.825 | 12.072 | 1.00 | 12.50 | BTKA |
| ATOM | 1364 | OE1 | GLU | 567 | 12.300 | 32.993 | 12.211 | 1.00 | 10.98 | BTKA |
| ATOM | 1365 | OE2 | GLU | 567 | 13.749 | 31.550 | 11.404 | 1.00 | 9.50 | BTKA |
| ATOM | 1366 | C | GLU | 567 | 11.908 | 30.685 | 15.769 | 1.00 | 11.95 | BTKA |
| ATOM | 1367 | O | GLU | 567 | 11.460 | 29.581 | 16.106 | 1.00 | 12.58 | BTKA |
| ATOM | 1368 | N | VAL | 568 | 13.134 | 31.083 | 16.078 | 1.00 | 10.44 | BTKA |
| ATOM | 1369 | CA | VAL | 568 | 13.994 | 30.274 | 16.926 | 1.00 | 9.53 | BTKA |
| ATOM | 1370 | CB | VAL | 568 | 15.473 | 30.421 | 16.541 | 1.00 | 4.21 | BTKA |
| ATOM | 1371 | CG1 | VAL | 568 | 16.331 | 30.518 | 17.751 | 1.00 | 6.04 | BTKA |
| ATOM | 1372 | CG2 | VAL | 568 | 15.902 | 29.228 | 15.729 | 1.00 | 5.60 | BTKA |
| ATOM | 1373 | C | VAL | 568 | 13.722 | 30.798 | 18.331 | 1.00 | 10.51 | BTKA |
| ATOM | 1374 | O | VAL | 568 | 13.608 | 30.029 | 19.285 | 1.00 | 9.31 | BTKA |
| ATOM | 1375 | N | LEU | 569 | 13.521 | 32.110 | 18.415 | 1.00 | 10.75 | BTKA |
| ATOM | 1376 | CA | LEU | 569 | 13.218 | 32.803 | 19.663 | 1.00 | 14.44 | BTKA |
| ATOM | 1377 | CB | LEU | 569 | 13.137 | 34.308 | 19.397 | 1.00 | 12.26 | BTKA |
| ATOM | 1378 | CG | LEU | 569 | 14.207 | 35.247 | 19.958 | 1.00 | 11.89 | BTKA |
| ATOM | 1379 | CD1 | LEU | 569 | 15.591 | 34.631 | 19.852 | 1.00 | 11.36 | BTKA |
| ATOM | 1380 | CD2 | LEU | 569 | 14.135 | 36.583 | 19.221 | 1.00 | 12.38 | BTKA |
| ATOM | 1381 | C | LEU | 569 | 11.885 | 32.333 | 20.244 | 1.00 | 17.10 | BTKA |
| ATOM | 1382 | O | LEU | 569 | 11.709 | 32.288 | 21.462 | 1.00 | 20.31 | BTKA |
| ATOM | 1383 | N | MET | 570 | 10.959 | 31.964 | 19.363 | 1.00 | 17.50 | BTKA |
| ATOM | 1384 | CA | MET | 570 | 9.629 | 31.528 | 19.769 | 1.00 | 16.12 | BTKA |
| ATOM | 1385 | CB | MET | 570 | 8.588 | 32.073 | 18.797 | 1.00 | 16.10 | BTKA |
| ATOM | 1386 | CG | MET | 570 | 8.490 | 33.593 | 18.830 | 1.00 | 17.26 | BTKA |
| ATOM | 1387 | SD | MET | 570 | 7.274 | 34.254 | 17.686 | 1.00 | 27.62 | BTKA |
| ATOM | 1388 | CE | MET | 570 | 8.296 | 34.747 | 16.354 | 1.00 | 23.00 | BTKA |
| ATOM | 1389 | C | MET | 570 | 9.404 | 30.043 | 20.028 | 1.00 | 12.94 | BTKA |
| ATOM | 1390 | O | MET | 570 | 8.637 | 29.689 | 20.923 | 1.00 | 15.56 | BTKA |
| ATOM | 1391 | N | TYR | 571 | 10.027 | 29.172 | 19.242 | 1.00 | 12.61 | BTKA |
| ATOM | 1392 | CA | TYR | 571 | 9.875 | 27.726 | 19.448 | 1.00 | 14.91 | BTKA |
| ATOM | 1393 | CB | TYR | 571 | 8.489 | 27.216 | 19.010 | 1.00 | 17.43 | BTKA |
| ATOM | 1394 | CG | TYR | 571 | 8.126 | 27.460 | 17.560 | 1.00 | 16.93 | BTKA |
| ATOM | 1395 | CD1 | TYR | 571 | 7.914 | 26.397 | 16.691 | 1.00 | 17.22 | BTKA |
| ATOM | 1396 | CE1 | TYR | 571 | 7.565 | 26.612 | 15.361 | 1.00 | 18.55 | BTKA |
| ATOM | 1397 | CD2 | TYR | 571 | 7.983 | 28.754 | 17.067 | 1.00 | 15.52 | BTKA |
| ATOM | 1398 | CE2 | TYR | 571 | 7.640 | 28.987 | 15.742 | 1.00 | 21.13 | BTKA |
| ATOM | 1399 | CZ | TYR | 571 | 7.428 | 27.912 | 14.891 | 1.00 | 22.58 | BTKA |
| ATOM | 1400 | OH | TYR | 571 | 7.070 | 28.139 | 13.582 | 1.00 | 18.95 | BTKA |
| ATOM | 1401 | C | TYR | 571 | 10.983 | 26.881 | 18.832 | 1.00 | 15.24 | BTKA |
| ATOM | 1402 | O | TYR | 571 | 10.734 | 25.810 | 18.286 | 1.00 | 17.04 | BTKA |
| ATOM | 1403 | N | SER | 572 | 12.204 | 27.398 | 18.894 | 1.00 | 19.07 | BTKA |
| ATOM | 1404 | CA | SER | 572 | 13.383 | 26.696 | 18.399 | 1.00 | 19.19 | BTKA |
| ATOM | 1405 | CB | SER | 572 | 13.759 | 25.611 | 19.399 | 1.00 | 16.09 | BTKA |
| ATOM | 1406 | OG | SER | 572 | 13.662 | 26.109 | 20.727 | 1.00 | 13.53 | BTKA |
| ATOM | 1407 | C | SER | 572 | 13.270 | 26.088 | 17.001 | 1.00 | 19.87 | BTKA |
| ATOM | 1408 | O | SER | 572 | 13.729 | 24.960 | 16.761 | 1.00 | 16.73 | BTKA |
| ATOM | 1409 | N | LYS | 573 | 12.679 | 26.838 | 16.076 | 1.00 | 22.09 | BTKA |
| ATOM | 1410 | CA | LYS | 573 | 12.535 | 26.364 | 14.707 | 1.00 | 23.82 | BTKA |
| ATOM | 1411 | CB | LYS | 573 | 11.304 | 27.005 | 14.060 | 1.00 | 24.62 | BTKA |
| ATOM | 1412 | CG | LYS | 573 | 10.870 | 26.369 | 12.755 | 1.00 | 24.09 | BTKA |
| ATOM | 1413 | CD | LYS | 573 | 9.561 | 26.969 | 12.289 | 1.00 | 27.34 | BTKA |
| ATOM | 1414 | CE | LYS | 573 | 8.914 | 26.145 | 11.197 | 1.00 | 27.52 | BTKA |
| ATOM | 1415 | NZ | LYS | 573 | 7.495 | 26.549 | 11.009 | 1.00 | 27.13 | BTKA |
| ATOM | 1416 | C | LYS | 573 | 13.810 | 26.672 | 13.898 | 1.00 | 24.62 | BTKA |
| ATOM | 1417 | O | LYS | 573 | 13.875 | 27.661 | 13.159 | 1.00 | 24.59 | BTKA |
| ATOM | 1418 | N | PHE | 574 | 14.842 | 25.857 | 14.093 | 1.00 | 21.87 | BTKA |
| ATOM | 1419 | CA | PHE | 574 | 16.096 | 26.042 | 13.379 | 1.00 | 17.67 | BTKA |
| ATOM | 1420 | CB | PHE | 574 | 17.238 | 25.268 | 14.055 | 1.00 | 18.29 | BTKA |
| ATOM | 1421 | CG | PHE | 574 | 17.489 | 25.649 | 15.493 | 1.00 | 17.48 | BTKA |
| ATOM | 1422 | CD1 | PHE | 574 | 17.065 | 24.823 | 16.527 | 1.00 | 20.56 | BTKA |
| ATOM | 1423 | CD2 | PHE | 574 | 18.200 | 26.803 | 15.813 | 1.00 | 19.16 | BTKA |
| ATOM | 1424 | CE1 | PHE | 574 | 17.345 | 25.132 | 17.860 | 1.00 | 20.84 | BTKA |
| ATOM | 1425 | CE2 | PHE | 574 | 18.485 | 27.122 | 17.144 | 1.00 | 19.40 | BTKA |
| ATOM | 1426 | CZ | PHE | 574 | 18.054 | 26.280 | 18.168 | 1.00 | 17.92 | BTKA |
| ATOM | 1427 | C | PHE | 574 | 15.941 | 25.514 | 11.960 | 1.00 | 13.16 | BTKA |
| ATOM | 1428 | O | PHE | 574 | 15.181 | 24.577 | 11.716 | 1.00 | 13.27 | BTKA |
| ATOM | 1429 | N | SER | 575 | 16.668 | 26.112 | 11.028 | 1.00 | 13.22 | BTKA |
| ATOM | 1430 | CA | SER | 575 | 16.658 | 25.682 | 9.634 | 1.00 | 12.31 | BTKA |
| ATOM | 1431 | CB | SER | 575 | 15.405 | 26.185 | 8.900 | 1.00 | 9.44 | BTKA |
| ATOM | 1432 | OG | SER | 575 | 15.558 | 27.510 | 8.406 | 1.00 | 5.32 | BTKA |
| ATOM | 1433 | C | SER | 575 | 17.915 | 26.275 | 9.017 | 1.00 | 13.34 | BTKA |
| ATOM | 1434 | O | SER | 575 | 18.848 | 26.636 | 9.738 | 1.00 | 16.79 | BTKA |
| ATOM | 1435 | N | SER | 576 | 17.953 | 26.378 | 7.696 | 1.00 | 12.36 | BTKA |
| ATOM | 1436 | CA | SER | 576 | 19.102 | 26.959 | 7.033 | 1.00 | 11.50 | BTKA |
| ATOM | 1437 | CB | SER | 576 | 19.033 | 26.673 | 5.534 | 1.00 | 17.69 | BTKA |
| ATOM | 1438 | OG | SER | 576 | 17.829 | 27.186 | 4.970 | 1.00 | 25.45 | BTKA |
| ATOM | 1439 | C | SER | 576 | 19.048 | 28.465 | 7.250 | 1.00 | 9.00 | BTKA |
| ATOM | 1440 | O | SER | 576 | 20.068 | 29.129 | 7.436 | 1.00 | 11.10 | BTKA |
| ATOM | 1441 | N | LYS | 577 | 17.833 | 28.995 | 7.247 | 1.00 | 9.50 | BTKA |
| ATOM | 1442 | CA | LYS | 577 | 17.604 | 30.422 | 7.402 | 1.00 | 9.17 | BTKA |
| ATOM | 1443 | CB | LYS | 577 | 16.190 | 30.765 | 6.925 | 1.00 | 12.09 | BTKA |
| ATOM | 1444 | CG | LYS | 577 | 15.892 | 30.249 | 5.508 | 1.00 | 8.29 | BTKA |
| ATOM | 1445 | CD | LYS | 577 | 16.752 | 30.956 | 4.471 | 1.00 | 19.55 | BTKA |
| ATOM | 1446 | CE | LYS | 577 | 16.830 | 30.179 | 3.159 | 1.00 | 15.68 | BTKA |
| ATOM | 1447 | NZ | LYS | 577 | 15.498 | 29.738 | 2.670 | 1.00 | 20.19 | BTKA |
| ATOM | 1448 | C | LYS | 577 | 17.878 | 30.940 | 8.812 | 1.00 | 8.67 | BTKA |
| ATOM | 1449 | O | LYS | 577 | 17.909 | 32.154 | 9.032 | 1.00 | 9.38 | BTKA |
| ATOM | 1450 | N | SER | 578 | 18.054 | 30.026 | 9.766 | 1.00 | 10.63 | BTKA |
| ATOM | 1451 | CA | SER | 578 | 18.378 | 30.401 | 11.141 | 1.00 | 7.64 | BTKA |
| ATOM | 1452 | CB | SER | 578 | 18.107 | 29.250 | 12.110 | 1.00 | 6.86 | BTKA |
| ATOM | 1453 | OG | SER | 578 | 16.743 | 28.879 | 12.106 | 1.00 | 18.48 | BTKA |
| ATOM | 1454 | C | SER | 578 | 19.862 | 30.743 | 11.139 | 1.00 | 3.05 | BTKA |
| ATOM | 1455 | O | SER | 578 | 20.281 | 31.699 | 11.785 | 1.00 | 3.62 | BTKA |
| ATOM | 1456 | N | ASP | 579 | 20.653 | 29.951 | 10.415 | 1.00 | 4.90 | BTKA |
| ATOM | 1457 | CA | ASP | 579 | 22.092 | 30.205 | 10.277 | 1.00 | 7.88 | BTKA |
| ATOM | 1458 | CB | ASP | 579 | 22.773 | 29.075 | 9.522 | 1.00 | 7.80 | BTKA |
| ATOM | 1459 | CG | ASP | 579 | 22.916 | 27.838 | 10.341 | 1.00 | 13.43 | BTKA |
| ATOM | 1460 | OD1 | ASP | 579 | 22.190 | 27.698 | 11.347 | 1.00 | 16.68 | BTKA |
| ATOM | 1461 | OD2 | ASP | 579 | 23.765 | 27.003 | 9.970 | 1.00 | 17.47 | BTKA |
| ATOM | 1462 | C | ASP | 579 | 22.330 | 31.496 | 9.500 | 1.00 | 5.97 | BTKA |
| ATOM | 1463 | O | ASP | 579 | 23.275 | 32.228 | 9.776 | 1.00 | 6.23 | BTKA |
| ATOM | 1464 | N | ILE | 580 | 21.515 | 31.715 | 8.472 | 1.00 | 6.76 | BTKA |
| ATOM | 1465 | CA | ILE | 580 | 21.607 | 32.921 | 7.652 | 1.00 | 8.95 | BTKA |
| ATOM | 1466 | CB | ILE | 580 | 20.548 | 32.921 | 6.497 | 1.00 | 7.78 | BTKA |
| ATOM | 1467 | CG2 | ILE | 580 | 19.670 | 34.171 | 6.529 | 1.00 | 9.49 | BTKA |
| ATOM | 1468 | CG1 | ILE | 580 | 21.252 | 32.861 | 5.136 | 1.00 | 6.38 | BTKA |
| ATOM | 1469 | CD | ILE | 580 | 21.449 | 31.477 | 4.617 | 1.00 | 12.06 | BTKA |
| ATOM | 1470 | C | ILE | 580 | 21.452 | 34.150 | 8.545 | 1.00 | 9.29 | BTKA |
| ATOM | 1471 | O | ILE | 580 | 22.247 | 35.087 | 8.449 | 1.00 | 14.35 | BTKA |
| ATOM | 1472 | N | TRP | 581 | 20.457 | 34.127 | 9.430 | 1.00 | 8.74 | BTKA |
| ATOM | 1473 | CA | TRP | 581 | 20.218 | 35.235 | 10.339 | 1.00 | 4.45 | BTKA |
| ATOM | 1474 | CB | TRP | 581 | 18.997 | 34.961 | 11.219 | 1.00 | 5.26 | BTKA |
| ATOM | 1475 | CG | TRP | 581 | 18.656 | 36.080 | 12.187 | 1.00 | 5.57 | BTKA |
| ATOM | 1476 | CD2 | TRP | 581 | 17.478 | 36.894 | 12.182 | 1.00 | 7.10 | BTKA |
| ATOM | 1477 | CE2 | TRP | 581 | 17.577 | 37.782 | 13.276 | 1.00 | 8.85 | BTKA |
| ATOM | 1478 | CE3 | TRP | 581 | 16.343 | 36.957 | 11.366 | 1.00 | 5.60 | BTKA |
| ATOM | 1479 | CD1 | TRP | 581 | 19.400 | 36.500 | 13.253 | 1.00 | 7.88 | BTKA |
| ATOM | 1480 | NE1 | TRP | 581 | 18.760 | 37.520 | 13.908 | 1.00 | 8.39 | BTKA |
| ATOM | 1481 | CZ2 | TRP | 581 | 16.590 | 38.716 | 13.571 | 1.00 | 7.26 | BTKA |
| ATOM | 1482 | CZ3 | TRP | 581 | 15.364 | 37.885 | 11.661 | 1.00 | 2.00 | BTKA |
| ATOM | 1483 | CH2 | TRP | 581 | 15.495 | 38.752 | 12.753 | 1.00 | 6.05 | BTKA |
| ATOM | 1484 | C | TRP | 581 | 21.442 | 35.446 | 11.195 | 1.00 | 4.23 | BTKA |
| ATOM | 1485 | O | TRP | 581 | 21.953 | 36.557 | 11.290 | 1.00 | 7.98 | BTKA |
| ATOM | 1486 | N | ALA | 582 | 21.907 | 34.371 | 11.821 | 1.00 | 9.71 | BTKA |
| ATOM | 1487 | CA | ALA | 582 | 23.083 | 34.408 | 12.690 | 1.00 | 7.74 | BTKA |
| ATOM | 1488 | CB | ALA | 582 | 23.304 | 33.041 | 13.314 | 1.00 | 5.41 | BTKA |
| ATOM | 1489 | C | ALA | 582 | 24.342 | 34.878 | 11.957 | 1.00 | 9.07 | BTKA |
| ATOM | 1490 | O | ALA | 582 | 25.189 | 35.551 | 12.543 | 1.00 | 8.09 | BTKA |
| ATOM | 1491 | N | PHE | 583 | 24.463 | 34.522 | 10.675 | 1.00 | 12.09 | BTKA |
| ATOM | 1492 | CA | PHE | 583 | 25.609 | 34.920 | 9.841 | 1.00 | 7.60 | BTKA |
| ATOM | 1493 | CB | PHE | 583 | 25.557 | 34.201 | 8.490 | 1.00 | 9.22 | BTKA |
| ATOM | 1494 | CG | PHE | 583 | 26.526 | 34.740 | 7.465 | 1.00 | 4.47 | BTKA |
| ATOM | 1495 | CD1 | PHE | 583 | 27.877 | 34.430 | 7.536 | 1.00 | 4.68 | BTKA |
| ATOM | 1496 | CD2 | PHE | 583 | 26.079 | 35.549 | 6.421 | 1.00 | 6.35 | BTKA |
| ATOM | 1497 | CE1 | PHE | 583 | 28.769 | 34.915 | 6.585 | 1.00 | 5.23 | BTKA |
| ATOM | 1498 | CE2 | PHE | 583 | 26.969 | 36.039 | 5.460 | 1.00 | 2.15 | BTKA |
| ATOM | 1499 | CZ | PHE | 583 | 28.313 | 35.719 | 5.545 | 1.00 | 5.61 | BTKA |
| ATOM | 1500 | C | PHE | 583 | 25.536 | 36.416 | 9.607 | 1.00 | 10.14 | BTKA |
| ATOM | 1501 | O | PHE | 583 | 26.556 | 37.111 | 9.615 | 1.00 | 11.69 | BTKA |
| ATOM | 1502 | N | GLY | 584 | 24.320 | 36.895 | 9.360 | 1.00 | 10.66 | BTKA |
| ATOM | 1503 | CA | GLY | 584 | 24.102 | 38.309 | 9.128 | 1.00 | 9.30 | BTKA |
| ATOM | 1504 | C | GLY | 584 | 24.514 | 39.107 | 10.339 | 1.00 | 7.76 | BTKA |
| ATOM | 1505 | O | GLY | 584 | 25.051 | 40.209 | 10.212 | 1.00 | 10.68 | BTKA |
| ATOM | 1506 | N | VAL | 585 | 24.259 | 38.559 | 11.525 | 1.00 | 7.75 | BTKA |
| ATOM | 1507 | CA | VAL | 585 | 24.644 | 39.246 | 12.750 | 1.00 | 6.19 | BTKA |
| ATOM | 1508 | CB | VAL | 585 | 23.995 | 38.628 | 14.015 | 1.00 | 5.76 | BTKA |
| ATOM | 1509 | CG1 | VAL | 585 | 24.471 | 39.358 | 15.246 | 1.00 | 8.91 | BTKA |
| ATOM | 1510 | CG2 | VAL | 585 | 22.478 | 38.711 | 13.945 | 1.00 | 4.10 | BTKA |
| ATOM | 1511 | C | VAL | 585 | 26.169 | 39.169 | 12.847 | 1.00 | 7.35 | BTKA |
| ATOM | 1512 | O | VAL | 585 | 26.818 | 40.145 | 13.201 | 1.00 | 10.25 | BTKA |
| ATOM | 1513 | N | LEU | 586 | 26.746 | 38.026 | 12.487 | 1.00 | 4.16 | BTKA |
| ATOM | 1514 | CA | LEU | 586 | 28.200 | 37.873 | 12.527 | 1.00 | 7.72 | BTKA |
| ATOM | 1515 | CB | LEU | 586 | 28.632 | 36.492 | 12.005 | 1.00 | 5.58 | BTKA |
| ATOM | 1516 | CG | LEU | 586 | 30.127 | 36.293 | 11.693 | 1.00 | 6.32 | BTKA |
| ATOM | 1517 | CD1 | LEU | 586 | 31.005 | 36.643 | 12.885 | 1.00 | 2.00 | BTKA |
| ATOM | 1518 | CD2 | LEU | 586 | 30.385 | 34.880 | 11.254 | 1.00 | 6.90 | BTKA |
| ATOM | 1519 | C | LEU | 586 | 28.857 | 38.965 | 11.695 | 1.00 | 6.55 | BTKA |
| ATOM | 1520 | O | LEU | 586 | 29.902 | 39.502 | 12.071 | 1.00 | 6.94 | BTKA |
| ATOM | 1521 | N | MET | 587 | 28.242 | 39.278 | 10.557 | 1.00 | 9.63 | BTKA |
| ATOM | 1522 | CA | MET | 587 | 28.745 | 40.310 | 9.660 | 1.00 | 5.25 | BTKA |
| ATOM | 1523 | CB | MET | 587 | 27.850 | 40.442 | 8.437 | 1.00 | 5.01 | BTKA |
| ATOM | 1524 | CG | MET | 587 | 27.974 | 39.317 | 7.443 | 1.00 | 4.97 | BTKA |
| ATOM | 1525 | SD | MET | 587 | 27.187 | 39.797 | 5.910 | 1.00 | 9.68 | BTKA |
| ATOM | 1526 | CE | MET | 587 | 28.480 | 40.808 | 5.189 | 1.00 | 3.35 | BTKA |
| ATOM | 1527 | C | MET | 587 | 28.755 | 41.626 | 10.400 | 1.00 | 6.90 | BTKA |
| ATOM | 1528 | O | MET | 587 | 29.716 | 42.387 | 10.318 | 1.00 | 4.22 | BTKA |
| ATOM | 1529 | N | TRP | 588 | 27.680 | 41.878 | 11.143 | 1.00 | 8.72 | BTKA |
| ATOM | 1530 | CA | TRP | 588 | 27.551 | 43.096 | 11.925 | 1.00 | 9.02 | BTKA |
| ATOM | 1531 | CB | TRP | 588 | 26.176 | 43.155 | 12.595 | 1.00 | 7.30 | BTKA |
| ATOM | 1532 | CG | TRP | 588 | 25.977 | 44.397 | 13.389 | 1.00 | 3.41 | BTKA |
| ATOM | 1533 | CD2 | TRP | 588 | 26.321 | 44.593 | 14.759 | 1.00 | 5.76 | BTKA |
| ATOM | 1534 | CE2 | TRP | 588 | 26.021 | 45.939 | 15.078 | 1.00 | 3.37 | BTKA |
| ATOM | 1535 | CE3 | TRP | 588 | 26.863 | 43.765 | 15.756 | 1.00 | 7.65 | BTKA |
| ATOM | 1536 | CD1 | TRP | 588 | 25.492 | 45.591 | 12.941 | 1.00 | 3.84 | BTKA |
| ATOM | 1537 | NE1 | TRP | 588 | 25.518 | 46.525 | 13.946 | 1.00 | 2.00 | BTKA |
| ATOM | 1538 | CZ2 | TRP | 588 | 26.244 | 46.470 | 16.348 | 1.00 | 3.23 | BTKA |
| ATOM | 1539 | CZ3 | TRP | 588 | 27.085 | 44.298 | 17.012 | 1.00 | 2.00 | BTKA |
| ATOM | 1540 | CH2 | TRP | 588 | 26.777 | 45.635 | 17.298 | 1.00 | 5.01 | BTKA |
| ATOM | 1541 | C | TRP | 588 | 28.655 | 43.122 | 12.980 | 1.00 | 11.28 | BTKA |
| ATOM | 1542 | O | TRP | 588 | 29.133 | 44.186 | 13.362 | 1.00 | 11.93 | BTKA |
| ATOM | 1543 | N | GLU | 589 | 29.058 | 41.942 | 13.439 | 1.00 | 11.46 | BTKA |
| ATOM | 1544 | CA | GLU | 589 | 30.116 | 41.828 | 14.430 | 1.00 | 10.29 | BTKA |
| ATOM | 1545 | CB | GLU | 589 | 30.177 | 40.409 | 15.010 | 1.00 | 10.54 | BTKA |
| ATOM | 1546 | CG | GLU | 589 | 28.996 | 40.024 | 15.873 | 1.00 | 6.40 | BTKA |
| ATOM | 1547 | CD | GLU | 589 | 29.184 | 38.674 | 16.522 | 1.00 | 5.99 | BTKA |
| ATOM | 1548 | OE1 | GLU | 589 | 29.688 | 38.628 | 17.661 | 1.00 | 10.51 | BTKA |
| ATOM | 1549 | OE2 | GLU | 589 | 28.835 | 37.654 | 15.890 | 1.00 | 5.23 | BTKA |
| ATOM | 1550 | C | GLU | 589 | 31.442 | 42.164 | 13.768 | 1.00 | 11.38 | BTKA |
| ATOM | 1551 | O | GLU | 589 | 32.287 | 42.844 | 14.349 | 1.00 | 9.66 | BTKA |
| ATOM | 1552 | N | ILE | 590 | 31.632 | 41.666 | 12.554 | 1.00 | 9.79 | BTKA |
| ATOM | 1553 | CA | ILE | 590 | 32.860 | 41.931 | 11.825 | 1.00 | 14.53 | BTKA |
| ATOM | 1554 | CB | ILE | 590 | 32.921 | 41.136 | 10.476 | 1.00 | 13.48 | BTKA |
| ATOM | 1555 | CG2 | ILE | 590 | 34.080 | 41.612 | 9.628 | 1.00 | 13.14 | BTKA |
| ATOM | 1556 | CG1 | ILE | 590 | 33.080 | 39.637 | 10.746 | 1.00 | 14.32 | BTKA |
| ATOM | 1557 | CD | ILE | 590 | 33.136 | 38.788 | 9.489 | 1.00 | 2.34 | BTKA |
| ATOM | 1558 | C | ILE | 590 | 32.982 | 43.426 | 11.552 | 1.00 | 14.06 | BTKA |
| ATOM | 1559 | O | ILE | 590 | 33.983 | 44.052 | 11.908 | 1.00 | 15.05 | BTKA |
| ATOM | 1560 | N | TYR | 591 | 31.931 | 44.007 | 10.982 | 1.00 | 14.63 | BTKA |
| ATOM | 1561 | CA | TYR | 591 | 31.939 | 45.423 | 10.642 | 1.00 | 10.81 | BTKA |
| ATOM | 1562 | CB | TYR | 591 | 30.878 | 45.721 | 9.592 | 1.00 | 11.09 | BTKA |
| ATOM | 1563 | CG | TYR | 591 | 31.410 | 45.434 | 8.213 | 1.00 | 11.87 | BTKA |
| ATOM | 1564 | CD1 | TYR | 591 | 31.381 | 44.144 | 7.684 | 1.00 | 15.61 | BTKA |
| ATOM | 1565 | CE1 | TYR | 591 | 31.955 | 43.863 | 6.446 | 1.00 | 11.16 | BTKA |
| ATOM | 1566 | CD2 | TYR | 591 | 32.022 | 46.437 | 7.466 | 1.00 | 10.34 | BTKA |
| ATOM | 1567 | CE2 | TYR | 591 | 32.598 | 46.170 | 6.235 | 1.00 | 10.30 | BTKA |
| ATOM | 1568 | CZ | TYR | 591 | 32.564 | 44.883 | 5.731 | 1.00 | 11.77 | BTKA |
| ATOM | 1569 | OH | TYR | 591 | 33.154 | 44.620 | 4.519 | 1.00 | 18.27 | BTKA |
| ATOM | 1570 | C | TYR | 591 | 31.964 | 46.440 | 11.780 | 1.00 | 8.81 | BTKA |
| ATOM | 1571 | O | TYR | 591 | 32.575 | 47.505 | 11.645 | 1.00 | 9.70 | BTKA |
| ATOM | 1572 | N | SER | 592 | 31.330 | 46.126 | 12.904 | 1.00 | 9.16 | BTKA |
| ATOM | 1573 | CA | SER | 592 | 31.377 | 47.033 | 14.051 | 1.00 | 10.14 | BTKA |
| ATOM | 1574 | CB | SER | 592 | 30.157 | 46.849 | 14.961 | 1.00 | 8.60 | BTKA |
| ATOM | 1575 | OG | SER | 592 | 30.143 | 45.577 | 15.572 | 1.00 | 10.12 | BTKA |
| ATOM | 1576 | C | SER | 592 | 32.684 | 46.776 | 14.823 | 1.00 | 11.91 | BTKA |
| ATOM | 1577 | O | SER | 592 | 32.904 | 47.341 | 15.894 | 1.00 | 12.09 | BTKA |
| ATOM | 1578 | N | LEU | 593 | 33.554 | 45.944 | 14.247 | 1.00 | 8.44 | BTKA |
| ATOM | 1579 | CA | LEU | 593 | 34.837 | 45.583 | 14.836 | 1.00 | 11.62 | BTKA |
| ATOM | 1580 | CB | LEU | 593 | 35.794 | 46.784 | 14.878 | 1.00 | 11.72 | BTKA |
| ATOM | 1581 | CG | LEU | 593 | 36.273 | 47.296 | 13.507 | 1.00 | 13.96 | BTKA |
| ATOM | 1582 | CD1 | LEU | 593 | 37.035 | 48.602 | 13.641 | 1.00 | 11.23 | BTKA |
| ATOM | 1583 | CD2 | LEU | 593 | 37.130 | 46.246 | 12.820 | 1.00 | 13.30 | BTKA |
| ATOM | 1584 | C | LEU | 593 | 34.757 | 44.901 | 16.197 | 1.00 | 13.49 | BTKA |
| ATOM | 1585 | O | LEU | 593 | 35.398 | 45.324 | 17.158 | 1.00 | 15.34 | BTKA |
| ATOM | 1586 | N | GLY | 594 | 33.984 | 43.820 | 16.249 | 1.00 | 13.34 | BTKA |
| ATOM | 1587 | CA | GLY | 594 | 33.842 | 43.027 | 17.460 | 1.00 | 13.43 | BTKA |
| ATOM | 1588 | C | GLY | 594 | 32.899 | 43.478 | 18.554 | 1.00 | 12.35 | BTKA |
| ATOM | 1589 | O | GLY | 594 | 33.053 | 43.074 | 19.702 | 1.00 | 16.26 | BTKA |
| ATOM | 1590 | N | LYS | 595 | 31.907 | 44.292 | 18.225 | 1.00 | 15.44 | BTKA |
| ATOM | 1591 | CA | LYS | 595 | 30.982 | 44.751 | 19.252 | 1.00 | 14.58 | BTKA |
| ATOM | 1592 | CB | LYS | 595 | 30.422 | 46.133 | 18.902 | 1.00 | 16.29 | BTKA |
| ATOM | 1593 | CG | LYS | 595 | 31.498 | 47.153 | 18.538 | 1.00 | 17.65 | BTKA |
| ATOM | 1594 | CD | LYS | 595 | 32.699 | 47.035 | 19.468 | 1.00 | 20.70 | BTKA |
| ATOM | 1595 | CE | LYS | 595 | 33.937 | 47.733 | 18.911 | 1.00 | 19.00 | BTKA |
| ATOM | 1596 | NZ | LYS | 595 | 35.142 | 47.357 | 19.707 | 1.00 | 21.27 | BTKA |
| ATOM | 1597 | C | LYS | 595 | 29.862 | 43.746 | 19.481 | 1.00 | 11.31 | BTKA |
| ATOM | 1598 | O | LYS | 595 | 29.561 | 42.941 | 18.600 | 1.00 | 11.64 | BTKA |
| ATOM | 1599 | N | MET | 596 | 29.311 | 43.743 | 20.695 | 1.00 | 13.47 | BTKA |
| ATOM | 1600 | CA | MET | 596 | 28.212 | 42.842 | 21.044 | 1.00 | 13.02 | BTKA |
| ATOM | 1601 | CB | MET | 596 | 28.097 | 42.639 | 22.569 | 1.00 | 17.06 | BTKA |
| ATOM | 1602 | CG | MET | 596 | 26.706 | 42.143 | 23.017 | 1.00 | 18.96 | BTKA |
| ATOM | 1603 | SD | MET | 596 | 26.594 | 41.087 | 24.493 | 1.00 | 25.10 | BTKA |
| ATOM | 1604 | CE | MET | 596 | 26.050 | 42.271 | 25.755 | 1.00 | 16.77 | BTKA |
| ATOM | 1605 | C | MET | 596 | 26.908 | 43.396 | 20.502 | 1.00 | 8.51 | BTKA |
| ATOM | 1606 | O | MET | 596 | 26.670 | 44.611 | 20.547 | 1.00 | 8.39 | BTKA |
| ATOM | 1607 | N | PRO | 597 | 26.042 | 42.511 | 19.986 | 1.00 | 6.79 | BTKA |
| ATOM | 1608 | CD | PRO | 597 | 26.280 | 41.091 | 19.679 | 1.00 | 2.92 | BTKA |
| ATOM | 1609 | CA | PRO | 597 | 24.760 | 42.938 | 19.441 | 1.00 | 7.77 | BTKA |
| ATOM | 1610 | CB | PRO | 597 | 24.283 | 41.694 | 18.679 | 1.00 | 2.29 | BTKA |
| ATOM | 1611 | CG | PRO | 597 | 25.559 | 40.945 | 18.387 | 1.00 | 2.89 | BTKA |
| ATOM | 1612 | C | PRO | 597 | 23.791 | 43.330 | 20.541 | 1.00 | 5.87 | BTKA |
| ATOM | 1613 | O | PRO | 597 | 23.608 | 42.598 | 21.504 | 1.00 | 8.10 | BTKA |
| ATOM | 1614 | N | TYR | 598 | 23.198 | 44.509 | 20.421 | 1.00 | 11.67 | BTKA |
| ATOM | 1615 | CA | TYR | 598 | 22.225 | 44.984 | 21.403 | 1.00 | 11.39 | BTKA |
| ATOM | 1616 | CB | TYR | 598 | 20.986 | 44.087 | 21.374 | 1.00 | 11.13 | BTKA |
| ATOM | 1617 | CG | TYR | 598 | 20.502 | 43.671 | 20.010 | 1.00 | 10.46 | BTKA |
| ATOM | 1618 | CD1 | TYR | 598 | 20.737 | 42.384 | 19.538 | 1.00 | 8.57 | BTKA |
| ATOM | 1619 | CE1 | TYR | 598 | 20.241 | 41.968 | 18.321 | 1.00 | 9.01 | BTKA |
| ATOM | 1620 | CD2 | TYR | 598 | 19.757 | 44.541 | 19.219 | 1.00 | 11.82 | BTKA |
| ATOM | 1621 | CE2 | TYR | 598 | 19.255 | 44.139 | 17.998 | 1.00 | 11.15 | BTKA |
| ATOM | 1622 | CZ | TYR | 598 | 19.499 | 42.850 | 17.553 | 1.00 | 10.70 | BTKA |
| ATOM | 1623 | OH | TYR | 598 | 19.007 | 42.443 | 16.335 | 1.00 | 12.06 | BTKA |
| ATOM | 1624 | C | TYR | 598 | 22.731 | 45.018 | 22.851 | 1.00 | 13.88 | BTKA |
| ATOM | 1625 | O | TYR | 598 | 21.993 | 44.656 | 23.762 | 1.00 | 14.96 | BTKA |
| ATOM | 1626 | N | GLU | 599 | 23.940 | 45.518 | 23.078 | 1.00 | 17.51 | BTKA |
| ATOM | 1627 | CA | GLU | 599 | 24.506 | 45.556 | 24.434 | 1.00 | 21.90 | BTKA |
| ATOM | 1628 | CB | GLU | 599 | 25.965 | 46.063 | 24.418 | 1.00 | 25.83 | BTKA |
| ATOM | 1629 | CG | GLU | 599 | 26.155 | 47.580 | 24.303 | 1.00 | 25.70 | BTKA |
| ATOM | 1630 | CD | GLU | 599 | 25.989 | 48.100 | 22.887 | 1.00 | 29.61 | BTKA |
| ATOM | 1631 | OE1 | GLU | 599 | 24.842 | 48.148 | 22.385 | 1.00 | 34.92 | BTKA |
| ATOM | 1632 | OE2 | GLU | 599 | 27.018 | 48.465 | 22.275 | 1.00 | 36.19 | BTKA |
| ATOM | 1633 | C | GLU | 599 | 23.707 | 46.290 | 25.523 | 1.00 | 22.83 | BTKA |
| ATOM | 1634 | O | GLU | 599 | 23.867 | 45.990 | 26.704 | 1.00 | 21.63 | BTKA |
| ATOM | 1635 | N | ARG | 600 | 22.903 | 47.285 | 25.157 | 1.00 | 23.83 | BTKA |
| ATOM | 1636 | CA | ARG | 600 | 22.120 | 47.991 | 26.170 | 1.00 | 25.44 | BTKA |
| ATOM | 1637 | CB | ARG | 600 | 21.977 | 49.477 | 25.857 | 1.00 | 23.52 | BTKA |
| ATOM | 1638 | CG | ARG | 600 | 22.569 | 50.385 | 26.941 | 1.00 | 27.22 | BTKA |
| ATOM | 1639 | CD | ARG | 600 | 21.939 | 50.168 | 28.326 | 1.00 | 31.56 | BTKA |
| ATOM | 1640 | NE | ARG | 600 | 22.425 | 51.152 | 29.296 | 1.00 | 30.45 | BTKA |
| ATOM | 1641 | CZ | ARG | 600 | 21.656 | 51.847 | 30.136 | 1.00 | 28.69 | BTKA |
| ATOM | 1642 | NH1 | ARG | 600 | 22.211 | 52.726 | 30.960 | 1.00 | 27.63 | BTKA |
| ATOM | 1643 | NH2 | ARG | 600 | 20.344 | 51.659 | 30.175 | 1.00 | 26.46 | BTKA |
| ATOM | 1644 | C | ARG | 600 | 20.750 | 47.351 | 26.356 | 1.00 | 26.74 | BTKA |
| ATOM | 1645 | O | ARG | 600 | 19.931 | 47.818 | 27.162 | 1.00 | 27.64 | BTKA |
| ATOM | 1646 | N | PHE | 601 | 20.492 | 46.309 | 25.571 | 1.00 | 24.54 | BTKA |
| ATOM | 1647 | CA | PHE | 601 | 19.243 | 45.573 | 25.664 | 1.00 | 20.59 | BTKA |
| ATOM | 1648 | CB | PHE | 601 | 18.841 | 44.989 | 24.305 | 1.00 | 22.31 | BTKA |
| ATOM | 1649 | CG | PHE | 601 | 18.485 | 46.006 | 23.266 | 1.00 | 22.63 | BTKA |
| ATOM | 1650 | CD1 | PHE | 601 | 17.177 | 46.128 | 22.823 | 1.00 | 23.34 | BTKA |
| ATOM | 1651 | CD2 | PHE | 601 | 19.460 | 46.791 | 22.677 | 1.00 | 20.08 | BTKA |
| ATOM | 1652 | CE1 | PHE | 601 | 16.852 | 47.011 | 21.809 | 1.00 | 20.90 | BTKA |
| ATOM | 1653 | CE2 | PHE | 601 | 19.138 | 47.674 | 21.660 | 1.00 | 20.46 | BTKA |
| ATOM | 1654 | CZ | PHE | 601 | 17.832 | 47.783 | 21.225 | 1.00 | 22.29 | BTKA |
| ATOM | 1655 | C | PHE | 601 | 19.511 | 44.399 | 26.588 | 1.00 | 20.01 | BTKA |
| ATOM | 1656 | O | PHE | 601 | 20.624 | 44.202 | 27.074 | 1.00 | 18.72 | BTKA |
| ATOM | 1657 | N | THR | 602 | 18.481 | 43.601 | 26.801 | 1.00 | 22.29 | BTKA |
| ATOM | 1658 | CA | THR | 602 | 18.582 | 42.400 | 27.604 | 1.00 | 23.72 | BTKA |
| ATOM | 1659 | CB | THR | 602 | 17.681 | 42.486 | 28.861 | 1.00 | 24.01 | BTKA |
| ATOM | 1660 | OG1 | THR | 602 | 16.339 | 42.823 | 28.478 | 1.00 | 20.42 | BTKA |
| ATOM | 1661 | CG2 | THR | 602 | 18.204 | 43.538 | 29.823 | 1.00 | 27.75 | BTKA |
| ATOM | 1662 | C | THR | 602 | 18.061 | 41.323 | 26.652 | 1.00 | 25.22 | BTKA |
| ATOM | 1663 | O | THR | 602 | 17.420 | 41.645 | 25.644 | 1.00 | 27.52 | BTKA |
| ATOM | 1664 | N | ASN | 603 | 18.333 | 40.055 | 26.939 | 1.00 | 26.65 | BTKA |
| ATOM | 1665 | CA | ASN | 603 | 17.844 | 38.979 | 26.072 | 1.00 | 25.54 | BTKA |
| ATOM | 1666 | CB | ASN | 603 | 18.192 | 37.607 | 26.662 | 1.00 | 20.91 | BTKA |
| ATOM | 1667 | CG | ASN | 603 | 19.674 | 37.298 | 26.576 | 1.00 | 25.12 | BTKA |
| ATOM | 1668 | OD1 | ASN | 603 | 20.490 | 38.194 | 26.385 | 1.00 | 25.37 | BTKA |
| ATOM | 1669 | ND2 | ASN | 603 | 20.026 | 36.028 | 26.704 | 1.00 | 26.15 | BTKA |
| ATOM | 1670 | C | ASN | 603 | 16.337 | 39.082 | 25.803 | 1.00 | 25.43 | BTKA |
| ATOM | 1671 | O | ASN | 603 | 15.864 | 38.698 | 24.736 | 1.00 | 23.83 | BTKA |
| ATOM | 1672 | N | SER | 604 | 15.597 | 39.655 | 26.747 | 1.00 | 26.48 | BTKA |
| ATOM | 1673 | CA | SER | 604 | 14.152 | 39.806 | 26.602 | 1.00 | 24.32 | BTKA |
| ATOM | 1674 | CB | SER | 604 | 13.487 | 39.881 | 27.977 | 1.00 | 24.89 | BTKA |
| ATOM | 1675 | OG | SER | 604 | 14.121 | 40.858 | 28.786 | 1.00 | 27.86 | BTKA |
| ATOM | 1676 | C | SER | 604 | 13.767 | 41.025 | 25.768 | 1.00 | 21.82 | BTKA |
| ATOM | 1677 | O | SER | 604 | 12.799 | 40.983 | 25.005 | 1.00 | 19.52 | BTKA |
| ATOM | 1678 | N | GLU | 605 | 14.505 | 42.118 | 25.933 | 1.00 | 23.74 | BTKA |
| ATOM | 1679 | CA | GLU | 605 | 14.229 | 43.343 | 25.181 | 1.00 | 21.76 | BTKA |
| ATOM | 1680 | CB | GLU | 605 | 14.937 | 44.543 | 25.815 | 1.00 | 19.06 | BTKA |
| ATOM | 1681 | CG | GLU | 605 | 14.413 | 44.880 | 27.213 | 1.00 | 21.14 | BTKA |
| ATOM | 1682 | CD | GLU | 605 | 15.253 | 45.914 | 27.930 | 1.00 | 18.46 | BTKA |
| ATOM | 1683 | OE1 | GLU | 605 | 14.730 | 46.576 | 28.850 | 1.00 | 22.19 | BTKA |
| ATOM | 1684 | OE2 | GLU | 605 | 16.443 | 46.064 | 27.583 | 1.00 | 25.21 | BTKA |
| ATOM | 1685 | C | GLU | 605 | 14.656 | 43.159 | 23.733 | 1.00 | 19.22 | BTKA |
| ATOM | 1686 | O | GLU | 605 | 14.020 | 43.688 | 22.820 | 1.00 | 17.88 | BTKA |
| ATOM | 1687 | N | THR | 606 | 15.710 | 42.374 | 23.534 | 1.00 | 19.68 | BTKA |
| ATOM | 1688 | CA | THR | 606 | 16.231 | 42.064 | 22.205 | 1.00 | 17.05 | BTKA |
| ATOM | 1689 | CB | THR | 606 | 17.471 | 41.148 | 22.306 | 1.00 | 15.00 | BTKA |
| ATOM | 1690 | OG1 | THR | 606 | 18.458 | 41.769 | 23.139 | 1.00 | 15.89 | BTKA |
| ATOM | 1691 | CG2 | THR | 606 | 18.056 | 40.871 | 20.938 | 1.00 | 12.52 | BTKA |
| ATOM | 1692 | C | THR | 606 | 15.138 | 41.339 | 21.418 | 1.00 | 16.80 | BTKA |
| ATOM | 1693 | O | THR | 606 | 14.900 | 41.637 | 20.243 | 1.00 | 18.76 | BTKA |
| ATOM | 1694 | N | ALA | 607 | 14.457 | 40.408 | 22.084 | 1.00 | 16.21 | BTKA |
| ATOM | 1695 | CA | ALA | 607 | 13.377 | 39.648 | 21.467 | 1.00 | 16.69 | BTKA |
| ATOM | 1696 | CB | ALA | 607 | 12.926 | 38.532 | 22.384 | 1.00 | 14.32 | BTKA |
| ATOM | 1697 | C | ALA | 607 | 12.211 | 40.573 | 21.138 | 1.00 | 17.62 | BTKA |
| ATOM | 1698 | O | ALA | 607 | 11.574 | 40.417 | 20.098 | 1.00 | 21.64 | BTKA |
| ATOM | 1699 | N | GLU | 608 | 11.940 | 41.540 | 22.015 | 1.00 | 16.80 | BTKA |
| ATOM | 1700 | CA | GLU | 608 | 10.855 | 42.497 | 21.792 | 1.00 | 17.47 | BTKA |
| ATOM | 1701 | CB | GLU | 608 | 10.574 | 43.312 | 23.064 | 1.00 | 24.26 | BTKA |
| ATOM | 1702 | CG | GLU | 608 | 9.979 | 42.503 | 24.219 | 1.00 | 31.87 | BTKA |
| ATOM | 1703 | CD | GLU | 608 | 9.562 | 43.363 | 25.414 | 1.00 | 32.17 | BTKA |
| ATOM | 1704 | OE1 | GLU | 608 | 8.411 | 43.210 | 25.892 | 1.00 | 33.50 | BTKA |
| ATOM | 1705 | OE2 | GLU | 608 | 10.386 | 44.183 | 25.886 | 1.00 | 34.89 | BTKA |
| ATOM | 1706 | C | GLU | 608 | 11.207 | 43.438 | 20.631 | 1.00 | 13.51 | BTKA |
| ATOM | 1707 | O | GLU | 608 | 10.370 | 43.714 | 19.765 | 1.00 | 5.86 | BTKA |
| ATOM | 1708 | N | HIS | 609 | 12.472 | 43.865 | 20.603 | 1.00 | 9.80 | BTKA |
| ATOM | 1709 | CA | HIS | 609 | 13.025 | 44.762 | 19.586 | 1.00 | 6.26 | BTKA |
| ATOM | 1710 | CB | HIS | 609 | 14.509 | 45.014 | 19.886 | 1.00 | 6.17 | BTKA |
| ATOM | 1711 | CG | HIS | 609 | 15.179 | 45.976 | 18.950 | 1.00 | 7.14 | BTKA |
| ATOM | 1712 | CD2 | HIS | 609 | 15.978 | 45.769 | 17.879 | 1.00 | 9.08 | BTKA |
| ATOM | 1713 | ND1 | HIS | 609 | 15.114 | 47.342 | 19.116 | 1.00 | 10.07 | BTKA |
| ATOM | 1714 | CE1 | HIS | 609 | 15.848 | 47.935 | 18.192 | 1.00 | 4.17 | BTKA |
| ATOM | 1715 | NE2 | HIS | 609 | 16.384 | 47.002 | 17.427 | 1.00 | 8.56 | BTKA |
| ATOM | 1716 | C | HIS | 609 | 12.881 | 44.121 | 18.221 | 1.00 | 9.48 | BTKA |
| ATOM | 1717 | O | HIS | 609 | 12.489 | 44.782 | 17.255 | 1.00 | 11.18 | BTKA |
| ATOM | 1718 | N | ILE | 610 | 13.214 | 42.839 | 18.138 | 1.00 | 8.84 | BTKA |
| ATOM | 1719 | CA | ILE | 610 | 13.106 | 42.113 | 16.881 | 1.00 | 11.99 | BTKA |
| ATOM | 1720 | CB | ILE | 610 | 13.869 | 40.758 | 16.945 | 1.00 | 13.75 | BTKA |
| ATOM | 1721 | CG2 | ILE | 610 | 13.211 | 39.701 | 16.048 | 1.00 | 13.99 | BTKA |
| ATOM | 1722 | CG1 | ILE | 610 | 15.345 | 40.991 | 16.594 | 1.00 | 6.66 | BTKA |
| ATOM | 1723 | CD | ILE | 610 | 16.188 | 39.734 | 16.592 | 1.00 | 8.43 | BTKA |
| ATOM | 1724 | C | ILE | 610 | 11.635 | 41.923 | 16.527 | 1.00 | 14.02 | BTKA |
| ATOM | 1725 | O | ILE | 610 | 11.257 | 41.964 | 15.356 | 1.00 | 12.60 | BTKA |
| ATOM | 1726 | N | ALA | 611 | 10.802 | 41.798 | 17.557 | 1.00 | 15.80 | BTKA |
| ATOM | 1727 | CA | ALA | 611 | 9.368 | 41.631 | 17.377 | 1.00 | 14.09 | BTKA |
| ATOM | 1728 | CB | ALA | 611 | 8.713 | 41.313 | 18.710 | 1.00 | 15.18 | BTKA |
| ATOM | 1729 | C | ALA | 611 | 8.759 | 42.889 | 16.767 | 1.00 | 13.18 | BTKA |
| ATOM | 1730 | O | ALA | 611 | 7.688 | 42.843 | 16.174 | 1.00 | 16.50 | BTKA |
| ATOM | 1731 | N | GLN | 612 | 9.448 | 44.012 | 16.918 | 1.00 | 9.26 | BTKA |
| ATOM | 1732 | CA | GLN | 612 | 8.996 | 45.288 | 16.369 | 1.00 | 9.32 | BTKA |
| ATOM | 1733 | CB | GLN | 612 | 9.538 | 46.438 | 17.214 | 1.00 | 12.00 | BTKA |
| ATOM | 1734 | CG | GLN | 612 | 9.266 | 46.315 | 18.699 | 1.00 | 15.05 | BTKA |
| ATOM | 1735 | CD | GLN | 612 | 8.041 | 47.065 | 19.115 | 1.00 | 19.92 | BTKA |
| ATOM | 1736 | OE1 | GLN | 612 | 6.918 | 46.656 | 18.833 | 1.00 | 26.26 | BTKA |
| ATOM | 1737 | NE2 | GLN | 612 | 8.246 | 48.194 | 19.773 | 1.00 | 24.14 | BTKA |
| ATOM | 1738 | C | GLN | 612 | 9.497 | 45.470 | 14.939 | 1.00 | 8.38 | BTKA |
| ATOM | 1739 | O | GLN | 612 | 9.184 | 46.471 | 14.285 | 1.00 | 8.92 | BTKA |
| ATOM | 1740 | N | GLY | 613 | 10.308 | 44.523 | 14.476 | 1.00 | 11.09 | BTKA |
| ATOM | 1741 | CA | GLY | 613 | 10.861 | 44.598 | 13.135 | 1.00 | 10.83 | BTKA |
| ATOM | 1742 | C | GLY | 613 | 12.164 | 45.381 | 13.076 | 1.00 | 12.46 | BTKA |
| ATOM | 1743 | O | GLY | 613 | 12.642 | 45.711 | 11.993 | 1.00 | 8.12 | BTKA |
| ATOM | 1744 | N | LEU | 614 | 12.736 | 45.690 | 14.237 | 1.00 | 10.93 | BTKA |
| ATOM | 1745 | CA | LEU | 614 | 13.988 | 46.439 | 14.300 | 1.00 | 11.92 | BTKA |
| ATOM | 1746 | CB | LEU | 614 | 14.012 | 47.375 | 15.518 | 1.00 | 9.21 | BTKA |
| ATOM | 1747 | CG | LEU | 614 | 13.103 | 48.609 | 15.581 | 1.00 | 11.29 | BTKA |
| ATOM | 1748 | CD1 | LEU | 614 | 12.426 | 48.880 | 14.249 | 1.00 | 11.46 | BTKA |
| ATOM | 1749 | CD2 | LEU | 614 | 12.078 | 48.433 | 16.656 | 1.00 | 4.88 | BTKA |
| ATOM | 1750 | C | LEU | 614 | 15.188 | 45.495 | 14.330 | 1.00 | 11.01 | BTKA |
| ATOM | 1751 | O | LEU | 614 | 15.148 | 44.424 | 14.947 | 1.00 | 9.79 | BTKA |
| ATOM | 1752 | N | ARG | 615 | 16.273 | 45.920 | 13.694 | 1.00 | 12.00 | BTKA |
| ATOM | 1753 | CA | ARG | 615 | 17.484 | 45.117 | 13.603 | 1.00 | 9.33 | BTKA |
| ATOM | 1754 | CB | ARG | 615 | 17.704 | 44.713 | 12.144 | 1.00 | 9.55 | BTKA |
| ATOM | 1755 | CG | ARG | 615 | 16.518 | 44.023 | 11.488 | 1.00 | 7.26 | BTKA |
| ATOM | 1756 | CD | ARG | 615 | 16.209 | 42.703 | 12.141 | 1.00 | 3.51 | BTKA |
| ATOM | 1757 | NE | ARG | 615 | 15.114 | 42.016 | 11.473 | 1.00 | 7.24 | BTKA |
| ATOM | 1758 | CZ | ARG | 615 | 13.889 | 41.891 | 11.976 | 1.00 | 8.86 | BTKA |
| ATOM | 1759 | NH1 | ARG | 615 | 13.582 | 42.421 | 13.156 | 1.00 | 7.13 | BTKA |
| ATOM | 1760 | NH2 | ARG | 615 | 12.986 | 41.170 | 11.329 | 1.00 | 8.16 | BTKA |
| ATOM | 1761 | C | ARG | 615 | 18.704 | 45.885 | 14.098 | 1.00 | 8.91 | BTKA |
| ATOM | 1762 | O | ARG | 615 | 18.570 | 46.921 | 14.762 | 1.00 | 7.60 | BTKA |
| ATOM | 1763 | N | LEU | 616 | 19.887 | 45.379 | 13.756 | 1.00 | 7.61 | BTKA |
| ATOM | 1764 | CA | LEU | 616 | 21.155 | 45.990 | 14.138 | 1.00 | 9.07 | BTKA |
| ATOM | 1765 | CB | LEU | 616 | 22.255 | 44.927 | 14.183 | 1.00 | 9.04 | BTKA |
| ATOM | 1766 | CG | LEU | 616 | 22.202 | 43.944 | 15.355 | 1.00 | 9.90 | BTKA |
| ATOM | 1767 | CD1 | LEU | 616 | 23.034 | 42.705 | 15.046 | 1.00 | 6.52 | BTKA |
| ATOM | 1768 | CD2 | LEU | 616 | 22.690 | 44.632 | 16.623 | 1.00 | 8.47 | BTKA |
| ATOM | 1769 | C | LEU | 616 | 21.545 | 47.094 | 13.162 | 1.00 | 7.88 | BTKA |
| ATOM | 1770 | O | LEU | 616 | 21.682 | 46.848 | 11.965 | 1.00 | 11.49 | BTKA |
| ATOM | 1771 | N | TYR | 617 | 21.770 | 48.295 | 13.696 | 1.00 | 9.86 | BTKA |
| ATOM | 1772 | CA | TYR | 617 | 22.133 | 49.480 | 12.913 | 1.00 | 6.95 | BTKA |
| ATOM | 1773 | CB | TYR | 617 | 22.440 | 50.653 | 13.853 | 1.00 | 8.23 | BTKA |
| ATOM | 1774 | CG | TYR | 617 | 23.719 | 50.515 | 14.674 | 1.00 | 11.82 | BTKA |
| ATOM | 1775 | CD1 | TYR | 617 | 24.923 | 51.090 | 14.244 | 1.00 | 11.55 | BTKA |
| ATOM | 1776 | CE1 | TYR | 617 | 26.086 | 51.011 | 15.021 | 1.00 | 9.18 | BTKA |
| ATOM | 1777 | CD2 | TYR | 617 | 23.714 | 49.856 | 15.897 | 1.00 | 10.14 | BTKA |
| ATOM | 1778 | CE2 | TYR | 617 | 24.869 | 49.777 | 16.682 | 1.00 | 15.29 | BTKA |
| ATOM | 1779 | CZ | TYR | 617 | 26.047 | 50.358 | 16.240 | 1.00 | 13.29 | BTKA |
| ATOM | 1780 | OH | TYR | 617 | 27.167 | 50.305 | 17.043 | 1.00 | 10.85 | BTKA |
| ATOM | 1781 | C | TYR | 617 | 23.315 | 49.298 | 11.962 | 1.00 | 7.09 | BTKA |
| ATOM | 1782 | O | TYR | 617 | 24.166 | 48.440 | 12.182 | 1.00 | 6.00 | BTKA |
| ATOM | 1783 | N | ARG | 618 | 23.391 | 50.146 | 10.938 | 1.00 | 7.06 | BTKA |
| ATOM | 1784 | CA | ARG | 618 | 24.498 | 50.072 | 9.990 | 1.00 | 13.07 | BTKA |
| ATOM | 1785 | CB | ARG | 618 | 24.271 | 50.947 | 8.752 | 1.00 | 11.95 | BTKA |
| ATOM | 1786 | CG | ARG | 618 | 25.389 | 50.761 | 7.721 | 1.00 | 12.02 | BTKA |
| ATOM | 1787 | CD | ARG | 618 | 25.823 | 52.046 | 7.028 | 1.00 | 12.70 | BTKA |
| ATOM | 1788 | NE | ARG | 618 | 24.896 | 52.504 | 5.996 | 1.00 | 10.05 | BTKA |
| ATOM | 1789 | CZ | ARG | 618 | 25.263 | 53.226 | 4.939 | 1.00 | 8.38 | BTKA |
| ATOM | 1790 | NH1 | ARG | 618 | 26.535 | 53.561 | 4.769 | 1.00 | 8.75 | BTKA |
| ATOM | 1791 | NH2 | ARG | 618 | 24.352 | 53.659 | 4.076 | 1.00 | 8.27 | BTKA |
| ATOM | 1792 | C | ARG | 618 | 25.767 | 50.547 | 10.668 | 1.00 | 15.11 | BTKA |
| ATOM | 1793 | O | ARG | 618 | 25.798 | 51.647 | 11.223 | 1.00 | 18.30 | BTKA |
| ATOM | 1794 | N | PRO | 619 | 26.803 | 49.694 | 10.713 | 1.00 | 17.30 | BTKA |
| ATOM | 1795 | CD | PRO | 619 | 26.790 | 48.234 | 10.509 | 1.00 | 15.23 | BTKA |
| ATOM | 1796 | CA | PRO | 619 | 28.045 | 50.130 | 11.353 | 1.00 | 16.98 | BTKA |
| ATOM | 1797 | CB | PRO | 619 | 28.740 | 48.808 | 11.683 | 1.00 | 16.78 | BTKA |
| ATOM | 1798 | CG | PRO | 619 | 28.237 | 47.876 | 10.651 | 1.00 | 13.87 | BTKA |
| ATOM | 1799 | C | PRO | 619 | 28.871 | 51.039 | 10.430 | 1.00 | 16.87 | BTKA |
| ATOM | 1800 | O | PRO | 619 | 28.956 | 50.805 | 9.229 | 1.00 | 15.47 | BTKA |
| ATOM | 1801 | N | HIS | 620 | 29.429 | 52.101 | 11.008 | 1.00 | 16.43 | BTKA |
| ATOM | 1802 | CA | HIS | 620 | 30.243 | 53.088 | 10.287 | 1.00 | 19.75 | BTKA |
| ATOM | 1803 | CB | HIS | 620 | 30.946 | 54.021 | 11.290 | 1.00 | 19.36 | BTKA |
| ATOM | 1804 | CG | HIS | 620 | 31.863 | 53.317 | 12.247 | 1.00 | 20.09 | BTKA |
| ATOM | 1805 | CD2 | HIS | 620 | 33.211 | 53.355 | 12.384 | 1.00 | 21.52 | BTKA |
| ATOM | 1806 | ND1 | HIS | 620 | 31.407 | 52.455 | 13.222 | 1.00 | 20.76 | BTKA |
| ATOM | 1807 | CE1 | HIS | 620 | 32.434 | 51.988 | 13.912 | 1.00 | 16.73 | BTKA |
| ATOM | 1808 | NE2 | HIS | 620 | 33.538 | 52.519 | 13.423 | 1.00 | 17.10 | BTKA |
| ATOM | 1809 | C | HIS | 620 | 31.257 | 52.589 | 9.239 | 1.00 | 21.42 | BTKA |
| ATOM | 1810 | O | HIS | 620 | 31.646 | 53.344 | 8.347 | 1.00 | 22.39 | BTKA |
| ATOM | 1811 | N | LEU | 621 | 31.695 | 51.338 | 9.346 | 1.00 | 19.75 | BTKA |
| ATOM | 1812 | CA | LEU | 621 | 32.661 | 50.793 | 8.396 | 1.00 | 16.60 | BTKA |
| ATOM | 1813 | CB | LEU | 621 | 33.712 | 49.948 | 9.118 | 1.00 | 13.75 | BTKA |
| ATOM | 1814 | CG | LEU | 621 | 34.539 | 50.612 | 10.226 | 1.00 | 9.81 | BTKA |
| ATOM | 1815 | CD1 | LEU | 621 | 35.542 | 49.613 | 10.742 | 1.00 | 4.63 | BTKA |
| ATOM | 1816 | CD2 | LEU | 621 | 35.248 | 51.863 | 9.713 | 1.00 | 9.43 | BTKA |
| ATOM | 1817 | C | LEU | 621 | 32.010 | 49.966 | 7.296 | 1.00 | 17.19 | BTKA |
| ATOM | 1818 | O | LEU | 621 | 32.694 | 49.260 | 6.558 | 1.00 | 18.71 | BTKA |
| ATOM | 1819 | N | ALA | 622 | 30.690 | 50.054 | 7.184 | 1.00 | 18.73 | BTKA |
| ATOM | 1820 | CA | ALA | 622 | 29.958 | 49.309 | 6.171 | 1.00 | 14.75 | BTKA |
| ATOM | 1821 | CB | ALA | 622 | 28.886 | 48.449 | 6.826 | 1.00 | 16.96 | BTKA |
| ATOM | 1822 | C | ALA | 622 | 29.322 | 50.248 | 5.158 | 1.00 | 14.61 | BTKA |
| ATOM | 1823 | O | ALA | 622 | 28.808 | 51.309 | 5.512 | 1.00 | 15.18 | BTKA |
| ATOM | 1824 | N | SER | 623 | 29.396 | 49.875 | 3.886 | 1.00 | 16.92 | BTKA |
| ATOM | 1825 | CA | SER | 623 | 28.793 | 50.670 | 2.825 | 1.00 | 14.96 | BTKA |
| ATOM | 1826 | CB | SER | 623 | 29.433 | 50.326 | 1.479 | 1.00 | 10.06 | BTKA |
| ATOM | 1827 | OG | SER | 623 | 29.253 | 48.958 | 1.163 | 1.00 | 7.67 | BTKA |
| ATOM | 1828 | C | SER | 623 | 27.309 | 50.336 | 2.785 | 1.00 | 13.91 | BTKA |
| ATOM | 1829 | O | SER | 623 | 26.867 | 49.392 | 3.433 | 1.00 | 15.97 | BTKA |
| ATOM | 1830 | N | GLU | 624 | 26.541 | 51.112 | 2.028 | 1.00 | 15.15 | BTKA |
| ATOM | 1831 | CA | GLU | 624 | 25.110 | 50.868 | 1.888 | 1.00 | 16.38 | BTKA |
| ATOM | 1832 | CB | GLU | 624 | 24.469 | 51.937 | 0.998 | 1.00 | 17.21 | BTKA |
| ATOM | 1833 | CG | GLU | 624 | 22.974 | 51.725 | 0.755 | 1.00 | 19.41 | BTKA |
| ATOM | 1834 | CD | GLU | 624 | 22.452 | 52.478 | −0.459 | 1.00 | 18.43 | BTKA |
| ATOM | 1835 | OE1 | GLU | 624 | 21.396 | 53.129 | −0.339 | 1.00 | 16.44 | BTKA |
| ATOM | 1836 | OE2 | GLU | 624 | 23.083 | 52.406 | −1.539 | 1.00 | 21.39 | BTKA |
| ATOM | 1837 | C | GLU | 624 | 24.937 | 49.499 | 1.241 | 1.00 | 16.27 | BTKA |
| ATOM | 1838 | O | GLU | 624 | 23.935 | 48.824 | 1.451 | 1.00 | 20.19 | BTKA |
| ATOM | 1839 | N | LYS | 625 | 25.933 | 49.111 | 0.447 | 1.00 | 15.04 | BTKA |
| ATOM | 1840 | CA | LYS | 625 | 25.955 | 47.832 | −0.251 | 1.00 | 14.80 | BTKA |
| ATOM | 1841 | CB | LYS | 625 | 26.989 | 47.874 | −1.379 | 1.00 | 15.10 | BTKA |
| ATOM | 1842 | CG | LYS | 625 | 26.462 | 48.451 | −2.693 | 1.00 | 19.31 | BTKA |
| ATOM | 1843 | CD | LYS | 625 | 25.903 | 49.878 | −2.565 | 1.00 | 22.31 | BTKA |
| ATOM | 1844 | CE | LYS | 625 | 26.977 | 50.965 | −2.680 | 1.00 | 21.63 | BTKA |
| ATOM | 1845 | NZ | LYS | 625 | 27.815 | 51.133 | −1.465 | 1.00 | 17.99 | BTKA |
| ATOM | 1846 | C | LYS | 625 | 26.253 | 46.667 | 0.702 | 1.00 | 15.85 | BTKA |
| ATOM | 1847 | O | LYS | 625 | 25.563 | 45.645 | 0.678 | 1.00 | 16.83 | BTKA |
| ATOM | 1848 | N | VAL | 626 | 27.275 | 46.828 | 1.539 | 1.00 | 13.15 | BTKA |
| ATOM | 1849 | CA | VAL | 626 | 27.648 | 45.803 | 2.502 | 1.00 | 8.16 | BTKA |
| ATOM | 1850 | CB | VAL | 626 | 28.954 | 46.166 | 3.228 | 1.00 | 4.19 | BTKA |
| ATOM | 1851 | CG1 | VAL | 626 | 29.170 | 45.266 | 4.437 | 1.00 | 9.56 | BTKA |
| ATOM | 1852 | CG2 | VAL | 626 | 30.127 | 46.037 | 2.264 | 1.00 | 2.00 | BTKA |
| ATOM | 1853 | C | VAL | 626 | 26.523 | 45.649 | 3.508 | 1.00 | 10.56 | BTKA |
| ATOM | 1854 | O | VAL | 626 | 26.211 | 44.538 | 3.947 | 1.00 | 15.38 | BTKA |
| ATOM | 1855 | N | TYR | 627 | 25.870 | 46.754 | 3.837 | 1.00 | 11.00 | BTKA |
| ATOM | 1856 | CA | TYR | 627 | 24.774 | 46.696 | 4.791 | 1.00 | 6.70 | BTKA |
| ATOM | 1857 | CB | TYR | 627 | 24.406 | 48.089 | 5.303 | 1.00 | 5.85 | BTKA |
| ATOM | 1858 | CG | TYR | 627 | 23.438 | 48.052 | 6.462 | 1.00 | 5.63 | BTKA |
| ATOM | 1859 | CD1 | TYR | 627 | 23.826 | 47.547 | 7.699 | 1.00 | 5.65 | BTKA |
| ATOM | 1860 | CE1 | TYR | 627 | 22.943 | 47.500 | 8.765 | 1.00 | 3.44 | BTKA |
| ATOM | 1861 | CD2 | TYR | 627 | 22.133 | 48.510 | 6.320 | 1.00 | 5.85 | BTKA |
| ATOM | 1862 | CE2 | TYR | 627 | 21.237 | 48.465 | 7.383 | 1.00 | 7.61 | BTKA |
| ATOM | 1863 | CZ | TYR | 627 | 21.649 | 47.960 | 8.601 | 1.00 | 4.93 | BTKA |
| ATOM | 1864 | OH | TYR | 627 | 20.768 | 47.925 | 9.657 | 1.00 | 9.76 | BTKA |
| ATOM | 1865 | C | TYR | 627 | 23.566 | 46.005 | 4.182 | 1.00 | 2.00 | BTKA |
| ATOM | 1866 | O | TYR | 627 | 22.816 | 45.355 | 4.884 | 1.00 | 7.93 | BTKA |
| ATOM | 1867 | N | THR | 628 | 23.407 | 46.106 | 2.871 | 1.00 | 4.56 | BTKA |
| ATOM | 1868 | CA | THR | 628 | 22.293 | 45.463 | 2.189 | 1.00 | 6.68 | BTKA |
| ATOM | 1869 | CB | THR | 628 | 22.226 | 45.882 | 0.704 | 1.00 | 12.25 | BTKA |
| ATOM | 1870 | OG1 | THR | 628 | 21.878 | 47.270 | 0.608 | 1.00 | 11.09 | BTKA |
| ATOM | 1871 | CG2 | THR | 628 | 21.196 | 45.057 | −0.043 | 1.00 | 12.83 | BTKA |
| ATOM | 1872 | C | THR | 628 | 22.382 | 43.938 | 2.284 | 1.00 | 3.84 | BTKA |
| ATOM | 1873 | O | THR | 628 | 21.372 | 43.266 | 2.450 | 1.00 | 2.00 | BTKA |
| ATOM | 1874 | N | ILE | 629 | 23.586 | 43.389 | 2.197 | 1.00 | 6.46 | BTKA |
| ATOM | 1875 | CA | ILE | 629 | 23.728 | 41.941 | 2.274 | 1.00 | 7.81 | BTKA |
| ATOM | 1876 | CB | ILE | 629 | 25.067 | 41.425 | 1.645 | 1.00 | 8.16 | BTKA |
| ATOM | 1877 | CG2 | ILE | 629 | 25.040 | 41.634 | 0.154 | 1.00 | 5.31 | BTKA |
| ATOM | 1878 | CG1 | ILE | 629 | 26.287 | 42.154 | 2.207 | 1.00 | 12.39 | BTKA |
| ATOM | 1879 | CD | ILE | 629 | 27.561 | 41.905 | 1.398 | 1.00 | 16.00 | BTKA |
| ATOM | 1880 | C | ILE | 629 | 23.482 | 41.373 | 3.676 | 1.00 | 7.41 | BTKA |
| ATOM | 1881 | O | ILE | 629 | 22.753 | 40.385 | 3.829 | 1.00 | 5.64 | BTKA |
| ATOM | 1882 | N | MET | 630 | 24.014 | 42.025 | 4.705 | 1.00 | 6.37 | BTKA |
| ATOM | 1883 | CA | MET | 630 | 23.790 | 41.527 | 6.052 | 1.00 | 8.30 | BTKA |
| ATOM | 1884 | CB | MET | 630 | 24.765 | 42.147 | 7.057 | 1.00 | 9.46 | BTKA |
| ATOM | 1885 | CG | MET | 630 | 24.540 | 43.605 | 7.371 | 1.00 | 12.86 | BTKA |
| ATOM | 1886 | SD | MET | 630 | 25.803 | 44.178 | 8.496 | 1.00 | 14.55 | BTKA |
| ATOM | 1887 | CE | MET | 630 | 24.866 | 44.657 | 9.833 | 1.00 | 15.03 | BTKA |
| ATOM | 1888 | C | MET | 630 | 22.344 | 41.777 | 6.472 | 1.00 | 9.78 | BTKA |
| ATOM | 1889 | O | MET | 630 | 21.709 | 40.917 | 7.084 | 1.00 | 13.22 | BTKA |
| ATOM | 1890 | N | TYR | 631 | 21.794 | 42.920 | 6.080 | 1.00 | 9.86 | BTKA |
| ATOM | 1891 | CA | TYR | 631 | 20.420 | 43.235 | 6.446 | 1.00 | 9.86 | BTKA |
| ATOM | 1892 | CB | TYR | 631 | 20.040 | 44.662 | 6.028 | 1.00 | 10.36 | BTKA |
| ATOM | 1893 | CG | TYR | 631 | 18.706 | 45.099 | 6.592 | 1.00 | 16.09 | BTKA |
| ATOM | 1894 | CD1 | TYR | 631 | 17.519 | 44.790 | 5.929 | 1.00 | 17.24 | BTKA |
| ATOM | 1895 | CE1 | TYR | 631 | 16.283 | 45.124 | 6.463 | 1.00 | 15.16 | BTKA |
| ATOM | 1896 | CD2 | TYR | 631 | 18.622 | 45.768 | 7.811 | 1.00 | 14.11 | BTKA |
| ATOM | 1897 | CE2 | TYR | 631 | 17.380 | 46.112 | 8.359 | 1.00 | 17.22 | BTKA |
| ATOM | 1898 | CZ | TYR | 631 | 16.218 | 45.784 | 7.674 | 1.00 | 16.41 | BTKA |
| ATOM | 1899 | OH | TYR | 631 | 14.975 | 46.120 | 8.166 | 1.00 | 18.16 | BTKA |
| ATOM | 1900 | C | TYR | 631 | 19.415 | 42.228 | 5.892 | 1.00 | 10.69 | BTKA |
| ATOM | 1901 | O | TYR | 631 | 18.403 | 41.949 | 6.532 | 1.00 | 12.38 | BTKA |
| ATOM | 1902 | N | SER | 632 | 19.698 | 41.666 | 4.721 | 1.00 | 9.17 | BTKA |
| ATOM | 1903 | CA | SER | 632 | 18.796 | 40.692 | 4.113 | 1.00 | 10.83 | BTKA |
| ATOM | 1904 | CB | SER | 632 | 19.314 | 40.280 | 2.728 | 1.00 | 6.49 | BTKA |
| ATOM | 1905 | OG | SER | 632 | 20.564 | 39.615 | 2.807 | 1.00 | 2.00 | BTKA |
| ATOM | 1906 | C | SER | 632 | 18.626 | 39.450 | 4.983 | 1.00 | 9.86 | BTKA |
| ATOM | 1907 | O | SER | 632 | 17.565 | 38.827 | 4.991 | 1.00 | 10.42 | BTKA |
| ATOM | 1908 | N | CYS | 633 | 19.667 | 39.117 | 5.740 | 1.00 | 11.03 | BTKA |
| ATOM | 1909 | CA | CYS | 633 | 19.677 | 37.933 | 6.599 | 1.00 | 7.43 | BTKA |
| ATOM | 1910 | CB | CYS | 633 | 21.060 | 37.759 | 7.231 | 1.00 | 6.63 | BTKA |
| ATOM | 1911 | SG | CYS | 633 | 22.373 | 37.428 | 6.045 | 1.00 | 4.09 | BTKA |
| ATOM | 1912 | C | CYS | 633 | 18.632 | 37.940 | 7.699 | 1.00 | 6.70 | BTKA |
| ATOM | 1913 | O | CYS | 633 | 18.215 | 36.888 | 8.177 | 1.00 | 8.11 | BTKA |
| ATOM | 1914 | N | TRP | 634 | 18.156 | 39.125 | 8.043 | 1.00 | 6.33 | BTKA |
| ATOM | 1915 | CA | TRP | 634 | 17.206 | 39.273 | 9.129 | 1.00 | 9.48 | BTKA |
| ATOM | 1916 | CB | TRP | 634 | 17.666 | 40.428 | 10.010 | 1.00 | 9.15 | BTKA |
| ATOM | 1917 | CG | TRP | 634 | 19.135 | 40.440 | 10.331 | 1.00 | 4.86 | BTKA |
| ATOM | 1918 | CD2 | TRP | 634 | 19.974 | 41.590 | 10.376 | 1.00 | 3.49 | BTKA |
| ATOM | 1919 | CE2 | TRP | 634 | 21.261 | 41.154 | 10.756 | 1.00 | 2.25 | BTKA |
| ATOM | 1920 | CE3 | TRP | 634 | 19.770 | 42.955 | 10.140 | 1.00 | 3.18 | BTKA |
| ATOM | 1921 | CD1 | TRP | 634 | 19.918 | 39.378 | 10.671 | 1.00 | 2.00 | BTKA |
| ATOM | 1922 | NE1 | TRP | 634 | 21.197 | 39.797 | 10.930 | 1.00 | 2.34 | BTKA |
| ATOM | 1923 | CZ2 | TRP | 634 | 22.332 | 42.035 | 10.904 | 1.00 | 2.00 | BTKA |
| ATOM | 1924 | CZ3 | TRP | 634 | 20.835 | 43.827 | 10.291 | 1.00 | 2.00 | BTKA |
| ATOM | 1925 | CH2 | TRP | 634 | 22.098 | 43.361 | 10.668 | 1.00 | 2.00 | BTKA |
| ATOM | 1926 | C | TRP | 634 | 15.768 | 39.537 | 8.697 | 1.00 | 10.94 | BTKA |
| ATOM | 1927 | O | TRP | 634 | 15.015 | 40.182 | 9.422 | 1.00 | 13.81 | BTKA |
| ATOM | 1928 | N | HIS | 635 | 15.340 | 38.956 | 7.588 | 1.00 | 11.56 | BTKA |
| ATOM | 1929 | CA | HIS | 635 | 13.997 | 39.231 | 7.090 | 1.00 | 13.40 | BTKA |
| ATOM | 1930 | CB | HIS | 635 | 13.833 | 38.692 | 5.670 | 1.00 | 8.93 | BTKA |
| ATOM | 1931 | CG | HIS | 635 | 12.712 | 39.336 | 4.923 | 1.00 | 7.82 | BTKA |
| ATOM | 1932 | CD2 | HIS | 635 | 12.720 | 40.264 | 3.936 | 1.00 | 9.65 | BTKA |
| ATOM | 1933 | ND1 | HIS | 635 | 11.386 | 39.124 | 5.236 | 1.00 | 6.04 | BTKA |
| ATOM | 1934 | CE1 | HIS | 635 | 10.627 | 39.896 | 4.484 | 1.00 | 6.47 | BTKA |
| ATOM | 1935 | NE2 | HIS | 635 | 11.410 | 40.598 | 3.687 | 1.00 | 11.66 | BTKA |
| ATOM | 1936 | C | HIS | 635 | 12.720 | 38.940 | 7.888 | 1.00 | 16.55 | BTKA |
| ATOM | 1937 | O | HIS | 635 | 11.689 | 39.543 | 7.589 | 1.00 | 21.21 | BTKA |
| ATOM | 1938 | N | GLU | 636 | 12.758 | 38.053 | 8.877 | 1.00 | 16.72 | BTKA |
| ATOM | 1939 | CA | GLU | 636 | 11.563 | 37.677 | 9.675 | 1.00 | 16.32 | BTKA |
| ATOM | 1940 | CB | GLU | 636 | 10.524 | 38.810 | 9.812 | 1.00 | 18.59 | BTKA |
| ATOM | 1941 | CG | GLU | 636 | 9.347 | 38.458 | 10.730 | 1.00 | 25.85 | BTKA |
| ATOM | 1942 | CD | GLU | 636 | 8.003 | 39.015 | 10.258 | 1.00 | 28.55 | BTKA |
| ATOM | 1943 | OE1 | GLU | 636 | 7.772 | 39.080 | 9.030 | 1.00 | 29.75 | BTKA |
| ATOM | 1944 | OE2 | GLU | 636 | 7.161 | 39.364 | 11.115 | 1.00 | 28.67 | BTKA |
| ATOM | 1945 | C | GLU | 636 | 10.925 | 36.492 | 8.966 | 1.00 | 16.71 | BTKA |
| ATOM | 1946 | O | GLU | 636 | 10.924 | 35.384 | 9.492 | 1.00 | 19.32 | BTKA |
| ATOM | 1947 | N | LYS | 637 | 10.385 | 36.728 | 7.773 | 1.00 | 16.09 | BTKA |
| ATOM | 1948 | CA | LYS | 637 | 9.798 | 35.654 | 6.979 | 1.00 | 15.70 | BTKA |
| ATOM | 1949 | CB | LYS | 637 | 9.072 | 36.210 | 5.740 | 1.00 | 14.04 | BTKA |
| ATOM | 1950 | CG | LYS | 637 | 8.061 | 37.320 | 6.012 | 1.00 | 14.27 | BTKA |
| ATOM | 1951 | CD | LYS | 637 | 6.625 | 36.817 | 6.109 | 1.00 | 21.34 | BTKA |
| ATOM | 1952 | CE | LYS | 637 | 6.472 | 35.700 | 7.120 | 1.00 | 21.37 | BTKA |
| ATOM | 1953 | NZ | LYS | 637 | 6.679 | 34.358 | 6.495 | 1.00 | 22.42 | BTKA |
| ATOM | 1954 | C | LYS | 637 | 10.996 | 34.830 | 6.510 | 1.00 | 15.02 | BTKA |
| ATOM | 1955 | O | LYS | 637 | 11.751 | 35.270 | 5.636 | 1.00 | 14.64 | BTKA |
| ATOM | 1956 | N | ALA | 638 | 11.190 | 33.665 | 7.116 | 1.00 | 12.71 | BTKA |
| ATOM | 1957 | CA | ALA | 638 | 12.294 | 32.778 | 6.769 | 1.00 | 9.49 | BTKA |
| ATOM | 1958 | CB | ALA | 638 | 12.074 | 31.417 | 7.398 | 1.00 | 11.57 | BTKA |
| ATOM | 1959 | C | ALA | 638 | 12.574 | 32.621 | 5.270 | 1.00 | 12.33 | BTKA |
| ATOM | 1960 | O | ALA | 638 | 13.724 | 32.764 | 4.840 | 1.00 | 9.51 | BTKA |
| ATOM | 1961 | N | ASP | 639 | 11.526 | 32.378 | 4.475 | 1.00 | 11.12 | BTKA |
| ATOM | 1962 | CA | ASP | 639 | 11.678 | 32.163 | 3.030 | 1.00 | 11.04 | BTKA |
| ATOM | 1963 | CB | ASP | 639 | 10.431 | 31.478 | 2.426 | 1.00 | 13.90 | BTKA |
| ATOM | 1964 | CG | ASP | 639 | 9.181 | 32.358 | 2.437 | 1.00 | 13.58 | BTKA |
| ATOM | 1965 | OD1 | ASP | 639 | 9.270 | 33.586 | 2.626 | 1.00 | 15.17 | BTKA |
| ATOM | 1966 | OD2 | ASP | 639 | 8.081 | 31.808 | 2.235 | 1.00 | 15.41 | BTKA |
| ATOM | 1967 | C | ASP | 639 | 12.074 | 33.366 | 2.185 | 1.00 | 10.51 | BTKA |
| ATOM | 1968 | O | ASP | 639 | 12.206 | 33.258 | 0.962 | 1.00 | 3.89 | BTKA |
| ATOM | 1969 | N | GLU | 640 | 12.203 | 34.517 | 2.836 | 1.00 | 10.52 | BTKA |
| ATOM | 1970 | CA | GLU | 640 | 12.575 | 35.754 | 2.169 | 1.00 | 11.19 | BTKA |
| ATOM | 1971 | CB | GLU | 640 | 11.673 | 36.892 | 2.640 | 1.00 | 14.28 | BTKA |
| ATOM | 1972 | CG | GLU | 640 | 11.180 | 37.808 | 1.531 | 1.00 | 24.80 | BTKA |
| ATOM | 1973 | CD | GLU | 640 | 9.741 | 37.523 | 1.125 | 1.00 | 30.36 | BTKA |
| ATOM | 1974 | OE1 | GLU | 640 | 9.499 | 37.238 | −0.072 | 1.00 | 34.01 | BTKA |
| ATOM | 1975 | OE2 | GLU | 640 | 8.849 | 37.602 | 2.002 | 1.00 | 36.33 | BTKA |
| ATOM | 1976 | C | GLU | 640 | 14.030 | 36.074 | 2.496 | 1.00 | 9.22 | BTKA |
| ATOM | 1977 | O | GLU | 640 | 14.556 | 37.117 | 2.120 | 1.00 | 10.28 | BTKA |
| ATOM | 1978 | N | ARG | 641 | 14.658 | 35.185 | 3.249 | 1.00 | 8.49 | BTKA |
| ATOM | 1979 | CA | ARG | 641 | 16.048 | 35.343 | 3.626 | 1.00 | 10.21 | BTKA |
| ATOM | 1980 | CB | ARG | 641 | 16.256 | 34.861 | 5.069 | 1.00 | 8.41 | BTKA |
| ATOM | 1981 | CG | ARG | 641 | 15.151 | 35.391 | 5.991 | 1.00 | 10.60 | BTKA |
| ATOM | 1982 | CD | ARG | 641 | 15.623 | 35.692 | 7.392 | 1.00 | 13.17 | BTKA |
| ATOM | 1983 | NE | ARG | 641 | 15.831 | 34.497 | 8.189 | 1.00 | 18.11 | BTKA |
| ATOM | 1984 | CZ | ARG | 641 | 14.942 | 34.007 | 9.045 | 1.00 | 15.39 | BTKA |
| ATOM | 1985 | NH1 | ARG | 641 | 13.776 | 34.608 | 9.222 | 1.00 | 14.59 | BTKA |
| ATOM | 1986 | NH2 | ARG | 641 | 15.228 | 32.917 | 9.734 | 1.00 | 13.36 | BTKA |
| ATOM | 1987 | C | ARG | 641 | 16.834 | 34.519 | 2.608 | 1.00 | 11.06 | BTKA |
| ATOM | 1988 | O | ARG | 641 | 16.490 | 33.367 | 2.320 | 1.00 | 8.77 | BTKA |
| ATOM | 1989 | N | PRO | 642 | 17.843 | 35.141 | 1.982 | 1.00 | 11.46 | BTKA |
| ATOM | 1990 | CD | PRO | 642 | 18.197 | 36.544 | 2.242 | 1.00 | 10.26 | BTKA |
| ATOM | 1991 | CA | PRO | 642 | 18.733 | 34.573 | 0.961 | 1.00 | 13.85 | BTKA |
| ATOM | 1992 | CB | PRO | 642 | 19.725 | 35.710 | 0.721 | 1.00 | 16.79 | BTKA |
| ATOM | 1993 | CG | PRO | 642 | 18.897 | 36.914 | 0.967 | 1.00 | 15.67 | BTKA |
| ATOM | 1994 | C | PRO | 642 | 19.471 | 33.301 | 1.348 | 1.00 | 12.77 | BTKA |
| ATOM | 1995 | O | PRO | 642 | 19.570 | 32.959 | 2.522 | 1.00 | 11.54 | BTKA |
| ATOM | 1996 | N | THR | 643 | 19.977 | 32.597 | 0.342 | 1.00 | 11.30 | BTKA |
| ATOM | 1997 | CA | THR | 643 | 20.747 | 31.389 | 0.580 | 1.00 | 10.65 | BTKA |
| ATOM | 1998 | CB | THR | 643 | 20.667 | 30.414 | −0.614 | 1.00 | 6.97 | BTKA |
| ATOM | 1999 | OG1 | THR | 643 | 21.115 | 31.067 | −1.807 | 1.00 | 10.04 | BTKA |
| ATOM | 2000 | CG2 | THR | 643 | 19.255 | 29.934 | −0.813 | 1.00 | 8.33 | BTKA |
| ATOM | 2001 | C | THR | 643 | 22.201 | 31.829 | 0.778 | 1.00 | 10.25 | BTKA |
| ATOM | 2002 | O | THR | 643 | 22.545 | 32.983 | 0.520 | 1.00 | 13.36 | BTKA |
| ATOM | 2003 | N | PHE | 644 | 23.044 | 30.920 | 1.255 | 1.00 | 9.85 | BTKA |
| ATOM | 2004 | CA | PHE | 644 | 24.454 | 31.223 | 1.459 | 1.00 | 8.80 | BTKA |
| ATOM | 2005 | CB | PHE | 644 | 25.125 | 30.152 | 2.314 | 1.00 | 6.23 | BTKA |
| ATOM | 2006 | CG | PHE | 644 | 24.910 | 30.343 | 3.782 | 1.00 | 7.15 | BTKA |
| ATOM | 2007 | CD1 | PHE | 644 | 25.331 | 31.515 | 4.405 | 1.00 | 9.73 | BTKA |
| ATOM | 2008 | CD2 | PHE | 644 | 24.268 | 29.367 | 4.543 | 1.00 | 4.29 | BTKA |
| ATOM | 2009 | CE1 | PHE | 644 | 25.117 | 31.715 | 5.766 | 1.00 | 9.97 | BTKA |
| ATOM | 2010 | CE2 | PHE | 644 | 24.048 | 29.554 | 5.900 | 1.00 | 2.00 | BTKA |
| ATOM | 2011 | CZ | PHE | 644 | 24.473 | 30.732 | 6.515 | 1.00 | 8.09 | BTKA |
| ATOM | 2012 | C | PHE | 644 | 25.175 | 31.387 | 0.136 | 1.00 | 6.83 | BTKA |
| ATOM | 2013 | O | PHE | 644 | 26.132 | 32.146 | 0.042 | 1.00 | 6.14 | BTKA |
| ATOM | 2014 | N | ALA | 645 | 24.679 | 30.703 | −0.893 | 1.00 | 10.54 | BTKA |
| ATOM | 2015 | CA | ALA | 645 | 25.247 | 30.791 | −2.241 | 1.00 | 9.84 | BTKA |
| ATOM | 2016 | CB | ALA | 645 | 24.635 | 29.730 | −3.150 | 1.00 | 14.63 | BTKA |
| ATOM | 2017 | C | ALA | 645 | 24.976 | 32.179 | −2.809 | 1.00 | 9.74 | BTKA |
| ATOM | 2018 | O | ALA | 645 | 25.815 | 32.751 | −3.512 | 1.00 | 11.66 | BTKA |
| ATOM | 2019 | N | ILE | 646 | 23.787 | 32.705 | −2.534 | 1.00 | 10.19 | BTKA |
| ATOM | 2020 | CA | ILE | 646 | 23.434 | 34.029 | −3.015 | 1.00 | 9.44 | BTKA |
| ATOM | 2021 | CB | ILE | 646 | 21.912 | 34.306 | −2.904 | 1.00 | 10.35 | BTKA |
| ATOM | 2022 | CG2 | ILE | 646 | 21.615 | 35.788 | −3.117 | 1.00 | 9.73 | BTKA |
| ATOM | 2023 | CG1 | ILE | 646 | 21.163 | 33.467 | −3.945 | 1.00 | 12.53 | BTKA |
| ATOM | 2024 | CD | ILE | 646 | 19.749 | 33.935 | −4.220 | 1.00 | 10.68 | BTKA |
| ATOM | 2025 | C | ILE | 646 | 24.237 | 35.057 | −2.234 | 1.00 | 8.64 | BTKA |
| ATOM | 2026 | O | ILE | 646 | 24.792 | 35.987 | −2.820 | 1.00 | 12.42 | BTKA |
| ATOM | 2027 | N | LEU | 647 | 24.329 | 34.870 | −0.923 | 1.00 | 5.16 | BTKA |
| ATOM | 2028 | CA | LEU | 647 | 25.096 | 35.777 | −0.076 | 1.00 | 5.21 | BTKA |
| ATOM | 2029 | CB | LEU | 647 | 25.049 | 35.331 | 1.383 | 1.00 | 6.62 | BTKA |
| ATOM | 2030 | CG | LEU | 647 | 23.782 | 35.668 | 2.152 | 1.00 | 2.00 | BTKA |
| ATOM | 2031 | CD1 | LEU | 647 | 23.785 | 34.910 | 3.454 | 1.00 | 7.11 | BTKA |
| ATOM | 2032 | CD2 | LEU | 647 | 23.705 | 37.180 | 2.390 | 1.00 | 6.32 | BTKA |
| ATOM | 2033 | C | LEU | 647 | 26.538 | 35.803 | −0.538 | 1.00 | 4.59 | BTKA |
| ATOM | 2034 | O | LEU | 647 | 27.151 | 36.859 | −0.615 | 1.00 | 9.34 | BTKA |
| ATOM | 2035 | N | LEU | 648 | 27.083 | 34.632 | −0.838 | 1.00 | 6.10 | BTKA |
| ATOM | 2036 | CA | LEU | 648 | 28.454 | 34.540 | −1.307 | 1.00 | 7.56 | BTKA |
| ATOM | 2037 | CB | LEU | 648 | 28.901 | 33.087 | −1.407 | 1.00 | 6.76 | BTKA |
| ATOM | 2038 | CG | LEU | 648 | 30.313 | 32.904 | −1.953 | 1.00 | 5.57 | BTKA |
| ATOM | 2039 | CD1 | LEU | 648 | 31.338 | 33.392 | −0.951 | 1.00 | 8.87 | BTKA |
| ATOM | 2040 | CD2 | LEU | 648 | 30.531 | 31.456 | −2.262 | 1.00 | 9.05 | BTKA |
| ATOM | 2041 | C | LEU | 648 | 28.603 | 35.218 | −2.662 | 1.00 | 7.15 | BTKA |
| ATOM | 2042 | O | LEU | 648 | 29.619 | 35.860 | −2.914 | 1.00 | 5.11 | BTKA |
| ATOM | 2043 | N | SER | 649 | 27.606 | 35.079 | −3.535 | 1.00 | 7.37 | BTKA |
| ATOM | 2044 | CA | SER | 649 | 27.684 | 35.715 | −4.848 | 1.00 | 11.39 | BTKA |
| ATOM | 2045 | CB | SER | 649 | 26.619 | 35.172 | −5.826 | 1.00 | 13.66 | BTKA |
| ATOM | 2046 | OG | SER | 649 | 25.352 | 35.803 | −5.718 | 1.00 | 18.95 | BTKA |
| ATOM | 2047 | C | SER | 649 | 27.588 | 37.226 | −4.674 | 1.00 | 13.90 | BTKA |
| ATOM | 2048 | O | SER | 649 | 28.255 | 37.979 | −5.384 | 1.00 | 14.33 | BTKA |
| ATOM | 2049 | N | ASN | 650 | 26.813 | 37.666 | −3.685 | 1.00 | 15.59 | BTKA |
| ATOM | 2050 | CA | ASN | 650 | 26.669 | 39.097 | −3.418 | 1.00 | 14.80 | BTKA |
| ATOM | 2051 | CB | ASN | 650 | 25.655 | 39.351 | −2.301 | 1.00 | 15.49 | BTKA |
| ATOM | 2052 | CG | ASN | 650 | 24.218 | 39.195 | −2.758 | 1.00 | 13.88 | BTKA |
| ATOM | 2053 | OD1 | ASN | 650 | 23.911 | 39.333 | −3.941 | 1.00 | 16.71 | BTKA |
| ATOM | 2054 | ND2 | ASN | 650 | 23.323 | 38.920 | −1.814 | 1.00 | 18.03 | BTKA |
| ATOM | 2055 | C | ASN | 650 | 28.035 | 39.590 | −2.975 | 1.00 | 16.71 | BTKA |
| ATOM | 2056 | O | ASN | 650 | 28.627 | 40.461 | −3.602 | 1.00 | 18.95 | BTKA |
| ATOM | 2057 | N | ILE | 651 | 28.553 | 38.972 | −1.920 | 1.00 | 17.37 | BTKA |
| ATOM | 2058 | CA | ILE | 651 | 29.856 | 39.315 | −1.368 | 1.00 | 16.71 | BTKA |
| ATOM | 2059 | CB | ILE | 651 | 30.300 | 38.269 | −0.330 | 1.00 | 16.01 | BTKA |
| ATOM | 2060 | CG2 | ILE | 651 | 31.766 | 38.473 | 0.049 | 1.00 | 16.81 | BTKA |
| ATOM | 2061 | CG1 | ILE | 651 | 29.397 | 38.333 | 0.902 | 1.00 | 18.86 | BTKA |
| ATOM | 2062 | CD | ILE | 651 | 29.787 | 37.353 | 1.998 | 1.00 | 18.13 | BTKA |
| ATOM | 2063 | C | ILE | 651 | 30.936 | 39.407 | −2.441 | 1.00 | 17.88 | BTKA |
| ATOM | 2064 | O | ILE | 651 | 31.730 | 40.340 | −2.442 | 1.00 | 17.27 | BTKA |
| ATOM | 2065 | N | LEU | 652 | 30.979 | 38.425 | −3.333 | 1.00 | 17.38 | BTKA |
| ATOM | 2066 | CA | LEU | 652 | 31.988 | 38.410 | −4.383 | 1.00 | 16.93 | BTKA |
| ATOM | 2067 | CB | LEU | 652 | 31.927 | 37.097 | −5.174 | 1.00 | 16.89 | BTKA |
| ATOM | 2068 | CG | LEU | 652 | 32.426 | 35.821 | −4.496 | 1.00 | 14.36 | BTKA |
| ATOM | 2069 | CD1 | LEU | 652 | 32.223 | 34.644 | −5.415 | 1.00 | 17.00 | BTKA |
| ATOM | 2070 | CD2 | LEU | 652 | 33.890 | 35.966 | −4.155 | 1.00 | 17.94 | BTKA |
| ATOM | 2071 | C | LEU | 652 | 31.864 | 39.593 | −5.339 | 1.00 | 17.77 | BTKA |
| ATOM | 2072 | O | LEU | 652 | 32.869 | 40.153 | −5.775 | 1.00 | 14.19 | BTKA |
| ATOM | 2073 | N | ASP | 653 | 30.626 | 39.958 | −5.660 | 1.00 | 16.94 | BTKA |
| ATOM | 2074 | CA | ASP | 653 | 30.338 | 41.052 | −6.583 | 1.00 | 18.91 | BTKA |
| ATOM | 2075 | CB | ASP | 653 | 28.839 | 41.029 | −6.933 | 1.00 | 22.19 | BTKA |
| ATOM | 2076 | CG | ASP | 653 | 28.511 | 41.768 | −8.220 | 1.00 | 27.15 | BTKA |
| ATOM | 2077 | OD1 | ASP | 653 | 28.383 | 43.014 | −8.190 | 1.00 | 26.08 | BTKA |
| ATOM | 2078 | OD2 | ASP | 653 | 28.338 | 41.094 | −9.261 | 1.00 | 29.58 | BTKA |
| ATOM | 2079 | C | ASP | 653 | 30.743 | 42.397 | −5.971 | 1.00 | 16.94 | BTKA |
| ATOM | 2080 | O | ASP | 653 | 30.021 | 42.968 | −5.153 | 1.00 | 18.07 | BTKA |
| ATOM | 2081 | N | VAL | 654 | 31.910 | 42.883 | −6.363 | 1.00 | 17.09 | BTKA |
| ATOM | 2082 | CA | VAL | 654 | 32.448 | 44.156 | −5.873 | 1.00 | 19.99 | BTKA |
| ATOM | 2083 | CB | VAL | 654 | 32.525 | 44.192 | −4.285 | 1.00 | 15.42 | BTKA |
| ATOM | 2084 | CG1 | VAL | 654 | 33.538 | 43.189 | −3.741 | 1.00 | 19.20 | BTKA |
| ATOM | 2085 | CG2 | VAL | 654 | 32.836 | 45.587 | −3.792 | 1.00 | 15.46 | BTKA |
| ATOM | 2086 | C | VAL | 654 | 33.831 | 44.409 | −6.521 | 1.00 | 21.38 | BTKA |
| ATOM | 2087 | OT1 | VAL | 654 | 34.043 | 45.515 | −7.073 | 1.00 | 20.29 | BTKA |
| ATOM | 2088 | OT2 | VAL | 654 | 34.668 | 43.475 | −6.533 | 1.00 | 23.32 | BTKA |
| TER | 2088 | | VAL | 654 | | | | | | BTKA |
| ATOM | 2089 | CB | ILE | 397 | 34.822 | 44.259 | 42.166 | 1.00 | 28.06 | BTKB |
| ATOM | 2090 | CG2 | ILE | 397 | 35.367 | 43.761 | 43.501 | 1.00 | 30.24 | BTKB |
| ATOM | 2091 | CG1 | ILE | 397 | 33.354 | 44.660 | 42.302 | 1.00 | 27.51 | BTKB |
| ATOM | 2092 | CD | ILE | 397 | 32.460 | 43.563 | 42.838 | 1.00 | 31.05 | BTKB |
| ATOM | 2093 | C | ILE | 397 | 37.125 | 45.091 | 41.610 | 1.00 | 29.53 | BTKB |
| ATOM | 2094 | O | ILE | 397 | 37.977 | 45.873 | 42.022 | 1.00 | 31.32 | BTKB |
| ATOM | 2095 | N | ILE | 397 | 35.200 | 45.884 | 40.296 | 1.00 | 27.80 | BTKB |
| ATOM | 2096 | CA | ILE | 397 | 35.647 | 45.461 | 41.653 | 1.00 | 29.30 | BTKB |
| ATOM | 2097 | N | ASP | 398 | 37.413 | 43.917 | 41.052 | 1.00 | 29.94 | BTKB |
| ATOM | 2098 | CA | ASP | 398 | 38.771 | 43.396 | 40.924 | 1.00 | 28.21 | BTKB |
| ATOM | 2099 | CB | ASP | 398 | 38.711 | 41.951 | 40.409 | 1.00 | 27.14 | BTKB |
| ATOM | 2100 | CG | ASP | 398 | 40.055 | 41.431 | 39.939 | 1.00 | 28.88 | BTKB |
| ATOM | 2101 | OD1 | ASP | 398 | 40.805 | 40.866 | 40.760 | 1.00 | 29.93 | BTKB |
| ATOM | 2102 | OD2 | ASP | 398 | 40.353 | 41.565 | 38.735 | 1.00 | 29.99 | BTKB |
| ATOM | 2103 | C | ASP | 398 | 39.631 | 44.248 | 39.999 | 1.00 | 30.83 | BTKB |
| ATOM | 2104 | O | ASP | 398 | 39.299 | 44.428 | 38.823 | 1.00 | 28.34 | BTKB |
| ATOM | 2105 | N | PRO | 399 | 40.726 | 44.819 | 40.529 | 1.00 | 30.97 | BTKB |
| ATOM | 2106 | CD | PRO | 399 | 41.104 | 44.844 | 41.953 | 1.00 | 29.33 | BTKB |
| ATOM | 2107 | CA | PRO | 399 | 41.633 | 45.654 | 39.735 | 1.00 | 30.72 | BTKB |
| ATOM | 2108 | CB | PRO | 399 | 42.689 | 46.073 | 40.760 | 1.00 | 33.06 | BTKB |
| ATOM | 2109 | CG | PRO | 399 | 41.908 | 46.118 | 42.044 | 1.00 | 32.66 | BTKB |
| ATOM | 2110 | C | PRO | 399 | 42.236 | 44.809 | 38.611 | 1.00 | 30.41 | BTKB |
| ATOM | 2111 | O | PRO | 399 | 43.229 | 44.106 | 38.808 | 1.00 | 27.77 | BTKB |
| ATOM | 2112 | N | LYS | 400 | 41.618 | 44.906 | 37.435 | 1.00 | 31.71 | BTKB |
| ATOM | 2113 | CA | LYS | 400 | 41.991 | 44.169 | 36.225 | 1.00 | 32.73 | BTKB |
| ATOM | 2114 | CB | LYS | 400 | 41.466 | 44.913 | 34.992 | 1.00 | 31.73 | BTKB |
| ATOM | 2115 | CG | LYS | 400 | 41.243 | 44.014 | 33.789 | 1.00 | 31.68 | BTKB |
| ATOM | 2116 | CD | LYS | 400 | 40.893 | 44.804 | 32.545 | 1.00 | 29.01 | BTKB |
| ATOM | 2117 | CE | LYS | 400 | 40.459 | 43.882 | 31.409 | 1.00 | 27.04 | BTKB |
| ATOM | 2118 | NZ | LYS | 400 | 39.176 | 43.195 | 31.722 | 1.00 | 25.44 | BTKB |
| ATOM | 2119 | C | LYS | 400 | 43.453 | 43.746 | 35.993 | 1.00 | 35.08 | BTKB |
| ATOM | 2120 | O | LYS | 400 | 43.710 | 42.872 | 35.157 | 1.00 | 32.76 | BTKB |
| ATOM | 2121 | N | ASP | 401 | 44.410 | 44.363 | 36.684 | 1.00 | 37.73 | BTKB |
| ATOM | 2122 | CA | ASP | 401 | 45.807 | 43.987 | 36.493 | 1.00 | 38.72 | BTKB |
| ATOM | 2123 | CB | ASP | 401 | 46.682 | 45.209 | 36.209 | 1.00 | 38.08 | BTKB |
| ATOM | 2124 | CG | ASP | 401 | 48.080 | 44.824 | 35.748 | 1.00 | 38.22 | BTKB |
| ATOM | 2125 | OD1 | ASP | 401 | 48.992 | 44.757 | 36.599 | 1.00 | 39.11 | BTKB |
| ATOM | 2126 | OD2 | ASP | 401 | 48.262 | 44.564 | 34.539 | 1.00 | 40.47 | BTKB |
| ATOM | 2127 | C | ASP | 401 | 46.375 | 43.195 | 37.668 | 1.00 | 40.61 | BTKB |
| ATOM | 2128 | O | ASP | 401 | 46.429 | 43.683 | 38.802 | 1.00 | 39.92 | BTKB |
| ATOM | 2129 | N | LEU | 402 | 46.832 | 41.982 | 37.365 | 1.00 | 39.68 | BTKB |
| ATOM | 2130 | CA | LEU | 402 | 47.412 | 41.076 | 38.347 | 1.00 | 35.79 | BTKB |
| ATOM | 2131 | CB | LEU | 402 | 46.300 | 40.318 | 39.083 | 1.00 | 30.36 | BTKB |
| ATOM | 2132 | CG | LEU | 402 | 45.296 | 39.475 | 38.286 | 1.00 | 25.43 | BTKB |
| ATOM | 2133 | CD1 | LEU | 402 | 44.710 | 38.416 | 39.200 | 1.00 | 25.89 | BTKB |
| ATOM | 2134 | CD2 | LEU | 402 | 44.189 | 40.326 | 37.678 | 1.00 | 19.90 | BTKB |
| ATOM | 2135 | C | LEU | 402 | 48.339 | 40.090 | 37.627 | 1.00 | 36.68 | BTKB |
| ATOM | 2136 | O | LEU | 402 | 48.350 | 40.034 | 36.396 | 1.00 | 37.32 | BTKB |
| ATOM | 2137 | N | THR | 403 | 49.128 | 39.332 | 38.385 | 1.00 | 38.28 | BTKB |
| ATOM | 2138 | CA | THR | 403 | 50.047 | 38.356 | 37.798 | 1.00 | 39.00 | BTKB |
| ATOM | 2139 | CB | THR | 403 | 50.922 | 37.688 | 38.885 | 1.00 | 38.43 | BTKB |
| ATOM | 2140 | OG1 | THR | 403 | 51.318 | 38.666 | 39.857 | 1.00 | 39.19 | BTKB |
| ATOM | 2141 | CG2 | THR | 403 | 52.165 | 37.073 | 38.262 | 1.00 | 34.76 | BTKB |
| ATOM | 2142 | C | THR | 403 | 49.258 | 37.270 | 37.050 | 1.00 | 41.52 | BTKB |
| ATOM | 2143 | O | THR | 403 | 48.103 | 36.993 | 37.392 | 1.00 | 43.01 | BTKB |
| ATOM | 2144 | N | PHE | 404 | 49.884 | 36.663 | 36.041 | 1.00 | 40.42 | BTKB |
| ATOM | 2145 | CA | PHE | 404 | 49.262 | 35.613 | 35.226 | 1.00 | 38.98 | BTKB |
| ATOM | 2146 | CB | PHE | 404 | 48.349 | 36.210 | 34.143 | 1.00 | 38.33 | BTKB |
| ATOM | 2147 | CG | PHE | 404 | 48.701 | 37.620 | 33.748 | 1.00 | 39.41 | BTKB |
| ATOM | 2148 | CD1 | PHE | 404 | 47.704 | 38.578 | 33.617 | 1.00 | 39.35 | BTKB |
| ATOM | 2149 | CD2 | PHE | 404 | 50.026 | 38.000 | 33.542 | 1.00 | 40.12 | BTKB |
| ATOM | 2150 | CE1 | PHE | 404 | 48.016 | 39.892 | 33.293 | 1.00 | 39.05 | BTKB |
| ATOM | 2151 | CE2 | PHE | 404 | 50.351 | 39.314 | 33.218 | 1.00 | 39.92 | BTKB |
| ATOM | 2152 | CZ | PHE | 404 | 49.344 | 40.261 | 33.094 | 1.00 | 41.14 | BTKB |
| ATOM | 2153 | C | PHE | 404 | 50.316 | 34.723 | 34.578 | 1.00 | 38.11 | BTKB |
| ATOM | 2154 | O | PHE | 404 | 51.263 | 35.217 | 33.974 | 1.00 | 38.10 | BTKB |
| ATOM | 2155 | N | LEU | 405 | 50.151 | 33.412 | 34.722 | 1.00 | 39.17 | BTKB |
| ATOM | 2156 | CA | LEU | 405 | 51.096 | 32.450 | 34.168 | 1.00 | 38.13 | BTKB |
| ATOM | 2157 | CB | LEU | 405 | 51.676 | 31.575 | 35.285 | 1.00 | 39.62 | BTKB |
| ATOM | 2158 | CG | LEU | 405 | 52.874 | 32.193 | 36.020 | 1.00 | 42.57 | BTKB |
| ATOM | 2159 | CD1 | LEU | 405 | 53.105 | 31.533 | 37.378 | 1.00 | 41.80 | BTKB |
| ATOM | 2160 | CD2 | LEU | 405 | 54.112 | 32.086 | 35.140 | 1.00 | 40.80 | BTKB |
| ATOM | 2161 | C | LEU | 405 | 50.504 | 31.596 | 33.050 | 1.00 | 36.37 | BTKB |
| ATOM | 2162 | O | LEU | 405 | 50.680 | 31.910 | 31.872 | 1.00 | 38.19 | BTKB |
| ATOM | 2163 | N | LYS | 406 | 49.832 | 30.506 | 33.398 | 1.00 | 33.49 | BTKB |
| ATOM | 2164 | CA | LYS | 406 | 49.229 | 29.657 | 32.379 | 1.00 | 31.11 | BTKB |
| ATOM | 2165 | CB | LYS | 406 | 49.715 | 28.201 | 32.487 | 1.00 | 32.83 | BTKB |
| ATOM | 2166 | CG | LYS | 406 | 49.155 | 27.413 | 33.671 | 1.00 | 31.89 | BTKB |
| ATOM | 2167 | CD | LYS | 406 | 49.709 | 25.994 | 33.712 | 1.00 | 29.73 | BTKB |
| ATOM | 2168 | CE | LYS | 406 | 49.132 | 25.197 | 34.874 | 1.00 | 25.18 | BTKB |
| ATOM | 2169 | NZ | LYS | 406 | 47.664 | 24.987 | 34.754 | 1.00 | 23.18 | BTKB |
| ATOM | 2170 | C | LYS | 406 | 47.721 | 29.727 | 32.544 | 1.00 | 31.03 | BTKB |
| ATOM | 2171 | O | LYS | 406 | 47.194 | 30.685 | 33.126 | 1.00 | 29.59 | BTKB |
| ATOM | 2172 | N | GLU | 407 | 47.038 | 28.689 | 32.075 | 1.00 | 28.47 | BTKB |
| ATOM | 2173 | CA | GLU | 407 | 45.592 | 28.615 | 32.145 | 1.00 | 24.70 | BTKB |
| ATOM | 2174 | CB | GLU | 407 | 45.028 | 28.721 | 30.723 | 1.00 | 25.09 | BTKB |
| ATOM | 2175 | CG | GLU | 407 | 43.575 | 29.148 | 30.632 | 1.00 | 27.67 | BTKB |
| ATOM | 2176 | CD | GLU | 407 | 42.666 | 28.087 | 30.062 | 1.00 | 28.92 | BTKB |
| ATOM | 2177 | OE1 | GLU | 407 | 41.443 | 28.231 | 30.229 | 1.00 | 30.56 | BTKB |
| ATOM | 2178 | OE2 | GLU | 407 | 43.157 | 27.114 | 29.450 | 1.00 | 32.96 | BTKB |
| ATOM | 2179 | C | GLU | 407 | 45.220 | 27.272 | 32.764 | 1.00 | 22.29 | BTKB |
| ATOM | 2180 | O | GLU | 407 | 46.096 | 26.485 | 33.116 | 1.00 | 24.55 | BTKB |
| ATOM | 2181 | N | LEU | 408 | 43.924 | 27.037 | 32.924 | 1.00 | 21.71 | BTKB |
| ATOM | 2182 | CA | LEU | 408 | 43.411 | 25.787 | 33.470 | 1.00 | 21.08 | BTKB |
| ATOM | 2183 | CB | LEU | 408 | 42.611 | 26.020 | 34.751 | 1.00 | 20.12 | BTKB |
| ATOM | 2184 | CG | LEU | 408 | 43.312 | 25.979 | 36.104 | 1.00 | 17.11 | BTKB |
| ATOM | 2185 | CD1 | LEU | 408 | 44.189 | 27.196 | 36.305 | 1.00 | 16.68 | BTKB |
| ATOM | 2186 | CD2 | LEU | 408 | 42.244 | 25.924 | 37.169 | 1.00 | 20.38 | BTKB |
| ATOM | 2187 | C | LEU | 408 | 42.513 | 25.113 | 32.444 | 1.00 | 21.69 | BTKB |
| ATOM | 2188 | O | LEU | 408 | 42.883 | 24.088 | 31.872 | 1.00 | 25.66 | BTKB |
| ATOM | 2189 | N | GLY | 409 | 41.345 | 25.697 | 32.191 | 1.00 | 26.47 | BTKB |
| ATOM | 2190 | CA | GLY | 409 | 40.426 | 25.099 | 31.233 | 1.00 | 30.31 | BTKB |
| ATOM | 2191 | C | GLY | 409 | 39.026 | 25.681 | 31.179 | 1.00 | 31.13 | BTKB |
| ATOM | 2192 | O | GLY | 409 | 38.864 | 26.839 | 30.800 | 1.00 | 28.40 | BTKB |
| ATOM | 2193 | N | THR | 410 | 38.023 | 24.897 | 31.586 | 1.00 | 34.19 | BTKB |
| ATOM | 2194 | CA | THR | 410 | 36.623 | 25.337 | 31.563 | 1.00 | 35.54 | BTKB |
| ATOM | 2195 | CB | THR | 410 | 35.974 | 25.015 | 30.188 | 1.00 | 39.00 | BTKB |
| ATOM | 2196 | OG1 | THR | 410 | 36.807 | 25.525 | 29.140 | 1.00 | 42.12 | BTKB |
| ATOM | 2197 | CG2 | THR | 410 | 34.576 | 25.635 | 30.069 | 1.00 | 36.90 | BTKB |
| ATOM | 2198 | C | THR | 410 | 35.758 | 24.713 | 32.673 | 1.00 | 34.43 | BTKB |
| ATOM | 2199 | O | THR | 410 | 36.028 | 23.601 | 33.141 | 1.00 | 31.80 | BTKB |
| ATOM | 2200 | N | GLY | 411 | 34.721 | 25.447 | 33.076 | 1.00 | 33.66 | BTKB |
| ATOM | 2201 | CA | GLY | 411 | 33.801 | 24.986 | 34.102 | 1.00 | 32.04 | BTKB |
| ATOM | 2202 | C | GLY | 411 | 32.351 | 25.093 | 33.645 | 1.00 | 32.34 | BTKB |
| ATOM | 2203 | O | GLY | 411 | 31.738 | 24.093 | 33.280 | 1.00 | 33.64 | BTKB |
| ATOM | 2204 | N | GLN | 412 | 31.811 | 26.308 | 33.623 | 1.00 | 31.61 | BTKB |
| ATOM | 2205 | CA | GLN | 412 | 30.426 | 26.513 | 33.207 | 1.00 | 28.88 | BTKB |
| ATOM | 2206 | CB | GLN | 412 | 29.567 | 26.874 | 34.418 | 1.00 | 30.03 | BTKB |
| ATOM | 2207 | CG | GLN | 412 | 28.212 | 26.192 | 34.445 | 1.00 | 31.90 | BTKB |
| ATOM | 2208 | CD | GLN | 412 | 28.294 | 24.745 | 34.891 | 1.00 | 31.28 | BTKB |
| ATOM | 2209 | OE1 | GLN | 412 | 29.317 | 24.084 | 34.715 | 1.00 | 31.63 | BTKB |
| ATOM | 2210 | NE2 | GLN | 412 | 27.217 | 24.250 | 35.493 | 1.00 | 28.67 | BTKB |
| ATOM | 2211 | C | GLN | 412 | 30.270 | 27.600 | 32.140 | 1.00 | 28.86 | BTKB |
| ATOM | 2212 | O | GLN | 412 | 29.267 | 27.638 | 31.418 | 1.00 | 28.11 | BTKB |
| ATOM | 2213 | N | PHE | 413 | 31.234 | 28.512 | 32.074 | 1.00 | 28.97 | BTKB |
| ATOM | 2214 | CA | PHE | 413 | 31.188 | 29.593 | 31.097 | 1.00 | 28.08 | BTKB |
| ATOM | 2215 | CB | PHE | 413 | 31.388 | 30.954 | 31.773 | 1.00 | 28.79 | BTKB |
| ATOM | 2216 | CG | PHE | 413 | 30.221 | 31.403 | 32.594 | 1.00 | 26.11 | BTKB |
| ATOM | 2217 | CD1 | PHE | 413 | 29.317 | 32.325 | 32.084 | 1.00 | 30.79 | BTKB |
| ATOM | 2218 | CD2 | PHE | 413 | 30.023 | 30.904 | 33.872 | 1.00 | 29.49 | BTKB |
| ATOM | 2219 | CE1 | PHE | 413 | 28.229 | 32.740 | 32.833 | 1.00 | 33.14 | BTKB |
| ATOM | 2220 | CE2 | PHE | 413 | 28.935 | 31.313 | 34.633 | 1.00 | 31.02 | BTKB |
| ATOM | 2221 | CZ | PHE | 413 | 28.036 | 32.234 | 34.113 | 1.00 | 33.86 | BTKB |
| ATOM | 2222 | C | PHE | 413 | 32.248 | 29.403 | 30.027 | 1.00 | 30.53 | BTKB |
| ATOM | 2223 | O | PHE | 413 | 31.989 | 28.797 | 28.987 | 1.00 | 34.18 | BTKB |
| ATOM | 2224 | N | GLY | 414 | 33.453 | 29.889 | 30.306 | 1.00 | 29.58 | BTKB |
| ATOM | 2225 | CA | GLY | 414 | 34.527 | 29.781 | 29.343 | 1.00 | 29.36 | BTKB |
| ATOM | 2226 | C | GLY | 414 | 35.824 | 29.289 | 29.938 | 1.00 | 30.73 | BTKB |
| ATOM | 2227 | O | GLY | 414 | 35.824 | 28.404 | 30.806 | 1.00 | 29.03 | BTKB |
| ATOM | 2228 | N | VAL | 415 | 36.918 | 29.917 | 29.507 | 1.00 | 31.12 | BTKB |
| ATOM | 2229 | CA | VAL | 415 | 38.273 | 29.569 | 29.930 | 1.00 | 29.86 | BTKB |
| ATOM | 2230 | CB | VAL | 415 | 39.292 | 29.895 | 28.818 | 1.00 | 28.63 | BTKB |
| ATOM | 2231 | CG1 | VAL | 415 | 39.093 | 28.976 | 27.636 | 1.00 | 20.44 | BTKB |
| ATOM | 2232 | CG2 | VAL | 415 | 39.158 | 31.341 | 28.388 | 1.00 | 28.08 | BTKB |
| ATOM | 2233 | C | VAL | 415 | 38.773 | 30.174 | 31.247 | 1.00 | 33.07 | BTKB |
| ATOM | 2234 | O | VAL | 415 | 38.753 | 31.395 | 31.441 | 1.00 | 34.75 | BTKB |
| ATOM | 2235 | N | VAL | 416 | 39.268 | 29.307 | 32.125 | 1.00 | 33.11 | BTKB |
| ATOM | 2236 | CA | VAL | 416 | 39.792 | 29.716 | 33.426 | 1.00 | 33.57 | BTKB |
| ATOM | 2237 | CB | VAL | 416 | 39.158 | 28.867 | 34.561 | 1.00 | 36.44 | BTKB |
| ATOM | 2238 | CG1 | VAL | 416 | 39.077 | 27.402 | 34.147 | 1.00 | 38.53 | BTKB |
| ATOM | 2239 | CG2 | VAL | 416 | 39.944 | 29.014 | 35.860 | 1.00 | 36.43 | BTKB |
| ATOM | 2240 | C | VAL | 416 | 41.326 | 29.621 | 33.437 | 1.00 | 32.35 | BTKB |
| ATOM | 2241 | O | VAL | 416 | 41.880 | 28.539 | 33.264 | 1.00 | 32.92 | BTKB |
| ATOM | 2242 | N | LYS | 417 | 42.000 | 30.762 | 33.595 | 1.00 | 28.13 | BTKB |
| ATOM | 2243 | CA | LYS | 417 | 43.470 | 30.834 | 33.601 | 1.00 | 22.76 | BTKB |
| ATOM | 2244 | CB | LYS | 417 | 43.920 | 32.071 | 32.810 | 1.00 | 21.32 | BTKB |
| ATOM | 2245 | CG | LYS | 417 | 43.060 | 32.342 | 31.574 | 1.00 | 24.74 | BTKB |
| ATOM | 2246 | CD | LYS | 417 | 43.513 | 33.567 | 30.806 | 1.00 | 23.99 | BTKB |
| ATOM | 2247 | CE | LYS | 417 | 42.534 | 33.946 | 29.710 | 1.00 | 15.58 | BTKB |
| ATOM | 2248 | NZ | LYS | 417 | 42.968 | 35.229 | 29.104 | 1.00 | 20.25 | BTKB |
| ATOM | 2249 | C | LYS | 417 | 44.014 | 30.895 | 35.031 | 1.00 | 18.43 | BTKB |
| ATOM | 2250 | O | LYS | 417 | 43.265 | 30.665 | 35.976 | 1.00 | 17.13 | BTKB |
| ATOM | 2251 | N | ALA | 418 | 45.298 | 31.214 | 35.195 | 1.00 | 14.44 | BTKB |
| ATOM | 2252 | CA | ALA | 418 | 45.898 | 31.301 | 36.533 | 1.00 | 18.16 | BTKB |
| ATOM | 2253 | CB | ALA | 418 | 46.737 | 30.057 | 36.834 | 1.00 | 18.21 | BTKB |
| ATOM | 2254 | C | ALA | 418 | 46.750 | 32.548 | 36.706 | 1.00 | 18.40 | BTKB |
| ATOM | 2255 | O | ALA | 418 | 47.448 | 32.962 | 35.783 | 1.00 | 19.90 | BTKB |
| ATOM | 2256 | N | GLY | 419 | 46.724 | 33.116 | 37.905 | 1.00 | 20.29 | BTKB |
| ATOM | 2257 | CA | GLY | 419 | 47.507 | 34.308 | 38.176 | 1.00 | 20.56 | BTKB |
| ATOM | 2258 | C | GLY | 419 | 47.617 | 34.626 | 39.651 | 1.00 | 22.74 | BTKB |
| ATOM | 2259 | O | GLY | 419 | 47.091 | 33.895 | 40.487 | 1.00 | 24.36 | BTKB |
| ATOM | 2260 | N | ALA | 420 | 48.305 | 35.712 | 39.982 | 1.00 | 23.32 | BTKB |
| ATOM | 2261 | CA | ALA | 420 | 48.460 | 36.110 | 41.378 | 1.00 | 23.35 | BTKB |
| ATOM | 2262 | CB | ALA | 420 | 49.927 | 36.125 | 41.774 | 1.00 | 23.90 | BTKB |
| ATOM | 2263 | C | ALA | 420 | 47.837 | 37.484 | 41.587 | 1.00 | 21.75 | BTKB |
| ATOM | 2264 | O | ALA | 420 | 48.198 | 38.448 | 40.912 | 1.00 | 24.80 | BTKB |
| ATOM | 2265 | N | TRP | 421 | 46.870 | 37.556 | 42.490 | 1.00 | 18.99 | BTKB |
| ATOM | 2266 | CA | TRP | 421 | 46.184 | 38.800 | 42.789 | 1.00 | 19.16 | BTKB |
| ATOM | 2267 | CB | TRP | 421 | 44.683 | 38.548 | 42.875 | 1.00 | 21.71 | BTKB |
| ATOM | 2268 | CG | TRP | 421 | 43.869 | 39.797 | 42.944 | 1.00 | 23.50 | BTKB |
| ATOM | 2269 | CD2 | TRP | 421 | 42.920 | 40.148 | 43.959 | 1.00 | 22.38 | BTKB |
| ATOM | 2270 | CE2 | TRP | 421 | 42.370 | 41.394 | 43.599 | 1.00 | 25.22 | BTKB |
| ATOM | 2271 | CE3 | TRP | 421 | 42.482 | 39.528 | 45.137 | 1.00 | 26.12 | BTKB |
| ATOM | 2272 | CD1 | TRP | 421 | 43.859 | 40.817 | 42.041 | 1.00 | 21.67 | BTKB |
| ATOM | 2273 | NE1 | TRP | 421 | 42.962 | 41.778 | 42.425 | 1.00 | 26.88 | BTKB |
| ATOM | 2274 | CZ2 | TRP | 421 | 41.402 | 42.036 | 44.372 | 1.00 | 26.92 | BTKB |
| ATOM | 2275 | CZ3 | TRP | 421 | 41.521 | 40.166 | 45.907 | 1.00 | 27.03 | BTKB |
| ATOM | 2276 | CH2 | TRP | 421 | 40.991 | 41.408 | 45.520 | 1.00 | 26.73 | BTKB |
| ATOM | 2277 | C | TRP | 421 | 46.694 | 39.348 | 44.114 | 1.00 | 20.06 | BTKB |
| ATOM | 2278 | O | TRP | 421 | 46.905 | 38.587 | 45.059 | 1.00 | 21.40 | BTKB |
| ATOM | 2279 | N | ARG | 422 | 46.907 | 40.664 | 44.166 | 1.00 | 22.24 | BTKB |
| ATOM | 2280 | CA | ARG | 422 | 47.394 | 41.358 | 45.366 | 1.00 | 24.43 | BTKB |
| ATOM | 2281 | CB | ARG | 422 | 46.387 | 41.228 | 46.520 | 1.00 | 25.85 | BTKB |
| ATOM | 2282 | CG | ARG | 422 | 44.960 | 41.672 | 46.222 | 1.00 | 27.33 | BTKB |
| ATOM | 2283 | CD | ARG | 422 | 44.832 | 43.176 | 46.095 | 1.00 | 29.62 | BTKB |
| ATOM | 2284 | NE | ARG | 422 | 43.473 | 43.622 | 46.388 | 1.00 | 29.09 | BTKB |
| ATOM | 2285 | CZ | ARG | 422 | 42.944 | 43.659 | 47.607 | 1.00 | 28.92 | BTKB |
| ATOM | 2286 | NH1 | ARG | 422 | 41.698 | 44.078 | 47.781 | 1.00 | 31.54 | BTKB |
| ATOM | 2287 | NH2 | ARG | 422 | 43.654 | 43.273 | 48.657 | 1.00 | 31.57 | BTKB |
| ATOM | 2288 | C | ARG | 422 | 48.761 | 40.854 | 45.841 | 1.00 | 26.12 | BTKB |
| ATOM | 2289 | O | ARG | 422 | 49.068 | 40.896 | 47.033 | 1.00 | 24.76 | BTKB |
| ATOM | 2290 | N | GLY | 423 | 49.551 | 40.328 | 44.911 | 1.00 | 27.65 | BTKB |
| ATOM | 2291 | CA | GLY | 423 | 50.879 | 39.826 | 45.235 | 1.00 | 27.28 | BTKB |
| ATOM | 2292 | C | GLY | 423 | 50.970 | 38.814 | 46.366 | 1.00 | 28.60 | BTKB |
| ATOM | 2293 | O | GLY | 423 | 52.057 | 38.569 | 46.892 | 1.00 | 28.45 | BTKB |
| ATOM | 2294 | N | ALA | 424 | 49.848 | 38.200 | 46.724 | 1.00 | 29.60 | BTKB |
| ATOM | 2295 | CA | ALA | 424 | 49.841 | 37.216 | 47.800 | 1.00 | 27.82 | BTKB |
| ATOM | 2296 | CB | ALA | 424 | 49.602 | 37.902 | 49.142 | 1.00 | 30.88 | BTKB |
| ATOM | 2297 | C | ALA | 424 | 48.807 | 36.124 | 47.584 | 1.00 | 25.78 | BTKB |
| ATOM | 2298 | O | ALA | 424 | 48.987 | 35.002 | 48.047 | 1.00 | 25.99 | BTKB |
| ATOM | 2299 | N | ALA | 425 | 47.739 | 36.442 | 46.862 | 1.00 | 24.03 | BTKB |
| ATOM | 2300 | CA | ALA | 425 | 46.677 | 35.476 | 46.604 | 1.00 | 24.48 | BTKB |
| ATOM | 2301 | CB | ALA | 425 | 45.313 | 36.151 | 46.782 | 1.00 | 20.10 | BTKB |
| ATOM | 2302 | C | ALA | 425 | 46.762 | 34.806 | 45.227 | 1.00 | 23.85 | BTKB |
| ATOM | 2303 | O | ALA | 425 | 46.766 | 35.479 | 44.194 | 1.00 | 23.75 | BTKB |
| ATOM | 2304 | N | ASP | 426 | 46.863 | 33.481 | 45.214 | 1.00 | 23.12 | BTKB |
| ATOM | 2305 | CA | ASP | 426 | 46.907 | 32.739 | 43.955 | 1.00 | 22.00 | BTKB |
| ATOM | 2306 | CB | ASP | 426 | 47.540 | 31.360 | 44.162 | 1.00 | 27.54 | BTKB |
| ATOM | 2307 | CG | ASP | 426 | 49.016 | 31.443 | 44.531 | 1.00 | 28.92 | BTKB |
| ATOM | 2308 | OD1 | ASP | 426 | 49.596 | 30.404 | 44.915 | 1.00 | 30.24 | BTKB |
| ATOM | 2309 | OD2 | ASP | 426 | 49.600 | 32.544 | 44.435 | 1.00 | 28.52 | BTKB |
| ATOM | 2310 | C | ASP | 426 | 45.469 | 32.615 | 43.458 | 1.00 | 17.08 | BTKB |
| ATOM | 2311 | O | ASP | 426 | 44.567 | 32.310 | 44.237 | 1.00 | 15.85 | BTKB |
| ATOM | 2312 | N | VAL | 427 | 45.255 | 32.841 | 42.166 | 1.00 | 13.56 | BTKB |
| ATOM | 2313 | CA | VAL | 427 | 43.909 | 32.810 | 41.603 | 1.00 | 12.45 | BTKB |
| ATOM | 2314 | CB | VAL | 427 | 43.340 | 34.246 | 41.418 | 1.00 | 10.01 | BTKB |
| ATOM | 2315 | CG1 | VAL | 427 | 43.282 | 34.991 | 42.741 | 1.00 | 12.17 | BTKB |
| ATOM | 2316 | CG2 | VAL | 427 | 44.184 | 35.015 | 40.409 | 1.00 | 12.71 | BTKB |
| ATOM | 2317 | C | VAL | 427 | 43.767 | 32.131 | 40.246 | 1.00 | 13.93 | BTKB |
| ATOM | 2318 | O | VAL | 427 | 44.742 | 31.905 | 39.528 | 1.00 | 15.85 | BTKB |
| ATOM | 2319 | N | ALA | 428 | 42.517 | 31.853 | 39.898 | 1.00 | 12.30 | BTKB |
| ATOM | 2320 | CA | ALA | 428 | 42.152 | 31.245 | 38.631 | 1.00 | 12.30 | BTKB |
| ATOM | 2321 | CB | ALA | 428 | 41.386 | 29.957 | 38.871 | 1.00 | 10.91 | BTKB |
| ATOM | 2322 | C | ALA | 428 | 41.238 | 32.288 | 38.023 | 1.00 | 10.93 | BTKB |
| ATOM | 2323 | O | ALA | 428 | 40.383 | 32.823 | 38.724 | 1.00 | 12.65 | BTKB |
| ATOM | 2324 | N | ILE | 429 | 41.436 | 32.618 | 36.750 | 1.00 | 12.14 | BTKB |
| ATOM | 2325 | CA | ILE | 429 | 40.597 | 33.622 | 36.110 | 1.00 | 14.63 | BTKB |
| ATOM | 2326 | CB | ILE | 429 | 41.441 | 34.832 | 35.525 | 1.00 | 13.45 | BTKB |
| ATOM | 2327 | CG2 | ILE | 429 | 42.825 | 34.396 | 35.083 | 1.00 | 15.33 | BTKB |
| ATOM | 2328 | CG1 | ILE | 429 | 40.693 | 35.558 | 34.415 | 1.00 | 7.39 | BTKB |
| ATOM | 2329 | CD | ILE | 429 | 40.969 | 35.023 | 33.036 | 1.00 | 16.26 | BTKB |
| ATOM | 2330 | C | ILE | 429 | 39.573 | 33.051 | 35.124 | 1.00 | 18.66 | BTKB |
| ATOM | 2331 | O | ILE | 429 | 39.934 | 32.469 | 34.095 | 1.00 | 19.46 | BTKB |
| ATOM | 2332 | N | LYS | 430 | 38.295 | 33.199 | 35.482 | 1.00 | 19.80 | BTKB |
| ATOM | 2333 | CA | LYS | 430 | 37.161 | 32.726 | 34.689 | 1.00 | 21.31 | BTKB |
| ATOM | 2334 | CB | LYS | 430 | 35.978 | 32.421 | 35.606 | 1.00 | 20.55 | BTKB |
| ATOM | 2335 | CG | LYS | 430 | 34.755 | 31.868 | 34.901 | 1.00 | 25.20 | BTKB |
| ATOM | 2336 | CD | LYS | 430 | 34.992 | 30.445 | 34.442 | 1.00 | 29.40 | BTKB |
| ATOM | 2337 | CE | LYS | 430 | 33.692 | 29.770 | 34.064 | 1.00 | 29.23 | BTKB |
| ATOM | 2338 | NZ | LYS | 430 | 32.682 | 29.854 | 35.162 | 1.00 | 33.94 | BTKB |
| ATOM | 2339 | C | LYS | 430 | 36.749 | 33.769 | 33.650 | 1.00 | 21.71 | BTKB |
| ATOM | 2340 | O | LYS | 430 | 36.214 | 34.820 | 33.994 | 1.00 | 17.94 | BTKB |
| ATOM | 2341 | N | MET | 431 | 36.968 | 33.438 | 32.382 | 1.00 | 24.22 | BTKB |
| ATOM | 2342 | CA | MET | 431 | 36.668 | 34.320 | 31.260 | 1.00 | 26.94 | BTKB |
| ATOM | 2343 | CB | MET | 431 | 37.814 | 34.211 | 30.257 | 1.00 | 29.16 | BTKB |
| ATOM | 2344 | CG | MET | 431 | 37.670 | 35.004 | 28.993 | 1.00 | 28.32 | BTKB |
| ATOM | 2345 | SD | MET | 431 | 38.671 | 34.218 | 27.732 | 1.00 | 29.24 | BTKB |
| ATOM | 2346 | CE | MET | 431 | 37.422 | 33.825 | 26.504 | 1.00 | 30.72 | BTKB |
| ATOM | 2347 | C | MET | 431 | 35.339 | 33.992 | 30.575 | 1.00 | 30.22 | BTKB |
| ATOM | 2348 | O | MET | 431 | 34.966 | 32.823 | 30.447 | 1.00 | 32.27 | BTKB |
| ATOM | 2349 | N | ILE | 432 | 34.632 | 35.031 | 30.129 | 1.00 | 34.07 | BTKB |
| ATOM | 2350 | CA | ILE | 432 | 33.343 | 34.858 | 29.452 | 1.00 | 35.28 | BTKB |
| ATOM | 2351 | CB | ILE | 432 | 32.218 | 35.740 | 30.089 | 1.00 | 34.75 | BTKB |
| ATOM | 2352 | CG2 | ILE | 432 | 30.852 | 35.123 | 29.802 | 1.00 | 33.26 | BTKB |
| ATOM | 2353 | CG1 | ILE | 432 | 32.396 | 35.858 | 31.606 | 1.00 | 33.83 | BTKB |
| ATOM | 2354 | CD | ILE | 432 | 32.347 | 34.538 | 32.352 | 1.00 | 35.34 | BTKB |
| ATOM | 2355 | C | ILE | 432 | 33.499 | 35.208 | 27.964 | 1.00 | 33.87 | BTKB |
| ATOM | 2356 | O | ILE | 432 | 34.414 | 34.717 | 27.309 | 1.00 | 34.55 | BTKB |
| ATOM | 2357 | N | LYS | 433 | 32.623 | 36.067 | 27.445 | 1.00 | 33.43 | BTKB |
| ATOM | 2358 | CA | LYS | 433 | 32.646 | 36.499 | 26.045 | 1.00 | 31.18 | BTKB |
| ATOM | 2359 | CB | LYS | 433 | 31.888 | 35.507 | 25.146 | 1.00 | 27.65 | BTKB |
| ATOM | 2360 | CG | LYS | 433 | 32.416 | 34.073 | 25.107 | 1.00 | 24.55 | BTKB |
| ATOM | 2361 | CD | LYS | 433 | 33.793 | 33.983 | 24.475 | 1.00 | 27.10 | BTKB |
| ATOM | 2362 | CE | LYS | 433 | 33.807 | 34.598 | 23.094 | 1.00 | 24.68 | BTKB |
| ATOM | 2363 | NZ | LYS | 433 | 35.145 | 34.557 | 22.439 | 1.00 | 18.58 | BTKB |
| ATOM | 2364 | C | LYS | 433 | 31.910 | 37.837 | 25.985 | 1.00 | 30.74 | BTKB |
| ATOM | 2365 | O | LYS | 433 | 30.782 | 37.944 | 26.483 | 1.00 | 29.85 | BTKB |
| ATOM | 2366 | N | GLU | 434 | 32.535 | 38.861 | 25.410 | 1.00 | 30.55 | BTKB |
| ATOM | 2367 | CA | GLU | 434 | 31.863 | 40.153 | 25.300 | 1.00 | 29.48 | BTKB |
| ATOM | 2368 | CB | GLU | 434 | 32.850 | 41.282 | 24.994 | 1.00 | 29.95 | BTKB |
| ATOM | 2369 | CG | GLU | 434 | 32.215 | 42.691 | 24.991 | 1.00 | 26.07 | BTKB |
| ATOM | 2370 | CD | GLU | 434 | 33.233 | 43.829 | 24.915 | 1.00 | 21.62 | BTKB |
| ATOM | 2371 | OE1 | GLU | 434 | 34.425 | 43.574 | 24.649 | 1.00 | 24.27 | BTKB |
| ATOM | 2372 | OE2 | GLU | 434 | 32.837 | 44.992 | 25.130 | 1.00 | 19.02 | BTKB |
| ATOM | 2373 | C | GLU | 434 | 30.835 | 40.005 | 24.189 | 1.00 | 31.41 | BTKB |
| ATOM | 2374 | O | GLU | 434 | 31.047 | 40.420 | 23.054 | 1.00 | 29.99 | BTKB |
| ATOM | 2375 | N | GLY | 435 | 29.742 | 39.334 | 24.526 | 1.00 | 35.03 | BTKB |
| ATOM | 2376 | CA | GLY | 435 | 28.671 | 39.085 | 23.581 | 1.00 | 37.48 | BTKB |
| ATOM | 2377 | C | GLY | 435 | 27.610 | 38.249 | 24.270 | 1.00 | 39.46 | BTKB |
| ATOM | 2378 | O | GLY | 435 | 26.795 | 37.605 | 23.613 | 1.00 | 40.93 | BTKB |
| ATOM | 2379 | N | SER | 436 | 27.626 | 38.257 | 25.603 | 1.00 | 37.43 | BTKB |
| ATOM | 2380 | CA | SER | 436 | 26.671 | 37.497 | 26.392 | 1.00 | 37.99 | BTKB |
| ATOM | 2381 | CB | SER | 436 | 27.107 | 36.030 | 26.476 | 1.00 | 41.23 | BTKB |
| ATOM | 2382 | OG | SER | 436 | 27.624 | 35.565 | 25.240 | 1.00 | 42.60 | BTKB |
| ATOM | 2383 | C | SER | 436 | 26.560 | 38.083 | 27.799 | 1.00 | 39.64 | BTKB |
| ATOM | 2384 | O | SER | 436 | 25.600 | 38.789 | 28.110 | 1.00 | 41.69 | BTKB |
| ATOM | 2385 | N | MET | 437 | 27.555 | 37.798 | 28.640 | 1.00 | 39.73 | BTKB |
| ATOM | 2386 | CA | MET | 437 | 27.580 | 38.277 | 30.024 | 1.00 | 38.94 | BTKB |
| ATOM | 2387 | CB | MET | 437 | 28.591 | 37.462 | 30.832 | 1.00 | 40.22 | BTKB |
| ATOM | 2388 | CG | MET | 437 | 28.318 | 37.367 | 32.325 | 1.00 | 35.33 | BTKB |
| ATOM | 2389 | SD | MET | 437 | 29.214 | 35.957 | 33.007 | 1.00 | 41.10 | BTKB |
| ATOM | 2390 | CE | MET | 437 | 29.769 | 36.561 | 34.621 | 1.00 | 31.14 | BTKB |
| ATOM | 2391 | C | MET | 437 | 27.923 | 39.767 | 30.095 | 1.00 | 39.09 | BTKB |
| ATOM | 2392 | O | MET | 437 | 28.389 | 40.353 | 29.114 | 1.00 | 36.63 | BTKB |
| ATOM | 2393 | N | SER | 438 | 27.723 | 40.367 | 31.267 | 1.00 | 39.27 | BTKB |
| ATOM | 2394 | CA | SER | 438 | 27.990 | 41.787 | 31.447 | 1.00 | 37.97 | BTKB |
| ATOM | 2395 | CB | SER | 438 | 26.845 | 42.593 | 30.835 | 1.00 | 38.54 | BTKB |
| ATOM | 2396 | OG | SER | 438 | 25.603 | 42.171 | 31.372 | 1.00 | 33.86 | BTKB |
| ATOM | 2397 | C | SER | 438 | 28.150 | 42.199 | 32.906 | 1.00 | 36.36 | BTKB |
| ATOM | 2398 | O | SER | 438 | 27.639 | 41.529 | 33.805 | 1.00 | 34.72 | BTKB |
| ATOM | 2399 | N | GLU | 439 | 28.807 | 43.339 | 33.117 | 1.00 | 37.58 | BTKB |
| ATOM | 2400 | CA | GLU | 439 | 29.034 | 43.904 | 34.452 | 1.00 | 37.09 | BTKB |
| ATOM | 2401 | CB | GLU | 439 | 29.945 | 45.139 | 34.361 | 1.00 | 37.29 | BTKB |
| ATOM | 2402 | CG | GLU | 439 | 29.372 | 46.302 | 33.537 | 1.00 | 37.02 | BTKB |
| ATOM | 2403 | CD | GLU | 439 | 30.217 | 47.572 | 33.606 | 1.00 | 36.40 | BTKB |
| ATOM | 2404 | OE1 | GLU | 439 | 30.640 | 47.957 | 34.719 | 1.00 | 36.07 | BTKB |
| ATOM | 2405 | OE2 | GLU | 439 | 30.450 | 48.195 | 32.544 | 1.00 | 35.38 | BTKB |
| ATOM | 2406 | C | GLU | 439 | 27.728 | 44.309 | 35.146 | 1.00 | 36.76 | BTKB |
| ATOM | 2407 | O | GLU | 439 | 27.696 | 44.522 | 36.363 | 1.00 | 32.43 | BTKB |
| ATOM | 2408 | N | ASP | 440 | 26.662 | 44.426 | 34.359 | 1.00 | 35.30 | BTKB |
| ATOM | 2409 | CA | ASP | 440 | 25.362 | 44.826 | 34.867 | 1.00 | 32.48 | BTKB |
| ATOM | 2410 | CB | ASP | 440 | 24.662 | 45.730 | 33.848 | 1.00 | 36.16 | BTKB |
| ATOM | 2411 | CG | ASP | 440 | 25.592 | 46.775 | 33.253 | 1.00 | 40.98 | BTKB |
| ATOM | 2412 | OD1 | ASP | 440 | 25.560 | 47.945 | 33.691 | 1.00 | 39.71 | BTKB |
| ATOM | 2413 | OD2 | ASP | 440 | 26.358 | 46.426 | 32.334 | 1.00 | 37.76 | BTKB |
| ATOM | 2414 | C | ASP | 440 | 24.480 | 43.626 | 35.179 | 1.00 | 31.97 | BTKB |
| ATOM | 2415 | O | ASP | 440 | 23.521 | 43.745 | 35.943 | 1.00 | 28.02 | BTKB |
| ATOM | 2416 | N | GLU | 441 | 24.783 | 42.477 | 34.574 | 1.00 | 33.36 | BTKB |
| ATOM | 2417 | CA | GLU | 441 | 23.978 | 41.278 | 34.815 | 1.00 | 33.34 | BTKB |
| ATOM | 2418 | CB | GLU | 441 | 24.000 | 40.317 | 33.601 | 1.00 | 33.19 | BTKB |
| ATOM | 2419 | CG | GLU | 441 | 25.090 | 39.216 | 33.599 | 1.00 | 34.36 | BTKB |
| ATOM | 2420 | CD | GLU | 441 | 24.702 | 37.934 | 34.361 | 1.00 | 36.00 | BTKB |
| ATOM | 2421 | OE1 | GLU | 441 | 25.523 | 36.989 | 34.398 | 1.00 | 35.37 | BTKB |
| ATOM | 2422 | OE2 | GLU | 441 | 23.585 | 37.858 | 34.918 | 1.00 | 34.93 | BTKB |
| ATOM | 2423 | C | GLU | 441 | 24.414 | 40.546 | 36.077 | 1.00 | 32.17 | BTKB |
| ATOM | 2424 | O | GLU | 441 | 23.586 | 40.191 | 36.930 | 1.00 | 29.45 | BTKB |
| ATOM | 2425 | N | PHE | 442 | 25.719 | 40.357 | 36.220 | 1.00 | 29.54 | BTKB |
| ATOM | 2426 | CA | PHE | 442 | 26.202 | 39.631 | 37.366 | 1.00 | 31.35 | BTKB |
| ATOM | 2427 | CB | PHE | 442 | 27.382 | 38.730 | 36.988 | 1.00 | 28.63 | BTKB |
| ATOM | 2428 | CG | PHE | 442 | 28.708 | 39.424 | 36.944 | 1.00 | 22.65 | BTKB |
| ATOM | 2429 | CD1 | PHE | 442 | 29.837 | 38.790 | 37.440 | 1.00 | 20.14 | BTKB |
| ATOM | 2430 | CD2 | PHE | 442 | 28.843 | 40.683 | 36.387 | 1.00 | 22.28 | BTKB |
| ATOM | 2431 | CE1 | PHE | 442 | 31.072 | 39.390 | 37.379 | 1.00 | 17.92 | BTKB |
| ATOM | 2432 | CE2 | PHE | 442 | 30.081 | 41.291 | 36.323 | 1.00 | 24.89 | BTKB |
| ATOM | 2433 | CZ | PHE | 442 | 31.198 | 40.641 | 36.822 | 1.00 | 18.41 | BTKB |
| ATOM | 2434 | C | PHE | 442 | 26.494 | 40.449 | 38.604 | 1.00 | 32.00 | BTKB |
| ATOM | 2435 | O | PHE | 442 | 26.809 | 39.879 | 39.634 | 1.00 | 34.02 | BTKB |
| ATOM | 2436 | N | ILE | 443 | 26.343 | 41.767 | 38.529 | 1.00 | 31.37 | BTKB |
| ATOM | 2437 | CA | ILE | 443 | 26.608 | 42.613 | 39.689 | 1.00 | 34.20 | BTKB |
| ATOM | 2438 | CB | ILE | 443 | 26.334 | 44.114 | 39.393 | 1.00 | 35.12 | BTKB |
| ATOM | 2439 | CG2 | ILE | 443 | 24.913 | 44.314 | 38.889 | 1.00 | 35.43 | BTKB |
| ATOM | 2440 | CG1 | ILE | 443 | 26.577 | 44.961 | 40.649 | 1.00 | 36.71 | BTKB |
| ATOM | 2441 | CD | ILE | 443 | 28.010 | 44.938 | 41.152 | 1.00 | 33.98 | BTKB |
| ATOM | 2442 | C | ILE | 443 | 25.758 | 42.142 | 40.866 | 1.00 | 34.50 | BTKB |
| ATOM | 2443 | O | ILE | 443 | 26.243 | 42.006 | 41.993 | 1.00 | 33.00 | BTKB |
| ATOM | 2444 | N | GLU | 444 | 24.499 | 41.840 | 40.577 | 1.00 | 33.96 | BTKB |
| ATOM | 2445 | CA | GLU | 444 | 23.576 | 41.364 | 41.593 | 1.00 | 33.66 | BTKB |
| ATOM | 2446 | CB | GLU | 444 | 22.199 | 41.122 | 40.955 | 1.00 | 34.83 | BTKB |
| ATOM | 2447 | CG | GLU | 444 | 21.019 | 41.072 | 41.920 | 1.00 | 34.28 | BTKB |
| ATOM | 2448 | CD | GLU | 444 | 20.937 | 39.782 | 42.712 | 1.00 | 35.97 | BTKB |
| ATOM | 2449 | OE1 | GLU | 444 | 21.288 | 38.710 | 42.167 | 1.00 | 32.68 | BTKB |
| ATOM | 2450 | OE2 | GLU | 444 | 20.501 | 39.841 | 43.881 | 1.00 | 36.01 | BTKB |
| ATOM | 2451 | C | GLU | 444 | 24.159 | 40.064 | 42.158 | 1.00 | 33.11 | BTKB |
| ATOM | 2452 | O | GLU | 444 | 24.610 | 40.024 | 43.305 | 1.00 | 30.69 | BTKB |
| ATOM | 2453 | N | GLU | 445 | 24.239 | 39.042 | 41.307 | 1.00 | 32.60 | BTKB |
| ATOM | 2454 | CA | GLU | 445 | 24.752 | 37.731 | 41.688 | 1.00 | 30.53 | BTKB |
| ATOM | 2455 | CB | GLU | 445 | 24.753 | 36.784 | 40.486 | 1.00 | 31.01 | BTKB |
| ATOM | 2456 | CG | GLU | 445 | 23.366 | 36.338 | 40.032 | 1.00 | 28.32 | BTKB |
| ATOM | 2457 | CD | GLU | 445 | 23.382 | 35.007 | 39.296 | 1.00 | 28.84 | BTKB |
| ATOM | 2458 | OE1 | GLU | 445 | 24.392 | 34.275 | 39.386 | 1.00 | 28.01 | BTKB |
| ATOM | 2459 | OE2 | GLU | 445 | 22.372 | 34.682 | 38.640 | 1.00 | 27.39 | BTKB |
| ATOM | 2460 | C | GLU | 445 | 26.134 | 37.732 | 42.328 | 1.00 | 31.12 | BTKB |
| ATOM | 2461 | O | GLU | 445 | 26.404 | 36.934 | 43.225 | 1.00 | 33.61 | BTKB |
| ATOM | 2462 | N | ALA | 446 | 26.993 | 38.643 | 41.888 | 1.00 | 30.01 | BTKB |
| ATOM | 2463 | CA | ALA | 446 | 28.351 | 38.739 | 42.410 | 1.00 | 29.17 | BTKB |
| ATOM | 2464 | CB | ALA | 446 | 29.145 | 39.800 | 41.655 | 1.00 | 27.22 | BTKB |
| ATOM | 2465 | C | ALA | 446 | 28.312 | 39.065 | 43.894 | 1.00 | 30.07 | BTKB |
| ATOM | 2466 | O | ALA | 446 | 28.845 | 38.317 | 44.712 | 1.00 | 31.57 | BTKB |
| ATOM | 2467 | N | LYS | 447 | 27.634 | 40.153 | 44.240 | 1.00 | 30.54 | BTKB |
| ATOM | 2468 | CA | LYS | 447 | 27.525 | 40.574 | 45.631 | 1.00 | 29.93 | BTKB |
| ATOM | 2469 | CB | LYS | 447 | 26.676 | 41.847 | 45.742 | 1.00 | 29.51 | BTKB |
| ATOM | 2470 | CG | LYS | 447 | 27.176 | 43.023 | 44.906 | 1.00 | 27.76 | BTKB |
| ATOM | 2471 | CD | LYS | 447 | 28.634 | 43.362 | 45.198 | 1.00 | 24.63 | BTKB |
| ATOM | 2472 | CE | LYS | 447 | 28.846 | 43.812 | 46.635 | 1.00 | 23.47 | BTKB |
| ATOM | 2473 | NZ | LYS | 447 | 30.298 | 43.984 | 46.929 | 1.00 | 24.76 | BTKB |
| ATOM | 2474 | C | LYS | 447 | 26.917 | 39.465 | 46.487 | 1.00 | 31.89 | BTKB |
| ATOM | 2475 | O | LYS | 447 | 27.393 | 39.194 | 47.597 | 1.00 | 31.20 | BTKB |
| ATOM | 2476 | N | VAL | 448 | 25.893 | 38.804 | 45.949 | 1.00 | 31.29 | BTKB |
| ATOM | 2477 | CA | VAL | 448 | 25.207 | 37.725 | 46.651 | 1.00 | 30.04 | BTKB |
| ATOM | 2478 | CB | VAL | 448 | 23.958 | 37.250 | 45.882 | 1.00 | 29.87 | BTKB |
| ATOM | 2479 | CG1 | VAL | 448 | 23.189 | 36.226 | 46.705 | 1.00 | 28.85 | BTKB |
| ATOM | 2480 | CG2 | VAL | 448 | 23.062 | 38.428 | 45.547 | 1.00 | 33.30 | BTKB |
| ATOM | 2481 | C | VAL | 448 | 26.126 | 36.531 | 46.881 | 1.00 | 30.34 | BTKB |
| ATOM | 2482 | O | VAL | 448 | 26.074 | 35.900 | 47.939 | 1.00 | 31.49 | BTKB |
| ATOM | 2483 | N | MET | 449 | 26.963 | 36.215 | 45.897 | 1.00 | 29.17 | BTKB |
| ATOM | 2484 | CA | MET | 449 | 27.882 | 35.091 | 46.027 | 1.00 | 27.85 | BTKB |
| ATOM | 2485 | CB | MET | 449 | 28.473 | 34.708 | 44.676 | 1.00 | 27.95 | BTKB |
| ATOM | 2486 | CG | MET | 449 | 27.570 | 33.842 | 43.834 | 1.00 | 25.86 | BTKB |
| ATOM | 2487 | SD | MET | 449 | 27.991 | 33.992 | 42.101 | 1.00 | 28.55 | BTKB |
| ATOM | 2488 | CE | MET | 449 | 29.603 | 33.105 | 42.046 | 1.00 | 24.75 | BTKB |
| ATOM | 2489 | C | MET | 449 | 28.994 | 35.425 | 46.997 | 1.00 | 29.13 | BTKB |
| ATOM | 2490 | O | MET | 449 | 29.497 | 34.551 | 47.694 | 1.00 | 32.51 | BTKB |
| ATOM | 2491 | N | MET | 450 | 29.376 | 36.696 | 47.046 | 1.00 | 27.71 | BTKB |
| ATOM | 2492 | CA | MET | 450 | 30.422 | 37.130 | 47.955 | 1.00 | 25.39 | BTKB |
| ATOM | 2493 | CB | MET | 450 | 30.926 | 38.512 | 47.558 | 1.00 | 24.26 | BTKB |
| ATOM | 2494 | CG | MET | 450 | 31.553 | 38.506 | 46.179 | 1.00 | 23.94 | BTKB |
| ATOM | 2495 | SD | MET | 450 | 32.473 | 39.970 | 45.741 | 1.00 | 32.01 | BTKB |
| ATOM | 2496 | CE | MET | 450 | 33.957 | 39.761 | 46.735 | 1.00 | 25.95 | BTKB |
| ATOM | 2497 | C | MET | 450 | 29.883 | 37.103 | 49.381 | 1.00 | 27.55 | BTKB |
| ATOM | 2498 | O | MET | 450 | 30.627 | 37.257 | 50.351 | 1.00 | 28.08 | BTKB |
| ATOM | 2499 | N | ASN | 451 | 28.573 | 36.904 | 49.499 | 1.00 | 24.81 | BTKB |
| ATOM | 2500 | CA | ASN | 451 | 27.919 | 36.800 | 50.796 | 1.00 | 23.63 | BTKB |
| ATOM | 2501 | CB | ASN | 451 | 26.414 | 37.017 | 50.662 | 1.00 | 22.42 | BTKB |
| ATOM | 2502 | CG | ASN | 451 | 25.999 | 38.446 | 50.915 | 1.00 | 20.08 | BTKB |
| ATOM | 2503 | OD1 | ASN | 451 | 24.868 | 38.701 | 51.326 | 1.00 | 22.32 | BTKB |
| ATOM | 2504 | ND2 | ASN | 451 | 26.896 | 39.390 | 50.656 | 1.00 | 17.73 | BTKB |
| ATOM | 2505 | C | ASN | 451 | 28.157 | 35.389 | 51.299 | 1.00 | 24.31 | BTKB |
| ATOM | 2506 | O | ASN | 451 | 27.841 | 35.065 | 52.447 | 1.00 | 23.55 | BTKB |
| ATOM | 2507 | N | LEU | 452 | 28.657 | 34.539 | 50.406 | 1.00 | 23.43 | BTKB |
| ATOM | 2508 | CA | LEU | 452 | 28.946 | 33.146 | 50.714 | 1.00 | 21.56 | BTKB |
| ATOM | 2509 | CB | LEU | 452 | 28.585 | 32.255 | 49.522 | 1.00 | 21.06 | BTKB |
| ATOM | 2510 | CG | LEU | 452 | 27.138 | 31.762 | 49.400 | 1.00 | 20.32 | BTKB |
| ATOM | 2511 | CD1 | LEU | 452 | 26.150 | 32.810 | 49.869 | 1.00 | 20.52 | BTKB |
| ATOM | 2512 | CD2 | LEU | 452 | 26.869 | 31.351 | 47.968 | 1.00 | 18.22 | BTKB |
| ATOM | 2513 | C | LEU | 452 | 30.414 | 32.985 | 51.050 | 1.00 | 22.10 | BTKB |
| ATOM | 2514 | O | LEU | 452 | 31.280 | 33.131 | 50.191 | 1.00 | 25.35 | BTKB |
| ATOM | 2515 | N | SER | 453 | 30.688 | 32.718 | 52.317 | 1.00 | 22.10 | BTKB |
| ATOM | 2516 | CA | SER | 453 | 32.047 | 32.525 | 52.784 | 1.00 | 21.51 | BTKB |
| ATOM | 2517 | CB | SER | 453 | 32.537 | 33.751 | 53.565 | 1.00 | 27.28 | BTKB |
| ATOM | 2518 | OG | SER | 453 | 32.794 | 34.849 | 52.707 | 1.00 | 32.08 | BTKB |
| ATOM | 2519 | C | SER | 453 | 32.051 | 31.314 | 53.690 | 1.00 | 20.26 | BTKB |
| ATOM | 2520 | O | SER | 453 | 31.201 | 31.182 | 54.576 | 1.00 | 21.49 | BTKB |
| ATOM | 2521 | N | HIS | 454 | 32.979 | 30.407 | 53.425 | 1.00 | 16.43 | BTKB |
| ATOM | 2522 | CA | HIS | 454 | 33.134 | 29.195 | 54.203 | 1.00 | 14.23 | BTKB |
| ATOM | 2523 | CB | HIS | 454 | 31.930 | 28.256 | 54.025 | 1.00 | 13.19 | BTKB |
| ATOM | 2524 | CG | HIS | 454 | 31.921 | 27.100 | 54.982 | 1.00 | 12.78 | BTKB |
| ATOM | 2525 | CD2 | HIS | 454 | 32.496 | 25.875 | 54.910 | 1.00 | 11.15 | BTKB |
| ATOM | 2526 | ND1 | HIS | 454 | 31.317 | 27.164 | 56.218 | 1.00 | 13.51 | BTKB |
| ATOM | 2527 | CE1 | HIS | 454 | 31.526 | 26.033 | 56.871 | 1.00 | 12.23 | BTKB |
| ATOM | 2528 | NE2 | HIS | 454 | 32.241 | 25.235 | 56.099 | 1.00 | 6.57 | BTKB |
| ATOM | 2529 | C | HIS | 454 | 34.378 | 28.535 | 53.666 | 1.00 | 13.02 | BTKB |
| ATOM | 2530 | O | HIS | 454 | 34.569 | 28.478 | 52.462 | 1.00 | 14.69 | BTKB |
| ATOM | 2531 | N | GLU | 455 | 35.225 | 28.064 | 54.572 | 1.00 | 17.21 | BTKB |
| ATOM | 2532 | CA | GLU | 455 | 36.472 | 27.373 | 54.247 | 1.00 | 19.94 | BTKB |
| ATOM | 2533 | CB | GLU | 455 | 37.152 | 26.954 | 55.569 | 1.00 | 25.60 | BTKB |
| ATOM | 2534 | CG | GLU | 455 | 37.914 | 25.623 | 55.594 | 1.00 | 26.02 | BTKB |
| ATOM | 2535 | CD | GLU | 455 | 39.213 | 25.637 | 54.816 | 1.00 | 23.62 | BTKB |
| ATOM | 2536 | OE1 | GLU | 455 | 39.525 | 24.609 | 54.188 | 1.00 | 26.91 | BTKB |
| ATOM | 2537 | OE2 | GLU | 455 | 39.926 | 26.661 | 54.834 | 1.00 | 26.89 | BTKB |
| ATOM | 2538 | C | GLU | 455 | 36.294 | 26.179 | 53.294 | 1.00 | 18.16 | BTKB |
| ATOM | 2539 | O | GLU | 455 | 37.260 | 25.701 | 52.706 | 1.00 | 17.46 | BTKB |
| ATOM | 2540 | N | LYS | 456 | 35.059 | 25.716 | 53.121 | 1.00 | 17.18 | BTKB |
| ATOM | 2541 | CA | LYS | 456 | 34.791 | 24.581 | 52.239 | 1.00 | 15.92 | BTKB |
| ATOM | 2542 | CB | LYS | 456 | 33.958 | 23.528 | 52.970 | 1.00 | 18.08 | BTKB |
| ATOM | 2543 | CG | LYS | 456 | 34.661 | 22.920 | 54.182 | 1.00 | 15.32 | BTKB |
| ATOM | 2544 | CD | LYS | 456 | 35.909 | 22.160 | 53.764 | 1.00 | 16.08 | BTKB |
| ATOM | 2545 | CE | LYS | 456 | 36.628 | 21.511 | 54.935 | 1.00 | 14.75 | BTKB |
| ATOM | 2546 | NZ | LYS | 456 | 37.413 | 22.460 | 55.750 | 1.00 | 15.29 | BTKB |
| ATOM | 2547 | C | LYS | 456 | 34.148 | 24.962 | 50.897 | 1.00 | 16.33 | BTKB |
| ATOM | 2548 | O | LYS | 456 | 33.760 | 24.091 | 50.108 | 1.00 | 11.16 | BTKB |
| ATOM | 2549 | N | LEU | 457 | 34.015 | 26.265 | 50.653 | 1.00 | 15.73 | BTKB |
| ATOM | 2550 | CA | LEU | 457 | 33.454 | 26.774 | 49.404 | 1.00 | 14.51 | BTKB |
| ATOM | 2551 | CB | LEU | 457 | 32.349 | 27.809 | 49.658 | 1.00 | 14.58 | BTKB |
| ATOM | 2552 | CG | LEU | 457 | 30.948 | 27.463 | 50.169 | 1.00 | 11.43 | BTKB |
| ATOM | 2553 | CD1 | LEU | 457 | 30.166 | 28.752 | 50.355 | 1.00 | 9.22 | BTKB |
| ATOM | 2554 | CD2 | LEU | 457 | 30.216 | 26.576 | 49.186 | 1.00 | 13.55 | BTKB |
| ATOM | 2555 | C | LEU | 457 | 34.613 | 27.473 | 48.722 | 1.00 | 11.29 | BTKB |
| ATOM | 2556 | O | LEU | 457 | 35.400 | 28.152 | 49.385 | 1.00 | 14.89 | BTKB |
| ATOM | 2557 | N | VAL | 458 | 34.756 | 27.282 | 47.417 | 1.00 | 14.60 | BTKB |
| ATOM | 2558 | CA | VAL | 458 | 35.831 | 27.945 | 46.690 | 1.00 | 13.47 | BTKB |
| ATOM | 2559 | CB | VAL | 458 | 36.015 | 27.338 | 45.292 | 1.00 | 10.95 | BTKB |
| ATOM | 2560 | CG1 | VAL | 458 | 37.107 | 28.080 | 44.523 | 1.00 | 8.36 | BTKB |
| ATOM | 2561 | CG2 | VAL | 458 | 36.377 | 25.870 | 45.425 | 1.00 | 4.94 | BTKB |
| ATOM | 2562 | C | VAL | 458 | 35.418 | 29.414 | 46.627 | 1.00 | 17.55 | BTKB |
| ATOM | 2563 | O | VAL | 458 | 34.339 | 29.744 | 46.129 | 1.00 | 19.50 | BTKB |
| ATOM | 2564 | N | GLN | 459 | 36.256 | 30.288 | 47.173 | 1.00 | 19.70 | BTKB |
| ATOM | 2565 | CA | GLN | 459 | 35.930 | 31.710 | 47.231 | 1.00 | 22.06 | BTKB |
| ATOM | 2566 | CB | GLN | 459 | 36.572 | 32.360 | 48.461 | 1.00 | 22.66 | BTKB |
| ATOM | 2567 | CG | GLN | 459 | 38.082 | 32.441 | 48.427 | 1.00 | 29.48 | BTKB |
| ATOM | 2568 | CD | GLN | 459 | 38.657 | 32.969 | 49.727 | 1.00 | 31.10 | BTKB |
| ATOM | 2569 | OE1 | GLN | 459 | 39.341 | 32.248 | 50.454 | 1.00 | 26.32 | BTKB |
| ATOM | 2570 | NE2 | GLN | 459 | 38.370 | 34.230 | 50.033 | 1.00 | 29.38 | BTKB |
| ATOM | 2571 | C | GLN | 459 | 36.165 | 32.594 | 46.019 | 1.00 | 21.06 | BTKB |
| ATOM | 2572 | O | GLN | 459 | 37.209 | 32.543 | 45.370 | 1.00 | 22.08 | BTKB |
| ATOM | 2573 | N | LEU | 460 | 35.177 | 33.435 | 45.754 | 1.00 | 21.47 | BTKB |
| ATOM | 2574 | CA | LEU | 460 | 35.224 | 34.395 | 44.665 | 1.00 | 23.95 | BTKB |
| ATOM | 2575 | CB | LEU | 460 | 33.786 | 34.778 | 44.269 | 1.00 | 22.50 | BTKB |
| ATOM | 2576 | CG | LEU | 460 | 33.466 | 35.635 | 43.034 | 1.00 | 22.00 | BTKB |
| ATOM | 2577 | CD1 | LEU | 460 | 31.973 | 35.572 | 42.746 | 1.00 | 24.79 | BTKB |
| ATOM | 2578 | CD2 | LEU | 460 | 33.895 | 37.074 | 43.243 | 1.00 | 22.74 | BTKB |
| ATOM | 2579 | C | LEU | 460 | 35.953 | 35.592 | 45.278 | 1.00 | 24.95 | BTKB |
| ATOM | 2580 | O | LEU | 460 | 35.416 | 36.254 | 46.165 | 1.00 | 25.13 | BTKB |
| ATOM | 2581 | N | TYR | 461 | 37.198 | 35.821 | 44.873 | 1.00 | 24.81 | BTKB |
| ATOM | 2582 | CA | TYR | 461 | 37.966 | 36.943 | 45.411 | 1.00 | 25.17 | BTKB |
| ATOM | 2583 | CB | TYR | 461 | 39.457 | 36.816 | 45.064 | 1.00 | 25.82 | BTKB |
| ATOM | 2584 | CG | TYR | 461 | 40.199 | 35.689 | 45.774 | 1.00 | 25.50 | BTKB |
| ATOM | 2585 | CD1 | TYR | 461 | 40.773 | 34.643 | 45.050 | 1.00 | 26.73 | BTKB |
| ATOM | 2586 | CE1 | TYR | 461 | 41.476 | 33.618 | 45.682 | 1.00 | 24.71 | BTKB |
| ATOM | 2587 | CD2 | TYR | 461 | 40.347 | 35.682 | 47.161 | 1.00 | 25.96 | BTKB |
| ATOM | 2588 | CE2 | TYR | 461 | 41.050 | 34.659 | 47.807 | 1.00 | 25.37 | BTKB |
| ATOM | 2589 | CZ | TYR | 461 | 41.612 | 33.631 | 47.058 | 1.00 | 25.30 | BTKB |
| ATOM | 2590 | OH | TYR | 461 | 42.317 | 32.622 | 47.681 | 1.00 | 25.28 | BTKB |
| ATOM | 2591 | C | TYR | 461 | 37.407 | 38.259 | 44.871 | 1.00 | 27.25 | BTKB |
| ATOM | 2592 | O | TYR | 461 | 37.293 | 39.246 | 45.596 | 1.00 | 26.29 | BTKB |
| ATOM | 2593 | N | GLY | 462 | 37.041 | 38.262 | 43.596 | 1.00 | 27.06 | BTKB |
| ATOM | 2594 | CA | GLY | 462 | 36.499 | 39.464 | 42.993 | 1.00 | 24.45 | BTKB |
| ATOM | 2595 | C | GLY | 462 | 36.196 | 39.275 | 41.523 | 1.00 | 22.73 | BTKB |
| ATOM | 2596 | O | GLY | 462 | 36.553 | 38.262 | 40.935 | 1.00 | 20.21 | BTKB |
| ATOM | 2597 | N | VAL | 463 | 35.527 | 40.253 | 40.930 | 1.00 | 24.33 | BTKB |
| ATOM | 2598 | CA | VAL | 463 | 35.182 | 40.198 | 39.523 | 1.00 | 24.62 | BTKB |
| ATOM | 2599 | CB | VAL | 463 | 33.697 | 39.891 | 39.319 | 1.00 | 27.36 | BTKB |
| ATOM | 2600 | CG1 | VAL | 463 | 33.368 | 38.519 | 39.854 | 1.00 | 33.31 | BTKB |
| ATOM | 2601 | CG2 | VAL | 463 | 32.843 | 40.935 | 40.011 | 1.00 | 30.70 | BTKB |
| ATOM | 2602 | C | VAL | 463 | 35.478 | 41.528 | 38.861 | 1.00 | 24.47 | BTKB |
| ATOM | 2603 | O | VAL | 463 | 35.418 | 42.584 | 39.504 | 1.00 | 24.70 | BTKB |
| ATOM | 2604 | N | CYS | 464 | 35.829 | 41.473 | 37.584 | 1.00 | 27.38 | BTKB |
| ATOM | 2605 | CA | CYS | 464 | 36.115 | 42.677 | 36.828 | 1.00 | 29.15 | BTKB |
| ATOM | 2606 | CB | CYS | 464 | 37.073 | 42.384 | 35.675 | 1.00 | 33.36 | BTKB |
| ATOM | 2607 | SG | CYS | 464 | 37.677 | 43.868 | 34.853 | 1.00 | 38.92 | BTKB |
| ATOM | 2608 | C | CYS | 464 | 34.779 | 43.162 | 36.302 | 1.00 | 29.60 | BTKB |
| ATOM | 2609 | O | CYS | 464 | 34.321 | 42.744 | 35.238 | 1.00 | 27.69 | BTKB |
| ATOM | 2610 | N | THR | 465 | 34.113 | 43.960 | 37.125 | 1.00 | 31.56 | BTKB |
| ATOM | 2611 | CA | THR | 465 | 32.815 | 44.528 | 36.807 | 1.00 | 30.98 | BTKB |
| ATOM | 2612 | CB | THR | 465 | 32.208 | 45.184 | 38.058 | 1.00 | 31.96 | BTKB |
| ATOM | 2613 | OG1 | THR | 465 | 33.219 | 45.959 | 38.723 | 1.00 | 30.73 | BTKB |
| ATOM | 2614 | CG2 | THR | 465 | 31.652 | 44.127 | 39.010 | 1.00 | 33.13 | BTKB |
| ATOM | 2615 | C | THR | 465 | 32.982 | 45.582 | 35.731 | 1.00 | 31.69 | BTKB |
| ATOM | 2616 | O | THR | 465 | 32.916 | 46.778 | 36.016 | 1.00 | 33.93 | BTKB |
| ATOM | 2617 | N | LYS | 466 | 33.238 | 45.138 | 34.508 | 1.00 | 32.07 | BTKB |
| ATOM | 2618 | CA | LYS | 466 | 33.420 | 46.040 | 33.379 | 1.00 | 31.90 | BTKB |
| ATOM | 2619 | CB | LYS | 466 | 34.908 | 46.348 | 33.185 | 1.00 | 29.79 | BTKB |
| ATOM | 2620 | CG | LYS | 466 | 35.199 | 47.726 | 32.597 | 1.00 | 28.18 | BTKB |
| ATOM | 2621 | CD | LYS | 466 | 34.692 | 48.854 | 33.507 | 1.00 | 28.96 | BTKB |
| ATOM | 2622 | CE | LYS | 466 | 33.405 | 49.481 | 32.968 | 1.00 | 30.68 | BTKB |
| ATOM | 2623 | NZ | LYS | 466 | 32.784 | 50.444 | 33.929 | 1.00 | 29.32 | BTKB |
| ATOM | 2624 | C | LYS | 466 | 32.844 | 45.326 | 32.162 | 1.00 | 35.07 | BTKB |
| ATOM | 2625 | O | LYS | 466 | 32.073 | 44.374 | 32.314 | 1.00 | 35.06 | BTKB |
| ATOM | 2626 | N | GLN | 467 | 33.184 | 45.770 | 30.956 | 1.00 | 39.06 | BTKB |
| ATOM | 2627 | CA | GLN | 467 | 32.647 | 45.103 | 29.782 | 1.00 | 40.26 | BTKB |
| ATOM | 2628 | CB | GLN | 467 | 31.613 | 45.967 | 29.057 | 1.00 | 40.39 | BTKB |
| ATOM | 2629 | CG | GLN | 467 | 30.831 | 45.184 | 28.005 | 1.00 | 39.46 | BTKB |
| ATOM | 2630 | CD | GLN | 467 | 30.526 | 43.757 | 28.451 | 1.00 | 38.12 | BTKB |
| ATOM | 2631 | OE1 | GLN | 467 | 31.174 | 42.807 | 28.017 | 1.00 | 31.06 | BTKB |
| ATOM | 2632 | NE2 | GLN | 467 | 29.560 | 43.608 | 29.344 | 1.00 | 37.49 | BTKB |
| ATOM | 2633 | C | GLN | 467 | 33.672 | 44.568 | 28.805 | 1.00 | 40.30 | BTKB |
| ATOM | 2634 | O | GLN | 467 | 34.461 | 45.314 | 28.224 | 1.00 | 41.34 | BTKB |
| ATOM | 2635 | N | ARG | 468 | 33.591 | 43.255 | 28.632 | 1.00 | 40.07 | BTKB |
| ATOM | 2636 | CA | ARG | 468 | 34.420 | 42.419 | 27.765 | 1.00 | 39.22 | BTKB |
| ATOM | 2637 | CB | ARG | 468 | 35.865 | 42.938 | 27.661 | 1.00 | 38.37 | BTKB |
| ATOM | 2638 | CG | ARG | 468 | 36.689 | 42.820 | 28.929 | 1.00 | 37.01 | BTKB |
| ATOM | 2639 | CD | ARG | 468 | 38.159 | 43.070 | 28.635 | 1.00 | 34.26 | BTKB |
| ATOM | 2640 | NE | ARG | 468 | 38.710 | 42.100 | 27.691 | 1.00 | 30.22 | BTKB |
| ATOM | 2641 | CZ | ARG | 468 | 38.997 | 40.835 | 27.989 | 1.00 | 33.17 | BTKB |
| ATOM | 2642 | NH1 | ARG | 468 | 38.778 | 40.370 | 29.209 | 1.00 | 34.06 | BTKB |
| ATOM | 2643 | NH2 | ARG | 468 | 39.537 | 40.043 | 27.073 | 1.00 | 32.25 | BTKB |
| ATOM | 2644 | C | ARG | 468 | 34.370 | 41.078 | 28.502 | 1.00 | 38.55 | BTKB |
| ATOM | 2645 | O | ARG | 468 | 33.602 | 40.937 | 29.462 | 1.00 | 40.13 | BTKB |
| ATOM | 2646 | N | PRO | 469 | 35.103 | 40.049 | 28.027 | 1.00 | 36.71 | BTKB |
| ATOM | 2647 | CD | PRO | 469 | 35.916 | 39.864 | 26.809 | 1.00 | 34.66 | BTKB |
| ATOM | 2648 | CA | PRO | 469 | 35.012 | 38.803 | 28.792 | 1.00 | 34.01 | BTKB |
| ATOM | 2649 | CB | PRO | 469 | 36.128 | 37.964 | 28.182 | 1.00 | 32.71 | BTKB |
| ATOM | 2650 | CG | PRO | 469 | 36.058 | 38.358 | 26.734 | 1.00 | 32.30 | BTKB |
| ATOM | 2651 | C | PRO | 469 | 35.277 | 39.133 | 30.259 | 1.00 | 34.23 | BTKB |
| ATOM | 2652 | O | PRO | 469 | 36.312 | 39.718 | 30.592 | 1.00 | 34.40 | BTKB |
| ATOM | 2653 | N | ILE | 470 | 34.279 | 38.884 | 31.101 | 1.00 | 33.52 | BTKB |
| ATOM | 2654 | CA | ILE | 470 | 34.393 | 39.182 | 32.523 | 1.00 | 33.35 | BTKB |
| ATOM | 2655 | CB | ILE | 470 | 33.078 | 38.936 | 33.271 | 1.00 | 31.28 | BTKB |
| ATOM | 2656 | CG2 | ILE | 470 | 33.265 | 39.263 | 34.737 | 1.00 | 32.57 | BTKB |
| ATOM | 2657 | CG1 | ILE | 470 | 31.945 | 39.779 | 32.669 | 1.00 | 31.65 | BTKB |
| ATOM | 2658 | CD | ILE | 470 | 32.076 | 41.265 | 32.915 | 1.00 | 29.56 | BTKB |
| ATOM | 2659 | C | ILE | 470 | 35.489 | 38.369 | 33.187 | 1.00 | 34.04 | BTKB |
| ATOM | 2660 | O | ILE | 470 | 35.424 | 37.145 | 33.247 | 1.00 | 36.53 | BTKB |
| ATOM | 2661 | N | PHE | 471 | 36.511 | 39.064 | 33.662 | 1.00 | 33.94 | BTKB |
| ATOM | 2662 | CA | PHE | 471 | 37.623 | 38.412 | 34.322 | 1.00 | 33.78 | BTKB |
| ATOM | 2663 | CB | PHE | 471 | 38.894 | 39.254 | 34.174 | 1.00 | 34.34 | BTKB |
| ATOM | 2664 | CG | PHE | 471 | 39.606 | 39.058 | 32.865 | 1.00 | 32.47 | BTKB |
| ATOM | 2665 | CD1 | PHE | 471 | 39.345 | 37.942 | 32.072 | 1.00 | 35.03 | BTKB |
| ATOM | 2666 | CD2 | PHE | 471 | 40.573 | 39.962 | 32.448 | 1.00 | 29.92 | BTKB |
| ATOM | 2667 | CE1 | PHE | 471 | 40.039 | 37.728 | 30.893 | 1.00 | 31.59 | BTKB |
| ATOM | 2668 | CE2 | PHE | 471 | 41.272 | 39.755 | 31.270 | 1.00 | 31.32 | BTKB |
| ATOM | 2669 | CZ | PHE | 471 | 41.005 | 38.632 | 30.491 | 1.00 | 30.64 | BTKB |
| ATOM | 2670 | C | PHE | 471 | 37.301 | 38.186 | 35.792 | 1.00 | 34.09 | BTKB |
| ATOM | 2671 | O | PHE | 471 | 37.641 | 39.016 | 36.648 | 1.00 | 34.23 | BTKB |
| ATOM | 2672 | N | ILE | 472 | 36.580 | 37.102 | 36.069 | 1.00 | 31.42 | BTKB |
| ATOM | 2673 | CA | ILE | 472 | 36.224 | 36.754 | 37.438 | 1.00 | 25.25 | BTKB |
| ATOM | 2674 | CB | ILE | 472 | 34.991 | 35.817 | 37.493 | 1.00 | 25.15 | BTKB |
| ATOM | 2675 | CG2 | ILE | 472 | 34.630 | 35.487 | 38.947 | 1.00 | 19.45 | BTKB |
| ATOM | 2676 | CG1 | ILE | 472 | 33.795 | 36.493 | 36.816 | 1.00 | 23.77 | BTKB |
| ATOM | 2677 | CD | ILE | 472 | 32.512 | 35.683 | 36.860 | 1.00 | 27.24 | BTKB |
| ATOM | 2678 | C | ILE | 472 | 37.442 | 36.058 | 38.021 | 1.00 | 24.31 | BTKB |
| ATOM | 2679 | O | ILE | 472 | 38.127 | 35.307 | 37.321 | 1.00 | 21.85 | BTKB |
| ATOM | 2680 | N | ILE | 473 | 37.734 | 36.346 | 39.284 | 1.00 | 24.30 | BTKB |
| ATOM | 2681 | CA | ILE | 473 | 38.885 | 35.767 | 39.965 | 1.00 | 22.53 | BTKB |
| ATOM | 2682 | CB | ILE | 473 | 39.907 | 36.880 | 40.353 | 1.00 | 19.77 | BTKB |
| ATOM | 2683 | CG2 | ILE | 473 | 40.447 | 36.702 | 41.771 | 1.00 | 21.00 | BTKB |
| ATOM | 2684 | CG1 | ILE | 473 | 41.050 | 36.916 | 39.337 | 1.00 | 19.04 | BTKB |
| ATOM | 2685 | CD | ILE | 473 | 40.677 | 37.512 | 38.014 | 1.00 | 11.67 | BTKB |
| ATOM | 2686 | C | ILE | 473 | 38.478 | 34.914 | 41.168 | 1.00 | 22.23 | BTKB |
| ATOM | 2687 | O | ILE | 473 | 37.846 | 35.401 | 42.112 | 1.00 | 18.66 | BTKB |
| ATOM | 2688 | N | THR | 474 | 38.840 | 33.635 | 41.116 | 1.00 | 22.87 | BTKB |
| ATOM | 2689 | CA | THR | 474 | 38.517 | 32.699 | 42.179 | 1.00 | 20.65 | BTKB |
| ATOM | 2690 | CB | THR | 474 | 37.578 | 31.586 | 41.668 | 1.00 | 21.70 | BTKB |
| ATOM | 2691 | OG1 | THR | 474 | 38.161 | 30.959 | 40.521 | 1.00 | 16.76 | BTKB |
| ATOM | 2692 | CG2 | THR | 474 | 36.211 | 32.158 | 41.296 | 1.00 | 19.49 | BTKB |
| ATOM | 2693 | C | THR | 474 | 39.749 | 32.055 | 42.811 | 1.00 | 19.89 | BTKB |
| ATOM | 2694 | O | THR | 474 | 40.882 | 32.284 | 42.379 | 1.00 | 19.24 | BTKB |
| ATOM | 2695 | N | GLU | 475 | 39.499 | 31.234 | 43.826 | 1.00 | 17.71 | BTKB |
| ATOM | 2696 | CA | GLU | 475 | 40.528 | 30.526 | 44.577 | 1.00 | 18.24 | BTKB |
| ATOM | 2697 | CB | GLU | 475 | 39.878 | 29.940 | 45.834 | 1.00 | 17.97 | BTKB |
| ATOM | 2698 | CG | GLU | 475 | 40.814 | 29.304 | 46.846 | 1.00 | 24.32 | BTKB |
| ATOM | 2699 | CD | GLU | 475 | 40.076 | 28.796 | 48.080 | 1.00 | 27.60 | BTKB |
| ATOM | 2700 | OE1 | GLU | 475 | 40.701 | 28.726 | 49.160 | 1.00 | 28.93 | BTKB |
| ATOM | 2701 | OE2 | GLU | 475 | 38.872 | 28.468 | 47.972 | 1.00 | 29.54 | BTKB |
| ATOM | 2702 | C | GLU | 475 | 41.199 | 29.425 | 43.742 | 1.00 | 19.31 | BTKB |
| ATOM | 2703 | O | GLU | 475 | 40.538 | 28.495 | 43.271 | 1.00 | 21.47 | BTKB |
| ATOM | 2704 | N | TYR | 476 | 42.508 | 29.548 | 43.537 | 1.00 | 18.96 | BTKB |
| ATOM | 2705 | CA | TYR | 476 | 43.253 | 28.559 | 42.760 | 1.00 | 19.32 | BTKB |
| ATOM | 2706 | CB | TYR | 476 | 44.673 | 29.054 | 42.436 | 1.00 | 18.59 | BTKB |
| ATOM | 2707 | CG | TYR | 476 | 45.437 | 28.101 | 41.545 | 1.00 | 16.62 | BTKB |
| ATOM | 2708 | CD1 | TYR | 476 | 45.097 | 27.963 | 40.204 | 1.00 | 16.76 | BTKB |
| ATOM | 2709 | CE1 | TYR | 476 | 45.703 | 27.000 | 39.396 | 1.00 | 17.33 | BTKB |
| ATOM | 2710 | CD2 | TYR | 476 | 46.419 | 27.259 | 42.064 | 1.00 | 18.86 | BTKB |
| ATOM | 2711 | CE2 | TYR | 476 | 47.031 | 26.286 | 41.262 | 1.00 | 20.19 | BTKB |
| ATOM | 2712 | CZ | TYR | 476 | 46.660 | 26.164 | 39.929 | 1.00 | 18.14 | BTKB |
| ATOM | 2713 | OH | TYR | 476 | 47.206 | 25.182 | 39.134 | 1.00 | 15.15 | BTKB |
| ATOM | 2714 | C | TYR | 476 | 43.329 | 27.242 | 43.524 | 1.00 | 20.78 | BTKB |
| ATOM | 2715 | O | TYR | 476 | 43.758 | 27.208 | 44.678 | 1.00 | 21.62 | BTKB |
| ATOM | 2716 | N | MET | 477 | 42.925 | 26.158 | 42.871 | 1.00 | 17.28 | BTKB |
| ATOM | 2717 | CA | MET | 477 | 42.947 | 24.844 | 43.499 | 1.00 | 13.93 | BTKB |
| ATOM | 2718 | CB | MET | 477 | 41.552 | 24.232 | 43.458 | 1.00 | 10.54 | BTKB |
| ATOM | 2719 | CG | MET | 477 | 40.506 | 25.096 | 44.152 | 1.00 | 13.09 | BTKB |
| ATOM | 2720 | SD | MET | 477 | 40.797 | 25.261 | 45.909 | 1.00 | 14.13 | BTKB |
| ATOM | 2721 | CE | MET | 477 | 39.882 | 23.854 | 46.522 | 1.00 | 10.24 | BTKB |
| ATOM | 2722 | C | MET | 477 | 43.960 | 23.955 | 42.812 | 1.00 | 11.92 | BTKB |
| ATOM | 2723 | O | MET | 477 | 43.679 | 23.356 | 41.787 | 1.00 | 15.97 | BTKB |
| ATOM | 2724 | N | ALA | 478 | 45.131 | 23.851 | 43.425 | 1.00 | 12.80 | BTKB |
| ATOM | 2725 | CA | ALA | 478 | 46.260 | 23.087 | 42.908 | 1.00 | 11.29 | BTKB |
| ATOM | 2726 | CB | ALA | 478 | 47.397 | 23.091 | 43.927 | 1.00 | 10.32 | BTKB |
| ATOM | 2727 | C | ALA | 478 | 46.042 | 21.673 | 42.382 | 1.00 | 12.60 | BTKB |
| ATOM | 2728 | O | ALA | 478 | 46.767 | 21.239 | 41.479 | 1.00 | 11.34 | BTKB |
| ATOM | 2729 | N | ASN | 479 | 45.075 | 20.946 | 42.934 | 1.00 | 12.61 | BTKB |
| ATOM | 2730 | CA | ASN | 479 | 44.852 | 19.578 | 42.477 | 1.00 | 15.20 | BTKB |
| ATOM | 2731 | CB | ASN | 479 | 44.737 | 18.616 | 43.658 | 1.00 | 13.56 | BTKB |
| ATOM | 2732 | CG | ASN | 479 | 46.096 | 18.176 | 44.181 | 1.00 | 11.21 | BTKB |
| ATOM | 2733 | OD1 | ASN | 479 | 46.963 | 17.763 | 43.418 | 1.00 | 11.71 | BTKB |
| ATOM | 2734 | ND2 | ASN | 479 | 46.284 | 18.265 | 45.484 | 1.00 | 13.16 | BTKB |
| ATOM | 2735 | C | ASN | 479 | 43.714 | 19.367 | 41.494 | 1.00 | 14.96 | BTKB |
| ATOM | 2736 | O | ASN | 479 | 43.370 | 18.230 | 41.181 | 1.00 | 16.66 | BTKB |
| ATOM | 2737 | N | GLY | 480 | 43.160 | 20.467 | 40.985 | 1.00 | 16.33 | BTKB |
| ATOM | 2738 | CA | GLY | 480 | 42.080 | 20.406 | 40.011 | 1.00 | 16.11 | BTKB |
| ATOM | 2739 | C | GLY | 480 | 40.744 | 19.930 | 40.535 | 1.00 | 15.18 | BTKB |
| ATOM | 2740 | O | GLY | 480 | 40.427 | 20.112 | 41.705 | 1.00 | 16.02 | BTKB |
| ATOM | 2741 | N | CYS | 481 | 39.954 | 19.318 | 39.660 | 1.00 | 15.40 | BTKB |
| ATOM | 2742 | CA | CYS | 481 | 38.631 | 18.818 | 40.032 | 1.00 | 16.10 | BTKB |
| ATOM | 2743 | CB | CYS | 481 | 37.726 | 18.767 | 38.804 | 1.00 | 17.50 | BTKB |
| ATOM | 2744 | SG | CYS | 481 | 38.151 | 17.459 | 37.637 | 1.00 | 26.46 | BTKB |
| ATOM | 2745 | C | CYS | 481 | 38.663 | 17.440 | 40.706 | 1.00 | 13.33 | BTKB |
| ATOM | 2746 | O | CYS | 481 | 39.484 | 16.586 | 40.359 | 1.00 | 9.78 | BTKB |
| ATOM | 2747 | N | LEU | 482 | 37.716 | 17.218 | 41.620 | 1.00 | 12.03 | BTKB |
| ATOM | 2748 | CA | LEU | 482 | 37.581 | 15.966 | 42.367 | 1.00 | 12.13 | BTKB |
| ATOM | 2749 | CB | LEU | 482 | 36.390 | 16.058 | 43.340 | 1.00 | 12.80 | BTKB |
| ATOM | 2750 | CG | LEU | 482 | 36.066 | 14.923 | 44.327 | 1.00 | 10.31 | BTKB |
| ATOM | 2751 | CD1 | LEU | 482 | 37.227 | 14.696 | 45.280 | 1.00 | 12.16 | BTKB |
| ATOM | 2752 | CD2 | LEU | 482 | 34.792 | 15.233 | 45.114 | 1.00 | 12.14 | BTKB |
| ATOM | 2753 | C | LEU | 482 | 37.439 | 14.740 | 41.461 | 1.00 | 10.56 | BTKB |
| ATOM | 2754 | O | LEU | 482 | 38.066 | 13.714 | 41.714 | 1.00 | 11.00 | BTKB |
| ATOM | 2755 | N | LEU | 483 | 36.649 | 14.849 | 40.394 | 1.00 | 10.48 | BTKB |
| ATOM | 2756 | CA | LEU | 483 | 36.451 | 13.733 | 39.456 | 1.00 | 9.01 | BTKB |
| ATOM | 2757 | CB | LEU | 483 | 35.600 | 14.167 | 38.261 | 1.00 | 4.67 | BTKB |
| ATOM | 2758 | CG | LEU | 483 | 34.174 | 13.645 | 38.133 | 1.00 | 6.66 | BTKB |
| ATOM | 2759 | CD1 | LEU | 483 | 33.737 | 13.825 | 36.691 | 1.00 | 7.76 | BTKB |
| ATOM | 2760 | CD2 | LEU | 483 | 34.097 | 12.179 | 38.517 | 1.00 | 6.83 | BTKB |
| ATOM | 2761 | C | LEU | 483 | 37.763 | 13.170 | 38.925 | 1.00 | 13.13 | BTKB |
| ATOM | 2762 | O | LEU | 483 | 37.927 | 11.957 | 38.815 | 1.00 | 15.11 | BTKB |
| ATOM | 2763 | N | ASN | 484 | 38.679 | 14.064 | 38.562 | 1.00 | 15.95 | BTKB |
| ATOM | 2764 | CA | ASN | 484 | 39.990 | 13.673 | 38.047 | 1.00 | 18.08 | BTKB |
| ATOM | 2765 | CB | ASN | 484 | 40.689 | 14.855 | 37.371 | 1.00 | 22.13 | BTKB |
| ATOM | 2766 | CG | ASN | 484 | 39.864 | 15.473 | 36.265 | 1.00 | 23.79 | BTKB |
| ATOM | 2767 | OD1 | ASN | 484 | 40.012 | 16.657 | 35.964 | 1.00 | 28.81 | BTKB |
| ATOM | 2768 | ND2 | ASN | 484 | 38.991 | 14.682 | 35.651 | 1.00 | 29.42 | BTKB |
| ATOM | 2769 | C | ASN | 484 | 40.867 | 13.194 | 39.184 | 1.00 | 16.80 | BTKB |
| ATOM | 2770 | O | ASN | 484 | 41.790 | 12.415 | 38.977 | 1.00 | 16.25 | BTKB |
| ATOM | 2771 | N | TYR | 485 | 40.597 | 13.696 | 40.382 | 1.00 | 16.90 | BTKB |
| ATOM | 2772 | CA | TYR | 485 | 41.368 | 13.317 | 41.552 | 1.00 | 15.39 | BTKB |
| ATOM | 2773 | CB | TYR | 485 | 41.053 | 14.260 | 42.713 | 1.00 | 15.38 | BTKB |
| ATOM | 2774 | CG | TYR | 485 | 42.182 | 14.400 | 43.692 | 1.00 | 10.91 | BTKB |
| ATOM | 2775 | CD1 | TYR | 485 | 43.446 | 14.782 | 43.263 | 1.00 | 10.29 | BTKB |
| ATOM | 2776 | CE1 | TYR | 485 | 44.496 | 14.935 | 44.160 | 1.00 | 9.78 | BTKB |
| ATOM | 2777 | CD2 | TYR | 485 | 41.989 | 14.166 | 45.050 | 1.00 | 13.87 | BTKB |
| ATOM | 2778 | CE2 | TYR | 485 | 43.032 | 14.316 | 45.957 | 1.00 | 13.73 | BTKB |
| ATOM | 2779 | CZ | TYR | 485 | 44.284 | 14.704 | 45.501 | 1.00 | 14.21 | BTKB |
| ATOM | 2780 | OH | TYR | 485 | 45.322 | 14.890 | 46.389 | 1.00 | 20.03 | BTKB |
| ATOM | 2781 | C | TYR | 485 | 41.059 | 11.881 | 41.943 | 1.00 | 16.45 | BTKB |
| ATOM | 2782 | O | TYR | 485 | 41.957 | 11.066 | 42.096 | 1.00 | 20.72 | BTKB |
| ATOM | 2783 | N | LEU | 486 | 39.779 | 11.566 | 42.067 | 1.00 | 16.22 | BTKB |
| ATOM | 2784 | CA | LEU | 486 | 39.349 | 10.228 | 42.445 | 1.00 | 14.56 | BTKB |
| ATOM | 2785 | CB | LEU | 486 | 37.821 | 10.172 | 42.439 | 1.00 | 8.92 | BTKB |
| ATOM | 2786 | CG | LEU | 486 | 37.188 | 10.923 | 43.611 | 1.00 | 9.93 | BTKB |
| ATOM | 2787 | CD1 | LEU | 486 | 35.698 | 11.154 | 43.365 | 1.00 | 6.93 | BTKB |
| ATOM | 2788 | CD2 | LEU | 486 | 37.444 | 10.155 | 44.911 | 1.00 | 2.28 | BTKB |
| ATOM | 2789 | C | LEU | 486 | 39.935 | 9.123 | 41.561 | 1.00 | 17.58 | BTKB |
| ATOM | 2790 | O | LEU | 486 | 40.287 | 8.037 | 42.042 | 1.00 | 17.82 | BTKB |
| ATOM | 2791 | N | ARG | 487 | 40.054 | 9.413 | 40.272 | 1.00 | 22.23 | BTKB |
| ATOM | 2792 | CA | ARG | 487 | 40.589 | 8.457 | 39.313 | 1.00 | 24.99 | BTKB |
| ATOM | 2793 | CB | ARG | 487 | 40.060 | 8.786 | 37.914 | 1.00 | 25.06 | BTKB |
| ATOM | 2794 | CG | ARG | 487 | 38.543 | 8.776 | 37.775 | 1.00 | 25.61 | BTKB |
| ATOM | 2795 | CD | ARG | 487 | 37.994 | 7.357 | 37.693 | 1.00 | 34.03 | BTKB |
| ATOM | 2796 | NE | ARG | 487 | 37.266 | 7.113 | 36.445 | 1.00 | 36.39 | BTKB |
| ATOM | 2797 | CZ | ARG | 487 | 36.565 | 6.009 | 36.184 | 1.00 | 33.29 | BTKB |
| ATOM | 2798 | NH1 | ARG | 487 | 36.492 | 5.034 | 37.083 | 1.00 | 31.06 | BTKB |
| ATOM | 2799 | NH2 | ARG | 487 | 35.926 | 5.886 | 35.026 | 1.00 | 32.25 | BTKB |
| ATOM | 2800 | C | ARG | 487 | 42.117 | 8.481 | 39.297 | 1.00 | 25.69 | BTKB |
| ATOM | 2801 | O | ARG | 487 | 42.738 | 7.695 | 38.582 | 1.00 | 27.68 | BTKB |
| ATOM | 2802 | N | GLU | 488 | 42.713 | 9.341 | 40.121 | 1.00 | 29.95 | BTKB |
| ATOM | 2803 | CA | GLU | 488 | 44.164 | 9.507 | 40.168 | 1.00 | 32.26 | BTKB |
| ATOM | 2804 | CB | GLU | 488 | 44.618 | 10.299 | 41.405 | 1.00 | 35.45 | BTKB |
| ATOM | 2805 | CG | GLU | 488 | 44.416 | 9.609 | 42.751 | 1.00 | 35.63 | BTKB |
| ATOM | 2806 | CD | GLU | 488 | 45.086 | 10.359 | 43.902 | 1.00 | 36.03 | BTKB |
| ATOM | 2807 | OE1 | GLU | 488 | 45.854 | 9.725 | 44.657 | 1.00 | 34.00 | BTKB |
| ATOM | 2808 | OE2 | GLU | 488 | 44.857 | 11.578 | 44.050 | 1.00 | 30.65 | BTKB |
| ATOM | 2809 | C | GLU | 488 | 44.967 | 8.234 | 40.046 | 1.00 | 35.02 | BTKB |
| ATOM | 2810 | O | GLU | 488 | 44.680 | 7.234 | 40.704 | 1.00 | 36.27 | BTKB |
| ATOM | 2811 | N | MET | 489 | 45.975 | 8.304 | 39.178 | 1.00 | 39.21 | BTKB |
| ATOM | 2812 | CA | MET | 489 | 46.892 | 7.209 | 38.881 | 1.00 | 40.20 | BTKB |
| ATOM | 2813 | CB | MET | 489 | 48.243 | 7.779 | 38.437 | 1.00 | 40.38 | BTKB |
| ATOM | 2814 | CG | MET | 489 | 48.173 | 9.022 | 37.536 | 1.00 | 41.55 | BTKB |
| ATOM | 2815 | SD | MET | 489 | 47.548 | 8.779 | 35.843 | 1.00 | 43.59 | BTKB |
| ATOM | 2816 | CE | MET | 489 | 46.615 | 10.283 | 35.628 | 1.00 | 40.20 | BTKB |
| ATOM | 2817 | C | MET | 489 | 47.087 | 6.305 | 40.101 | 1.00 | 40.72 | BTKB |
| ATOM | 2818 | O | MET | 489 | 46.473 | 5.239 | 40.204 | 1.00 | 42.05 | BTKB |
| ATOM | 2819 | N | ARG | 490 | 47.930 | 6.744 | 41.028 | 1.00 | 40.79 | BTKB |
| ATOM | 2820 | CA | ARG | 490 | 48.195 | 5.995 | 42.245 | 1.00 | 37.42 | BTKB |
| ATOM | 2821 | CB | ARG | 490 | 49.437 | 6.553 | 42.932 | 1.00 | 33.39 | BTKB |
| ATOM | 2822 | CG | ARG | 490 | 50.727 | 6.263 | 42.195 | 1.00 | 33.75 | BTKB |
| ATOM | 2823 | CD | ARG | 490 | 51.220 | 4.849 | 42.479 | 1.00 | 29.04 | BTKB |
| ATOM | 2824 | NE | ARG | 490 | 52.596 | 4.854 | 42.970 | 1.00 | 27.07 | BTKB |
| ATOM | 2825 | CZ | ARG | 490 | 52.969 | 5.326 | 44.159 | 1.00 | 25.71 | BTKB |
| ATOM | 2826 | NH1 | ARG | 490 | 52.067 | 5.827 | 44.996 | 1.00 | 21.84 | BTKB |
| ATOM | 2827 | NH2 | ARG | 490 | 54.255 | 5.355 | 44.487 | 1.00 | 21.92 | BTKB |
| ATOM | 2828 | C | ARG | 490 | 46.983 | 6.127 | 43.156 | 1.00 | 37.49 | BTKB |
| ATOM | 2829 | O | ARG | 490 | 46.884 | 7.076 | 43.933 | 1.00 | 33.88 | BTKB |
| ATOM | 2830 | N | HIS | 491 | 46.063 | 5.172 | 43.047 | 1.00 | 37.72 | BTKB |
| ATOM | 2831 | CA | HIS | 491 | 44.826 | 5.166 | 43.833 | 1.00 | 35.57 | BTKB |
| ATOM | 2832 | CB | HIS | 491 | 43.863 | 4.096 | 43.288 | 1.00 | 34.70 | BTKB |
| ATOM | 2833 | CG | HIS | 491 | 43.450 | 4.303 | 41.859 | 1.00 | 34.85 | BTKB |
| ATOM | 2834 | CD2 | HIS | 491 | 42.253 | 4.634 | 41.321 | 1.00 | 33.61 | BTKB |
| ATOM | 2835 | ND1 | HIS | 491 | 44.312 | 4.133 | 40.797 | 1.00 | 34.49 | BTKB |
| ATOM | 2836 | CE1 | HIS | 491 | 43.664 | 4.348 | 39.665 | 1.00 | 32.88 | BTKB |
| ATOM | 2837 | NE2 | HIS | 491 | 42.413 | 4.654 | 39.956 | 1.00 | 33.96 | BTKB |
| ATOM | 2838 | C | HIS | 491 | 45.068 | 4.915 | 45.327 | 1.00 | 33.29 | BTKB |
| ATOM | 2839 | O | HIS | 491 | 44.479 | 4.007 | 45.913 | 1.00 | 34.31 | BTKB |
| ATOM | 2840 | N | ARG | 492 | 45.921 | 5.723 | 45.948 | 1.00 | 31.88 | BTKB |
| ATOM | 2841 | CA | ARG | 492 | 46.228 | 5.561 | 47.362 | 1.00 | 31.93 | BTKB |
| ATOM | 2842 | CB | ARG | 492 | 47.566 | 6.224 | 47.697 | 1.00 | 32.51 | BTKB |
| ATOM | 2843 | CG | ARG | 492 | 48.746 | 5.765 | 46.847 | 1.00 | 35.36 | BTKB |
| ATOM | 2844 | CD | ARG | 492 | 50.013 | 6.544 | 47.196 | 1.00 | 33.65 | BTKB |
| ATOM | 2845 | NE | ARG | 492 | 49.859 | 7.979 | 46.958 | 1.00 | 33.39 | BTKB |
| ATOM | 2846 | CZ | ARG | 492 | 50.807 | 8.886 | 47.169 | 1.00 | 29.63 | BTKB |
| ATOM | 2847 | NH1 | ARG | 492 | 51.994 | 8.525 | 47.626 | 1.00 | 22.52 | BTKB |
| ATOM | 2848 | NH2 | ARG | 492 | 50.559 | 10.163 | 46.941 | 1.00 | 28.36 | BTKB |
| ATOM | 2849 | C | ARG | 492 | 45.123 | 6.221 | 48.165 | 1.00 | 33.93 | BTKB |
| ATOM | 2850 | O | ARG | 492 | 45.323 | 7.295 | 48.730 | 1.00 | 36.87 | BTKB |
| ATOM | 2851 | N | PHE | 493 | 43.953 | 5.591 | 48.201 | 1.00 | 32.16 | BTKB |
| ATOM | 2852 | CA | PHE | 493 | 42.824 | 6.148 | 48.932 | 1.00 | 30.58 | BTKB |
| ATOM | 2853 | CB | PHE | 493 | 41.708 | 6.587 | 47.974 | 1.00 | 28.91 | BTKB |
| ATOM | 2854 | CG | PHE | 493 | 41.862 | 7.993 | 47.466 | 1.00 | 22.07 | BTKB |
| ATOM | 2855 | CD1 | PHE | 493 | 42.001 | 8.247 | 46.110 | 1.00 | 23.04 | BTKB |
| ATOM | 2856 | CD2 | PHE | 493 | 41.880 | 9.062 | 48.352 | 1.00 | 20.91 | BTKB |
| ATOM | 2857 | CE1 | PHE | 493 | 42.159 | 9.549 | 45.642 | 1.00 | 21.21 | BTKB |
| ATOM | 2858 | CE2 | PHE | 493 | 42.037 | 10.361 | 47.898 | 1.00 | 18.07 | BTKB |
| ATOM | 2859 | CZ | PHE | 493 | 42.178 | 10.606 | 46.536 | 1.00 | 23.31 | BTKB |
| ATOM | 2860 | C | PHE | 493 | 42.256 | 5.207 | 49.976 | 1.00 | 30.49 | BTKB |
| ATOM | 2861 | O | PHE | 493 | 41.858 | 4.079 | 49.676 | 1.00 | 30.95 | BTKB |
| ATOM | 2862 | N | GLN | 494 | 42.240 | 5.679 | 51.213 | 1.00 | 29.40 | BTKB |
| ATOM | 2863 | CA | GLN | 494 | 41.702 | 4.907 | 52.320 | 1.00 | 28.93 | BTKB |
| ATOM | 2864 | CB | GLN | 494 | 42.640 | 4.985 | 53.529 | 1.00 | 31.31 | BTKB |
| ATOM | 2865 | CG | GLN | 494 | 44.047 | 4.427 | 53.298 | 1.00 | 35.24 | BTKB |
| ATOM | 2866 | CD | GLN | 494 | 45.041 | 5.495 | 52.888 | 1.00 | 36.73 | BTKB |
| ATOM | 2867 | OE1 | GLN | 494 | 44.778 | 6.289 | 51.984 | 1.00 | 36.12 | BTKB |
| ATOM | 2868 | NE2 | GLN | 494 | 46.184 | 5.529 | 53.559 | 1.00 | 34.99 | BTKB |
| ATOM | 2869 | C | GLN | 494 | 40.337 | 5.499 | 52.660 | 1.00 | 26.79 | BTKB |
| ATOM | 2870 | O | GLN | 494 | 40.132 | 6.707 | 52.516 | 1.00 | 30.22 | BTKB |
| ATOM | 2871 | N | THR | 495 | 39.405 | 4.660 | 53.104 | 1.00 | 24.49 | BTKB |
| ATOM | 2872 | CA | THR | 495 | 38.059 | 5.107 | 53.453 | 1.00 | 18.71 | BTKB |
| ATOM | 2873 | CB | THR | 495 | 37.261 | 3.983 | 54.136 | 1.00 | 15.78 | BTKB |
| ATOM | 2874 | OG1 | THR | 495 | 37.282 | 2.823 | 53.302 | 1.00 | 17.79 | BTKB |
| ATOM | 2875 | CG2 | THR | 495 | 35.814 | 4.405 | 54.359 | 1.00 | 12.00 | BTKB |
| ATOM | 2876 | C | THR | 495 | 38.083 | 6.320 | 54.372 | 1.00 | 19.04 | BTKB |
| ATOM | 2877 | O | THR | 495 | 37.209 | 7.182 | 54.299 | 1.00 | 19.97 | BTKB |
| ATOM | 2878 | N | GLN | 496 | 39.112 | 6.393 | 55.207 | 1.00 | 20.71 | BTKB |
| ATOM | 2879 | CA | GLN | 496 | 39.285 | 7.488 | 56.156 | 1.00 | 20.37 | BTKB |
| ATOM | 2880 | CB | GLN | 496 | 40.605 | 7.320 | 56.930 | 1.00 | 24.38 | BTKB |
| ATOM | 2881 | CG | GLN | 496 | 40.686 | 6.083 | 57.852 | 1.00 | 31.81 | BTKB |
| ATOM | 2882 | CD | GLN | 496 | 40.842 | 4.747 | 57.107 | 1.00 | 35.85 | BTKB |
| ATOM | 2883 | OE1 | GLN | 496 | 41.121 | 4.714 | 55.909 | 1.00 | 33.78 | BTKB |
| ATOM | 2884 | NE2 | GLN | 496 | 40.660 | 3.643 | 57.825 | 1.00 | 34.70 | BTKB |
| ATOM | 2885 | C | GLN | 496 | 39.282 | 8.825 | 55.419 | 1.00 | 18.15 | BTKB |
| ATOM | 2886 | O | GLN | 496 | 38.519 | 9.725 | 55.748 | 1.00 | 18.72 | BTKB |
| ATOM | 2887 | N | GLN | 497 | 40.114 | 8.935 | 54.393 | 1.00 | 18.73 | BTKB |
| ATOM | 2888 | CA | GLN | 497 | 40.205 | 10.162 | 53.615 | 1.00 | 14.92 | BTKB |
| ATOM | 2889 | CB | GLN | 497 | 41.534 | 10.196 | 52.862 | 1.00 | 21.68 | BTKB |
| ATOM | 2890 | CG | CLN | 497 | 41.752 | 11.477 | 52.090 | 1.00 | 25.81 | BTKB |
| ATOM | 2891 | CD | GLN | 497 | 41.416 | 12.694 | 52.912 | 1.00 | 25.45 | BTKB |
| ATOM | 2892 | OE1 | GLN | 497 | 41.984 | 12.911 | 53.985 | 1.00 | 30.13 | BTKB |
| ATOM | 2893 | NE2 | GLN | 497 | 40.462 | 13.480 | 52.434 | 1.00 | 22.53 | BTKB |
| ATOM | 2894 | C | GLN | 497 | 39.031 | 10.315 | 52.647 | 1.00 | 11.54 | BTKB |
| ATOM | 2895 | O | GLN | 497 | 38.656 | 11.425 | 52.272 | 1.00 | 10.10 | BTKB |
| ATOM | 2896 | N | LEU | 498 | 38.463 | 9.189 | 52.240 | 1.00 | 9.12 | BTKB |
| ATOM | 2897 | CA | LEU | 498 | 37.327 | 9.173 | 51.336 | 1.00 | 8.57 | BTKB |
| ATOM | 2898 | CB | LEU | 498 | 36.961 | 7.721 | 51.021 | 1.00 | 11.37 | BTKB |
| ATOM | 2899 | CG | LEU | 498 | 36.951 | 7.223 | 49.575 | 1.00 | 9.98 | BTKB |
| ATOM | 2900 | CD1 | LEU | 498 | 38.079 | 7.838 | 48.767 | 1.00 | 8.19 | BTKB |
| ATOM | 2901 | CD2 | LEU | 498 | 37.050 | 5.711 | 49.570 | 1.00 | 7.91 | BTKB |
| ATOM | 2902 | C | LEU | 498 | 36.167 | 9.868 | 52.043 | 1.00 | 10.48 | BTKB |
| ATOM | 2903 | O | LEU | 498 | 35.469 | 10.702 | 51.458 | 1.00 | 11.51 | BTKB |
| ATOM | 2904 | N | LEU | 499 | 36.016 | 9.569 | 53.331 | 1.00 | 12.46 | BTKB |
| ATOM | 2905 | CA | LEU | 499 | 34.949 | 10.146 | 54.133 | 1.00 | 10.83 | BTKB |
| ATOM | 2906 | CB | LEU | 499 | 34.815 | 9.375 | 55.454 | 1.00 | 12.04 | BTKB |
| ATOM | 2907 | CG | LEU | 499 | 33.474 | 9.303 | 56.205 | 1.00 | 8.53 | BTKB |
| ATOM | 2908 | CD1 | LEU | 499 | 33.416 | 10.349 | 57.280 | 1.00 | 8.11 | BTKB |
| ATOM | 2909 | CD2 | LEU | 499 | 32.286 | 9.415 | 55.263 | 1.00 | 5.87 | BTKB |
| ATOM | 2910 | C | LEU | 499 | 35.229 | 11.622 | 54.365 | 1.00 | 7.37 | BTKB |
| ATOM | 2911 | O | LEU | 499 | 34.327 | 12.449 | 54.275 | 1.00 | 10.84 | BTKB |
| ATOM | 2912 | N | GLU | 500 | 36.491 | 11.960 | 54.607 | 1.00 | 8.33 | BTKB |
| ATOM | 2913 | CA | GLU | 500 | 36.878 | 13.353 | 54.821 | 1.00 | 10.19 | BTKB |
| ATOM | 2914 | CB | GLU | 500 | 38.407 | 13.498 | 54.933 | 1.00 | 11.71 | BTKB |
| ATOM | 2915 | CG | GLU | 500 | 38.988 | 13.251 | 56.326 | 1.00 | 15.65 | BTKB |
| ATOM | 2916 | CD | GLU | 500 | 38.574 | 14.307 | 57.342 | 1.00 | 16.85 | BTKB |
| ATOM | 2917 | OE1 | GLU | 500 | 38.534 | 13.992 | 58.550 | 1.00 | 17.26 | BTKB |
| ATOM | 2918 | OE2 | GLU | 500 | 38.290 | 15.454 | 56.940 | 1.00 | 21.13 | BTKB |
| ATOM | 2919 | C | GLU | 500 | 36.377 | 14.220 | 53.679 | 1.00 | 10.00 | BTKB |
| ATOM | 2920 | O | GLU | 500 | 35.914 | 15.328 | 53.898 | 1.00 | 12.10 | BTKB |
| ATOM | 2921 | N | MET | 501 | 36.453 | 13.701 | 52.459 | 1.00 | 12.06 | BTKB |
| ATOM | 2922 | CA | MET | 501 | 36.009 | 14.434 | 51.284 | 1.00 | 11.31 | BTKB |
| ATOM | 2923 | CB | MET | 501 | 36.314 | 13.637 | 50.015 | 1.00 | 10.59 | BTKB |
| ATOM | 2924 | CG | MET | 501 | 37.792 | 13.351 | 49.803 | 1.00 | 11.63 | BTKB |
| ATOM | 2925 | SD | MET | 501 | 38.095 | 12.353 | 48.335 | 1.00 | 16.64 | BTKB |
| ATOM | 2926 | CE | MET | 501 | 39.512 | 13.180 | 47.631 | 1.00 | 19.96 | BTKB |
| ATOM | 2927 | C | MET | 501 | 34.522 | 14.720 | 51.400 | 1.00 | 11.74 | BTKB |
| ATOM | 2928 | O | MET | 501 | 34.098 | 15.865 | 51.257 | 1.00 | 12.06 | BTKB |
| ATOM | 2929 | N | CYS | 502 | 33.740 | 13.685 | 51.707 | 1.00 | 11.26 | BTKB |
| ATOM | 2930 | CA | CYS | 502 | 32.290 | 13.818 | 51.879 | 1.00 | 10.20 | BTKB |
| ATOM | 2931 | CB | CYS | 502 | 31.673 | 12.473 | 52.290 | 1.00 | 12.20 | BTKB |
| ATOM | 2932 | SG | CYS | 502 | 31.964 | 11.065 | 51.174 | 1.00 | 14.20 | BTKB |
| ATOM | 2933 | C | CYS | 502 | 31.986 | 14.848 | 52.976 | 1.00 | 9.69 | BTKB |
| ATOM | 2934 | O | CYS | 502 | 30.976 | 15.549 | 52.923 | 1.00 | 7.12 | BTKB |
| ATOM | 2935 | N | LYS | 503 | 32.872 | 14.924 | 53.967 | 1.00 | 11.41 | BTKB |
| ATOM | 2936 | CA | LYS | 503 | 32.735 | 15.838 | 55.095 | 1.00 | 7.79 | BTKB |
| ATOM | 2937 | CB | LYS | 503 | 33.700 | 15.431 | 56.217 | 1.00 | 4.87 | BTKB |
| ATOM | 2938 | CG | LYS | 503 | 33.559 | 16.217 | 57.519 | 1.00 | 7.28 | BTKB |
| ATOM | 2939 | CD | LYS | 503 | 34.929 | 16.669 | 58.024 | 1.00 | 12.59 | BTKB |
| ATOM | 2940 | CE | LYS | 503 | 34.879 | 17.121 | 59.483 | 1.00 | 12.64 | BTKB |
| ATOM | 2941 | NZ | LYS | 503 | 36.120 | 17.842 | 59.917 | 1.00 | 16.35 | BTKB |
| ATOM | 2942 | C | LYS | 503 | 32.993 | 17.280 | 54.656 | 1.00 | 7.32 | BTKB |
| ATOM | 2943 | O | LYS | 503 | 32.235 | 18.185 | 54.997 | 1.00 | 9.67 | BTKB |
| ATOM | 2944 | N | ASP | 504 | 34.051 | 17.484 | 53.883 | 1.00 | 4.47 | BTKB |
| ATOM | 2945 | CA | ASP | 504 | 34.401 | 18.801 | 53.387 | 1.00 | 2.00 | BTKB |
| ATOM | 2946 | CB | ASP | 504 | 35.649 | 18.712 | 52.505 | 1.00 | 5.96 | BTKB |
| ATOM | 2947 | CG | ASP | 504 | 36.876 | 18.212 | 53.253 | 1.00 | 5.96 | BTKB |
| ATOM | 2948 | OD1 | ASP | 504 | 36.861 | 18.172 | 54.500 | 1.00 | 6.58 | BTKB |
| ATOM | 2949 | OD2 | ASP | 504 | 37.863 | 17.856 | 52.580 | 1.00 | 11.77 | BTKB |
| ATOM | 2950 | C | ASP | 504 | 33.244 | 19.350 | 52.566 | 1.00 | 6.17 | BTKB |
| ATOM | 2951 | O | ASP | 504 | 32.857 | 20.512 | 52.723 | 1.00 | 4.62 | BTKB |
| ATOM | 2952 | N | VAL | 505 | 32.680 | 18.499 | 51.705 | 1.00 | 8.12 | BTKB |
| ATOM | 2953 | CA | VAL | 505 | 31.560 | 18.880 | 50.841 | 1.00 | 5.94 | BTKB |
| ATOM | 2954 | CB | VAL | 505 | 31.241 | 17.800 | 49.763 | 1.00 | 2.00 | BTKB |
| ATOM | 2955 | CG1 | VAL | 505 | 30.116 | 18.283 | 48.867 | 1.00 | 3.28 | BTKB |
| ATOM | 2956 | CG2 | VAL | 505 | 32.466 | 17.495 | 48.918 | 1.00 | 5.68 | BTKB |
| ATOM | 2957 | C | VAL | 505 | 30.294 | 19.109 | 51.652 | 1.00 | 7.08 | BTKB |
| ATOM | 2958 | O | VAL | 505 | 29.560 | 20.069 | 51.415 | 1.00 | 9.90 | BTKB |
| ATOM | 2959 | N | CYS | 506 | 30.050 | 18.235 | 52.622 | 1.00 | 8.09 | BTKB |
| ATOM | 2960 | CA | CYS | 506 | 28.861 | 18.336 | 53.444 | 1.00 | 8.75 | BTKB |
| ATOM | 2961 | CB | CYS | 506 | 28.728 | 17.112 | 54.355 | 1.00 | 7.47 | BTKB |
| ATOM | 2962 | SG | CYS | 506 | 27.033 | 16.791 | 54.882 | 1.00 | 8.97 | BTKB |
| ATOM | 2963 | C | CYS | 506 | 28.856 | 19.621 | 54.257 | 1.00 | 8.23 | BTKB |
| ATOM | 2964 | O | CYS | 506 | 27.822 | 20.272 | 54.378 | 1.00 | 10.16 | BTKB |
| ATOM | 2965 | N | GLU | 507 | 30.010 | 20.000 | 54.796 | 1.00 | 8.06 | BTKB |
| ATOM | 2966 | CA | GLU | 507 | 30.112 | 21.226 | 55.588 | 1.00 | 8.72 | BTKB |
| ATOM | 2967 | CB | GLU | 507 | 31.532 | 21.409 | 56.118 | 1.00 | 5.94 | BTKB |
| ATOM | 2968 | CG | GLU | 507 | 31.986 | 20.276 | 57.018 | 1.00 | 8.44 | BTKB |
| ATOM | 2969 | CD | GLU | 507 | 33.360 | 20.479 | 57.612 | 1.00 | 5.09 | BTKB |
| ATOM | 2970 | OE1 | GLU | 507 | 34.090 | 21.392 | 57.197 | 1.00 | 12.55 | BTKB |
| ATOM | 2971 | OE2 | GLU | 507 | 33.714 | 19.708 | 58.514 | 1.00 | 12.74 | BTKB |
| ATOM | 2972 | C | GLU | 507 | 29.727 | 22.422 | 54.730 | 1.00 | 9.87 | BTKB |
| ATOM | 2973 | O | GLU | 507 | 28.948 | 23.286 | 55.152 | 1.00 | 14.35 | BTKB |
| ATOM | 2974 | N | ALA | 508 | 30.272 | 22.460 | 53.518 | 1.00 | 10.59 | BTKB |
| ATOM | 2975 | CA | ALA | 508 | 30.001 | 23.530 | 52.568 | 1.00 | 10.69 | BTKB |
| ATOM | 2976 | CB | ALA | 508 | 30.859 | 23.337 | 51.343 | 1.00 | 11.50 | BTKB |
| ATOM | 2977 | C | ALA | 508 | 28.521 | 23.542 | 52.183 | 1.00 | 10.30 | BTKB |
| ATOM | 2978 | O | ALA | 508 | 27.885 | 24.597 | 52.110 | 1.00 | 7.78 | BTKB |
| ATOM | 2979 | N | MET | 509 | 27.983 | 22.355 | 51.929 | 1.00 | 11.26 | BTKB |
| ATOM | 2980 | CA | MET | 509 | 26.582 | 22.212 | 51.556 | 1.00 | 13.26 | BTKB |
| ATOM | 2981 | CB | MET | 509 | 26.301 | 20.796 | 51.053 | 1.00 | 12.49 | BTKB |
| ATOM | 2982 | CG | MET | 509 | 26.833 | 20.504 | 49.671 | 1.00 | 9.20 | BTKB |
| ATOM | 2983 | SD | MET | 509 | 26.595 | 21.913 | 48.607 | 1.00 | 9.16 | BTKB |
| ATOM | 2984 | CE | MET | 509 | 24.812 | 21.976 | 48.456 | 1.00 | 5.81 | BTKB |
| ATOM | 2985 | C | MET | 509 | 25.679 | 22.514 | 52.738 | 1.00 | 17.44 | BTKB |
| ATOM | 2986 | O | MET | 509 | 24.539 | 22.961 | 52.570 | 1.00 | 19.95 | BTKB |
| ATOM | 2987 | N | GLU | 510 | 26.198 | 22.251 | 53.932 | 1.00 | 18.37 | BTKB |
| ATOM | 2988 | CA | GLU | 510 | 25.467 | 22.479 | 55.159 | 1.00 | 17.75 | BTKB |
| ATOM | 2989 | CB | GLU | 510 | 26.217 | 21.858 | 56.336 | 1.00 | 21.22 | BTKB |
| ATOM | 2990 | CG | GLU | 510 | 25.370 | 21.629 | 57.572 | 1.00 | 19.10 | BTKB |
| ATOM | 2991 | CD | GLU | 510 | 25.692 | 22.587 | 58.687 | 1.00 | 21.96 | BTKB |
| ATOM | 2992 | OE1 | GLU | 510 | 26.774 | 22.452 | 59.296 | 1.00 | 20.26 | BTKB |
| ATOM | 2993 | OE2 | GLU | 510 | 24.859 | 23.472 | 58.958 | 1.00 | 24.99 | BTKB |
| ATOM | 2994 | C | GLU | 510 | 25.293 | 23.981 | 55.335 | 1.00 | 19.15 | BTKB |
| ATOM | 2995 | O | GLU | 510 | 24.178 | 24.446 | 55.579 | 1.00 | 21.52 | BTKB |
| ATOM | 2996 | N | TYR | 511 | 26.368 | 24.751 | 55.169 | 1.00 | 19.18 | BTKB |
| ATOM | 2997 | CA | TYR | 511 | 26.251 | 26.204 | 55.299 | 1.00 | 22.64 | BTKB |
| ATOM | 2998 | CB | TYR | 511 | 27.612 | 26.885 | 55.456 | 1.00 | 28.60 | BTKB |
| ATOM | 2999 | CG | TYR | 511 | 27.764 | 27.549 | 56.815 | 1.00 | 38.15 | BTKB |
| ATOM | 3000 | CD1 | TYR | 511 | 27.179 | 26.988 | 57.958 | 1.00 | 38.05 | BTKB |
| ATOM | 3001 | CE1 | TYR | 511 | 27.291 | 27.597 | 59.199 | 1.00 | 39.84 | BTKB |
| ATOM | 3002 | CD2 | TYR | 511 | 28.469 | 28.745 | 56.958 | 1.00 | 37.94 | BTKB |
| ATOM | 3003 | CE2 | TYR | 511 | 28.589 | 29.365 | 58.206 | 1.00 | 38.73 | BTKB |
| ATOM | 3004 | CZ | TYR | 511 | 27.995 | 28.786 | 59.319 | 1.00 | 40.24 | BTKB |
| ATOM | 3005 | OH | TYR | 511 | 28.100 | 29.397 | 60.550 | 1.00 | 37.79 | BTKB |
| ATOM | 3006 | C | TYR | 511 | 25.441 | 26.838 | 54.168 | 1.00 | 20.77 | BTKB |
| ATOM | 3007 | O | TYR | 511 | 24.824 | 27.881 | 54.353 | 1.00 | 19.57 | BTKB |
| ATOM | 3008 | N | LEU | 512 | 25.460 | 26.223 | 52.991 | 1.00 | 21.25 | BTKB |
| ATOM | 3009 | CA | LEU | 512 | 24.662 | 26.734 | 51.885 | 1.00 | 18.82 | BTKB |
| ATOM | 3010 | CB | LEU | 512 | 24.985 | 26.022 | 50.569 | 1.00 | 17.08 | BTKB |
| ATOM | 3011 | CG | LEU | 512 | 26.256 | 26.418 | 49.811 | 1.00 | 16.90 | BTKB |
| ATOM | 3012 | CD1 | LEU | 512 | 26.165 | 25.864 | 48.375 | 1.00 | 10.52 | BTKB |
| ATOM | 3013 | CD2 | LEU | 512 | 26.414 | 27.937 | 49.783 | 1.00 | 10.86 | BTKB |
| ATOM | 3014 | C | LEU | 512 | 23.217 | 26.473 | 52.271 | 1.00 | 19.11 | BTKB |
| ATOM | 3015 | O | LEU | 512 | 22.354 | 27.328 | 52.097 | 1.00 | 23.50 | BTKB |
| ATOM | 3016 | N | GLU | 513 | 22.972 | 25.306 | 52.851 | 1.00 | 19.26 | BTKB |
| ATOM | 3017 | CA | GLU | 513 | 21.640 | 24.930 | 53.283 | 1.00 | 18.74 | BTKB |
| ATOM | 3018 | CE | GLU | 513 | 21.665 | 23.554 | 53.944 | 1.00 | 16.46 | BTKB |
| ATOM | 3019 | CG | GLU | 513 | 20.296 | 23.079 | 54.393 | 1.00 | 18.68 | BTKB |
| ATOM | 3020 | CD | GLU | 513 | 20.327 | 21.695 | 55.003 | 1.00 | 17.60 | BTKB |
| ATOM | 3021 | OE1 | GLU | 513 | 19.879 | 20.746 | 54.322 | 1.00 | 17.80 | BTKB |
| ATOM | 3022 | OE2 | GLU | 513 | 20.792 | 21.561 | 56.159 | 1.00 | 12.10 | BTKB |
| ATOM | 3023 | C | GLU | 513 | 21.123 | 25.962 | 54.269 | 1.00 | 22.83 | BTKB |
| ATOM | 3024 | O | GLU | 513 | 20.033 | 26.514 | 54.079 | 1.00 | 26.96 | BTKB |
| ATOM | 3025 | N | SER | 514 | 21.924 | 26.241 | 55.300 | 1.00 | 24.79 | BTKB |
| ATOM | 3026 | CA | SER | 514 | 21.573 | 27.212 | 56.338 | 1.00 | 24.05 | BTKB |
| ATOM | 3027 | CB | SER | 514 | 22.693 | 27.314 | 57.376 | 1.00 | 23.79 | BTKB |
| ATOM | 3028 | OG | SER | 514 | 23.886 | 27.829 | 56.799 | 1.00 | 22.03 | BTKB |
| ATOM | 3029 | C | SER | 514 | 21.325 | 28.589 | 55.733 | 1.00 | 22.16 | BTKB |
| ATOM | 3030 | O | SER | 514 | 20.357 | 29.263 | 56.080 | 1.00 | 21.49 | BTKB |
| ATOM | 3031 | N | LYS | 515 | 22.195 | 28.974 | 54.805 | 1.00 | 21.78 | BTKB |
| ATOM | 3032 | CA | LYS | 515 | 22.128 | 30.263 | 54.122 | 1.00 | 19.84 | BTKB |
| ATOM | 3033 | CB | LYS | 515 | 23.522 | 30.574 | 53.541 | 1.00 | 15.28 | BTKB |
| ATOM | 3034 | CG | LYS | 515 | 23.593 | 31.460 | 52.308 | 1.00 | 22.32 | BTKB |
| ATOM | 3035 | CD | LYS | 515 | 23.373 | 32.930 | 52.616 | 1.00 | 20.90 | BTKB |
| ATOM | 3036 | CE | LYS | 515 | 23.192 | 33.703 | 51.322 | 1.00 | 15.11 | BTKB |
| ATOM | 3037 | NZ | LYS | 515 | 22.035 | 33.175 | 50.535 | 1.00 | 9.55 | BTKB |
| ATOM | 3038 | C | LYS | 515 | 21.008 | 30.302 | 53.072 | 1.00 | 20.47 | BTKB |
| ATOM | 3039 | O | LYS | 515 | 20.769 | 31.325 | 52.433 | 1.00 | 22.75 | BTKB |
| ATOM | 3040 | N | GLN | 516 | 20.267 | 29.206 | 52.956 | 1.00 | 19.94 | BTKB |
| ATOM | 3041 | CA | GLN | 516 | 19.165 | 29.111 | 52.000 | 1.00 | 19.73 | BTKB |
| ATOM | 3042 | CB | GLN | 516 | 18.085 | 30.162 | 52.314 | 1.00 | 23.43 | BTKB |
| ATOM | 3043 | CG | GLN | 516 | 17.672 | 30.281 | 53.783 | 1.00 | 22.95 | BTKB |
| ATOM | 3044 | CD | GLN | 516 | 16.919 | 29.073 | 54.296 | 1.00 | 30.24 | BTKB |
| ATOM | 3045 | OE1 | GLN | 516 | 15.719 | 28.929 | 54.063 | 1.00 | 32.30 | BTKB |
| ATOM | 3046 | NE2 | GLN | 516 | 17.612 | 28.212 | 55.026 | 1.00 | 32.64 | BTKB |
| ATOM | 3047 | C | GLN | 516 | 19.658 | 29.287 | 50.554 | 1.00 | 17.78 | BTKB |
| ATOM | 3048 | O | GLN | 516 | 19.000 | 29.935 | 49.733 | 1.00 | 15.79 | BTKB |
| ATOM | 3049 | N | PHE | 517 | 20.788 | 28.669 | 50.231 | 1.00 | 13.90 | BTKB |
| ATOM | 3050 | CA | PHE | 517 | 21.353 | 28.773 | 48.891 | 1.00 | 16.72 | BTKB |
| ATOM | 3051 | CB | PHE | 517 | 22.719 | 29.453 | 48.963 | 1.00 | 13.45 | BTKB |
| ATOM | 3052 | CG | PHE | 517 | 23.245 | 29.919 | 47.628 | 1.00 | 13.82 | BTKB |
| ATOM | 3053 | CD1 | PHE | 517 | 23.067 | 31.246 | 47.220 | 1.00 | 11.73 | BTKB |
| ATOM | 3054 | CD2 | PHE | 517 | 23.957 | 29.049 | 46.800 | 1.00 | 12.17 | BTKB |
| ATOM | 3055 | CE1 | PHE | 517 | 23.596 | 31.699 | 46.002 | 1.00 | 11.02 | BTKB |
| ATOM | 3056 | CE2 | PHE | 517 | 24.491 | 29.486 | 45.584 | 1.00 | 9.71 | BTKB |
| ATOM | 3057 | CZ | PHE | 517 | 24.312 | 30.815 | 45.183 | 1.00 | 13.73 | BTKB |
| ATOM | 3058 | C | PHE | 517 | 21.477 | 27.412 | 48.197 | 1.00 | 17.18 | BTKB |
| ATOM | 3059 | O | PHE | 517 | 22.222 | 26.540 | 48.658 | 1.00 | 20.28 | BTKB |
| ATOM | 3060 | N | LEU | 518 | 20.749 | 27.242 | 47.093 | 1.00 | 15.31 | BTKB |
| ATOM | 3061 | CA | LEU | 518 | 20.766 | 26.003 | 46.312 | 1.00 | 13.37 | BTKB |
| ATOM | 3062 | CB | LEU | 518 | 19.474 | 25.864 | 45.508 | 1.00 | 10.07 | BTKB |
| ATOM | 3063 | CG | LEU | 518 | 18.227 | 25.283 | 46.168 | 1.00 | 12.29 | BTKB |
| ATOM | 3064 | CD1 | LEU | 518 | 17.018 | 25.488 | 45.274 | 1.00 | 9.13 | BTKB |
| ATOM | 3065 | CD2 | LEU | 518 | 18.455 | 23.817 | 46.434 | 1.00 | 14.04 | BTKB |
| ATOM | 3066 | C | LEU | 518 | 21.936 | 25.979 | 45.334 | 1.00 | 13.97 | BTKB |
| ATOM | 3067 | O | LEU | 518 | 22.236 | 26.979 | 44.692 | 1.00 | 19.55 | BTKB |
| ATOM | 3068 | N | HIS | 519 | 22.557 | 24.820 | 45.165 | 1.00 | 13.91 | BTKB |
| ATOM | 3069 | CA | HIS | 519 | 23.684 | 24.700 | 44.250 | 1.00 | 15.81 | BTKB |
| ATOM | 3070 | CB | HIS | 519 | 24.659 | 23.631 | 44.743 | 1.00 | 12.29 | BTKB |
| ATOM | 3071 | CG | HIS | 519 | 26.008 | 23.706 | 44.101 | 1.00 | 9.16 | BTKB |
| ATOM | 3072 | CD2 | HIS | 519 | 27.239 | 23.881 | 44.637 | 1.00 | 9.86 | BTKB |
| ATOM | 3073 | ND1 | HIS | 519 | 26.198 | 23.578 | 42.741 | 1.00 | 7.69 | BTKB |
| ATOM | 3074 | CE1 | HIS | 519 | 27.489 | 23.664 | 42.468 | 1.00 | 8.06 | BTKB |
| ATOM | 3075 | NE2 | HIS | 519 | 28.142 | 23.850 | 43.602 | 1.00 | 9.01 | BTKB |
| ATOM | 3076 | C | HIS | 519 | 23.256 | 24.396 | 42.811 | 1.00 | 18.77 | BTKB |
| ATOM | 3077 | O | HIS | 519 | 23.987 | 24.714 | 41.877 | 1.00 | 19.63 | BTKB |
| ATOM | 3078 | N | ARG | 520 | 22.116 | 23.724 | 42.645 | 1.00 | 21.67 | BTKB |
| ATOM | 3079 | CA | ARG | 520 | 21.572 | 23.372 | 41.329 | 1.00 | 21.63 | BTKB |
| ATOM | 3080 | CB | ARG | 520 | 21.501 | 24.620 | 40.438 | 1.00 | 21.69 | BTKB |
| ATOM | 3081 | CG | ARG | 520 | 20.865 | 24.389 | 39.081 | 1.00 | 24.47 | BTKB |
| ATOM | 3082 | CD | ARG | 520 | 21.521 | 25.238 | 38.001 | 1.00 | 26.51 | BTKB |
| ATOM | 3083 | NE | ARG | 520 | 21.186 | 26.658 | 38.074 | 1.00 | 33.49 | BTKB |
| ATOM | 3084 | CZ | ARG | 520 | 20.034 | 27.185 | 37.667 | 1.00 | 32.52 | BTKB |
| ATOM | 3085 | NH1 | ARG | 520 | 19.078 | 26.412 | 37.169 | 1.00 | 35.39 | BTKB |
| ATOM | 3086 | NH2 | ARG | 520 | 19.870 | 28.502 | 37.675 | 1.00 | 36.18 | BTKB |
| ATOM | 3087 | C | ARG | 520 | 22.295 | 22.232 | 40.582 | 1.00 | 23.98 | BTKB |
| ATOM | 3088 | O | ARG | 520 | 21.646 | 21.366 | 39.987 | 1.00 | 23.54 | BTKB |
| ATOM | 3089 | N | ASP | 521 | 23.625 | 22.221 | 40.629 | 1.00 | 21.56 | BTKB |
| ATOM | 3090 | CA | ASP | 521 | 24.423 | 21.210 | 39.935 | 1.00 | 19.42 | BTKB |
| ATOM | 3091 | CB | ASP | 521 | 25.033 | 21.861 | 38.676 | 1.00 | 20.57 | BTKB |
| ATOM | 3092 | CG | ASP | 521 | 25.309 | 20.867 | 37.552 | 1.00 | 18.16 | BTKB |
| ATOM | 3093 | OD1 | ASP | 521 | 26.470 | 20.777 | 37.099 | 1.00 | 19.83 | BTKB |
| ATOM | 3094 | OD2 | ASP | 521 | 24.359 | 20.205 | 37.092 | 1.00 | 21.97 | BTKB |
| ATOM | 3095 | C | ASP | 521 | 25.533 | 20.709 | 40.872 | 1.00 | 17.08 | BTKB |
| ATOM | 3096 | O | ASP | 521 | 26.655 | 21.199 | 40.830 | 1.00 | 19.34 | BTKB |
| ATOM | 3097 | N | LEU | 522 | 25.206 | 19.781 | 41.763 | 1.00 | 14.72 | BTKB |
| ATOM | 3098 | CA | LEU | 522 | 26.197 | 19.252 | 42.704 | 1.00 | 12.04 | BTKB |
| ATOM | 3099 | CB | LEU | 522 | 25.612 | 19.164 | 44.117 | 1.00 | 15.80 | BTKB |
| ATOM | 3100 | CG | LEU | 522 | 26.527 | 18.661 | 45.235 | 1.00 | 15.39 | BTKB |
| ATOM | 3101 | CD1 | LEU | 522 | 27.670 | 19.639 | 45.486 | 1.00 | 14.85 | BTKB |
| ATOM | 3102 | CD2 | LEU | 522 | 25.709 | 18.455 | 46.490 | 1.00 | 14.89 | BTKB |
| ATOM | 3103 | C | LEU | 522 | 26.724 | 17.892 | 42.249 | 1.00 | 11.47 | BTKB |
| ATOM | 3104 | O | LEU | 522 | 25.991 | 16.902 | 42.200 | 1.00 | 8.58 | BTKB |
| ATOM | 3105 | N | ALA | 523 | 28.015 | 17.851 | 41.949 | 1.00 | 9.69 | BTKB |
| ATOM | 3106 | CA | ALA | 523 | 28.650 | 16.637 | 41.467 | 1.00 | 7.49 | BTKB |
| ATOM | 3107 | CB | ALA | 523 | 28.332 | 16.457 | 39.968 | 1.00 | 8.23 | BTKB |
| ATOM | 3108 | C | ALA | 523 | 30.150 | 16.780 | 41.677 | 1.00 | 3.64 | BTKB |
| ATOM | 3109 | O | ALA | 523 | 30.643 | 17.892 | 41.858 | 1.00 | 5.50 | BTKB |
| ATOM | 3110 | N | ALA | 524 | 30.880 | 15.671 | 41.609 | 1.00 | 2.00 | BTKB |
| ATOM | 3111 | CA | ALA | 524 | 32.328 | 15.695 | 41.804 | 1.00 | 3.96 | BTKB |
| ATOM | 3112 | CB | ALA | 524 | 32.890 | 14.289 | 41.820 | 1.00 | 2.49 | BTKB |
| ATOM | 3113 | C | ALA | 524 | 33.040 | 16.533 | 40.753 | 1.00 | 3.94 | BTKB |
| ATOM | 3114 | O | ALA | 524 | 34.159 | 16.994 | 40.974 | 1.00 | 2.00 | BTKB |
| ATOM | 3115 | N | ARG | 525 | 32.409 | 16.696 | 39.596 | 1.00 | 6.91 | BTKB |
| ATOM | 3116 | CA | ARG | 525 | 32.979 | 17.497 | 38.515 | 1.00 | 11.27 | BTKB |
| ATOM | 3117 | CB | ARG | 525 | 32.152 | 17.328 | 37.246 | 1.00 | 13.87 | BTKB |
| ATOM | 3118 | CG | ARG | 525 | 30.741 | 17.870 | 37.362 | 1.00 | 18.97 | BTKB |
| ATOM | 3119 | CD | ARG | 525 | 30.214 | 18.263 | 35.997 | 1.00 | 23.23 | BTKB |
| ATOM | 3120 | NE | ARG | 525 | 31.138 | 19.182 | 35.332 | 1.00 | 29.34 | BTKB |
| ATOM | 3121 | CZ | ARG | 525 | 30.855 | 20.439 | 35.010 | 1.00 | 30.92 | BTKB |
| ATOM | 3122 | NH1 | ARG | 525 | 29.662 | 20.956 | 35.283 | 1.00 | 31.96 | BTKB |
| ATOM | 3123 | NH2 | ARG | 525 | 31.773 | 21.183 | 34.412 | 1.00 | 28.24 | BTKB |
| ATOM | 3124 | C | ARG | 525 | 33.002 | 18.983 | 38.891 | 1.00 | 11.79 | BTKB |
| ATOM | 3125 | O | ARG | 525 | 33.802 | 19.760 | 38.376 | 1.00 | 11.27 | BTKB |
| ATOM | 3126 | N | ASN | 526 | 32.099 | 19.364 | 39.785 | 1.00 | 11.62 | BTKB |
| ATOM | 3127 | CA | ASN | 526 | 31.982 | 20.734 | 40.250 | 1.00 | 8.74 | BTKB |
| ATOM | 3128 | CB | ASN | 526 | 30.504 | 21.093 | 40.372 | 1.00 | 6.93 | BTKB |
| ATOM | 3129 | CG | ASN | 526 | 29.781 | 21.003 | 39.047 | 1.00 | 8.76 | BTKB |
| ATOM | 3130 | OD1 | ASN | 526 | 28.796 | 20.275 | 38.909 | 1.00 | 13.72 | BTKB |
| ATOM | 3131 | ND2 | ASN | 526 | 30.283 | 21.725 | 38.051 | 1.00 | 11.51 | BTKB |
| ATOM | 3132 | C | ASN | 526 | 32.698 | 20.956 | 41.585 | 1.00 | 5.24 | BTKB |
| ATOM | 3133 | O | ASN | 526 | 32.471 | 21.956 | 42.255 | 1.00 | 4.05 | BTKB |
| ATOM | 3134 | N | CYS | 527 | 33.549 | 20.013 | 41.977 | 1.00 | 7.78 | BTKB |
| ATOM | 3135 | CA | CYS | 527 | 34.302 | 20.128 | 43.218 | 1.00 | 9.39 | BTKB |
| ATOM | 3136 | CB | CYS | 527 | 34.046 | 18.934 | 44.147 | 1.00 | 8.89 | BTKB |
| ATOM | 3137 | SG | CYS | 527 | 32.439 | 18.919 | 44.970 | 1.00 | 9.13 | BTKB |
| ATOM | 3138 | C | CYS | 527 | 35.777 | 20.186 | 42.865 | 1.00 | 6.94 | BTKB |
| ATOM | 3139 | O | CYS | 527 | 36.225 | 19.505 | 41.942 | 1.00 | 9.40 | BTKB |
| ATOM | 3140 | N | LEU | 528 | 36.520 | 21.006 | 43.600 | 1.00 | 10.86 | BTKB |
| ATOM | 3141 | CA | LEU | 528 | 37.953 | 21.176 | 43.376 | 1.00 | 12.73 | BTKB |
| ATOM | 3142 | CB | LEU | 528 | 38.276 | 22.646 | 43.097 | 1.00 | 12.37 | BTKB |
| ATOM | 3143 | CG | LEU | 528 | 37.598 | 23.307 | 41.892 | 1.00 | 12.90 | BTKB |
| ATOM | 3144 | CD1 | LEU | 528 | 37.402 | 24.781 | 42.185 | 1.00 | 19.16 | BTKB |
| ATOM | 3145 | CD2 | LEU | 528 | 38.419 | 23.099 | 40.614 | 1.00 | 17.35 | BTKB |
| ATOM | 3146 | C | LEU | 528 | 38.734 | 20.710 | 44.591 | 1.00 | 12.40 | BTKB |
| ATOM | 3147 | O | LEU | 528 | 38.197 | 20.638 | 45.696 | 1.00 | 14.00 | BTKB |
| ATOM | 3148 | N | VAL | 529 | 40.009 | 20.405 | 44.381 | 1.00 | 13.66 | BTKB |
| ATOM | 3149 | CA | VAL | 529 | 40.884 | 19.939 | 45.446 | 1.00 | 16.38 | BTKB |
| ATOM | 3150 | CB | VAL | 529 | 41.324 | 18.477 | 45.205 | 1.00 | 13.16 | BTKB |
| ATOM | 3151 | CG1 | VAL | 529 | 42.156 | 17.971 | 46.387 | 1.00 | 7.91 | BTKB |
| ATOM | 3152 | CG2 | VAL | 529 | 40.105 | 17.586 | 44.943 | 1.00 | 15.41 | BTKB |
| ATOM | 3153 | C | VAL | 529 | 42.122 | 20.820 | 45.442 | 1.00 | 17.99 | BTKB |
| ATOM | 3154 | O | VAL | 529 | 42.673 | 21.120 | 44.381 | 1.00 | 19.19 | BTKB |
| ATOM | 3155 | N | ASN | 530 | 42.549 | 21.276 | 46.612 | 1.00 | 18.86 | BTKB |
| ATOM | 3156 | CA | ASN | 530 | 43.741 | 22.109 | 46.653 | 1.00 | 18.14 | BTKB |
| ATOM | 3157 | CB | ASN | 530 | 43.551 | 23.328 | 47.570 | 1.00 | 17.83 | BTKB |
| ATOM | 3158 | CG | ASN | 530 | 43.511 | 22.976 | 49.043 | 1.00 | 13.97 | BTKB |
| ATOM | 3159 | OD1 | ASN | 530 | 43.831 | 21.862 | 49.446 | 1.00 | 14.33 | BTKB |
| ATOM | 3160 | ND2 | ASN | 530 | 43.137 | 23.946 | 49.860 | 1.00 | 11.50 | BTKB |
| ATOM | 3161 | C | ASN | 530 | 44.977 | 21.294 | 47.024 | 1.00 | 17.68 | BTKB |
| ATOM | 3162 | O | ASN | 530 | 44.895 | 20.080 | 47.213 | 1.00 | 18.38 | BTKB |
| ATOM | 3163 | N | ASP | 531 | 46.119 | 21.964 | 47.104 | 1.00 | 20.29 | BTKB |
| ATOM | 3164 | CA | ASP | 531 | 47.392 | 21.329 | 47.433 | 1.00 | 24.52 | BTKB |
| ATOM | 3165 | CB | ASP | 531 | 48.498 | 22.388 | 47.520 | 1.00 | 26.76 | BTKB |
| ATOM | 3166 | CG | ASP | 531 | 47.956 | 23.787 | 47.753 | 1.00 | 26.59 | BTKB |
| ATOM | 3167 | OD1 | ASP | 531 | 47.289 | 24.012 | 48.788 | 1.00 | 26.97 | BTKB |
| ATOM | 3168 | OD2 | ASP | 531 | 48.180 | 24.652 | 46.879 | 1.00 | 28.82 | BTKB |
| ATOM | 3169 | C | ASP | 531 | 47.412 | 20.465 | 48.693 | 1.00 | 24.27 | BTKB |
| ATOM | 3170 | O | ASP | 531 | 48.269 | 19.596 | 48.838 | 1.00 | 28.06 | BTKB |
| ATOM | 3171 | N | GLN | 532 | 46.471 | 20.699 | 49.597 | 1.00 | 25.23 | BTKB |
| ATOM | 3172 | CA | GLN | 532 | 46.406 | 19.951 | 50.846 | 1.00 | 23.53 | BTKB |
| ATOM | 3173 | CB | GLN | 532 | 45.856 | 20.846 | 51.953 | 1.00 | 25.74 | BTKB |
| ATOM | 3174 | CG | GLN | 532 | 46.669 | 20.828 | 53.233 | 1.00 | 28.15 | BTKB |
| ATOM | 3175 | CD | GLN | 532 | 47.984 | 21.563 | 53.089 | 1.00 | 28.65 | BTKB |
| ATOM | 3176 | OE1 | GLN | 532 | 48.062 | 22.769 | 53.342 | 1.00 | 29.05 | BTKB |
| ATOM | 3177 | NE2 | GLN | 532 | 49.027 | 20.846 | 52.679 | 1.00 | 30.50 | BTKB |
| ATOM | 3178 | C | GLN | 532 | 45.556 | 18.686 | 50.772 | 1.00 | 22.38 | BTKB |
| ATOM | 3179 | O | GLN | 532 | 45.635 | 17.837 | 51.659 | 1.00 | 24.02 | BTKB |
| ATOM | 3180 | N | GLY | 533 | 44.751 | 18.557 | 49.722 | 1.00 | 18.67 | BTKB |
| ATOM | 3181 | CA | GLY | 533 | 43.877 | 17.402 | 49.601 | 1.00 | 13.49 | BTKB |
| ATOM | 3182 | C | GLY | 533 | 42.471 | 17.764 | 50.054 | 1.00 | 12.87 | BTKB |
| ATOM | 3183 | O | GLY | 533 | 41.574 | 16.915 | 50.094 | 1.00 | 13.75 | BTKB |
| ATOM | 3184 | N | VAL | 534 | 42.282 | 19.034 | 50.412 | 1.00 | 10.21 | BTKB |
| ATOM | 3185 | CA | VAL | 534 | 40.987 | 19.536 | 50.852 | 1.00 | 9.08 | BTKB |
| ATOM | 3186 | CB | VAL | 534 | 41.117 | 20.895 | 51.588 | 1.00 | 8.03 | BTKB |
| ATOM | 3187 | CG1 | VAL | 534 | 39.739 | 21.465 | 51.918 | 1.00 | 9.23 | BTKB |
| ATOM | 3188 | CG2 | VAL | 534 | 41.923 | 20.731 | 52.872 | 1.00 | 6.92 | BTKB |
| ATOM | 3189 | C | VAL | 534 | 40.062 | 19.712 | 49.655 | 1.00 | 8.68 | BTKB |
| ATOM | 3190 | O | VAL | 534 | 40.432 | 20.320 | 48.646 | 1.00 | 11.10 | BTKB |
| ATOM | 3191 | N | VAL | 535 | 38.860 | 19.158 | 49.777 | 1.00 | 10.57 | BTKB |
| ATOM | 3192 | CA | VAL | 535 | 37.848 | 19.239 | 48.732 | 1.00 | 9.29 | BTKB |
| ATOM | 3193 | CB | VAL | 535 | 37.058 | 17.918 | 48.622 | 1.00 | 8.60 | BTKB |
| ATOM | 3194 | CG1 | VAL | 535 | 36.015 | 18.001 | 47.522 | 1.00 | 8.21 | BTKB |
| ATOM | 3195 | CG2 | VAL | 535 | 38.002 | 16.776 | 48.351 | 1.00 | 4.73 | BTKB |
| ATOM | 3196 | C | VAL | 535 | 36.900 | 20.385 | 49.071 | 1.00 | 9.08 | BTKB |
| ATOM | 3197 | O | VAL | 535 | 36.415 | 20.493 | 50.200 | 1.00 | 9.32 | BTKB |
| ATOM | 3198 | N | LYS | 536 | 36.709 | 21.281 | 48.109 | 1.00 | 10.56 | BTKB |
| ATOM | 3199 | CA | LYS | 536 | 35.829 | 22.432 | 48.279 | 1.00 | 6.61 | BTKB |
| ATOM | 3200 | CB | LYS | 536 | 36.640 | 23.722 | 48.326 | 1.00 | 5.90 | BTKB |
| ATOM | 3201 | CG | LYS | 536 | 37.657 | 23.823 | 49.437 | 1.00 | 5.31 | BTKB |
| ATOM | 3202 | CD | LYS | 536 | 38.204 | 25.241 | 49.517 | 1.00 | 6.28 | BTKB |
| ATOM | 3203 | CE | LYS | 536 | 39.257 | 25.356 | 50.607 | 1.00 | 7.99 | BTKB |
| ATOM | 3204 | NZ | LYS | 536 | 39.577 | 26.769 | 50.898 | 1.00 | 2.74 | BTKB |
| ATOM | 3205 | C | LYS | 536 | 34.878 | 22.511 | 47.092 | 1.00 | 5.48 | BTKB |
| ATOM | 3206 | O | LYS | 536 | 35.288 | 22.309 | 45.942 | 1.00 | 3.48 | BTKB |
| ATOM | 3207 | N | VAL | 537 | 33.615 | 22.818 | 47.367 | 1.00 | 7.47 | BTKB |
| ATOM | 3208 | CA | VAL | 537 | 32.619 | 22.927 | 46.314 | 1.00 | 8.81 | BTKB |
| ATOM | 3209 | CB | VAL | 537 | 31.188 | 22.635 | 46.835 | 1.00 | 8.72 | BTKB |
| ATOM | 3210 | CG1 | VAL | 537 | 30.952 | 23.324 | 48.142 | 1.00 | 11.64 | BTKB |
| ATOM | 3211 | CG2 | VAL | 537 | 30.137 | 23.054 | 45.813 | 1.00 | 8.15 | BTKB |
| ATOM | 3212 | C | VAL | 537 | 32.700 | 24.273 | 45.616 | 1.00 | 9.66 | BTKB |
| ATOM | 3213 | O | VAL | 537 | 32.947 | 25.305 | 46.243 | 1.00 | 8.25 | BTKB |
| ATOM | 3214 | N | SER | 538 | 32.524 | 24.233 | 44.302 | 1.00 | 15.29 | BTKB |
| ATOM | 3215 | CA | SER | 538 | 32.588 | 25.410 | 43.456 | 1.00 | 20.41 | BTKB |
| ATOM | 3216 | CB | SER | 538 | 33.965 | 25.468 | 42.779 | 1.00 | 25.58 | BTKB |
| ATOM | 3217 | OG | SER | 538 | 34.210 | 26.711 | 42.133 | 1.00 | 28.06 | BTKB |
| ATOM | 3218 | C | SER | 538 | 31.478 | 25.310 | 42.407 | 1.00 | 20.96 | BTKB |
| ATOM | 3219 | O | SER | 538 | 30.516 | 24.555 | 42.573 | 1.00 | 20.92 | BTKB |
| ATOM | 3220 | N | ASP | 539 | 31.613 | 26.075 | 41.330 | 1.00 | 21.84 | BTKB |
| ATOM | 3221 | CA | ASP | 539 | 30.625 | 26.092 | 40.255 | 1.00 | 26.29 | BTKB |
| ATOM | 3222 | CB | ASP | 539 | 30.569 | 24.750 | 39.531 | 1.00 | 29.26 | BTKB |
| ATOM | 3223 | CG | ASP | 539 | 31.510 | 24.691 | 38.352 | 1.00 | 32.89 | BTKB |
| ATOM | 3224 | OD1 | ASP | 539 | 31.014 | 24.523 | 37.217 | 1.00 | 32.96 | BTKB |
| ATOM | 3225 | OD2 | ASP | 539 | 32.738 | 24.820 | 38.556 | 1.00 | 31.54 | BTKB |
| ATOM | 3226 | C | ASP | 539 | 29.236 | 26.497 | 40.719 | 1.00 | 25.45 | BTKB |
| ATOM | 3227 | O | ASP | 539 | 28.232 | 26.073 | 40.142 | 1.00 | 28.05 | BTKB |
| ATOM | 3228 | N | PHE | 540 | 29.187 | 27.276 | 41.793 | 1.00 | 23.51 | BTKB |
| ATOM | 3229 | CA | PHE | 540 | 27.931 | 27.777 | 42.318 | 1.00 | 22.85 | BTKB |
| ATOM | 3230 | CB | PHE | 540 | 27.860 | 27.612 | 43.846 | 1.00 | 17.06 | BTKB |
| ATOM | 3231 | CG | PHE | 540 | 28.988 | 28.269 | 44.586 | 1.00 | 14.42 | BTKB |
| ATOM | 3232 | CD1 | PHE | 540 | 30.168 | 27.581 | 44.822 | 1.00 | 16.97 | BTKB |
| ATOM | 3233 | CD2 | PHE | 540 | 28.878 | 29.583 | 45.030 | 1.00 | 16.54 | BTKB |
| ATOM | 3234 | CE1 | PHE | 540 | 31.227 | 28.190 | 45.487 | 1.00 | 16.68 | BTKB |
| ATOM | 3235 | CE2 | PHE | 540 | 29.928 | 30.197 | 45.694 | 1.00 | 17.48 | BTKB |
| ATOM | 3236 | CZ | PHE | 540 | 31.107 | 29.498 | 45.922 | 1.00 | 15.86 | BTKB |
| ATOM | 3237 | C | PHE | 540 | 27.912 | 29.244 | 41.902 | 1.00 | 24.94 | BTKB |
| ATOM | 3238 | O | PHE | 540 | 28.964 | 29.811 | 41.600 | 1.00 | 26.86 | BTKB |
| ATOM | 3239 | N | GLY | 541 | 26.733 | 29.849 | 41.844 | 1.00 | 25.65 | BTKB |
| ATOM | 3240 | CA | GLY | 541 | 26.643 | 31.243 | 41.437 | 1.00 | 30.15 | BTKB |
| ATOM | 3241 | C | GLY | 541 | 25.289 | 31.471 | 40.809 | 1.00 | 32.87 | BTKB |
| ATOM | 3242 | O | GLY | 541 | 24.421 | 32.123 | 41.387 | 1.00 | 34.13 | BTKB |
| ATOM | 3243 | N | LEU | 542 | 25.108 | 30.932 | 39.608 | 1.00 | 33.81 | BTKB |
| ATOM | 3244 | CA | LEU | 542 | 23.824 | 31.022 | 38.929 | 1.00 | 34.85 | BTKB |
| ATOM | 3245 | CB | LEU | 542 | 23.973 | 30.692 | 37.439 | 1.00 | 33.89 | BTKB |
| ATOM | 3246 | CG | LEU | 542 | 24.372 | 29.277 | 37.014 | 1.00 | 31.57 | BTKB |
| ATOM | 3247 | CD1 | LEU | 542 | 23.136 | 28.530 | 36.552 | 1.00 | 30.77 | BTKB |
| ATOM | 3248 | CD2 | LEU | 542 | 25.400 | 29.340 | 35.884 | 1.00 | 31.85 | BTKB |
| ATOM | 3249 | C | LEU | 542 | 22.935 | 29.998 | 39.648 | 1.00 | 36.33 | BTKB |
| ATOM | 3250 | O | LEU | 542 | 21.762 | 29.819 | 39.318 | 1.00 | 36.07 | BTKB |
| ATOM | 3251 | N | SER | 543 | 23.547 | 29.300 | 40.605 | 1.00 | 36.24 | BTKB |
| ATOM | 3252 | CA | SER | 543 | 22.895 | 28.316 | 41.450 | 1.00 | 34.49 | BTKB |
| ATOM | 3253 | CB | SER | 543 | 23.929 | 27.733 | 42.393 | 1.00 | 31.44 | BTKB |
| ATOM | 3254 | OG | SER | 543 | 25.053 | 27.284 | 41.653 | 1.00 | 26.36 | BTKB |
| ATOM | 3255 | C | SER | 543 | 21.837 | 29.077 | 42.230 | 1.00 | 36.96 | BTKB |
| ATOM | 3256 | O | SER | 543 | 22.137 | 30.058 | 42.908 | 1.00 | 39.90 | BTKB |
| ATOM | 3257 | N | ARG | 544 | 20.601 | 28.614 | 42.128 | 1.00 | 38.02 | BTKB |
| ATOM | 3258 | CA | ARG | 544 | 19.478 | 29.279 | 42.758 | 1.00 | 40.54 | BTKB |
| ATOM | 3259 | CB | ARG | 544 | 18.172 | 28.682 | 42.241 | 1.00 | 38.19 | BTKB |
| ATOM | 3260 | CG | ARG | 544 | 17.913 | 28.975 | 40.773 | 1.00 | 39.29 | BTKB |
| ATOM | 3261 | CD | ARG | 544 | 17.874 | 30.471 | 40.482 | 1.00 | 37.23 | BTKB |
| ATOM | 3262 | NE | ARG | 544 | 19.173 | 30.999 | 40.068 | 1.00 | 34.52 | BTKB |
| ATOM | 3263 | CZ | ARG | 544 | 19.810 | 32.003 | 40.668 | 1.00 | 33.10 | BTKB |
| ATOM | 3264 | NH1 | ARG | 544 | 19.288 | 32.605 | 41.729 | 1.00 | 31.47 | BTKB |
| ATOM | 3265 | NH2 | ARG | 544 | 20.965 | 32.431 | 40.183 | 1.00 | 33.04 | BTKB |
| ATOM | 3266 | C | ARG | 544 | 19.399 | 29.478 | 44.265 | 1.00 | 43.19 | BTKB |
| ATOM | 3267 | O | ARG | 544 | 20.208 | 28.975 | 45.047 | 1.00 | 41.34 | BTKB |
| ATOM | 3268 | N | TYR | 545 | 18.407 | 30.285 | 44.630 | 1.00 | 46.71 | BTKB |
| ATOM | 3269 | CA | TYR | 545 | 18.074 | 30.644 | 46.001 | 1.00 | 47.03 | BTKB |
| ATOM | 3270 | CB | TYR | 545 | 17.896 | 32.176 | 46.075 | 1.00 | 47.74 | BTKB |
| ATOM | 3271 | CG | TYR | 545 | 18.059 | 32.828 | 47.440 | 1.00 | 46.83 | BTKB |
| ATOM | 3272 | CD1 | TYR | 545 | 17.606 | 32.210 | 48.606 | 1.00 | 47.23 | BTKB |
| ATOM | 3273 | CE1 | TYR | 545 | 17.742 | 32.829 | 49.853 | 1.00 | 49.06 | BTKB |
| ATOM | 3274 | CD2 | TYR | 545 | 18.653 | 34.085 | 47.557 | 1.00 | 48.20 | BTKB |
| ATOM | 3275 | CE2 | TYR | 545 | 18.794 | 34.712 | 48.796 | 1.00 | 48.88 | BTKB |
| ATOM | 3276 | CZ | TYR | 545 | 18.338 | 34.080 | 49.937 | 1.00 | 48.87 | BTKB |
| ATOM | 3277 | OH | TYR | 545 | 18.491 | 34.695 | 51.158 | 1.00 | 49.33 | BTKB |
| ATOM | 3278 | C | TYR | 545 | 16.722 | 29.937 | 46.170 | 1.00 | 46.78 | BTKB |
| ATOM | 3279 | O | TYR | 545 | 16.490 | 28.886 | 45.570 | 1.00 | 46.40 | BTKB |
| ATOM | 3280 | N | VAL | 546 | 15.818 | 30.542 | 46.928 | 1.00 | 46.89 | BTKB |
| ATOM | 3281 | CA | VAL | 546 | 14.487 | 29.994 | 47.154 | 1.00 | 47.78 | BTKB |
| ATOM | 3282 | CB | VAL | 546 | 14.440 | 29.047 | 48.385 | 1.00 | 48.44 | BTKB |
| ATOM | 3283 | CG1 | VAL | 546 | 15.047 | 27.696 | 48.038 | 1.00 | 49.26 | BTKB |
| ATOM | 3284 | CG2 | VAL | 546 | 15.174 | 29.668 | 49.569 | 1.00 | 49.28 | BTKB |
| ATOM | 3285 | C | VAL | 546 | 13.555 | 31.180 | 47.374 | 1.00 | 47.25 | BTKB |
| ATOM | 3286 | O | VAL | 546 | 13.997 | 32.331 | 47.375 | 1.00 | 46.90 | BTKB |
| ATOM | 3287 | N | LEU | 547 | 12.269 | 30.908 | 47.546 | 1.00 | 47.06 | BTKB |
| ATOM | 3288 | CA | LEU | 547 | 11.291 | 31.964 | 47.764 | 1.00 | 47.07 | BTKB |
| ATOM | 3289 | CB | LEU | 547 | 10.767 | 32.492 | 46.419 | 1.00 | 46.14 | BTKB |
| ATOM | 3290 | CG | LEU | 547 | 9.916 | 33.768 | 46.428 | 1.00 | 46.13 | BTKB |
| ATOM | 3291 | CD1 | LEU | 547 | 10.754 | 34.945 | 46.906 | 1.00 | 47.63 | BTKB |
| ATOM | 3292 | CD2 | LEU | 547 | 9.359 | 34.035 | 45.035 | 1.00 | 47.76 | BTKB |
| ATOM | 3293 | C | LEU | 547 | 10.169 | 31.363 | 48.602 | 1.00 | 48.24 | BTKB |
| ATOM | 3294 | O | LEU | 547 | 10.439 | 30.733 | 49.623 | 1.00 | 48.69 | BTKB |
| ATOM | 3295 | N | ASP | 548 | 8.927 | 31.495 | 48.141 | 1.00 | 48.47 | BTKB |
| ATOM | 3296 | CA | ASP | 548 | 7.786 | 30.966 | 48.871 | 1.00 | 47.63 | BTKB |
| ATOM | 3297 | CB | ASP | 548 | 6.480 | 31.345 | 48.164 | 1.00 | 47.12 | BTKB |
| ATOM | 3298 | CG | ASP | 548 | 5.272 | 31.257 | 49.078 | 1.00 | 47.82 | BTKB |
| ATOM | 3299 | OD1 | ASP | 548 | 4.251 | 30.681 | 48.653 | 1.00 | 47.08 | BTKB |
| ATOM | 3300 | OD2 | ASP | 548 | 5.335 | 31.775 | 50.216 | 1.00 | 48.07 | BTKB |
| ATOM | 3301 | C | ASP | 548 | 7.907 | 29.451 | 49.017 | 1.00 | 46.66 | BTKB |
| ATOM | 3302 | O | ASP | 548 | 8.000 | 28.944 | 50.131 | 1.00 | 45.89 | BTKB |
| ATOM | 3303 | N | ASP | 549 | 7.954 | 28.735 | 47.896 | 1.00 | 47.04 | BTKB |
| ATOM | 3304 | CA | ASP | 549 | 8.064 | 27.278 | 47.937 | 1.00 | 45.92 | BTKB |
| ATOM | 3305 | CB | ASP | 549 | 6.683 | 26.643 | 48.100 | 1.00 | 44.19 | BTKB |
| ATOM | 3306 | CG | ASP | 549 | 6.155 | 26.745 | 49.517 | 1.00 | 43.52 | BTKB |
| ATOM | 3307 | OD1 | ASP | 549 | 5.403 | 27.697 | 49.809 | 1.00 | 39.96 | BTKB |
| ATOM | 3308 | OD2 | ASP | 549 | 6.494 | 25.870 | 50.340 | 1.00 | 43.98 | BTKB |
| ATOM | 3309 | C | ASP | 549 | 8.787 | 26.633 | 46.755 | 1.00 | 46.60 | BTKB |
| ATOM | 3310 | O | ASP | 549 | 9.179 | 25.470 | 46.835 | 1.00 | 48.82 | BTKB |
| ATOM | 3311 | N | GLU | 550 | 8.931 | 27.366 | 45.653 | 1.00 | 46.02 | BTKB |
| ATOM | 3312 | CA | GLU | 550 | 9.611 | 26.852 | 44.459 | 1.00 | 44.77 | BTKB |
| ATOM | 3313 | CB | GLU | 550 | 8.867 | 25.635 | 43.881 | 1.00 | 44.82 | BTKB |
| ATOM | 3314 | CG | GLU | 550 | 7.334 | 25.707 | 43.878 | 1.00 | 40.66 | BTKB |
| ATOM | 3315 | CD | GLU | 550 | 6.772 | 26.820 | 43.009 | 1.00 | 39.18 | BTKB |
| ATOM | 3316 | OE1 | GLU | 550 | 6.074 | 27.701 | 43.554 | 1.00 | 38.17 | BTKB |
| ATOM | 3317 | OE2 | GLU | 550 | 7.016 | 26.810 | 41.784 | 1.00 | 38.80 | BTKB |
| ATOM | 3318 | C | GLU | 550 | 9.775 | 27.931 | 43.389 | 1.00 | 45.63 | BTKB |
| ATOM | 3319 | O | GLU | 550 | 9.743 | 29.123 | 43.701 | 1.00 | 42.92 | BTKB |
| ATOM | 3320 | N | TYR | 551 | 9.969 | 27.509 | 42.141 | 1.00 | 45.07 | BTKB |
| ATOM | 3321 | CA | TYR | 551 | 10.119 | 28.427 | 41.016 | 1.00 | 46.80 | BTKB |
| ATOM | 3322 | CB | TYR | 551 | 11.410 | 29.243 | 41.147 | 1.00 | 47.58 | BTKB |
| ATOM | 3323 | CG | TYR | 551 | 12.684 | 28.433 | 41.176 | 1.00 | 46.34 | BTKB |
| ATOM | 3324 | CD1 | TYR | 551 | 13.471 | 28.303 | 40.035 | 1.00 | 44.53 | BTKB |
| ATOM | 3325 | CE1 | TYR | 551 | 14.668 | 27.596 | 40.063 | 1.00 | 45.32 | BTKB |
| ATOM | 3326 | CD2 | TYR | 551 | 13.126 | 27.831 | 42.354 | 1.00 | 46.94 | BTKB |
| ATOM | 3327 | CE2 | TYR | 551 | 14.324 | 27.120 | 42.394 | 1.00 | 46.70 | BTKB |
| ATOM | 3328 | CZ | TYR | 551 | 15.090 | 27.008 | 41.244 | 1.00 | 46.98 | BTKB |
| ATOM | 3329 | OH | TYR | 551 | 16.275 | 26.309 | 41.268 | 1.00 | 47.08 | BTKB |
| ATOM | 3330 | C | TYR | 551 | 10.061 | 27.697 | 39.665 | 1.00 | 47.92 | BTKB |
| ATOM | 3331 | O | TYR | 551 | 9.575 | 26.567 | 39.583 | 1.00 | 48.34 | BTKB |
| ATOM | 3332 | N | THR | 552 | 10.539 | 28.344 | 38.606 | 1.00 | 47.56 | BTKB |
| ATOM | 3333 | CA | THR | 552 | 10.519 | 27.736 | 37.279 | 1.00 | 47.33 | BTKB |
| ATOM | 3334 | CB | THR | 552 | 9.352 | 28.283 | 36.436 | 1.00 | 44.44 | BTKB |
| ATOM | 3335 | OG1 | THR | 552 | 8.153 | 28.291 | 37.219 | 1.00 | 43.39 | BTKB |
| ATOM | 3336 | CG2 | THR | 552 | 9.139 | 27.423 | 35.204 | 1.00 | 43.18 | BTKB |
| ATOM | 3337 | C | THR | 552 | 11.823 | 27.975 | 36.521 | 1.00 | 48.87 | BTKB |
| ATOM | 3338 | O | THR | 552 | 12.474 | 27.030 | 36.080 | 1.00 | 49.47 | BTKB |
| ATOM | 3339 | N | SER | 553 | 12.203 | 29.242 | 36.382 | 1.00 | 50.63 | BTKB |
| ATOM | 3340 | CA | SER | 553 | 13.425 | 29.614 | 35.674 | 1.00 | 51.18 | BTKB |
| ATOM | 3341 | CB | SER | 553 | 14.655 | 29.070 | 36.414 | 1.00 | 48.79 | BTKB |
| ATOM | 3342 | OG | SER | 553 | 14.750 | 29.635 | 37.713 | 1.00 | 43.88 | BTKB |
| ATOM | 3343 | C | SER | 553 | 13.395 | 29.148 | 34.210 | 1.00 | 51.91 | BTKB |
| ATOM | 3344 | O | SER | 553 | 12.331 | 29.148 | 33.584 | 1.00 | 52.66 | BTKB |
| ATOM | 3345 | N | SER | 554 | 14.552 | 28.772 | 33.667 | 1.00 | 50.57 | BTKB |
| ATOM | 3346 | CA | SER | 554 | 14.656 | 28.321 | 32.283 | 1.00 | 48.71 | BTKB |
| ATOM | 3347 | CB | SER | 554 | 16.121 | 28.056 | 31.928 | 1.00 | 49.93 | BTKB |
| ATOM | 3348 | OG | SER | 554 | 16.904 | 29.219 | 32.142 | 1.00 | 48.05 | BTKB |
| ATOM | 3349 | C | SER | 554 | 13.811 | 27.086 | 31.977 | 1.00 | 47.80 | BTKB |
| ATOM | 3350 | O | SER | 554 | 14.228 | 25.955 | 32.228 | 1.00 | 49.68 | BTKB |
| ATOM | 3351 | N | VAL | 555 | 12.624 | 27.319 | 31.427 | 1.00 | 45.84 | BTKB |
| ATOM | 3352 | CA | VAL | 555 | 11.702 | 26.247 | 31.067 | 1.00 | 42.92 | BTKB |
| ATOM | 3353 | CB | VAL | 555 | 10.362 | 26.821 | 30.570 | 1.00 | 38.45 | BTKB |
| ATOM | 3354 | CG1 | VAL | 555 | 9.363 | 25.703 | 30.345 | 1.00 | 41.05 | BTKB |
| ATOM | 3355 | CG2 | VAL | 555 | 9.819 | 27.822 | 31.577 | 1.00 | 38.78 | BTKB |
| ATOM | 3356 | C | VAL | 555 | 12.319 | 25.381 | 29.969 | 1.00 | 43.08 | BTKB |
| ATOM | 3357 | O | VAL | 555 | 12.798 | 25.904 | 28.963 | 1.00 | 45.11 | BTKB |
| ATOM | 3358 | N | GLY | 556 | 12.284 | 24.062 | 30.157 | 1.00 | 40.17 | BTKB |
| ATOM | 3359 | CA | GLY | 556 | 12.860 | 23.144 | 29.185 | 1.00 | 36.28 | BTKB |
| ATOM | 3360 | C | GLY | 556 | 14.375 | 23.211 | 29.259 | 1.00 | 34.46 | BTKB |
| ATOM | 3361 | O | GLY | 556 | 14.937 | 24.302 | 29.258 | 1.00 | 34.17 | BTKB |
| ATOM | 3362 | N | SER | 557 | 15.042 | 22.065 | 29.357 | 1.00 | 37.09 | BTKB |
| ATOM | 3363 | CA | SER | 557 | 16.501 | 22.047 | 29.451 | 1.00 | 39.60 | BTKB |
| ATOM | 3364 | CB | SER | 557 | 16.959 | 22.896 | 30.650 | 1.00 | 42.29 | BTKB |
| ATOM | 3365 | OG | SER | 557 | 18.341 | 23.216 | 30.573 | 1.00 | 42.78 | BTKB |
| ATOM | 3366 | C | SER | 557 | 17.084 | 20.634 | 29.581 | 1.00 | 39.69 | BTKB |
| ATOM | 3367 | O | SER | 557 | 16.355 | 19.635 | 29.559 | 1.00 | 37.28 | BTKB |
| ATOM | 3368 | N | LYS | 558 | 18.408 | 20.572 | 29.710 | 1.00 | 38.88 | BTKB |
| ATOM | 3369 | CA | LYS | 558 | 19.131 | 19.317 | 29.861 | 1.00 | 38.70 | BTKB |
| ATOM | 3370 | CB | LYS | 558 | 20.122 | 19.107 | 28.711 | 1.00 | 40.51 | BTKB |
| ATOM | 3371 | CG | LYS | 558 | 19.507 | 19.185 | 27.328 | 1.00 | 39.86 | BTKB |
| ATOM | 3372 | CD | LYS | 558 | 19.318 | 20.630 | 26.895 | 1.00 | 40.70 | BTKB |
| ATOM | 3373 | CE | LYS | 558 | 18.358 | 20.719 | 25.736 | 1.00 | 38.88 | BTKB |
| ATOM | 3374 | NZ | LYS | 558 | 17.034 | 20.178 | 26.138 | 1.00 | 36.01 | BTKB |
| ATOM | 3375 | C | LYS | 558 | 19.883 | 19.350 | 31.188 | 1.00 | 38.02 | BTKB |
| ATOM | 3376 | O | LYS | 558 | 20.904 | 20.019 | 31.327 | 1.00 | 38.61 | BTKB |
| ATOM | 3377 | N | PHE | 559 | 19.342 | 18.648 | 32.171 | 1.00 | 35.11 | BTKB |
| ATOM | 3378 | CA | PHE | 559 | 19.935 | 18.570 | 33.499 | 1.00 | 32.80 | BTKB |
| ATOM | 3379 | CB | PHE | 559 | 18.836 | 18.268 | 34.530 | 1.00 | 35.42 | BTKB |
| ATOM | 3380 | CG | PHE | 559 | 17.431 | 18.422 | 33.991 | 1.00 | 38.43 | BTKB |
| ATOM | 3381 | CD1 | PHE | 559 | 16.647 | 17.302 | 33.728 | 1.00 | 35.87 | BTKB |
| ATOM | 3382 | CD2 | PHE | 559 | 16.902 | 19.685 | 33.730 | 1.00 | 35.45 | BTKB |
| ATOM | 3383 | CE1 | PHE | 559 | 15.360 | 17.434 | 33.212 | 1.00 | 38.06 | BTKB |
| ATOM | 3384 | CE2 | PHE | 559 | 15.616 | 19.827 | 33.214 | 1.00 | 33.30 | BTKB |
| ATOM | 3385 | CZ | PHE | 559 | 14.843 | 18.696 | 32.954 | 1.00 | 36.08 | BTKB |
| ATOM | 3386 | C | PHE | 559 | 20.947 | 17.423 | 33.473 | 1.00 | 30.75 | BTKB |
| ATOM | 3387 | O | PHE | 559 | 20.919 | 16.597 | 32.556 | 1.00 | 30.59 | BTKB |
| ATOM | 3388 | N | PRO | 560 | 21.843 | 17.341 | 34.478 | 1.00 | 27.32 | BTKB |
| ATOM | 3389 | CD | PRO | 560 | 22.005 | 18.208 | 35.658 | 1.00 | 23.09 | BTKB |
| ATOM | 3390 | CA | PRO | 560 | 22.832 | 16.255 | 34.503 | 1.00 | 23.53 | BTKB |
| ATOM | 3391 | CB | PRO | 560 | 23.640 | 16.561 | 35.765 | 1.00 | 20.79 | BTKB |
| ATOM | 3392 | CG | PRO | 560 | 22.668 | 17.275 | 36.631 | 1.00 | 19.96 | BTKB |
| ATOM | 3393 | C | PRO | 560 | 22.166 | 14.872 | 34.563 | 1.00 | 23.08 | BTKB |
| ATOM | 3394 | O | PRO | 560 | 22.778 | 13.857 | 34.212 | 1.00 | 21.72 | BTKB |
| ATOM | 3395 | N | VAL | 561 | 20.921 | 14.846 | 35.037 | 1.00 | 22.54 | BTKB |
| ATOM | 3396 | CA | VAL | 561 | 20.118 | 13.629 | 35.138 | 1.00 | 21.12 | BTKB |
| ATOM | 3397 | CB | VAL | 561 | 20.065 | 12.847 | 33.786 | 1.00 | 24.08 | BTKB |
| ATOM | 3398 | CG1 | VAL | 561 | 19.095 | 11.656 | 33.890 | 1.00 | 22.39 | BTKB |
| ATOM | 3399 | CG2 | VAL | 561 | 19.654 | 13.778 | 32.641 | 1.00 | 24.23 | BTKB |
| ATOM | 3400 | C | VAL | 561 | 20.510 | 12.666 | 36.259 | 1.00 | 18.16 | BTKB |
| ATOM | 3401 | O | VAL | 561 | 19.709 | 12.404 | 37.162 | 1.00 | 18.62 | BTKB |
| ATOM | 3402 | N | ARG | 562 | 21.729 | 12.141 | 36.210 | 1.00 | 13.44 | BTKB |
| ATOM | 3403 | CA | ARG | 562 | 22.182 | 11.192 | 37.221 | 1.00 | 14.89 | BTKB |
| ATOM | 3404 | CB | ARG | 562 | 23.606 | 10.726 | 36.919 | 1.00 | 18.46 | BTKB |
| ATOM | 3405 | CG | ARG | 562 | 23.764 | 9.858 | 35.677 | 1.00 | 20.23 | BTKB |
| ATOM | 3406 | CD | ARG | 562 | 23.654 | 10.637 | 34.387 | 1.00 | 19.70 | BTKB |
| ATOM | 3407 | NE | ARG | 562 | 24.262 | 9.895 | 33.284 | 1.00 | 26.34 | BTKB |
| ATOM | 3408 | CZ | ARG | 562 | 23.614 | 9.045 | 32.495 | 1.00 | 23.35 | BTKB |
| ATOM | 3409 | NH1 | ARG | 562 | 22.320 | 8.821 | 32.670 | 1.00 | 25.49 | BTKB |
| ATOM | 3410 | NH2 | ARG | 562 | 24.277 | 8.394 | 31.549 | 1.00 | 20.77 | BTKB |
| ATOM | 3411 | C | ARG | 562 | 22.129 | 11.748 | 38.639 | 1.00 | 14.76 | BTKB |
| ATOM | 3412 | O | ARG | 562 | 21.745 | 11.051 | 39.584 | 1.00 | 15.25 | BTKB |
| ATOM | 3413 | N | TRP | 563 | 22.510 | 13.012 | 38.769 | 1.00 | 13.56 | BTKB |
| ATOM | 3414 | CA | TRP | 563 | 22.544 | 13.707 | 40.050 | 1.00 | 15.17 | BTKB |
| ATOM | 3415 | CB | TRP | 563 | 23.680 | 14.733 | 40.021 | 1.00 | 17.58 | BTKB |
| ATOM | 3416 | CG | TRP | 563 | 25.024 | 14.120 | 39.845 | 1.00 | 16.68 | BTKB |
| ATOM | 3417 | CD2 | TRP | 563 | 25.614 | 13.658 | 38.619 | 1.00 | 18.94 | BTKB |
| ATOM | 3418 | CE2 | TRP | 563 | 26.870 | 13.106 | 38.945 | 1.00 | 19.94 | BTKB |
| ATOM | 3419 | CE3 | TRP | 563 | 25.202 | 13.651 | 37.280 | 1.00 | 18.43 | BTKB |
| ATOM | 3420 | CD1 | TRP | 563 | 25.924 | 13.848 | 40.830 | 1.00 | 12.29 | BTKB |
| ATOM | 3421 | NE1 | TRP | 563 | 27.028 | 13.233 | 40.300 | 1.00 | 18.47 | BTKB |
| ATOM | 3422 | CZ2 | TRP | 563 | 27.724 | 12.552 | 37.979 | 1.00 | 20.36 | BTKB |
| ATOM | 3423 | CZ3 | TRP | 563 | 26.056 | 13.097 | 36.314 | 1.00 | 17.95 | BTKB |
| ATOM | 3424 | CH2 | TRP | 563 | 27.298 | 12.555 | 36.674 | 1.00 | 17.39 | BTKB |
| ATOM | 3425 | C | TRP | 563 | 21.236 | 14.431 | 40.346 | 1.00 | 16.25 | BTKB |
| ATOM | 3426 | O | TRP | 563 | 21.202 | 15.361 | 41.157 | 1.00 | 15.08 | BTKB |
| ATOM | 3427 | N | SER | 564 | 20.153 | 13.979 | 39.728 | 1.00 | 16.15 | BTKB |
| ATOM | 3428 | CA | SER | 564 | 18.877 | 14.647 | 39.894 | 1.00 | 15.25 | BTKB |
| ATOM | 3429 | CB | SER | 564 | 18.450 | 15.226 | 38.540 | 1.00 | 12.15 | BTKB |
| ATOM | 3430 | OG | SER | 564 | 19.456 | 16.046 | 37.961 | 1.00 | 15.22 | BTKB |
| ATOM | 3431 | C | SER | 564 | 17.732 | 13.812 | 40.466 | 1.00 | 18.06 | BTKB |
| ATOM | 3432 | O | SER | 564 | 17.643 | 12.604 | 40.245 | 1.00 | 18.41 | BTKB |
| ATOM | 3433 | N | PRO | 565 | 16.852 | 14.453 | 41.250 | 1.00 | 19.48 | BTKB |
| ATOM | 3434 | CD | PRO | 565 | 17.013 | 15.807 | 41.805 | 1.00 | 16.11 | BTKB |
| ATOM | 3435 | CA | PRO | 565 | 15.699 | 13.784 | 41.854 | 1.00 | 20.89 | BTKB |
| ATOM | 3436 | CB | PRO | 565 | 15.154 | 14.852 | 42.801 | 1.00 | 20.19 | BTKB |
| ATOM | 3437 | CG | PRO | 565 | 16.331 | 15.681 | 43.117 | 1.00 | 16.32 | BTKB |
| ATOM | 3438 | C | PRO | 565 | 14.678 | 13.499 | 40.748 | 1.00 | 24.06 | BTKB |
| ATOM | 3439 | O | PRO | 565 | 14.578 | 14.260 | 39.778 | 1.00 | 25.01 | BTKB |
| ATOM | 3440 | N | PRO | 566 | 13.878 | 12.433 | 40.892 | 1.00 | 25.49 | BTKB |
| ATOM | 3441 | CD | PRO | 566 | 13.859 | 11.410 | 41.947 | 1.00 | 24.25 | BTKB |
| ATOM | 3442 | CA | PRO | 566 | 12.887 | 12.126 | 39.853 | 1.00 | 27.84 | BTKB |
| ATOM | 3443 | CB | PRO | 566 | 12.114 | 10.966 | 40.470 | 1.00 | 26.01 | BTKB |
| ATOM | 3444 | CG | PRO | 566 | 13.169 | 10.267 | 41.256 | 1.00 | 28.12 | BTKB |
| ATOM | 3445 | C | PRO | 566 | 11.985 | 13.330 | 39.585 | 1.00 | 29.05 | BTKB |
| ATOM | 3446 | O | PRO | 566 | 11.516 | 13.537 | 38.465 | 1.00 | 31.25 | BTKB |
| ATOM | 3447 | N | GLU | 567 | 11.805 | 14.151 | 40.613 | 1.00 | 30.57 | BTKB |
| ATOM | 3448 | CA | GLU | 567 | 10.982 | 15.348 | 40.518 | 1.00 | 31.66 | BTKB |
| ATOM | 3449 | CB | GLU | 567 | 11.006 | 16.119 | 41.841 | 1.00 | 30.30 | BTKB |
| ATOM | 3450 | CG | GLU | 567 | 10.491 | 15.346 | 43.051 | 1.00 | 31.92 | BTKB |
| ATOM | 3451 | CD | GLU | 567 | 11.493 | 14.337 | 43.594 | 1.00 | 30.79 | BTKB |
| ATOM | 3452 | OE1 | GLU | 567 | 12.348 | 14.722 | 44.424 | 1.00 | 29.93 | BTKB |
| ATOM | 3453 | OE2 | GLU | 567 | 11.412 | 13.156 | 43.200 | 1.00 | 33.30 | BTKB |
| ATOM | 3454 | C | GLU | 567 | 11.483 | 16.253 | 39.394 | 1.00 | 29.70 | BTKB |
| ATOM | 3455 | O | GLU | 567 | 10.723 | 16.629 | 38.494 | 1.00 | 30.08 | BTKB |
| ATOM | 3456 | N | VAL | 568 | 12.779 | 16.553 | 39.421 | 1.00 | 30.70 | BTKB |
| ATOM | 3457 | CA | VAL | 568 | 13.382 | 17.422 | 38.419 | 1.00 | 30.50 | BTKB |
| ATOM | 3458 | CB | VAL | 568 | 14.737 | 17.976 | 38.885 | 1.00 | 30.30 | BTKB |
| ATOM | 3459 | CG1 | VAL | 568 | 14.584 | 18.738 | 40.177 | 1.00 | 28.02 | BTKB |
| ATOM | 3460 | CG2 | VAL | 568 | 15.718 | 16.866 | 39.048 | 1.00 | 24.92 | BTKB |
| ATOM | 3461 | C | VAL | 568 | 13.553 | 16.767 | 37.052 | 1.00 | 31.05 | BTKB |
| ATOM | 3462 | O | VAL | 568 | 13.494 | 17.444 | 36.028 | 1.00 | 33.18 | BTKB |
| ATOM | 3463 | N | LEU | 569 | 13.746 | 15.454 | 37.039 | 1.00 | 33.44 | BTKB |
| ATOM | 3464 | CA | LEU | 569 | 13.924 | 14.711 | 35.791 | 1.00 | 34.63 | BTKB |
| ATOM | 3465 | CB | LEU | 569 | 14.322 | 13.266 | 36.091 | 1.00 | 32.99 | BTKB |
| ATOM | 3466 | CG | LEU | 569 | 15.657 | 13.167 | 36.828 | 1.00 | 31.76 | BTKB |
| ATOM | 3467 | CD1 | LEU | 569 | 15.945 | 11.733 | 37.268 | 1.00 | 26.25 | BTKB |
| ATOM | 3468 | CD2 | LEU | 569 | 16.742 | 13.705 | 35.902 | 1.00 | 28.56 | BTKB |
| ATOM | 3469 | C | LEU | 569 | 12.687 | 14.733 | 34.899 | 1.00 | 36.45 | BTKB |
| ATOM | 3470 | O | LEU | 569 | 12.741 | 14.313 | 33.745 | 1.00 | 36.82 | BTKB |
| ATOM | 3471 | N | MET | 570 | 11.568 | 15.192 | 35.451 | 1.00 | 39.88 | BTKB |
| ATOM | 3472 | CA | MET | 570 | 10.325 | 13.276 | 34.700 | 1.00 | 39.46 | BTKB |
| ATOM | 3473 | CB | MET | 570 | 9.255 | 14.381 | 35.332 | 1.00 | 39.18 | BTKB |
| ATOM | 3474 | CG | MET | 570 | 9.607 | 12.895 | 35.325 | 1.00 | 38.41 | BTKB |
| ATOM | 3475 | SD | MET | 570 | 8.311 | 11.837 | 36.007 | 1.00 | 37.44 | BTKB |
| ATOM | 3476 | CE | MET | 570 | 8.698 | 11.878 | 37.754 | 1.00 | 37.14 | BTKB |
| ATOM | 3477 | C | MET | 570 | 9.831 | 16.719 | 34.592 | 1.00 | 40.77 | BTKB |
| ATOM | 3478 | O | MET | 570 | 9.802 | 17.287 | 33.499 | 1.00 | 41.26 | BTKB |
| ATOM | 3479 | N | TYR | 571 | 9.493 | 17.327 | 35.727 | 1.00 | 42.07 | BTKB |
| ATOM | 3480 | CA | TYR | 571 | 8.985 | 18.703 | 35.739 | 1.00 | 43.90 | BTKB |
| ATOM | 3481 | CB | TYR | 571 | 7.560 | 18.741 | 36.322 | 1.00 | 44.93 | BTKB |
| ATOM | 3482 | CG | TYR | 571 | 7.386 | 17.933 | 37.593 | 1.00 | 45.07 | BTKB |
| ATOM | 3483 | CD1 | TYR | 571 | 6.986 | 16.595 | 37.542 | 1.00 | 45.70 | BTKB |
| ATOM | 3484 | CE1 | TYR | 571 | 6.863 | 15.833 | 38.706 | 1.00 | 46.81 | BTKB |
| ATOM | 3485 | CD2 | TYR | 571 | 7.653 | 18.493 | 38.842 | 1.00 | 46.94 | BTKB |
| ATOM | 3486 | CE2 | TYR | 571 | 7.531 | 17.740 | 40.011 | 1.00 | 46.48 | BTKB |
| ATOM | 3487 | CZ | TYR | 571 | 7.139 | 16.412 | 39.937 | 1.00 | 46.81 | BTKB |
| ATOM | 3488 | OH | TYR | 571 | 7.043 | 15.662 | 41.089 | 1.00 | 45.30 | BTKB |
| ATOM | 3489 | C | TYR | 571 | 9.886 | 19.698 | 36.469 | 1.00 | 42.72 | BTKB |
| ATOM | 3490 | O | TYR | 571 | 9.411 | 20.690 | 37.014 | 1.00 | 42.57 | BTKB |
| ATOM | 3491 | N | SER | 572 | 11.183 | 19.410 | 36.490 | 1.00 | 42.42 | BTKB |
| ATOM | 3492 | CA | SER | 572 | 12.181 | 20.260 | 37.136 | 1.00 | 40.33 | BTKB |
| ATOM | 3493 | CB | SER | 572 | 12.616 | 21.368 | 36.175 | 1.00 | 41.33 | BTKB |
| ATOM | 3494 | OG | SER | 572 | 13.136 | 20.813 | 34.978 | 1.00 | 43.33 | BTKB |
| ATOM | 3495 | C | SER | 572 | 11.803 | 20.849 | 38.500 | 1.00 | 37.82 | BTKB |
| ATOM | 3496 | O | SER | 572 | 11.843 | 22.062 | 38.693 | 1.00 | 36.53 | BTKB |
| ATOM | 3497 | N | LYS | 573 | 11.481 | 19.980 | 39.454 | 1.00 | 36.19 | BTKB |
| ATOM | 3498 | CA | LYS | 573 | 11.116 | 20.421 | 40.798 | 1.00 | 34.19 | BTKB |
| ATOM | 3499 | CB | LYS | 573 | 10.084 | 19.476 | 41.406 | 1.00 | 31.01 | BTKB |
| ATOM | 3500 | CG | LYS | 573 | 9.519 | 19.960 | 42.718 | 1.00 | 33.03 | BTKB |
| ATOM | 3501 | CD | LYS | 573 | 8.828 | 21.296 | 42.551 | 1.00 | 26.52 | BTKB |
| ATOM | 3502 | CE | LYS | 573 | 9.322 | 22.299 | 43.578 | 1.00 | 24.03 | BTKB |
| ATOM | 3503 | NZ | LYS | 573 | 10.709 | 22.745 | 43.291 | 1.00 | 17.04 | BTKB |
| ATOM | 3504 | C | LYS | 573 | 12.335 | 20.550 | 41.720 | 1.00 | 35.59 | BTKB |
| ATOM | 3505 | O | LYS | 573 | 12.666 | 19.636 | 42.481 | 1.00 | 33.63 | BTKB |
| ATOM | 3506 | N | PHE | 574 | 13.001 | 21.696 | 41.620 | 1.00 | 38.79 | BTKB |
| ATOM | 3507 | CA | PHE | 574 | 14.198 | 22.030 | 42.404 | 1.00 | 36.81 | BTKB |
| ATOM | 3508 | CB | PHE | 574 | 15.061 | 23.057 | 41.657 | 1.00 | 37.78 | BTKB |
| ATOM | 3509 | CG | PHE | 574 | 14.330 | 23.776 | 40.555 | 1.00 | 36.05 | BTKB |
| ATOM | 3510 | CD1 | PHE | 574 | 13.099 | 24.384 | 40.792 | 1.00 | 37.69 | BTKB |
| ATOM | 3511 | CD2 | PHE | 574 | 14.856 | 23.811 | 39.269 | 1.00 | 38.01 | BTKB |
| ATOM | 3512 | CE1 | PHE | 574 | 12.405 | 25.007 | 39.768 | 1.00 | 37.96 | BTKB |
| ATOM | 3513 | CE2 | PHE | 574 | 14.168 | 24.435 | 38.236 | 1.00 | 38.31 | BTKB |
| ATOM | 3514 | CZ | PHE | 574 | 12.938 | 25.034 | 38.487 | 1.00 | 39.59 | BTKB |
| ATOM | 3515 | C | PHE | 574 | 13.865 | 22.573 | 43.785 | 1.00 | 34.93 | BTKB |
| ATOM | 3516 | O | PHE | 574 | 13.125 | 23.546 | 43.917 | 1.00 | 33.39 | BTKB |
| ATOM | 3517 | N | SER | 575 | 14.473 | 21.988 | 44.806 | 1.00 | 32.93 | BTKB |
| ATOM | 3518 | CA | SER | 575 | 14.229 | 22.405 | 46.178 | 1.00 | 31.78 | BTKB |
| ATOM | 3519 | CB | SER | 575 | 13.053 | 21.607 | 46.740 | 1.00 | 30.36 | BTKB |
| ATOM | 3520 | OG | SER | 575 | 13.264 | 20.216 | 46.563 | 1.00 | 31.46 | BTKB |
| ATOM | 3521 | C | SER | 575 | 15.459 | 22.157 | 47.040 | 1.00 | 32.41 | BTKB |
| ATOM | 3522 | O | SER | 575 | 16.492 | 21.704 | 46.542 | 1.00 | 32.95 | BTKB |
| ATOM | 3523 | N | SER | 576 | 15.338 | 22.453 | 48.333 | 1.00 | 31.52 | BTKB |
| ATOM | 3524 | CA | SER | 576 | 16.414 | 22.242 | 49.297 | 1.00 | 26.86 | BTKB |
| ATOM | 3525 | CB | SER | 576 | 15.917 | 22.607 | 50.695 | 1.00 | 30.38 | BTKB |
| ATOM | 3526 | OG | SER | 576 | 14.621 | 22.070 | 50.923 | 1.00 | 33.61 | BTKB |
| ATOM | 3527 | C | SER | 576 | 16.827 | 20.772 | 49.275 | 1.00 | 23.09 | BTKB |
| ATOM | 3528 | O | SER | 576 | 18.003 | 20.432 | 49.457 | 1.00 | 18.87 | BTKB |
| ATOM | 3529 | N | LYS | 577 | 15.840 | 19.913 | 49.036 | 1.00 | 19.70 | BTKB |
| ATOM | 3530 | CA | LYS | 577 | 16.048 | 18.477 | 48.973 | 1.00 | 15.88 | BTKB |
| ATOM | 3531 | CB | LYS | 577 | 14.753 | 17.732 | 49.286 | 1.00 | 15.45 | BTKB |
| ATOM | 3532 | CG | LYS | 577 | 14.598 | 17.347 | 50.748 | 1.00 | 14.36 | BTKB |
| ATOM | 3533 | CD | LYS | 577 | 14.604 | 18.548 | 51.663 | 1.00 | 10.74 | BTKB |
| ATOM | 3534 | CE | LYS | 577 | 14.692 | 18.116 | 53.102 | 1.00 | 8.91 | BTKB |
| ATOM | 3535 | NZ | LYS | 577 | 15.888 | 17.272 | 53.339 | 1.00 | 13.99 | BTKB |
| ATOM | 3536 | C | LYS | 577 | 16.627 | 17.985 | 47.659 | 1.00 | 15.31 | BTKB |
| ATOM | 3537 | O | LYS | 577 | 16.966 | 16.815 | 47.550 | 1.00 | 18.88 | BTKB |
| ATOM | 3538 | N | SER | 578 | 16.737 | 18.855 | 46.660 | 1.00 | 15.22 | BTKB |
| ATOM | 3539 | CA | SER | 578 | 17.316 | 18.453 | 45.382 | 1.00 | 14.59 | BTKB |
| ATOM | 3540 | CB | SER | 578 | 17.043 | 19.491 | 44.290 | 1.00 | 18.56 | BTKB |
| ATOM | 3541 | OG | SER | 578 | 15.680 | 19.499 | 43.903 | 1.00 | 23.33 | BTKB |
| ATOM | 3542 | C | SER | 578 | 18.821 | 18.282 | 45.557 | 1.00 | 15.07 | BTKB |
| ATOM | 3543 | O | SER | 578 | 19.398 | 17.307 | 45.075 | 1.00 | 16.57 | BTKB |
| ATOM | 3544 | N | ASP | 579 | 19.448 | 19.220 | 46.267 | 1.00 | 15.79 | BTKB |
| ATOM | 3545 | CA | ASP | 579 | 20.888 | 19.162 | 46.517 | 1.00 | 15.96 | BTKB |
| ATOM | 3546 | CB | ASP | 579 | 21.387 | 20.432 | 47.217 | 1.00 | 15.23 | BTKB |
| ATOM | 3547 | CG | ASP | 579 | 21.587 | 21.610 | 46.261 | 1.00 | 16.89 | BTKB |
| ATOM | 3548 | OD1 | ASP | 579 | 21.403 | 22.748 | 46.724 | 1.00 | 14.46 | BTKB |
| ATOM | 3549 | OD2 | ASP | 579 | 21.953 | 21.414 | 45.075 | 1.00 | 17.09 | BTKB |
| ATOM | 3550 | C | ASP | 579 | 21.218 | 17.955 | 47.378 | 1.00 | 18.59 | BTKB |
| ATOM | 3551 | O | ASP | 579 | 22.329 | 17.422 | 47.313 | 1.00 | 20.19 | BTKB |
| ATOM | 3552 | N | ILE | 580 | 20.255 | 17.532 | 48.194 | 1.00 | 18.03 | BTKB |
| ATOM | 3553 | CA | ILE | 580 | 20.447 | 16.373 | 49.054 | 1.00 | 17.50 | BTKB |
| ATOM | 3554 | CB | ILE | 580 | 19.272 | 16.189 | 50.035 | 1.00 | 15.12 | BTKB |
| ATOM | 3555 | CG2 | ILE | 580 | 19.377 | 14.837 | 50.760 | 1.00 | 14.16 | BTKB |
| ATOM | 3556 | CG1 | ILE | 580 | 19.274 | 17.327 | 51.057 | 1.00 | 13.57 | BTKB |
| ATOM | 3557 | CD | ILE | 580 | 20.508 | 17.347 | 51.943 | 1.00 | 12.21 | BTKB |
| ATOM | 3558 | C | ILE | 580 | 20.620 | 15.127 | 48.193 | 1.00 | 19.53 | BTKB |
| ATOM | 3559 | O | ILE | 580 | 21.548 | 14.353 | 48.411 | 1.00 | 26.22 | BTKB |
| ATOM | 3560 | N | TRP | 581 | 19.754 | 14.960 | 47.194 | 1.00 | 16.88 | BTKB |
| ATOM | 3561 | CA | TRP | 581 | 19.838 | 13.815 | 46.291 | 1.00 | 11.89 | BTKB |
| ATOM | 3562 | CB | TRP | 581 | 18.753 | 13.885 | 45.217 | 1.00 | 8.40 | BTKB |
| ATOM | 3563 | CG | TRP | 581 | 18.746 | 12.692 | 44.284 | 1.00 | 7.47 | BTKB |
| ATOM | 3564 | CD2 | TRP | 581 | 17.741 | 11.675 | 44.201 | 1.00 | 6.04 | BTKB |
| ATOM | 3565 | CE2 | TRP | 581 | 18.130 | 10.788 | 43.165 | 1.00 | 6.60 | BTKB |
| ATOM | 3566 | CE3 | TRP | 581 | 16.547 | 11.429 | 44.890 | 1.00 | 9.89 | BTKB |
| ATOM | 3567 | CD1 | TRP | 581 | 19.675 | 12.386 | 43.329 | 1.00 | 5.91 | BTKB |
| ATOM | 3568 | NE1 | TRP | 581 | 19.310 | 11.247 | 42.654 | 1.00 | 4.72 | BTKB |
| ATOM | 3569 | CZ2 | TRP | 581 | 17.362 | 9.676 | 42.805 | 1.00 | 7.81 | BTKB |
| ATOM | 3570 | CZ3 | TRP | 581 | 15.783 | 10.324 | 44.532 | 1.00 | 6.46 | BTKB |
| ATOM | 3571 | CH2 | TRP | 581 | 16.195 | 9.462 | 43.498 | 1.00 | 13.07 | BTKB |
| ATOM | 3572 | C | TRP | 581 | 21.206 | 13.785 | 45.625 | 1.00 | 10.29 | BTKB |
| ATOM | 3573 | O | TRP | 581 | 21.909 | 12.770 | 45.678 | 1.00 | 11.92 | BTKB |
| ATOM | 3574 | N | ALA | 582 | 21.590 | 14.911 | 45.022 | 1.00 | 10.55 | BTKB |
| ATOM | 3575 | CA | ALA | 582 | 22.872 | 15.023 | 44.341 | 1.00 | 4.48 | BTKB |
| ATOM | 3576 | CB | ALA | 582 | 23.003 | 16.387 | 43.684 | 1.00 | 4.04 | BTKB |
| ATOM | 3577 | C | ALA | 582 | 24.058 | 14.758 | 45.261 | 1.00 | 6.67 | BTKB |
| ATOM | 3578 | O | ALA | 582 | 25.056 | 14.185 | 44.835 | 1.00 | 8.34 | BTKB |
| ATOM | 3579 | N | PHE | 583 | 23.943 | 15.132 | 46.531 | 1.00 | 9.39 | BTKB |
| ATOM | 3580 | CA | PHE | 583 | 25.038 | 14.908 | 47.478 | 1.00 | 8.21 | BTKB |
| ATOM | 3581 | CB | PHE | 583 | 24.742 | 15.558 | 48.833 | 1.00 | 10.48 | BTKB |
| ATOM | 3582 | CG | PHE | 583 | 25.754 | 15.223 | 49.901 | 1.00 | 8.42 | BTKB |
| ATOM | 3583 | CD1 | PHE | 583 | 27.045 | 15.745 | 49.847 | 1.00 | 7.25 | BTKB |
| ATOM | 3584 | CD2 | PHE | 583 | 25.425 | 14.361 | 50.936 | 1.00 | 7.08 | BTKB |
| ATOM | 3585 | CE1 | PHE | 583 | 27.995 | 15.408 | 50.807 | 1.00 | 4.56 | BTKB |
| ATOM | 3586 | CE2 | PHE | 583 | 26.361 | 14.020 | 51.895 | 1.00 | 6.14 | BTKB |
| ATOM | 3587 | CZ | PHE | 583 | 27.654 | 14.543 | 51.830 | 1.00 | 8.80 | BTKB |
| ATOM | 3588 | C | PHE | 583 | 25.295 | 13.424 | 47.682 | 1.00 | 9.03 | BTKB |
| ATOM | 3589 | O | PHE | 583 | 26.435 | 13.005 | 47.927 | 1.00 | 3.30 | BTKB |
| ATOM | 3590 | N | GLY | 584 | 24.225 | 12.640 | 47.618 | 1.00 | 10.23 | BTKB |
| ATOM | 3591 | CA | GLY | 584 | 24.340 | 11.203 | 47.786 | 1.00 | 9.17 | BTKB |
| ATOM | 3592 | C | GLY | 584 | 25.042 | 10.597 | 46.593 | 1.00 | 7.39 | BTKB |
| ATOM | 3593 | O | GLY | 584 | 25.857 | 9.690 | 46.735 | 1.00 | 10.68 | BTKB |
| ATOM | 3594 | N | VAL | 585 | 24.728 | 11.112 | 45.408 | 1.00 | 9.02 | BTKB |
| ATOM | 3595 | CA | VAL | 585 | 25.350 | 10.624 | 44.186 | 1.00 | 7.71 | BTKB |
| ATOM | 3596 | CB | VAL | 585 | 24.658 | 11.184 | 42.925 | 1.00 | 6.04 | BTKB |
| ATOM | 3597 | CG1 | VAL | 585 | 25.359 | 10.699 | 41.669 | 1.00 | 4.31 | BTKB |
| ATOM | 3598 | CG2 | VAL | 585 | 23.208 | 10.747 | 42.896 | 1.00 | 6.37 | BTKB |
| ATOM | 3599 | C | VAL | 585 | 26.818 | 11.010 | 44.211 | 1.00 | 7.83 | BTKB |
| ATOM | 3600 | O | VAL | 585 | 27.665 | 10.264 | 43.720 | 1.00 | 10.87 | BTKB |
| ATOM | 3601 | N | LEU | 586 | 27.112 | 12.183 | 44.778 | 1.00 | 7.00 | BTKB |
| ATOM | 3602 | CA | LEU | 586 | 28.484 | 12.670 | 44.899 | 1.00 | 4.53 | BTKB |
| ATOM | 3603 | CB | LEU | 586 | 28.522 | 14.102 | 45.455 | 1.00 | 2.00 | BTKB |
| ATOM | 3604 | CG | LEU | 586 | 29.880 | 14.670 | 45.894 | 1.00 | 2.12 | BTKB |
| ATOM | 3605 | CD1 | LEU | 586 | 30.010 | 16.106 | 45.440 | 1.00 | 7.20 | BTKB |
| ATOM | 3606 | CD2 | LEU | 586 | 30.029 | 14.602 | 47.413 | 1.00 | 4.41 | BTKB |
| ATOM | 3607 | C | LEU | 586 | 29.247 | 11.724 | 45.812 | 1.00 | 5.96 | BTKB |
| ATOM | 3608 | O | LEU | 586 | 30.365 | 11.323 | 45.493 | 1.00 | 8.74 | BTKB |
| ATOM | 3609 | N | MET | 587 | 28.625 | 11.339 | 46.924 | 1.00 | 6.85 | BTKB |
| ATOM | 3610 | CA | MET | 587 | 29.250 | 10.419 | 47.872 | 1.00 | 8.03 | BTKB |
| ATOM | 3611 | CB | MET | 587 | 28.314 | 10.108 | 49.042 | 1.00 | 7.46 | BTKB |
| ATOM | 3612 | CG | MET | 587 | 28.255 | 11.188 | 50.097 | 1.00 | 11.21 | BTKB |
| ATOM | 3613 | SD | MET | 587 | 27.548 | 10.557 | 51.621 | 1.00 | 9.37 | BTKB |
| ATOM | 3614 | CE | MET | 587 | 25.884 | 10.932 | 51.370 | 1.00 | 7.42 | BTKB |
| ATOM | 3615 | C | MET | 587 | 29.559 | 9.139 | 47.130 | 1.00 | 8.01 | BTKB |
| ATOM | 3616 | O | MET | 587 | 30.618 | 8.534 | 47.314 | 1.00 | 9.09 | BTKB |
| ATOM | 3617 | N | TRP | 588 | 28.613 | 8.733 | 46.290 | 1.00 | 6.67 | BTKB |
| ATOM | 3618 | CA | TRP | 588 | 28.754 | 7.332 | 45.484 | 1.00 | 4.52 | BTKB |
| ATOM | 3619 | CB | TRP | 588 | 27.460 | 7.282 | 44.706 | 1.00 | 2.40 | BTKB |
| ATOM | 3620 | CG | TRP | 588 | 27.421 | 5.979 | 44.025 | 1.00 | 4.75 | BTKB |
| ATOM | 3621 | CD2 | TRP | 588 | 27.806 | 5.713 | 42.674 | 1.00 | 7.11 | BTKB |
| ATOM | 3622 | CE2 | TRP | 588 | 27.660 | 4.323 | 42.468 | 1.00 | 6.92 | BTKB |
| ATOM | 3623 | CE3 | TRP | 588 | 28.264 | 6.509 | 41.618 | 1.00 | 8.00 | BTKB |
| ATOM | 3624 | CD1 | TRP | 588 | 27.059 | 4.787 | 44.566 | 1.00 | 4.69 | BTKB |
| ATOM | 3625 | NE1 | TRP | 588 | 27.204 | 3.786 | 43.641 | 1.00 | 6.93 | BTKB |
| ATOM | 3626 | CZ2 | TRP | 588 | 27.956 | 3.711 | 41.252 | 1.00 | 11.08 | BTKB |
| ATOM | 3627 | CZ3 | TRP | 588 | 28.563 | 5.904 | 40.409 | 1.00 | 10.43 | BTKB |
| ATOM | 3628 | CH2 | TRP | 588 | 28.406 | 4.511 | 40.235 | 1.00 | 14.47 | BTKB |
| ATOM | 3629 | C | TRP | 588 | 29.955 | 7.700 | 44.542 | 1.00 | 5.71 | BTKB |
| ATOM | 3630 | O | TRP | 588 | 30.785 | 6.780 | 44.407 | 1.00 | 2.00 | BTKB |
| ATOM | 3631 | N | GLU | 589 | 30.085 | 8.893 | 43.951 | 1.00 | 6.18 | BTKB |
| ATOM | 3632 | CA | GLU | 589 | 31.198 | 9.194 | 43.050 | 1.00 | 9.53 | BTKB |
| ATOM | 3633 | CB | GLU | 589 | 31.105 | 10.614 | 42.497 | 1.00 | 6.96 | BTKB |
| ATOM | 3634 | CG | GLU | 589 | 29.917 | 10.879 | 41.608 | 1.00 | 10.53 | BTKB |
| ATOM | 3635 | CD | GLU | 589 | 29.935 | 12.283 | 41.037 | 1.00 | 10.94 | BTKB |
| ATOM | 3636 | OE1 | GLU | 589 | 29.344 | 13.189 | 41.665 | 1.00 | 11.25 | BTKB |
| ATOM | 3637 | OE2 | GLU | 589 | 30.537 | 12.479 | 39.959 | 1.00 | 15.70 | BTKB |
| ATOM | 3638 | C | GLU | 589 | 32.507 | 9.070 | 43.823 | 1.00 | 11.10 | BTKB |
| ATOM | 3639 | O | GLU | 589 | 33.519 | 8.622 | 43.280 | 1.00 | 15.09 | BTKB |
| ATOM | 3640 | N | ILE | 590 | 32.485 | 9.475 | 45.087 | 1.00 | 9.46 | BTKB |
| ATOM | 3641 | CA | ILE | 390 | 33.668 | 9.413 | 45.928 | 1.00 | 8.87 | BTKB |
| ATOM | 3642 | CB | ILE | 590 | 33.453 | 10.208 | 47.246 | 1.00 | 3.55 | BTKB |
| ATOM | 3643 | CG2 | ILE | 590 | 34.623 | 10.013 | 48.187 | 1.00 | 2.00 | BTKB |
| ATOM | 3644 | CG1 | ILE | 590 | 33.296 | 11.696 | 46.913 | 1.00 | 2.10 | BTKB |
| ATOM | 3645 | CD | ILE | 590 | 32.967 | 12.585 | 48.066 | 1.00 | 2.00 | BTKB |
| ATOM | 3646 | C | ILE | 590 | 34.078 | 7.960 | 46.194 | 1.00 | 12.45 | BTKB |
| ATOM | 3647 | O | ILE | 590 | 35.167 | 7.525 | 45.780 | 1.00 | 11.72 | BTKB |
| ATOM | 3648 | N | TYR | 591 | 33.184 | 7.192 | 46.816 | 1.00 | 15.34 | BTKB |
| ATOM | 3649 | CA | TYR | 591 | 33.461 | 5.791 | 47.111 | 1.00 | 13.05 | BTKB |
| ATOM | 3650 | CB | TYR | 591 | 32.425 | 5.230 | 48.090 | 1.00 | 12.55 | BTKB |
| ATOM | 3651 | CG | TYR | 591 | 32.669 | 5.737 | 49.488 | 1.00 | 10.68 | BTKB |
| ATOM | 3652 | CD1 | TYR | 591 | 32.075 | 6.918 | 49.936 | 1.00 | 9.88 | BTKB |
| ATOM | 3653 | CE1 | TYR | 591 | 32.415 | 7.474 | 51.175 | 1.00 | 8.76 | BTKB |
| ATOM | 3654 | CD2 | TYR | 591 | 33.598 | 5.110 | 50.321 | 1.00 | 13.54 | BTKB |
| ATOM | 3655 | CE2 | TYR | 591 | 33.940 | 5.653 | 51.564 | 1.00 | 10.89 | BTKB |
| ATOM | 3656 | CZ | TYR | 591 | 33.349 | 6.836 | 51.978 | 1.00 | 9.81 | BTKB |
| ATOM | 3657 | OH | TYR | 591 | 33.712 | 7.402 | 53.171 | 1.00 | 9.51 | BTKB |
| ATOM | 3658 | C | TYR | 591 | 33.585 | 4.938 | 45.850 | 1.00 | 12.23 | BTKB |
| ATOM | 3659 | O | TYR | 591 | 34.024 | 3.790 | 45.909 | 1.00 | 17.98 | BTKB |
| ATOM | 3660 | N | SER | 592 | 33.185 | 5.499 | 44.713 | 1.00 | 11.77 | BTKB |
| ATOM | 3661 | CA | SER | 592 | 33.290 | 4.803 | 43.438 | 1.00 | 11.46 | BTKB |
| ATOM | 3662 | CB | SER | 592 | 32.104 | 5.134 | 42.543 | 1.00 | 9.01 | BTKB |
| ATOM | 3663 | OG | SER | 592 | 30.947 | 4.504 | 43.038 | 1.00 | 12.06 | BTKB |
| ATOM | 3664 | C | SER | 592 | 34.589 | 5.180 | 42.739 | 1.00 | 12.26 | BTKB |
| ATOM | 3665 | O | SER | 592 | 34.888 | 4.685 | 41.654 | 1.00 | 11.21 | BTKB |
| ATOM | 3666 | N | LEU | 593 | 35.342 | 6.075 | 43.367 | 1.00 | 11.73 | BTKB |
| ATOM | 3667 | CA | LEU | 593 | 36.608 | 6.528 | 42.835 | 1.00 | 13.65 | BTKB |
| ATOM | 3668 | CB | LEU | 593 | 37.646 | 5.408 | 42.919 | 1.00 | 14.04 | BTKB |
| ATOM | 3669 | CG | LEU | 593 | 38.563 | 5.446 | 44.143 | 1.00 | 15.85 | BTKB |
| ATOM | 3670 | CD1 | LEU | 593 | 37.764 | 5.702 | 45.398 | 1.00 | 12.60 | BTKB |
| ATOM | 3671 | CD2 | LEU | 593 | 39.346 | 4.161 | 44.251 | 1.00 | 12.99 | BTKB |
| ATOM | 3672 | C | LEU | 593 | 36.494 | 7.086 | 41.422 | 1.00 | 14.18 | BTKB |
| ATOM | 3673 | O | LEU | 593 | 37.319 | 6.795 | 40.554 | 1.00 | 16.11 | BTKB |
| ATOM | 3674 | N | GLY | 594 | 35.464 | 7.899 | 41.200 | 1.00 | 14.51 | BTKB |
| ATOM | 3675 | CA | GLY | 594 | 35.274 | 8.508 | 39.897 | 1.00 | 13.72 | BTKB |
| ATOM | 3676 | C | GLY | 594 | 34.316 | 7.825 | 38.946 | 1.00 | 14.52 | BTKB |
| ATOM | 3677 | O | GLY | 594 | 34.060 | 8.347 | 37.868 | 1.00 | 16.91 | BTKB |
| ATOM | 3678 | N | LYS | 595 | 33.794 | 6.662 | 39.321 | 1.00 | 13.58 | BTKB |
| ATOM | 3679 | CA | LYS | 595 | 32.846 | 5.947 | 38.470 | 1.00 | 13.97 | BTKB |
| ATOM | 3680 | CB | LYS | 595 | 32.358 | 4.663 | 39.138 | 1.00 | 13.75 | BTKB |
| ATOM | 3681 | CG | LYS | 595 | 33.049 | 3.412 | 38.664 | 1.00 | 13.60 | BTKB |
| ATOM | 3682 | CD | LYS | 595 | 32.338 | 2.180 | 39.200 | 1.00 | 17.92 | BTKB |
| ATOM | 3683 | CE | LYS | 595 | 32.493 | 2.037 | 40.711 | 1.00 | 17.12 | BTKB |
| ATOM | 3684 | NZ | LYS | 595 | 31.791 | 0.819 | 41.230 | 1.00 | 20.68 | BTKB |
| ATOM | 3685 | C | LYS | 595 | 31.644 | 6.812 | 38.144 | 1.00 | 14.65 | BTKB |
| ATOM | 3686 | O | LYS | 595 | 31.032 | 7.404 | 39.031 | 1.00 | 17.03 | BTKB |
| ATOM | 3687 | N | MET | 596 | 31.300 | 6.865 | 36.866 | 1.00 | 17.91 | BTKB |
| ATOM | 3688 | CA | MET | 596 | 30.170 | 7.656 | 36.406 | 1.00 | 18.99 | BTKB |
| ATOM | 3689 | CB | MET | 596 | 30.204 | 7.773 | 34.877 | 1.00 | 20.77 | BTKB |
| ATOM | 3690 | CG | MET | 596 | 29.765 | 9.127 | 34.318 | 1.00 | 27.47 | BTKB |
| ATOM | 3691 | SD | MET | 596 | 28.020 | 9.269 | 33.847 | 1.00 | 36.00 | BTKB |
| ATOM | 3692 | CE | MET | 596 | 27.936 | 8.063 | 32.504 | 1.00 | 33.44 | BTKB |
| ATOM | 3693 | C | MET | 596 | 28.908 | 6.942 | 36.856 | 1.00 | 18.04 | BTKB |
| ATOM | 3694 | O | MET | 596 | 28.785 | 5.731 | 36.683 | 1.00 | 17.78 | BTKB |
| ATOM | 3695 | N | PRO | 597 | 27.968 | 7.677 | 37.469 | 1.00 | 16.14 | BTKB |
| ATOM | 3696 | CD | PRO | 597 | 28.043 | 9.106 | 37.815 | 1.00 | 17.99 | BTKB |
| ATOM | 3697 | CA | PRO | 597 | 26.712 | 7.101 | 37.947 | 1.00 | 15.23 | BTKB |
| ATOM | 3698 | CB | PRO | 597 | 26.015 | 8.295 | 38.602 | 1.00 | 15.62 | BTKB |
| ATOM | 3699 | CG | PRO | 597 | 27.134 | 9.181 | 39.007 | 1.00 | 17.68 | BTKB |
| ATOM | 3700 | C | PRO | 597 | 25.866 | 6.546 | 36.811 | 1.00 | 16.15 | BTKB |
| ATOM | 3701 | O | PRO | 597 | 25.801 | 7.123 | 35.726 | 1.00 | 17.62 | BTKB |
| ATOM | 3702 | N | TYR | 598 | 25.222 | 5.417 | 37.081 | 1.00 | 15.61 | BTKB |
| ATOM | 3703 | CA | TYR | 598 | 24.338 | 4.759 | 36.126 | 1.00 | 15.98 | BTKB |
| ATOM | 3704 | CB | TYR | 598 | 23.083 | 5.610 | 35.896 | 1.00 | 15.24 | BTKB |
| ATOM | 3705 | CG | TYR | 598 | 22.397 | 5.998 | 37.185 | 1.00 | 14.48 | BTKB |
| ATOM | 3706 | CD1 | TYR | 598 | 21.945 | 5.028 | 38.073 | 1.00 | 13.82 | BTKB |
| ATOM | 3707 | CE1 | TYR | 598 | 21.387 | 5.377 | 39.290 | 1.00 | 11.98 | BTKB |
| ATOM | 3708 | CD2 | TYR | 598 | 22.261 | 7.332 | 37.551 | 1.00 | 14.46 | BTKB |
| ATOM | 3709 | CE2 | TYR | 598 | 21.701 | 7.685 | 38.766 | 1.00 | 10.83 | BTKB |
| ATOM | 3710 | CZ | TYR | 598 | 21.267 | 6.704 | 39.629 | 1.00 | 5.19 | BTKB |
| ATOM | 3711 | OH | TYR | 598 | 20.703 | 7.054 | 40.837 | 1.00 | 8.13 | BTKB |
| ATOM | 3712 | C | TYR | 598 | 24.985 | 4.381 | 34.801 | 1.00 | 19.01 | BTKB |
| ATOM | 3713 | O | TYR | 598 | 24.347 | 4.425 | 33.757 | 1.00 | 23.02 | BTKB |
| ATOM | 3714 | N | GLU | 599 | 26.262 | 4.022 | 34.847 | 1.00 | 21.18 | BTKB |
| ATOM | 3715 | CA | GLU | 599 | 26.990 | 3.602 | 33.653 | 1.00 | 20.51 | BTKB |
| ATOM | 3716 | CB | GLU | 599 | 28.421 | 3.189 | 34.006 | 1.00 | 25.07 | BTKB |
| ATOM | 3717 | CG | GLU | 599 | 28.524 | 2.125 | 35.102 | 1.00 | 30.47 | BTKB |
| ATOM | 3718 | CD | GLU | 599 | 28.319 | 2.690 | 36.504 | 1.00 | 26.36 | BTKB |
| ATOM | 3719 | OE1 | GLU | 599 | 29.312 | 3.160 | 37.099 | 1.00 | 28.29 | BTKB |
| ATOM | 3720 | OE2 | GLU | 599 | 27.171 | 2.665 | 37.008 | 1.00 | 27.77 | BTKB |
| ATOM | 3721 | C | GLU | 599 | 26.245 | 2.417 | 33.071 | 1.00 | 21.77 | BTKB |
| ATOM | 3722 | O | GLU | 599 | 25.609 | 1.661 | 33.806 | 1.00 | 21.45 | BTKB |
| ATOM | 3723 | N | ARG | 600 | 26.332 | 2.250 | 31.757 | 1.00 | 24.08 | BTKB |
| ATOM | 3724 | CA | ARG | 600 | 25.637 | 1.164 | 31.062 | 1.00 | 27.74 | BTKB |
| ATOM | 3725 | CB | ARG | 600 | 25.784 | −0.179 | 31.806 | 1.00 | 31.38 | BTKB |
| ATOM | 3726 | CG | ARG | 600 | 26.892 | −1.065 | 31.269 | 1.00 | 33.68 | BTKB |
| ATOM | 3727 | CD | ARG | 600 | 26.456 | −1.680 | 29.954 | 1.00 | 34.17 | BTKB |
| ATOM | 3728 | NE | ARG | 600 | 27.570 | −1.896 | 29.039 | 1.00 | 38.49 | BTKB |
| ATOM | 3729 | CZ | ARG | 600 | 27.804 | −1.145 | 27.968 | 1.00 | 37.73 | BTKB |
| ATOM | 3730 | NH1 | ARG | 600 | 28.841 | −1.413 | 27.179 | 1.00 | 37.45 | BTKB |
| ATOM | 3731 | NH2 | ARG | 600 | 27.001 | −0.123 | 27.685 | 1.00 | 39.50 | BTKB |
| ATOM | 3732 | C | ARG | 600 | 24.165 | 1.520 | 30.860 | 1.00 | 27.26 | BTKB |
| ATOM | 3733 | O | ARG | 600 | 23.356 | 0.679 | 30.479 | 1.00 | 25.64 | BTKB |
| ATOM | 3734 | N | PHE | 601 | 23.830 | 2.775 | 31.133 | 1.00 | 27.59 | BTKB |
| ATOM | 3735 | CA | PHE | 601 | 22.479 | 3.289 | 30.962 | 1.00 | 26.40 | BTKB |
| ATOM | 3736 | CB | PHE | 601 | 21.869 | 3.704 | 32.302 | 1.00 | 23.15 | BTKB |
| ATOM | 3737 | CG | PHE | 601 | 21.279 | 2.576 | 33.091 | 1.00 | 17.15 | BTKB |
| ATOM | 3738 | CD1 | PHE | 601 | 19.961 | 2.190 | 32.887 | 1.00 | 20.02 | BTKB |
| ATOM | 3739 | CD2 | PHE | 601 | 22.017 | 1.944 | 34.083 | 1.00 | 19.77 | BTKB |
| ATOM | 3740 | CE1 | PHE | 601 | 19.381 | 1.194 | 33.665 | 1.00 | 17.60 | BTKB |
| ATOM | 3741 | CE2 | PHE | 601 | 21.449 | 0.951 | 34.864 | 1.00 | 16.15 | BTKB |
| ATOM | 3742 | CZ | PHE | 601 | 20.126 | 0.575 | 34.655 | 1.00 | 20.16 | BTKB |
| ATOM | 3743 | C | PHE | 601 | 22.617 | 4.534 | 30.105 | 1.00 | 28.08 | BTKB |
| ATOM | 3744 | O | PHE | 601 | 23.705 | 5.111 | 30.011 | 1.00 | 28.57 | BTKB |
| ATOM | 3745 | N | THR | 602 | 21.534 | 4.915 | 29.436 | 1.00 | 30.07 | BTKB |
| ATOM | 3746 | CA | THR | 602 | 21.530 | 6.116 | 28.610 | 1.00 | 30.13 | BTKB |
| ATOM | 3747 | CB | THR | 602 | 20.733 | 5.924 | 27.293 | 1.00 | 28.51 | BTKB |
| ATOM | 3748 | OG1 | THR | 602 | 19.384 | 5.543 | 27.592 | 1.00 | 27.73 | BTKB |
| ATOM | 3749 | CG2 | THR | 602 | 21.379 | 4.867 | 26.417 | 1.00 | 32.12 | BTKB |
| ATOM | 3750 | C | THR | 602 | 20.820 | 7.140 | 29.476 | 1.00 | 31.17 | BTKB |
| ATOM | 3751 | O | THR | 602 | 20.274 | 6.785 | 30.524 | 1.00 | 32.73 | BTKB |
| ATOM | 3752 | N | ASN | 603 | 20.787 | 8.394 | 29.042 | 1.00 | 30.48 | BTKB |
| ATOM | 3753 | CA | ASN | 603 | 20.117 | 9.419 | 29.827 | 1.00 | 30.44 | BTKB |
| ATOM | 3754 | CB | ASN | 603 | 20.389 | 10.807 | 29.261 | 1.00 | 32.39 | BTKB |
| ATOM | 3755 | CG | ASN | 603 | 21.239 | 11.644 | 30.186 | 1.00 | 30.79 | BTKB |
| ATOM | 3756 | OD1 | ASN | 603 | 21.885 | 11.123 | 31.095 | 1.00 | 31.05 | BTKB |
| ATOM | 3757 | ND2 | ASN | 603 | 21.238 | 12.950 | 29.968 | 1.00 | 32.87 | BTKB |
| ATOM | 3758 | C | ASN | 603 | 18.623 | 9.167 | 29.925 | 1.00 | 29.48 | BTKB |
| ATOM | 3759 | O | ASN | 603 | 18.041 | 9.265 | 31.009 | 1.00 | 24.85 | BTKB |
| ATOM | 3760 | N | SER | 604 | 18.011 | 8.817 | 28.797 | 1.00 | 29.99 | BTKB |
| ATOM | 3761 | CA | SER | 604 | 16.578 | 8.537 | 28.755 | 1.00 | 31.56 | BTKB |
| ATOM | 3762 | CB | SER | 604 | 16.102 | 8.397 | 27.304 | 1.00 | 32.42 | BTKB |
| ATOM | 3763 | OG | SER | 604 | 16.425 | 9.551 | 26.541 | 1.00 | 31.79 | BTKB |
| ATOM | 3764 | C | SER | 604 | 16.245 | 7.270 | 29.545 | 1.00 | 32.01 | BTKB |
| ATOM | 3765 | O | SER | 604 | 15.194 | 7.182 | 30.181 | 1.00 | 34.74 | BTKB |
| ATOM | 3766 | N | GLU | 605 | 17.158 | 6.303 | 29.533 | 1.00 | 31.68 | BTKB |
| ATOM | 3767 | CA | GLU | 605 | 16.954 | 5.052 | 30.252 | 1.00 | 29.12 | BTKB |
| ATOM | 3768 | CB | GLU | 605 | 17.914 | 3.982 | 29.734 | 1.00 | 30.98 | BTKB |
| ATOM | 3769 | CG | GLU | 605 | 17.561 | 2.565 | 30.172 | 1.00 | 35.34 | BTKB |
| ATOM | 3770 | CD | GLU | 605 | 18.487 | 1.517 | 29.576 | 1.00 | 36.90 | BTKB |
| ATOM | 3771 | OE1 | GLU | 605 | 19.699 | 1.805 | 29.453 | 1.00 | 38.25 | BTKB |
| ATOM | 3772 | OE2 | GLU | 605 | 18.005 | 0.412 | 29.231 | 1.00 | 34.49 | BTKB |
| ATOM | 3773 | C | GLU | 605 | 17.148 | 5.256 | 31.753 | 1.00 | 29.75 | BTKB |
| ATOM | 3774 | O | GLU | 605 | 16.421 | 4.677 | 32.565 | 1.00 | 32.16 | BTKB |
| ATOM | 3775 | N | THR | 606 | 18.131 | 6.074 | 32.116 | 1.00 | 26.59 | BTKB |
| ATOM | 3776 | CA | THR | 606 | 18.422 | 6.373 | 33.512 | 1.00 | 20.20 | BTKB |
| ATOM | 3777 | CB | THR | 606 | 19.691 | 7.243 | 33.621 | 1.00 | 18.39 | BTKB |
| ATOM | 3778 | OG1 | THR | 606 | 20.802 | 6.526 | 33.066 | 1.00 | 16.50 | BTKB |
| ATOM | 3779 | CG2 | THR | 606 | 19.987 | 7.603 | 35.079 | 1.00 | 13.19 | BTKB |
| ATOM | 3780 | C | THR | 606 | 17.233 | 7.103 | 34.139 | 1.00 | 20.29 | BTKB |
| ATOM | 3781 | O | THR | 606 | 16.828 | 6.813 | 35.274 | 1.00 | 20.04 | BTKB |
| ATOM | 3782 | N | ALA | 607 | 16.663 | 8.029 | 33.371 | 1.00 | 22.46 | BTKB |
| ATOM | 3783 | CA | ALA | 607 | 15.510 | 8.816 | 33.801 | 1.00 | 22.03 | BTKB |
| ATOM | 3784 | CB | ALA | 607 | 15.165 | 9.859 | 32.730 | 1.00 | 21.28 | BTKB |
| ATOM | 3785 | C | ALA | 607 | 14.291 | 7.932 | 34.108 | 1.00 | 21.12 | BTKB |
| ATOM | 3786 | O | ALA | 607 | 13.614 | 8.121 | 35.123 | 1.00 | 21.53 | BTKB |
| ATOM | 3787 | N | GLU | 608 | 14.036 | 6.950 | 33.247 | 1.00 | 21.20 | BTKB |
| ATOM | 3788 | CA | GLU | 608 | 12.897 | 6.052 | 33.439 | 1.00 | 22.97 | BTKB |
| ATOM | 3789 | CB | GLU | 608 | 12.661 | 5.208 | 32.176 | 1.00 | 23.32 | BTKB |
| ATOM | 3790 | CG | GLU | 608 | 11.367 | 4.380 | 32.194 | 1.00 | 27.71 | BTKB |
| ATOM | 3791 | CD | GLU | 608 | 11.607 | 2.865 | 32.186 | 1.00 | 30.68 | BTKB |
| ATOM | 3792 | OE1 | GLU | 608 | 12.115 | 2.334 | 31.172 | 1.00 | 33.86 | BTKB |
| ATOM | 3793 | OE2 | GLU | 608 | 11.273 | 2.203 | 33.195 | 1.00 | 29.86 | BTKB |
| ATOM | 3794 | C | GLU | 608 | 13.096 | 5.136 | 34.651 | 1.00 | 23.05 | BTKB |
| ATOM | 3795 | O | GLU | 608 | 12.171 | 4.902 | 35.445 | 1.00 | 21.90 | BTKB |
| ATOM | 3796 | N | HIS | 609 | 14.316 | 4.633 | 34.789 | 1.00 | 20.59 | BTKB |
| ATOM | 3797 | CA | HIS | 609 | 14.652 | 3.729 | 35.870 | 1.00 | 18.86 | BTKB |
| ATOM | 3798 | CB | HIS | 609 | 15.996 | 3.060 | 35.595 | 1.00 | 21.17 | BTKB |
| ATOM | 3799 | CG | HIS | 609 | 15.908 | 1.918 | 34.634 | 1.00 | 23.56 | BTKB |
| ATOM | 3800 | CD2 | HIS | 609 | 15.503 | 0.640 | 34.810 | 1.00 | 28.37 | BTKB |
| ATOM | 3801 | ND1 | HIS | 609 | 16.253 | 2.032 | 33.306 | 1.00 | 27.89 | BTKB |
| ATOM | 3802 | CE1 | HIS | 609 | 16.064 | 0.872 | 32.702 | 1.00 | 30.83 | BTKB |
| ATOM | 3803 | NE2 | HIS | 609 | 15.610 | 0.010 | 33.594 | 1.00 | 30.60 | BTKB |
| ATOM | 3804 | C | HIS | 609 | 14.602 | 4.306 | 37.281 | 1.00 | 16.90 | BTKB |
| ATOM | 3805 | O | HIS | 609 | 14.136 | 3.629 | 38.199 | 1.00 | 21.44 | BTKB |
| ATOM | 3806 | N | ILE | 610 | 15.061 | 5.541 | 37.466 | 1.00 | 15.25 | BTKB |
| ATOM | 3807 | CA | ILE | 610 | 15.039 | 6.149 | 38.795 | 1.00 | 10.99 | BTKB |
| ATOM | 3808 | CB | ILE | 610 | 15.687 | 7.562 | 38.817 | 1.00 | 10.27 | BTKB |
| ATOM | 3809 | CG2 | ILE | 610 | 15.697 | 8.115 | 40.226 | 1.00 | 10.71 | BTKB |
| ATOM | 3810 | CG1 | ILE | 610 | 17.131 | 7.499 | 38.329 | 1.00 | 9.01 | BTKB |
| ATOM | 3811 | CD | ILE | 610 | 17.972 | 6.524 | 39.067 | 1.00 | 13.90 | BTKB |
| ATOM | 3812 | C | ILE | 610 | 13.608 | 6.244 | 39.312 | 1.00 | 11.94 | BTKB |
| ATOM | 3813 | O | ILE | 610 | 13.335 | 5.926 | 40.462 | 1.00 | 15.24 | BTKB |
| ATOM | 3814 | N | ALA | 611 | 12.690 | 6.638 | 38.442 | 1.00 | 15.61 | BTKB |
| ATOM | 3815 | CA | ALA | 611 | 11.291 | 6.767 | 38.823 | 1.00 | 18.54 | BTKB |
| ATOM | 3816 | CB | ALA | 611 | 10.484 | 7.345 | 37.668 | 1.00 | 18.01 | BTKB |
| ATOM | 3817 | C | ALA | 611 | 10.697 | 5.431 | 39.270 | 1.00 | 21.52 | BTKB |
| ATOM | 3818 | O | ALA | 611 | 9.972 | 5.365 | 40.267 | 1.00 | 24.49 | BTKB |
| ATOM | 3819 | N | GLN | 612 | 11.023 | 4.366 | 38.546 | 1.00 | 23.83 | BTKB |
| ATOM | 3820 | CA | GLN | 612 | 10.507 | 3.038 | 38.865 | 1.00 | 22.70 | BTKB |
| ATOM | 3821 | CB | GLN | 612 | 10.736 | 2.091 | 37.691 | 1.00 | 26.14 | BTKB |
| ATOM | 3822 | CG | GLN | 612 | 10.119 | 2.574 | 36.374 | 1.00 | 29.22 | BTKB |
| ATOM | 3823 | CD | GLN | 612 | 8.597 | 2.714 | 36.418 | 1.00 | 34.44 | BTKB |
| ATOM | 3824 | OE1 | GLN | 612 | 7.956 | 2.447 | 37.434 | 1.00 | 32.43 | BTKB |
| ATOM | 3825 | NE2 | GLN | 612 | 8.017 | 3.128 | 35.299 | 1.00 | 33.12 | BTKB |
| ATOM | 3826 | C | GLN | 612 | 11.084 | 2.453 | 40.151 | 1.00 | 21.66 | BTKB |
| ATOM | 3827 | O | GLN | 612 | 10.501 | 1.541 | 40.741 | 1.00 | 23.34 | BTKB |
| ATOM | 3828 | N | GLY | 613 | 12.224 | 2.975 | 40.593 | 1.00 | 20.85 | BTKB |
| ATOM | 3829 | CA | GLY | 613 | 12.816 | 2.488 | 41.824 | 1.00 | 17.52 | BTKB |
| ATOM | 3830 | C | GLY | 613 | 14.300 | 2.188 | 41.764 | 1.00 | 17.19 | BTKB |
| ATOM | 3831 | O | GLY | 613 | 14.892 | 1.783 | 42.769 | 1.00 | 16.74 | BTKB |
| ATOM | 3832 | N | LEU | 614 | 14.902 | 2.366 | 40.594 | 1.00 | 14.38 | BTKB |
| ATOM | 3833 | CA | LEU | 614 | 16.327 | 2.105 | 40.427 | 1.00 | 13.63 | BTKB |
| ATOM | 3834 | CB | LEU | 614 | 16.701 | 2.152 | 38.947 | 1.00 | 11.11 | BTKB |
| ATOM | 3835 | CG | LEU | 614 | 18.127 | 1.939 | 38.433 | 1.00 | 10.02 | BTKB |
| ATOM | 3836 | CD1 | LEU | 614 | 18.886 | 3.239 | 38.427 | 1.00 | 8.37 | BTKB |
| ATOM | 3837 | CD2 | LEU | 614 | 18.842 | 0.853 | 39.211 | 1.00 | 13.13 | BTKB |
| ATOM | 3838 | C | LEU | 614 | 17.183 | 3.065 | 41.248 | 1.00 | 13.27 | BTKB |
| ATOM | 3839 | O | LEU | 614 | 16.938 | 4.274 | 41.278 | 1.00 | 11.31 | BTKB |
| ATOM | 3840 | N | ARG | 615 | 18.206 | 2.502 | 41.889 | 1.00 | 13.39 | BTKB |
| ATOM | 3841 | CA | ARG | 615 | 19.130 | 3.240 | 42.735 | 1.00 | 11.32 | BTKB |
| ATOM | 3842 | CB | ARG | 615 | 18.781 | 3.016 | 44.206 | 1.00 | 13.75 | BTKB |
| ATOM | 3843 | CG | ARG | 615 | 17.381 | 3.443 | 44.614 | 1.00 | 14.44 | BTKB |
| ATOM | 3844 | CD | ARG | 615 | 17.228 | 4.946 | 44.642 | 1.00 | 12.94 | BTKB |
| ATOM | 3845 | NE | ARG | 615 | 15.899 | 5.303 | 45.124 | 1.00 | 11.71 | BTKB |
| ATOM | 3846 | CZ | ARG | 615 | 14.893 | 5.685 | 44.345 | 1.00 | 11.61 | BTKB |
| ATOM | 3847 | NH1 | ARG | 615 | 15.058 | 5.772 | 43.036 | 1.00 | 13.30 | BTKB |
| ATOM | 3848 | NH2 | ARG | 615 | 13.708 | 5.949 | 44.876 | 1.00 | 16.80 | BTKB |
| ATOM | 3849 | C | ARG | 615 | 20.564 | 2.773 | 42.501 | 1.00 | 12.33 | BTKB |
| ATOM | 3850 | O | ARG | 615 | 20.807 | 1.751 | 41.850 | 1.00 | 14.35 | BTKB |
| ATOM | 3851 | N | LEU | 616 | 21.504 | 3.526 | 43.066 | 1.00 | 17.12 | BTKB |
| ATOM | 3852 | CA | LEU | 616 | 22.936 | 3.251 | 42.967 | 1.00 | 13.55 | BTKB |
| ATOM | 3853 | CB | LEU | 616 | 23.730 | 4.525 | 43.258 | 1.00 | 12.49 | BTKB |
| ATOM | 3854 | CG | LEU | 616 | 24.452 | 5.273 | 42.132 | 1.00 | 15.47 | BTKB |
| ATOM | 3855 | CD1 | LEU | 616 | 24.288 | 4.570 | 40.788 | 1.00 | 20.12 | BTKB |
| ATOM | 3856 | CD2 | LEU | 616 | 23.990 | 6.720 | 42.086 | 1.00 | 11.52 | BTKB |
| ATOM | 3857 | C | LEU | 616 | 23.337 | 2.177 | 43.961 | 1.00 | 12.84 | BTKB |
| ATOM | 3858 | O | LEU | 616 | 22.963 | 2.234 | 45.135 | 1.00 | 11.91 | BTKB |
| ATOM | 3859 | N | TYR | 617 | 24.100 | 1.200 | 43.483 | 1.00 | 12.87 | BTKB |
| ATOM | 3860 | CA | TYR | 617 | 24.561 | 0.109 | 44.322 | 1.00 | 8.22 | BTKB |
| ATOM | 3861 | CB | TYR | 617 | 25.037 | −1.067 | 43.453 | 1.00 | 7.64 | BTKB |
| ATOM | 3862 | CG | TYR | 617 | 26.116 | −0.724 | 42.444 | 1.00 | 4.38 | BTKB |
| ATOM | 3863 | CD1 | TYR | 617 | 27.400 | −0.394 | 42.852 | 1.00 | 5.18 | BTKB |
| ATOM | 3864 | CE1 | TYR | 617 | 28.382 | −0.065 | 41.935 | 1.00 | 5.80 | BTKB |
| ATOM | 3865 | CD2 | TYR | 617 | 25.843 | −0.722 | 41.082 | 1.00 | 6.41 | BTKB |
| ATOM | 3866 | CE2 | TYR | 617 | 26.827 | −0.395 | 40.148 | 1.00 | 7.22 | BTKB |
| ATOM | 3867 | CZ | TYR | 617 | 28.092 | −0.070 | 40.584 | 1.00 | 7.64 | BTKB |
| ATOM | 3868 | OH | TYR | 617 | 29.082 | 0.223 | 39.674 | 1.00 | 9.96 | BTKB |
| ATOM | 3869 | C | TYR | 617 | 25.679 | 0.600 | 45.239 | 1.00 | 8.77 | BTKB |
| ATOM | 3870 | O | TYR | 617 | 26.286 | 1.631 | 44.990 | 1.00 | 7.36 | BTKB |
| ATOM | 3871 | N | ARG | 618 | 25.952 | −0.147 | 46.295 | 1.00 | 8.18 | BTKB |
| ATOM | 3872 | CA | ARG | 618 | 27.001 | 0.219 | 47.229 | 1.00 | 8.36 | BTKB |
| ATOM | 3873 | CB | ARG | 618 | 26.879 | −0.618 | 48.511 | 1.00 | 6.48 | BTKB |
| ATOM | 3874 | CG | ARG | 618 | 27.967 | −0.360 | 49.527 | 1.00 | 6.90 | BTKB |
| ATOM | 3875 | CD | ARG | 618 | 27.593 | −0.867 | 50.907 | 1.00 | 9.82 | BTKB |
| ATOM | 3876 | NE | ARG | 618 | 27.871 | −2.281 | 51.118 | 1.00 | 14.88 | BTKB |
| ATOM | 3877 | CZ | ARG | 618 | 28.894 | −2.735 | 51.835 | 1.00 | 13.15 | BTKB |
| ATOM | 3878 | NH1 | ARG | 618 | 29.733 | −1.888 | 52.394 | 1.00 | 14.59 | BTKB |
| ATOM | 3879 | NH2 | ARG | 618 | 29.052 | −4.033 | 52.047 | 1.00 | 13.61 | BTKB |
| ATOM | 3880 | C | ARG | 618 | 28.383 | 0.013 | 46.616 | 1.00 | 8.62 | BTKB |
| ATOM | 3881 | O | ARG | 618 | 28.688 | −1.062 | 46.086 | 1.00 | 10.77 | BTKB |
| ATOM | 3882 | N | PRO | 619 | 29.220 | 1.057 | 46.623 | 1.00 | 6.53 | BTKB |
| ATOM | 3883 | CD | PRO | 619 | 28.936 | 2.487 | 46.840 | 1.00 | 3.47 | BTKB |
| ATOM | 3884 | CA | PRO | 619 | 30.553 | 0.869 | 46.051 | 1.00 | 8.75 | BTKB |
| ATOM | 3885 | CB | PRO | 619 | 31.116 | 2.286 | 46.032 | 1.00 | 5.78 | BTKB |
| ATOM | 3886 | CG | PRO | 619 | 29.882 | 3.142 | 45.874 | 1.00 | 5.70 | BTKB |
| ATOM | 3887 | C | PRO | 619 | 31.382 | −0.040 | 46.962 | 1.00 | 11.49 | BTKB |
| ATOM | 3888 | O | PRO | 619 | 31.251 | −0.002 | 48.188 | 1.00 | 8.13 | BTKB |
| ATOM | 3889 | N | HIS | 620 | 32.192 | −0.896 | 46.351 | 1.00 | 13.39 | BTKB |
| ATOM | 3890 | CA | HIS | 620 | 33.058 | −1.805 | 47.090 | 1.00 | 16.43 | BTKB |
| ATOM | 3891 | CB | HIS | 620 | 34.061 | −2.507 | 46.145 | 1.00 | 23.71 | BTKB |
| ATOM | 3892 | CG | HIS | 620 | 34.288 | −1.798 | 44.833 | 1.00 | 29.79 | BTKB |
| ATOM | 3893 | CD2 | HIS | 620 | 34.138 | −0.503 | 44.468 | 1.00 | 31.60 | BTKB |
| ATOM | 3894 | ND1 | HIS | 620 | 34.719 | −2.464 | 43.704 | 1.00 | 31.86 | BTKB |
| ATOM | 3895 | CE1 | HIS | 620 | 34.820 | −1.607 | 42.699 | 1.00 | 33.57 | BTKB |
| ATOM | 3896 | NE2 | HIS | 620 | 34.471 | −0.411 | 43.140 | 1.00 | 35.83 | BTKB |
| ATOM | 3897 | C | HIS | 620 | 33.804 | −1.148 | 48.267 | 1.00 | 15.65 | BTKB |
| ATOM | 3898 | O | HIS | 620 | 33.963 | −1.764 | 49.328 | 1.00 | 14.45 | BTKB |
| ATOM | 3899 | N | LEU | 621 | 34.235 | 0.100 | 48.080 | 1.00 | 11.67 | BTKB |
| ATOM | 3900 | CA | LEU | 621 | 34.974 | 0.834 | 49.103 | 1.00 | 9.74 | BTKB |
| ATOM | 3901 | CB | LEU | 621 | 35.792 | 1.955 | 48.464 | 1.00 | 12.10 | BTKB |
| ATOM | 3902 | CG | LEU | 621 | 36.869 | 1.540 | 47.459 | 1.00 | 10.83 | BTKB |
| ATOM | 3903 | CD1 | LEU | 621 | 36.318 | 1.644 | 46.047 | 1.00 | 12.43 | BTKB |
| ATOM | 3904 | CD2 | LEU | 621 | 38.078 | 2.426 | 47.601 | 1.00 | 13.18 | BTKB |
| ATOM | 3905 | C | LEU | 621 | 34.155 | 1.404 | 50.261 | 1.00 | 10.03 | BTKB |
| ATOM | 3906 | O | LEU | 621 | 34.717 | 1.801 | 51.284 | 1.00 | 10.92 | BTKB |
| ATOM | 3907 | N | ALA | 622 | 32.836 | 1.436 | 50.101 | 1.00 | 10.32 | BTKB |
| ATOM | 3908 | CA | ALA | 622 | 31.944 | 1.964 | 51.120 | 1.00 | 11.25 | BTKB |
| ATOM | 3909 | CB | ALA | 622 | 30.700 | 2.563 | 50.460 | 1.00 | 12.08 | BTKB |
| ATOM | 3910 | C | ALA | 622 | 31.536 | 0.862 | 52.085 | 1.00 | 13.04 | BTKB |
| ATOM | 3911 | O | ALA | 622 | 31.502 | −0.307 | 51.719 | 1.00 | 15.23 | BTKB |
| ATOM | 3912 | N | SER | 623 | 31.279 | 1.230 | 53.332 | 1.00 | 14.15 | BTKB |
| ATOM | 3913 | CA | SER | 623 | 30.837 | 0.270 | 54.325 | 1.00 | 11.19 | BTKB |
| ATOM | 3914 | CB | SER | 623 | 31.416 | 0.611 | 55.696 | 1.00 | 9.54 | BTKB |
| ATOM | 3915 | OG | SER | 623 | 30.949 | 1.864 | 56.170 | 1.00 | 8.50 | BTKB |
| ATOM | 3916 | C | SER | 623 | 29.312 | 0.331 | 54.349 | 1.00 | 10.40 | BTKB |
| ATOM | 3917 | O | SER | 623 | 28.702 | 1.234 | 53.768 | 1.00 | 14.47 | BTKB |
| ATOM | 3918 | N | GLU | 624 | 28.692 | −0.651 | 54.985 | 1.00 | 10.03 | BTKB |
| ATOM | 3919 | CA | GLU | 624 | 27.238 | −0.714 | 55.093 | 1.00 | 10.70 | BTKB |
| ATOM | 3920 | CB | GLU | 624 | 26.869 | −1.867 | 56.027 | 1.00 | 15.43 | BTKB |
| ATOM | 3921 | CG | GLU | 624 | 25.423 | −1.946 | 56.491 | 1.00 | 15.78 | BTKB |
| ATOM | 3922 | CD | GLU | 624 | 25.244 | −2.977 | 57.601 | 1.00 | 19.80 | BTKB |
| ATOM | 3923 | OE1 | GLU | 624 | 26.105 | −3.060 | 58.503 | 1.00 | 22.20 | BTKB |
| ATOM | 3924 | OE2 | GLU | 624 | 24.240 | −3.709 | 57.574 | 1.00 | 22.73 | BTKB |
| ATOM | 3925 | C | GLU | 624 | 26.660 | 0.605 | 55.612 | 1.00 | 10.33 | BTKB |
| ATOM | 3926 | O | GLU | 624 | 25.627 | 1.078 | 55.124 | 1.00 | 12.18 | BTKB |
| ATOM | 3927 | N | LYS | 625 | 27.335 | 1.202 | 56.591 | 1.00 | 13.68 | BTKB |
| ATOM | 3928 | CA | LYS | 625 | 26.886 | 2.461 | 57.172 | 1.00 | 13.17 | BTKB |
| ATOM | 3929 | CB | LYS | 625 | 27.641 | 2.761 | 58.470 | 1.00 | 13.89 | BTKB |
| ATOM | 3930 | CG | LYS | 625 | 26.960 | 3.806 | 59.351 | 1.00 | 19.82 | BTKB |
| ATOM | 3931 | CD | LYS | 625 | 27.145 | 3.522 | 60.843 | 1.00 | 21.52 | BTKB |
| ATOM | 3932 | CE | LYS | 625 | 26.398 | 2.261 | 61.281 | 1.00 | 24.13 | BTKB |
| ATOM | 3933 | NZ | LYS | 625 | 26.554 | 1.946 | 62.735 | 1.00 | 26.73 | BTKB |
| ATOM | 3934 | C | LYS | 625 | 27.007 | 3.608 | 56.175 | 1.00 | 12.94 | BTKB |
| ATOM | 3935 | O | LYS | 625 | 26.074 | 4.393 | 56.023 | 1.00 | 17.08 | BTKB |
| ATOM | 3936 | N | VAL | 626 | 28.118 | 3.671 | 55.446 | 1.00 | 13.16 | BTKB |
| ATOM | 3937 | CA | VAL | 626 | 28.310 | 4.726 | 54.445 | 1.00 | 10.56 | BTKB |
| ATOM | 3938 | CB | VAL | 626 | 29.698 | 4.646 | 53.758 | 1.00 | 10.58 | BTKB |
| ATOM | 3939 | CG1 | VAL | 626 | 29.775 | 5.671 | 52.634 | 1.00 | 13.96 | BTKB |
| ATOM | 3940 | CG2 | VAL | 626 | 30.802 | 4.904 | 54.763 | 1.00 | 6.81 | BTKB |
| ATOM | 3941 | C | VAL | 626 | 27.221 | 4.653 | 53.368 | 1.00 | 8.76 | BTKB |
| ATOM | 3942 | O | VAL | 626 | 26.757 | 5.681 | 52.870 | 1.00 | 6.95 | BTKB |
| ATOM | 3943 | N | TYR | 627 | 26.827 | 3.438 | 53.001 | 1.00 | 7.31 | BTKB |
| ATOM | 3944 | CA | TYR | 627 | 25.780 | 3.243 | 52.001 | 1.00 | 7.99 | BTKB |
| ATOM | 3945 | CB | TYR | 627 | 25.674 | 1.773 | 51.598 | 1.00 | 2.15 | BTKB |
| ATOM | 3946 | CG | TYR | 627 | 24.723 | 1.544 | 50.454 | 1.00 | 4.61 | BTKB |
| ATOM | 3947 | CD1 | TYR | 627 | 24.718 | 2.400 | 49.352 | 1.00 | 10.72 | BTKB |
| ATOM | 3948 | CE1 | TYR | 627 | 23.845 | 2.201 | 48.298 | 1.00 | 9.43 | BTKB |
| ATOM | 3949 | CD2 | TYR | 627 | 23.828 | 0.482 | 50.468 | 1.00 | 5.07 | BTKB |
| ATOM | 3950 | CE2 | TYR | 627 | 22.946 | 0.273 | 49.410 | 1.00 | 6.61 | BTKB |
| ATOM | 3951 | CZ | TYR | 627 | 22.962 | 1.139 | 48.334 | 1.00 | 5.55 | BTKB |
| ATOM | 3952 | OH | TYR | 627 | 22.092 | 0.961 | 47.297 | 1.00 | 11.16 | BTKB |
| ATOM | 3953 | C | TYR | 627 | 24.429 | 3.715 | 52.518 | 1.00 | 9.39 | BTKB |
| ATOM | 3954 | O | TYR | 627 | 23.599 | 4.187 | 51.754 | 1.00 | 11.16 | BTKB |
| ATOM | 3955 | N | THR | 628 | 24.201 | 3.546 | 53.813 | 1.00 | 9.89 | BTKB |
| ATOM | 3956 | CA | THR | 628 | 22.961 | 3.976 | 54.447 | 1.00 | 9.04 | BTKB |
| ATOM | 3957 | CB | THR | 628 | 22.989 | 3.636 | 55.961 | 1.00 | 10.10 | BTKB |
| ATOM | 3958 | OG1 | THR | 628 | 23.201 | 2.229 | 56.132 | 1.00 | 13.62 | BTKB |
| ATOM | 3959 | CG2 | THR | 628 | 21.691 | 4.033 | 56.633 | 1.00 | 6.51 | BTKB |
| ATOM | 3960 | C | THR | 628 | 22.868 | 5.490 | 54.289 | 1.00 | 7.33 | BTKB |
| ATOM | 3961 | O | THR | 628 | 21.856 | 6.023 | 53.853 | 1.00 | 6.66 | BTKB |
| ATOM | 3962 | N | ILE | 629 | 23.967 | 6.167 | 54.589 | 1.00 | 6.78 | BTKB |
| ATOM | 3963 | CA | ILE | 629 | 24.031 | 7.617 | 54.493 | 1.00 | 8.03 | BTKB |
| ATOM | 3964 | CB | ILE | 629 | 25.417 | 8.151 | 54.964 | 1.00 | 6.17 | BTKB |
| ATOM | 3965 | CG2 | ILE | 629 | 25.531 | 9.649 | 54.709 | 1.00 | 5.05 | BTKB |
| ATOM | 3966 | CG1 | ILE | 629 | 25.593 | 7.854 | 56.459 | 1.00 | 5.15 | BTKB |
| ATOM | 3967 | CD | ILE | 629 | 26.999 | 8.035 | 56.969 | 1.00 | 2.34 | BTKB |
| ATOM | 3968 | C | ILE | 629 | 23.690 | 8.120 | 53.085 | 1.00 | 12.02 | BTKB |
| ATOM | 3969 | O | ILE | 629 | 22.702 | 8.842 | 52.900 | 1.00 | 13.66 | BTKB |
| ATOM | 3970 | N | MET | 630 | 24.439 | 7.676 | 52.085 | 1.00 | 11.42 | BTKB |
| ATOM | 3971 | CA | MET | 630 | 24.185 | 8.124 | 50.727 | 1.00 | 7.89 | BTKB |
| ATOM | 3972 | CB | MET | 630 | 25.249 | 7.600 | 49.756 | 1.00 | 11.44 | BTKB |
| ATOM | 3973 | CG | MET | 630 | 25.288 | 6.095 | 49.574 | 1.00 | 14.27 | BTKB |
| ATOM | 3974 | SD | MET | 630 | 26.438 | 5.613 | 48.277 | 1.00 | 14.21 | BTKB |
| ATOM | 3975 | CE | MET | 630 | 27.987 | 5.700 | 49.104 | 1.00 | 3.93 | BTKB |
| ATOM | 3976 | C | MET | 630 | 22.798 | 7.766 | 50.246 | 1.00 | 6.76 | BTKB |
| ATOM | 3977 | O | MET | 630 | 22.164 | 8.542 | 49.525 | 1.00 | 9.02 | BTKB |
| ATOM | 3978 | N | TYR | 631 | 22.303 | 6.618 | 50.696 | 1.00 | 9.53 | BTKB |
| ATOM | 3979 | CA | TYR | 631 | 20.981 | 6.143 | 50.301 | 1.00 | 9.62 | BTKB |
| ATOM | 3980 | CB | TYR | 631 | 20.837 | 4.637 | 50.583 | 1.00 | 7.23 | BTKB |
| ATOM | 3981 | CG | TYR | 631 | 19.708 | 3.978 | 49.819 | 1.00 | 9.49 | BTKB |
| ATOM | 3982 | CD1 | TYR | 631 | 18.385 | 4.120 | 50.232 | 1.00 | 12.76 | BTKB |
| ATOM | 3983 | CE1 | TYR | 631 | 17.343 | 3.539 | 49.518 | 1.00 | 12.89 | BTKB |
| ATOM | 3984 | CD2 | TYR | 631 | 19.962 | 3.233 | 48.670 | 1.00 | 8.23 | BTKB |
| ATOM | 3985 | CE2 | TYR | 631 | 18.929 | 2.646 | 47.951 | 1.00 | 11.55 | BTKB |
| ATOM | 3986 | CZ | TYR | 631 | 17.625 | 2.803 | 48.381 | 1.00 | 13.98 | BTKB |
| ATOM | 3987 | OH | TYR | 631 | 16.598 | 2.225 | 47.683 | 1.00 | 16.12 | BTKB |
| ATOM | 3988 | C | TYR | 631 | 19.844 | 6.937 | 50.950 | 1.00 | 7.30 | BTKB |
| ATOM | 3989 | O | TYR | 631 | 18.719 | 6.932 | 50.451 | 1.00 | 11.12 | BTKB |
| ATOM | 3990 | N | SER | 632 | 20.119 | 7.635 | 52.048 | 1.00 | 9.61 | BTKB |
| ATOM | 3991 | CA | SER | 632 | 19.067 | 8.423 | 52.678 | 1.00 | 9.92 | BTKB |
| ATOM | 3992 | CB | SER | 632 | 19.446 | 8.838 | 54.108 | 1.00 | 7.09 | BTKB |
| ATOM | 3993 | OG | SER | 632 | 20.592 | 9.664 | 54.130 | 1.00 | 8.65 | BTKB |
| ATOM | 3994 | C | SER | 632 | 18.759 | 9.642 | 51.800 | 1.00 | 10.16 | BTKB |
| ATOM | 3995 | O | SER | 632 | 17.648 | 10.189 | 51.820 | 1.00 | 8.62 | BTKB |
| ATOM | 3996 | N | CYS | 633 | 19.734 | 10.013 | 50.979 | 1.00 | 13.59 | BTKB |
| ATOM | 3997 | CA | CYS | 633 | 19.606 | 11.146 | 50.065 | 1.00 | 13.01 | BTKB |
| ATOM | 3998 | CB | CYS | 633 | 20.988 | 11.537 | 49.522 | 1.00 | 9.51 | BTKB |
| ATOM | 3999 | SG | CYS | 633 | 22.269 | 11.873 | 50.748 | 1.00 | 10.09 | BTKB |
| ATOM | 4000 | C | CYS | 633 | 18.688 | 10.861 | 48.869 | 1.00 | 11.24 | BTKB |
| ATOM | 4001 | O | CYS | 633 | 18.167 | 11.785 | 48.264 | 1.00 | 11.58 | BTKB |
| ATOM | 4002 | N | TRP | 634 | 18.448 | 9.590 | 48.565 | 1.00 | 12.35 | BTKB |
| ATOM | 4003 | CA | TRP | 634 | 17.649 | 9.225 | 47.396 | 1.00 | 16.45 | BTKB |
| ATOM | 4004 | CB | TRP | 634 | 18.354 | 8.109 | 46.629 | 1.00 | 14.41 | BTKB |
| ATOM | 4005 | CG | TRP | 634 | 19.803 | 8.397 | 46.406 | 1.00 | 16.37 | BTKB |
| ATOM | 4006 | CD2 | TRP | 634 | 20.885 | 7.465 | 46.489 | 1.00 | 13.01 | BTKB |
| ATOM | 4007 | CE2 | TRP | 634 | 22.067 | 8.178 | 46.219 | 1.00 | 13.10 | BTKB |
| ATOM | 4008 | CE3 | TRP | 634 | 20.965 | 6.095 | 46.765 | 1.00 | 12.84 | BTKB |
| ATOM | 4009 | CD1 | TRP | 634 | 20.361 | 9.604 | 46.101 | 1.00 | 12.67 | BTKB |
| ATOM | 4010 | NE1 | TRP | 634 | 21.717 | 9.482 | 45.987 | 1.00 | 11.33 | BTKB |
| ATOM | 4011 | CZ2 | TRP | 634 | 23.325 | 7.566 | 46.214 | 1.00 | 10.17 | BTKB |
| ATOM | 4012 | CZ3 | TRP | 634 | 22.213 | 5.490 | 46.761 | 1.00 | 12.02 | BTKB |
| ATOM | 4013 | CH2 | TRP | 634 | 23.376 | 6.227 | 46.486 | 1.00 | 4.94 | BTKB |
| ATOM | 4014 | C | TRP | 634 | 16.171 | 8.881 | 47.541 | 1.00 | 20.48 | BTKB |
| ATOM | 4015 | O | TRP | 634 | 15.588 | 8.288 | 46.633 | 1.00 | 24.09 | BTKB |
| ATOM | 4016 | N | HIS | 635 | 15.550 | 9.253 | 48.654 | 1.00 | 22.92 | BTKB |
| ATOM | 4017 | CA | HIS | 635 | 14.128 | 8.968 | 48.833 | 1.00 | 24.53 | BTKB |
| ATOM | 4018 | CB | HIS | 635 | 13.670 | 9.356 | 50.234 | 1.00 | 25.26 | BTKB |
| ATOM | 4019 | CG | HIS | 635 | 14.517 | 8.796 | 51.331 | 1.00 | 34.35 | BTKB |
| ATOM | 4020 | CD2 | HIS | 635 | 14.645 | 9.161 | 52.628 | 1.00 | 37.85 | BTKB |
| ATOM | 4021 | ND1 | HIS | 635 | 15.373 | 7.731 | 51.144 | 1.00 | 40.01 | BTKB |
| ATOM | 4022 | CE1 | HIS | 635 | 15.990 | 7.465 | 52.282 | 1.00 | 41.13 | BTKB |
| ATOM | 4023 | NE2 | HIS | 635 | 15.567 | 8.319 | 53.198 | 1.00 | 40.19 | BTKB |
| ATOM | 4024 | C | HIS | 635 | 13.361 | 9.805 | 47.810 | 1.00 | 24.66 | BTKB |
| ATOM | 4025 | O | HIS | 635 | 13.551 | 11.016 | 47.734 | 1.00 | 23.32 | BTKB |
| ATOM | 4026 | N | GLU | 636 | 12.488 | 9.179 | 47.031 | 1.00 | 26.44 | BTKB |
| ATOM | 4027 | CA | GLU | 636 | 11.734 | 9.939 | 46.043 | 1.00 | 26.89 | BTKB |
| ATOM | 4028 | CB | GLU | 636 | 10.949 | 9.006 | 45.110 | 1.00 | 28.33 | BTKB |
| ATOM | 4029 | CG | GLU | 636 | 10.700 | 9.603 | 43.709 | 1.00 | 28.10 | BTKB |
| ATOM | 4030 | CD | GLU | 636 | 10.649 | 8.559 | 42.592 | 1.00 | 22.86 | BTKB |
| ATOM | 4031 | OE1 | GLU | 636 | 9.696 | 8.602 | 41.786 | 1.00 | 21.50 | BTKB |
| ATOM | 4032 | OE2 | GLU | 636 | 11.572 | 7.715 | 42.501 | 1.00 | 21.58 | BTKB |
| ATOM | 4033 | C | GLU | 636 | 10.831 | 10.959 | 46.753 | 1.00 | 27.63 | BTKB |
| ATOM | 4034 | O | GLU | 636 | 10.420 | 11.954 | 46.156 | 1.00 | 30.62 | BTKB |
| ATOM | 4035 | N | LYS | 637 | 10.529 | 10.714 | 48.026 | 1.00 | 25.98 | BTKB |
| ATOM | 4036 | CA | LYS | 637 | 9.724 | 11.642 | 48.817 | 1.00 | 26.32 | BTKB |
| ATOM | 4037 | CB | LYS | 637 | 8.924 | 10.898 | 49.896 | 1.00 | 28.32 | BTKB |
| ATOM | 4038 | CG | LYS | 637 | 8.125 | 11.800 | 50.857 | 1.00 | 29.37 | BTKB |
| ATOM | 4039 | CD | LYS | 637 | 6.661 | 11.997 | 50.426 | 1.00 | 31.37 | BTKB |
| ATOM | 4040 | CE | LYS | 637 | 6.512 | 12.837 | 49.155 | 1.00 | 33.19 | BTKB |
| ATOM | 4041 | NZ | LYS | 637 | 5.123 | 12.793 | 48.607 | 1.00 | 32.03 | BTKB |
| ATOM | 4042 | C | LYS | 637 | 10.701 | 12.606 | 49.485 | 1.00 | 26.98 | BTKB |
| ATOM | 4043 | O | LYS | 637 | 11.212 | 12.330 | 50.575 | 1.00 | 26.07 | BTKB |
| ATOM | 4044 | N | ALA | 638 | 10.960 | 13.729 | 48.825 | 1.00 | 28.49 | BTKB |
| ATOM | 4045 | CA | ALA | 638 | 11.879 | 14.746 | 49.333 | 1.00 | 28.72 | BTKB |
| ATOM | 4046 | CB | ALA | 638 | 11.658 | 16.048 | 48.586 | 1.00 | 31.16 | BTKB |
| ATOM | 4047 | C | ALA | 638 | 11.779 | 14.985 | 50.844 | 1.00 | 28.22 | BTKB |
| ATOM | 4048 | O | ALA | 638 | 12.791 | 15.134 | 51.529 | 1.00 | 25.48 | BTKB |
| ATOM | 4049 | N | ASP | 639 | 10.554 | 15.003 | 51.355 | 1.00 | 29.23 | BTKB |
| ATOM | 4050 | CA | ASP | 639 | 10.295 | 15.234 | 52.774 | 1.00 | 28.43 | BTKB |
| ATOM | 4051 | CB | ASP | 639 | 8.779 | 15.248 | 53.031 | 1.00 | 31.75 | BTKB |
| ATOM | 4052 | CG | ASP | 639 | 8.058 | 16.380 | 52.288 | 1.00 | 34.13 | BTKB |
| ATOM | 4053 | OD1 | ASP | 639 | 6.951 | 16.765 | 52.735 | 1.00 | 33.09 | BTKB |
| ATOM | 4054 | OD2 | ASP | 639 | 8.586 | 16.881 | 51.266 | 1.00 | 36.59 | BTKB |
| ATOM | 4055 | C | ASP | 639 | 10.970 | 14.230 | 53.718 | 1.00 | 28.45 | BTKB |
| ATOM | 4056 | O | ASP | 639 | 11.329 | 14.574 | 54.848 | 1.00 | 25.09 | BTKB |
| ATOM | 4057 | N | GLU | 640 | 11.146 | 12.996 | 53.244 | 1.00 | 24.72 | BTKB |
| ATOM | 4058 | CA | GLU | 640 | 11.754 | 11.931 | 54.038 | 1.00 | 18.61 | BTKB |
| ATOM | 4059 | CB | GLU | 640 | 11.297 | 10.563 | 53.513 | 1.00 | 20.35 | BTKB |
| ATOM | 4060 | CG | GLU | 640 | 11.609 | 9.361 | 54.422 | 1.00 | 22.93 | BTKB |
| ATOM | 4061 | CD | GLU | 640 | 11.329 | 8.007 | 53.762 | 1.00 | 21.79 | BTKB |
| ATOM | 4062 | OE1 | GLU | 640 | 10.997 | 7.043 | 54.486 | 1.00 | 26.11 | BTKB |
| ATOM | 4063 | OE2 | GLU | 640 | 11.465 | 7.895 | 52.522 | 1.00 | 15.76 | BTKB |
| ATOM | 4064 | C | GLU | 640 | 13.276 | 12.021 | 54.030 | 1.00 | 18.11 | BTKB |
| ATOM | 4065 | O | GLU | 640 | 13.950 | 11.266 | 54.734 | 1.00 | 19.30 | BTKB |
| ATOM | 4066 | N | ARG | 641 | 13.821 | 12.935 | 53.233 | 1.00 | 16.37 | BTKB |
| ATOM | 4067 | CA | ARG | 641 | 15.273 | 13.108 | 53.155 | 1.00 | 14.52 | BTKB |
| ATOM | 4068 | CB | ARG | 641 | 15.682 | 13.695 | 51.803 | 1.00 | 12.65 | BTKB |
| ATOM | 4069 | CG | ARG | 641 | 15.358 | 12.831 | 50.610 | 1.00 | 8.88 | BTKB |
| ATOM | 4070 | CD | ARG | 641 | 15.886 | 13.474 | 49.360 | 1.00 | 11.30 | BTKB |
| ATOM | 4071 | NE | ARG | 641 | 15.231 | 12.947 | 48.171 | 1.00 | 11.72 | BTKB |
| ATOM | 4072 | CZ | ARG | 641 | 14.731 | 13.710 | 47.206 | 1.00 | 13.82 | BTKB |
| ATOM | 4073 | NH1 | ARG | 641 | 14.827 | 15.031 | 47.288 | 1.00 | 12.20 | BTKB |
| ATOM | 4074 | NH2 | ARG | 641 | 14.071 | 13.160 | 46.198 | 1.00 | 16.65 | BTKB |
| ATOM | 4075 | C | ARG | 641 | 15.825 | 13.988 | 54.279 | 1.00 | 14.27 | BTKB |
| ATOM | 4076 | O | ARG | 641 | 15.198 | 14.964 | 54.692 | 1.00 | 16.92 | BTKB |
| ATOM | 4077 | N | PRO | 642 | 17.017 | 13.642 | 54.787 | 1.00 | 11.73 | BTKB |
| ATOM | 4078 | CD | PRO | 642 | 17.769 | 12.438 | 54.386 | 1.00 | 8.97 | BTKB |
| ATOM | 4079 | CA | PRO | 642 | 17.706 | 14.357 | 55.863 | 1.00 | 11.49 | BTKB |
| ATOM | 4080 | CB | PRO | 642 | 18.793 | 13.371 | 56.269 | 1.00 | 10.83 | BTKB |
| ATOM | 4081 | CG | PRO | 642 | 19.134 | 12.705 | 54.949 | 1.00 | 11.19 | BTKB |
| ATOM | 4082 | C | PRO | 642 | 18.313 | 15.686 | 55.423 | 1.00 | 12.59 | BTKB |
| ATOM | 4083 | O | PRO | 642 | 18.396 | 15.982 | 54.235 | 1.00 | 14.26 | BTKB |
| ATOM | 4084 | N | THR | 643 | 18.687 | 16.513 | 56.391 | 1.00 | 13.95 | BTKB |
| ATOM | 4085 | CA | THR | 643 | 19.310 | 17.790 | 56.081 | 1.00 | 11.71 | BTKB |
| ATOM | 4086 | CB | THR | 643 | 19.003 | 18.856 | 57.151 | 1.00 | 9.31 | BTKB |
| ATOM | 4087 | OG1 | THR | 643 | 19.178 | 18.295 | 58.453 | 1.00 | 13.11 | BTKB |
| ATOM | 4088 | CG2 | THR | 643 | 17.585 | 19.370 | 57.013 | 1.00 | 16.43 | BTKB |
| ATOM | 4089 | C | THR | 643 | 20.815 | 17.574 | 56.032 | 1.00 | 9.69 | BTKB |
| ATOM | 4090 | O | THR | 643 | 21.310 | 16.524 | 56.455 | 1.00 | 10.98 | BTKB |
| ATOM | 4091 | N | PHE | 644 | 21.542 | 18.544 | 55.489 | 1.00 | 8.04 | BTKB |
| ATOM | 4092 | CA | PHE | 644 | 22.994 | 18.461 | 55.430 | 1.00 | 7.60 | BTKB |
| ATOM | 4093 | CB | PHE | 644 | 23.564 | 19.613 | 54.593 | 1.00 | 9.27 | BTKB |
| ATOM | 4094 | CG | PHE | 644 | 23.518 | 19.373 | 53.106 | 1.00 | 11.88 | BTKB |
| ATOM | 4095 | CD1 | PHE | 644 | 24.271 | 18.353 | 52.530 | 1.00 | 10.79 | BTKB |
| ATOM | 4096 | CD2 | PHE | 644 | 22.749 | 20.185 | 52.275 | 1.00 | 17.35 | BTKB |
| ATOM | 4097 | CE1 | PHE | 644 | 24.262 | 18.145 | 51.147 | 1.00 | 15.55 | BTKB |
| ATOM | 4098 | CE2 | PHE | 644 | 22.733 | 19.984 | 50.885 | 1.00 | 14.48 | BTKB |
| ATOM | 4099 | CZ | PHE | 644 | 23.495 | 18.959 | 50.324 | 1.00 | 11.64 | BTKB |
| ATOM | 4100 | C | PHE | 644 | 23.524 | 18.508 | 56.866 | 1.00 | 8.95 | BTKB |
| ATOM | 4101 | O | PHE | 644 | 24.601 | 17.996 | 57.163 | 1.00 | 10.35 | BTKB |
| ATOM | 4102 | N | LYS | 645 | 22.736 | 19.096 | 57.760 | 1.00 | 10.75 | BTKB |
| ATOM | 4103 | CA | LYS | 645 | 23.098 | 19.189 | 59.168 | 1.00 | 14.25 | BTKB |
| ATOM | 4104 | CB | LYS | 645 | 22.094 | 20.076 | 59.914 | 1.00 | 20.40 | BTKB |
| ATOM | 4105 | CG | LYS | 645 | 22.396 | 20.305 | 61.404 | 1.00 | 19.51 | BTKB |
| ATOM | 4106 | CD | LYS | 645 | 23.765 | 20.942 | 61.644 | 1.00 | 21.82 | BTKB |
| ATOM | 4107 | CE | LYS | 645 | 24.857 | 19.891 | 61.826 | 1.00 | 18.46 | BTKB |
| ATOM | 4108 | NZ | LYS | 645 | 26.212 | 20.491 | 61.891 | 1.00 | 19.87 | BTKB |
| ATOM | 4109 | C | LYS | 645 | 23.121 | 17.795 | 59.784 | 1.00 | 12.92 | BTKB |
| ATOM | 4110 | O | LYS | 645 | 24.061 | 17.433 | 60.491 | 1.00 | 16.72 | BTKB |
| ATOM | 4111 | N | ILE | 646 | 22.067 | 17.027 | 59.528 | 1.00 | 12.66 | BTKB |
| ATOM | 4112 | CA | ILE | 646 | 21.949 | 15.658 | 60.026 | 1.00 | 12.65 | BTKB |
| ATOM | 4113 | CB | ILE | 646 | 20.527 | 15.115 | 59.763 | 1.00 | 16.20 | BTKB |
| ATOM | 4114 | CG2 | ILE | 646 | 20.494 | 13.594 | 59.881 | 1.00 | 18.66 | BTKB |
| ATOM | 4115 | CG1 | ILE | 646 | 19.536 | 15.781 | 60.726 | 1.00 | 16.13 | BTKB |
| ATOM | 4116 | CD | ILE | 646 | 18.071 | 15.534 | 60.391 | 1.00 | 16.31 | BTKB |
| ATOM | 4117 | C | ILE | 646 | 22.996 | 14.806 | 59.305 | 1.00 | 14.43 | BTKB |
| ATOM | 4118 | O | ILE | 646 | 23.772 | 14.089 | 59.946 | 1.00 | 13.14 | BTKB |
| ATOM | 4119 | N | LEU | 647 | 23.054 | 14.957 | 57.979 | 1.00 | 14.30 | BTKB |
| ATOM | 4120 | CA | LEU | 647 | 24.006 | 14.254 | 57.113 | 1.00 | 14.73 | BTKB |
| ATOM | 4121 | CB | LEU | 647 | 23.879 | 14.795 | 55.682 | 1.00 | 16.19 | BTKB |
| ATOM | 4122 | CG | LEU | 647 | 23.584 | 13.896 | 54.473 | 1.00 | 13.29 | BTKB |
| ATOM | 4123 | CD1 | LEU | 647 | 22.737 | 12.680 | 54.829 | 1.00 | 12.13 | BTKB |
| ATOM | 4124 | CD2 | LEU | 647 | 22.884 | 14.734 | 53.433 | 1.00 | 9.14 | BTKB |
| ATOM | 4125 | C | LEU | 647 | 25.427 | 14.468 | 57.633 | 1.00 | 12.35 | BTKB |
| ATOM | 4126 | O | LEU | 647 | 26.247 | 13.556 | 57.613 | 1.00 | 10.67 | BTKB |
| ATOM | 4127 | N | LEU | 648 | 25.692 | 15.666 | 58.142 | 1.00 | 11.64 | BTKB |
| ATOM | 4128 | CA | LEU | 648 | 26.996 | 15.998 | 58.702 | 1.00 | 12.42 | BTKB |
| ATOM | 4129 | CB | LEU | 648 | 27.125 | 17.515 | 58.914 | 1.00 | 9.53 | BTKB |
| ATOM | 4130 | CG | LEU | 648 | 28.505 | 18.085 | 59.272 | 1.00 | 10.27 | BTKB |
| ATOM | 4131 | CD1 | LEU | 648 | 29.570 | 17.603 | 58.313 | 1.00 | 9.87 | BTKB |
| ATOM | 4132 | CD2 | LEU | 648 | 28.441 | 19.577 | 59.254 | 1.00 | 7.44 | BTKB |
| ATOM | 4133 | C | LEU | 648 | 27.269 | 15.241 | 60.009 | 1.00 | 13.83 | BTKB |
| ATOM | 4134 | O | LEU | 648 | 28.369 | 14.729 | 60.210 | 1.00 | 12.04 | BTKB |
| ATOM | 4135 | N | SER | 649 | 26.271 | 15.152 | 60.886 | 1.00 | 13.67 | BTKB |
| ATOM | 4136 | CA | SER | 649 | 26.439 | 14.440 | 62.158 | 1.00 | 11.87 | BTKB |
| ATOM | 4137 | CB | SER | 649 | 25.187 | 14.575 | 63.035 | 1.00 | 10.55 | BTKB |
| ATOM | 4138 | OG | SER | 649 | 24.887 | 15.929 | 63.307 | 1.00 | 12.87 | BTKB |
| ATOM | 4139 | C | SER | 649 | 26.711 | 12.958 | 61.910 | 1.00 | 11.53 | BTKB |
| ATOM | 4140 | O | SER | 649 | 27.719 | 12.413 | 62.366 | 1.00 | 14.67 | BTKB |
| ATOM | 4141 | N | ASN | 650 | 25.808 | 12.317 | 61.175 | 1.00 | 12.17 | BTKB |
| ATOM | 4142 | CA | ASN | 650 | 25.918 | 10.897 | 60.849 | 1.00 | 10.92 | BTKB |
| ATOM | 4143 | CB | ASN | 650 | 24.804 | 10.487 | 59.880 | 1.00 | 11.30 | BTKB |
| ATOM | 4144 | CG | ASN | 650 | 23.414 | 10.618 | 60.498 | 1.00 | 14.31 | BTKB |
| ATOM | 4145 | OD1 | ASN | 650 | 23.236 | 11.298 | 61.506 | 1.00 | 16.85 | BTKB |
| ATOM | 4146 | ND2 | ASN | 650 | 22.429 | 9.962 | 59.902 | 1.00 | 15.42 | BTKB |
| ATOM | 4147 | C | ASN | 650 | 27.280 | 10.549 | 60.272 | 1.00 | 11.25 | BTKB |
| ATOM | 4148 | O | ASN | 650 | 27.843 | 9.516 | 60.592 | 1.00 | 11.41 | BTKB |
| ATOM | 4149 | N | ILE | 651 | 27.809 | 11.425 | 59.425 | 1.00 | 11.24 | BTKB |
| ATOM | 4150 | CA | ILE | 651 | 29.119 | 11.227 | 58.823 | 1.00 | 11.82 | BTKB |
| ATOM | 4151 | CB | ILE | 651 | 29.364 | 12.262 | 57.707 | 1.00 | 10.65 | BTKB |
| ATOM | 4152 | CG2 | ILE | 651 | 30.825 | 12.736 | 57.688 | 1.00 | 13.31 | BTKB |
| ATOM | 4153 | CG1 | ILE | 651 | 28.933 | 11.679 | 56.358 | 1.00 | 9.82 | BTKB |
| ATOM | 4154 | CD | ILE | 651 | 28.910 | 12.690 | 55.219 | 1.00 | 9.11 | BTKB |
| ATOM | 4155 | C | ILE | 651 | 30.214 | 11.306 | 59.884 | 1.00 | 15.31 | BTKB |
| ATOM | 4156 | O | ILE | 651 | 31.166 | 10.527 | 59.854 | 1.00 | 17.19 | BTKB |
| ATOM | 4157 | N | LEU | 652 | 30.069 | 12.230 | 60.831 | 1.00 | 17.10 | BTKB |
| ATOM | 4158 | CA | LEU | 652 | 31.059 | 12.394 | 61.888 | 1.00 | 14.72 | BTKB |
| ATOM | 4159 | CB | LEU | 652 | 30.796 | 13.654 | 62.717 | 1.00 | 13.15 | BTKB |
| ATOM | 4160 | CG | LEU | 652 | 31.745 | 14.827 | 62.444 | 1.00 | 8.61 | BTKB |
| ATOM | 4161 | CD1 | LEU | 652 | 32.998 | 14.338 | 61.694 | 1.00 | 10.58 | BTKB |
| ATOM | 4162 | CD2 | LEU | 652 | 31.041 | 15.927 | 61.663 | 1.00 | 9.95 | BTKB |
| ATOM | 4163 | C | LEU | 652 | 31.233 | 11.180 | 62.790 | 1.00 | 16.01 | BTKB |
| ATOM | 4164 | O | LEU | 652 | 32.285 | 11.022 | 63.398 | 1.00 | 16.51 | BTKB |
| ATOM | 4165 | N | ASP | 653 | 30.209 | 10.345 | 62.920 | 1.00 | 21.54 | BTKB |
| ATOM | 4166 | CA | ASP | 653 | 30.356 | 9.145 | 63.732 | 1.00 | 24.79 | BTKB |
| ATOM | 4167 | CB | ASP | 653 | 29.651 | 9.264 | 65.099 | 1.00 | 28.24 | BTKB |
| ATOM | 4168 | CG | ASP | 653 | 28.171 | 9.543 | 64.990 | 1.00 | 24.00 | BTKB |
| ATOM | 4169 | OD1 | ASP | 653 | 27.389 | 8.597 | 64.778 | 1.00 | 22.42 | BTKB |
| ATOM | 4170 | OD2 | ASP | 653 | 27.780 | 10.711 | 65.170 | 1.00 | 31.86 | BTKB |
| ATOM | 4171 | C | ASP | 653 | 29.989 | 7.854 | 62.996 | 1.00 | 29.44 | BTKB |
| ATOM | 4172 | O | ASP | 653 | 28.983 | 7.207 | 63.287 | 1.00 | 31.02 | BTKB |
| ATOM | 4173 | N | VAL | 654 | 30.804 | 7.508 | 62.006 | 1.00 | 33.61 | BTKB |
| ATOM | 4174 | CA | VAL | 654 | 30.603 | 6.289 | 61.233 | 1.00 | 34.38 | BTKB |
| ATOM | 4175 | CB | VAL | 654 | 30.615 | 6.548 | 59.700 | 1.00 | 33.33 | BTKB |
| ATOM | 4176 | CG1 | VAL | 654 | 30.247 | 5.284 | 58.958 | 1.00 | 31.87 | BTKB |
| ATOM | 4177 | CG2 | VAL | 654 | 29.668 | 7.660 | 59.331 | 1.00 | 28.22 | BTKB |
| ATOM | 4178 | C | VAL | 654 | 31.766 | 5.368 | 61.580 | 1.00 | 37.78 | BTKB |
| ATOM | 4179 | OT1 | VAL | 654 | 31.521 | 4.332 | 62.236 | 1.00 | 39.69 | BTKB |
| ATOM | 4180 | OT2 | VAL | 654 | 32.916 | 5.722 | 61.230 | 1.00 | 36.40 | BTKB |
| END | | | | | | | | | | |

1. A crystal of BTK kinase domain having a space group symmetry of P2₁2₁2₁ and comprising a unit cell having the dimensions of a, b, and c, where a is about 45 Å, b is about 104 Å, and c is about 116 Å.

2. The crystal of claim 1, having an amino acid sequence of SEQ ID NO:4.

3. A molecule or molecular complex comprising at least a portion of the BTK kinase domain binding pocket, wherein the binding pocket comprises the amino acids listed in Table 2, the binding pocket defined by a set of points having a root mean square deviation of less than about 0.70 Å from points representing the backbone atoms of said amino acids as represented by the structure coordinates listed in Table 1.

4. A scalable three-dimensional configuration of points, at least a portion of said points derived from structure coordinates listed in Table 1, comprising a BTK kinase domain binding pocket, wherein the BTK kinase domain forms a crystal having the space group symmetry P2₁2₁2₁2.

5. The scalable three-dimensional configuration of points of claim 4, wherein substantially all of the points are derived from structure coordinates listed in Table 1.

6. The scalable three-dimensional configuration of points of claim 4, wherein at least a portion of the points are derived from structure coordinates representing locations of at least the backbone atoms of amino acids defining the BTK kinase domain binding pocket.

7. The scalable three-dimensional configuration of points of claim 4, wherein the binding pocket comprises the amino acids listed in Table 2.

8. A scalable three-dimensional configuration of points displayed as a holographic image, a stereodiagram, a model, or a computer-displayed image, at least a portion of said points derived from structure coordinates listed in Table 1, comprising a BTK kinase domain binding pocket, wherein the BTK kinase domain forms a crystal having the space group symmetry P2₁2₁2₁.

9. A machine-readable data storage medium comprising a data storage material encoded with machine-readable data, wherein a machine programmed with instructions for using such data displays a graphical three-dimensional representation of at least one molecule or molecular complex comprising at least a portion of a BTK kinase domain binding pocket, the binding pocket defined by a set of points having a root mean square deviation of less than about 0.05 Å from points representing the atoms of said amino acids as represented by the structure coordinates listed in Table 1.

10. A machine readable data storage medium comprising data storage material encoded with a first set of machine readable data which is combined with a second set of machine readable data using a machine programmed with instructions for using said first and second sets of data, determines at least a portion of the structure coordinates corresponding to the second set of data, wherein the first set of data comprises a Fourier transform of at least a portion of the BTK kinase domain structural coordinates of Table 1, and wherein the second set of data comprises an X-ray diffraction pattern of an unknown molecule or molecular complex.

11. A method for obtaining structural information about a molecule or molecular complex comprising application of at least a portion of the BTK kinase domain structure coordinates of Table 1 to an X-ray diffraction pattern of the molecule or molecular complex's crystal structure to generate a three-dimensional electron density map of at least a portion of the molecule or molecular complex.

12. A computer-assisted method for identifying an agent that modulates BTK activity comprising:
(a) providing a computer modeling application with a set of structure coordinates of Table 1 defining at least a portion of a BTK kinase domain;
(b) providing the computer modeling application with a set of structure coordinates for a test compound; and
(c) modeling the structure of (a) complexed with (b) to determine if the test compound binds to the BTK kinase domain binding pocket.

13. The method of claim 12, where the binding pocket comprises the amino acids listed in Table 2.

14. A computer-assisted method for designing a compound that binds the BTK kinase domain binding pocket, comprising:
(a) providing a computer modeling application with a set of structural coordinates of Table 1 defining at least a portion of the BTK kinase domain; and
(b) modeling the structural coordinates of (a) to determine a complementary molecule that binds the BTK kinase domain pocket.

15. A molecular complex comprising a BTK kinase domain binding pocket defined by at least a portion of the structural coordinates of Table 1 complexed with a compound having complimentarily to at least a portion of the structural coordinates of Table 1.


SEQUENCE LISTING (7 sequences)

SEQ ID NO: 1 - protein, 659 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 2 - protein, 659 aa, Murine
MAAVILESIFLKRSQQKKKTSPLNFKKRLFLLTVHKLSYYEYDFERGRRGSKKGSIDVEKITCVETVIPEKNPPPERQIPRRGEESSEMEQISIIERFPYPFQVVYDEGPLYVFSPTEELRKPWIHQLKNVIRYNSDLVQKYHPCFWIDGQYLCCSQTAKNAMGCQILENRNGSLKPGSSHRKTKKPLPPTPEEDQILKKPLPPEPTAAPISTTELKKVVALYDYMPMNANDLQLRKGEEYFILEESNLPWWRARDKNGQEGYIPSNYITEAEDSIEMYEWYSKHMTRSQAEQLLKQEGKEGGFIVRDSSKAGKYTVSVFAKSTGEPQGVIRHYVVCSTPQSQYYLAEKHLFSTIPELINYHQHNSAGLISRLKYPVSKQNKNAPSTAGLGYGSWEIDPKDLTFLKELGTGQFGVVKYGKWRGQYDVAIKMIREGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASERVYTIMYSCWHEKADERPSFKILLSNILDVMDEES

SEQ ID NO: 3 - protein, 263 aa, Homo sapiens
IDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 4 - protein, 263 aa, Murine
IDPKDLTFLKELGTGQFGVVKYGKWRGQYDVAIKMIREGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASERVYTIMYSCWHEKADERPSFKILLSNILDVMDEES

SEQ ID NO: 5 - DNA, 2591 bp, Homo sapiens
ctcagactgtccttcctctctggactgtaagaatatgtctccagggccagtgtctgctgcgatcgagtcccaccttccaagtcctggcatctcaatgcatctgggaagctacctgcattaagtcaggactgagcacacaggtgaactccagaaagaagaagctatggccgcagtgattctggagagcatctttctgaagcgatcccaacagaaaaagaaaacatcacctctaaacttcaagaagcgcctgtttctcttgaccgtgcacaaactctcctactatgagtatgactttgaacgtgggagaagaggcagtaagaagggttcaatagatgttgagaagatcacttgtgttgaaacagtggttcctgaaaaaaatcctcctccagaaagacagattccgagaagaggtgaagagtccagtgaaatggagcaaatttcaatcattgaaaggttcccttatcccttccaggttgtatatgatgaagggcctctctacgtcttctccccaactgaagaactaaggaagcggtggattcaccagctcaaaaacgtaatccggtacaacagtgatctggttcagaaatatcacccttgcttctggatcgatgggcagtatctctgctgctctcagacagccaaaaatgctatgggctgccaaattttggagaacaggaatggaagcttaaaacctgggagttctcaccggaagacaaaaaagcctcttcccccaacgcctgaggaggaccagatcttgaaaaagccactaccgcctgagccagcagcagcaccagtctccacaagtgagctgaaaaaggttgtggccctttatgattacatgccaatgaatgcaaatgatctacagctgcggaagggtgatgaatattttatcttggaggaaagcaacttaccatggtggagagcacgagataaaaatgggcaggaaggctacattcctagtaactatgtcactgaagcagaagactccatagaaatgtatgagtggtattccaaacacatgactcggagtcaggctgagcaactgctaaagcaagaggggaaagaaggaggtttcattgtcagagactccagcaaagctggcaaatatacagtgtctgtgtttgctaaatccacaggggaccctcaaggggtgatacgtcattatgttgtgtgttccacacctcagagccagtattacctggctgagaagcaccttttcagcaccatccctgagctcattaactaccatcagcacaactctgcaggactcatatccaggctcaaatatccagtgtctcaacaaaacaagaatgcaccttccactgcaggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgttggcatgagaaagcagatgagcgtcccactttcaaaattcttctgagcaatattctagatgtcatggatgaagaatcctgagctcgccaataagcttcttggttctacttctcttctccacaagccccaatttcactttctcagaggaaatcccaagcttaggagccctggagcctttgtgctcccactcaatacaaaaaggcccctctctacatctggggatgcacctcttctttgattccctgggatagtggcttctgagcaaaggccaaaaaattattgtgcctgaaatttcccgagagaattaagacagactgaatttgcgatgaaaatattttttaggagggaggatgtaaatagccgcacaaaggggtccaacagctctttgagtaggcatttggtagagcttgggggtgtgtgtgtgggggtggaccgaatttggcaagaatgaaatggtgtcataaagatgggaggggagggtgttttgataaaataaattctagaaagcttaaaaaaaaaaaaaaaaaaa

SEQ ID NO: 6 - DNA, 2485 bp, Murine
taagaatatgtctccaggtccagagtcttcagagatcaagtcccaccttccaagtcctggcatctcacgacgtctggggagctacctgcattaagtcagaactgagtacacaaacaagttccagagagaggaagccatggctgcagtgatactggagagcatctttctgaagcgctcccagcagaaaaagaaaacatcacctttaaacttcaagaagcgcctgtttctcttgactgtacacaaactttcatactatgaatatgactttgaacgtgggagaagaggcagtaagaaaggttcaatagatgttgagaagatcacctgtgttgaaacagtaattcctgaaaaaaatcccccaccagaaagacagattccgaggagaggtgaggagtctagtgaaatggaacagatttcaatcattgaaaggttcccgtacccattccaggttgtatatgatgaaggacctctctatgttttctccccaactgaagagctgagaaagccgtggattcaccagctcaaaaatgtaatccggtacaatagtgacctggtacagaaataccatccttgcttctggattgatggacagtatctctgctgctctcagacagccaagaatgctatgggctgccaaattttggagaacaggaatggaagcttaaaacctgggagttctcatcgaaaaacgaaaaagcctcttccccctaccccagaggaagatcagatcttgaaaaaaccgcttcccccggagccaacagcagcaccaatctccacaaccgagctgaaaaaggtcgtggccctttatgattacatgccaatgaacgcaaatgacttacaattgcgaaagggcgaggagtattttatcctggaggagagcaacctaccgtggtggcgagcacgagataaaaatgggcaggaaggctacatcccaagtaactatatcactgaagctgaggactccatagagatgtatgagtggtattccaagcacatgactcgaagtcaagctgagcaactgctaaagcaagaggggaaagaaggaggtttcattgtcagagactccagcaaagctggaaaatacaccgtgtctgtgtttgctaaatctactggggagcctcaaggggtgatccgccattacgttgtgtgttccacgccacagagccagtattacctggctgagaaacacctcttcagcaccatccctgagctcattaactaccatcaacacaactctgcaggcctcatatccaggctgaaatatcctgtgtctaaacaaaacaaaaacgcgccttctactgcaggcctgggctatggatcatgggaaattgatccaaaggacctcaccttcttgaaggagcttgggactggacaattcggtgtcgtgaaatatgggaagtggaggggccaatatgatgtggccatcaagatgatcagagaaggttccatgtcggaggatgaattcattgaagaagccaaagtcatgatgaatctttcccatgagaagctggtgcagttgtatggcgtctgcaccaaacaacgccccatcttcatcatcaccgagtacatggctaatggctgcctcttgaactacctgagggagatgcggcaccgcttccagacacagcagctgcttgagatgtgcaaagatgtctgtgaagcaatggaatacttggagtcgaagcagttccttcacagagacctggcagctcgaaactgtttggtaaacgatcaaggagttgtgaaagtatctgactttggcctgtctaggtatgtccttgatgatgagtacaccagctctgtaggctccaagtttccagttcggtggtctccaccagaagtgcttatgtatagcaagttcagcagcaaatctgacatctgggcttttggggttttaatgtgggagatctactccctggggaagatgccgtatgagagatttactaacagtgagacagcagaacacattgctcaaggcttacgtctctacaggcctcatctggcatcagagagggtatataccatcatgtacagctgctggcacgagaaagcagatgaacgtcctagtttcaaaattctcttgagtaacattctagatgtgatggatgaagaatcctgagctggctcctaacctccgtggatctcctcctctctcctacaaaacctaattccatgtttcctgaggagttccctggctgcagggctctagcttccatgcgcctactgaatgcatcaagagccctggacatctaggaatgcctttcttctctcgttccctggcatactgctctaagcaaaggtcaagggatttctgtgcctagtattacccataacttcaagactccaacagactgaattggggatgggaacactttgggggagggaaaactgtaaatagctcactagttgtcaacagcttgttgttagtgttaagagtgtgtgtgtgggggtaggaatgtgcataaatggtgttgtaactaatatgaag

SEQ ID NO: 7 - protein, 659 aa, Homo sapiens; identical to SEQ ID NO: 1